(19) United States
(12) Patent Application Publication
(10) Pub. No.: US 2025/0057041 A1
(43) Pub. Date: Feb. 13, 2025

(54) COMPOUND, ORGANIC-ELECTROLUMINESCENCE-DEVICE MATERIAL, ORGANIC ELECTROLUMINESCENCE DEVICE, AND ELECTRONIC DEVICE

(71) Applicant: IDEMITSU KOSAN CO.,LTD., Tokyo (JP)

(72) Inventors: Ryo NAGATA, Tokyo (JP); Shintaro BAN, Tokyo (JP); Yuki NAKANO, Tokyo (JP); Ryota TAKAHASHI, Tokyo (JP); Kiyoshi IKEDA, Tokyo (JP); Tomokatsu KUSHIDA, Tokyo (JP); Sigma HASHIMOTO, Tokyo (JP); Yuichiro KAWAMURA, Tokyo (JP)

(73) Assignee: IDEMITSU KOSAN CO.,LTD., Tokyo (JP)

(21) Appl. No.: 18/774,694

(22) Filed: Jul. 16, 2024

(30) Foreign Application Priority Data
Jul. 31, 2023 (JP) 2023-124803

(51) Int. Cl. H10K 85/60; C07D 471/04; C09K 11/06; H10K 50/11; H10K 101/25; H10K 101/30

(52) U.S. Cl. CPC H10K 85/6572; C07D 471/04; C09K 11/06; H10K 85/654; C09K 2211/1018; H10K 50/11; H10K 2101/25; H10K 2101/30

(57) ABSTRACT

A compound represented by a formula (1).

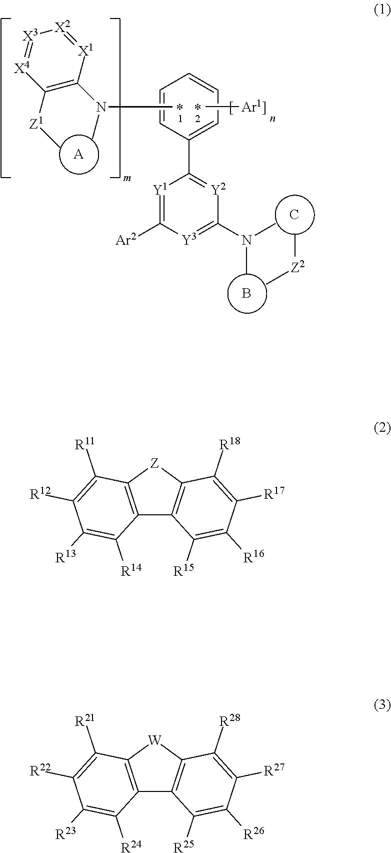

In the formula (1): X¹ to X⁴ are each independently a nitrogen atom or CRx; at least one of X¹ to X⁴ is a nitrogen atom; a ring A, a ring B, and a ring C are each independently an aromatic hydrocarbon ring or a heterocycle; Ar¹ is a hydrogen atom, an aryl group, or a group represented by a formula (2); and Ar² is an aryl group or a group represented by a formula (3).

### CROSS REFERENCE TO RELATED APPLICATIONS

The entire disclosure of Japanese Patent Application No. 2023-124803, filed Jul. 31, 2023 is expressly incorporated by reference herein.

### TECHNICAL FIELD

The present invention relates to a compound, an organic-electroluminescence-device material, an organic electroluminescence device, and an electronic device.

### BACKGROUND ART

When voltage is applied to an organic electroluminescence device (hereinafter, occasionally referred to as “organic EL device”), holes are injected from an anode and electrons are injected from a cathode into an emitting layer. The injected holes and electrons are recombined in the emitting layer to form excitons. Specifically, according to the electron spin statistics theory, singlet excitons and triplet excitons are generated at a ratio of 25%:75%.

A fluorescent organic EL device using light emission from singlet excitons has been applied to a full-color display such as a mobile phone and a television, but an internal quantum efficiency is said to be at a limit of 25%. Various studies have been made on a compound to be used for an organic EL device in order to enhance the performance of the organic EL device (see, for instance, Literature 1: Specification of U.S. Patent Application Publication No. 2022/0177492 A1 and Literature 2: Specification of U.S. Patent Application Publication No. 2021/0066613 A1). The performance of the organic EL device is evaluable in terms of, for instance, the luminance, emission wavelength, chromaticity, luminous efficiency, drive voltage, and lifetime.

A further improvement in performance of the organic EL device has been demanded for an improvement in performance of an electronic device such as a display.

### SUMMARY OF THE INVENTION

An object of the invention is to provide a compound that enables an organic electroluminescence device to emit light with high efficiency. Another object of the invention is to provide an organic-electroluminescence-device material containing the compound. A still another object of the invention is to provide an organic electroluminescence device that emits light with high efficiency and an electronic device including the organic electroluminescence device.

According to an aspect of the invention, a compound represented by a formula (1) below is provided.

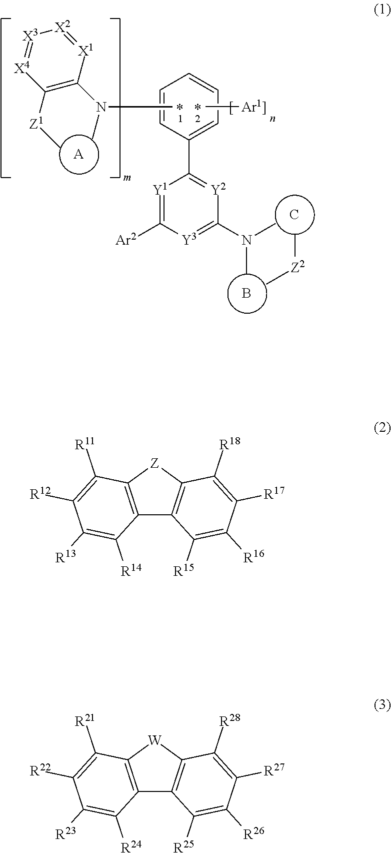

In the formula (1):
X¹ to X⁴ are each independently a nitrogen atom or CRx;
at least one of X¹ to X⁴ is a nitrogen atom;
when a plurality of Rx are present, the plurality of Rx are mutually the same or different;
Y¹ to Y³ are each independently a nitrogen atom or CH;
two or more of Y¹ to Y³ are each a nitrogen atom;
a ring A, a ring B, and a ring C are each independently a substituted or unsubstituted aromatic hydrocarbon ring having 6 to 60 ring carbon atoms, or a substituted or unsubstituted heterocycle having 5 to 60 ring atoms;
Z¹ and Z² are each independently a single bond, C(R₁)(R²), NR³, an oxygen atom, or a sulfur atom;
Ar¹ is a hydrogen atom, a substituted or unsubstituted aryl group having 6 to 50 ring carbon atoms, or a group represented by a formula (2) above;
Ar² is a substituted or unsubstituted aryl group having 6 to 50 ring carbon atoms, or a group represented by a formula (3) above;
m is 1, 2, 3, 4, or 5;
n is 0, 1, 2, 3, or 4;
m+n=5 is satisfied;
when m is 2, 3, 4, or 5,
a plurality of X¹ are mutually the same or different;
a plurality of X² are mutually the same or different;
a plurality of X³ are mutually the same or different;
a plurality of X⁴ are mutually the same or different;
a plurality of rings A are mutually the same or different; and
a plurality of Z¹ are mutually the same or different; and when n is 2, 3, or 4,
a plurality of Ar¹ are mutually the same or different; and
*1 and *2 each represent a bonding position to a benzene ring, in the formula (2):
Z is C(R_{1A})(R^{2A}), NR^{3A}, an oxygen atom, or a sulfur atom;
at least one combination of adjacent two or more of R₁₁ to R₁₈ are mutually bonded to form a substituted or unsubstituted monocyclic ring, mutually bonded to form a substituted or unsubstituted fused ring, or not mutually bonded; and
when Ar¹ is a group represented by the formula (2), one of R₁₁ to R₁₈ and R^{3A} is a single bond with a benzene ring in the formula (1),
in the formula (3):
W is C(R^{21A})(R^{22A}), an oxygen atom, or a sulfur atom;
at least one combination of adjacent two or more of R²¹ to R²⁸ are mutually bonded to form a substituted or unsubstituted monocyclic ring, mutually bonded to form a substituted or unsubstituted fused ring, or not mutually bonded;
when Ar² is a group represented by the formula (3), one of R²¹ to R²⁸ is a single bond with a carbon atom between Y¹ and Y³ in the formula (1);
Rx, R₁, R², R³, R⁴, R^{2A}, R^{21A}, R^{22A}, and R^{3A}, R₁₁ to R₁₈ and R²¹ to R²⁸ not being the single bond with the benzene ring, not being the single bond with the carbon atom between Y¹ and Y³, and forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms, and in the compound represented by the formula (1), R₉₀₆ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

According to another aspect of the invention, there is provided an organic-electroluminescence-device material containing the compound according to the aspect of the invention.

According to a still another aspect of the invention, there is provided an organic electroluminescence device including a cathode, an anode, and an organic layer disposed between the cathode and the anode, in which the organic layer contains, as a compound M2, the compound according to the aspect of the invention.

According to a further aspect of the invention, there is provided an electronic device including the organic electroluminescence device according to the aspect of the invention.

According to the aspect of the invention, there can be provided a compound that enables an organic electroluminescence device to emit light with high efficiency. According to the aspect of the invention, there can be provided an organic-electroluminescence-device material containing the compound. According to the aspect of the invention, there can be provided an organic electroluminescence device that emits light with high efficiency and an electronic device including the organic electroluminescence device.

### BRIEF DESCRIPTION OF DRAWINGS

FIG. 1 schematically depicts an exemplary arrangement of an organic electroluminescence device according to a first exemplary embodiment of the invention.

FIG. 2 schematically depicts an apparatus for measuring transient PL.

FIG. 3 illustrates an example of decay curves of the transient PL.

FIG. 4 schematically illustrates a relationship in energy level and energy transfer between a sensitizing material and a fluorescent material in an emitting layer of an exemplary organic electroluminescence device according to the first exemplary embodiment of the invention.

FIG. 5 schematically illustrates a relationship in energy level and energy transfer between a second host material, the sensitizing material, and the fluorescent material in an emitting layer of an exemplary organic electroluminescence device according to a second exemplary embodiment of the invention.

### DETAILED DESCRIPTION

### Definitions

Herein, a hydrogen atom includes isotope having different numbers of neutrons, specifically, protium, deuterium and tritium.

In chemical formulae herein, it is assumed that a hydrogen atom (i.e. protium, deuterium and tritium) is bonded to each of bondable positions that are not annexed with signs “R” or the like or “D” representing a deuterium.

Herein, the ring carbon atoms refer to the number of carbon atoms among atoms forming a ring of a compound (e.g., a monocyclic compound, fused-ring compound, cross-linking compound, carbon ring compound, and heterocyclic compound) in which the atoms are bonded to each other to form the ring. When the ring is substituted by a substituent(s), carbon atom(s) contained in the substituent(s) is not counted in the ring carbon atoms. Unless otherwise specified, the same applies to the “ring carbon atoms” described later. For instance, a benzene ring has 6 ring carbon atoms, a naphthalene ring has 10 ring carbon atoms, a pyridine ring has 5 ring carbon atoms, and a furan ring has 4 ring carbon atoms. Further, for instance, 9,9-diphenylfluorenyl group has 13 ring carbon atoms and 9,9′-spirobifluorenyl group has 25 ring carbon atoms.

When a benzene ring is substituted by a substituent in a form of, for instance, an alkyl group, the number of carbon atoms of the alkyl group is not counted in the number of the ring carbon atoms of the benzene ring. Accordingly, the benzene ring substituted by an alkyl group has 6 ring carbon atoms. When a naphthalene ring is substituted by a substituent in a form of, for instance, an alkyl group, the number of carbon atoms of the alkyl group is not counted in the number of the ring carbon atoms of the naphthalene ring. Accordingly, the naphthalene ring substituted by an alkyl group has 10 ring carbon atoms.

Herein, the ring atoms refer to the number of atoms forming a ring of a compound (e.g., a monocyclic compound, fused-ring compound, cross-linking compound, carbon ring compound, and heterocyclic compound) in which the atoms are bonded to each other to form the ring (e.g., monocyclic ring, fused ring, and ring assembly). Atom(s) not forming the ring (e.g., hydrogen atom(s) for saturating the valence of the atom which forms the ring) and atom(s) in a substituent by which the ring is substituted are not counted as the ring atoms. Unless otherwise specified, the same applies to the “ring atoms” described later. For instance, a pyridine ring has 6 ring atoms, a quinazoline ring has 10 ring atoms, and a furan ring has 5 ring atoms. For instance, the number of hydrogen atom(s) bonded to a pyridine ring or the number of atoms forming a substituent is not counted as the pyridine ring atoms. Accordingly, a pyridine ring bonded to a hydrogen atom(s) or a substituent(s) has 6 ring atoms. For instance, the hydrogen atom(s) bonded to carbon atom(s) of a quinazoline ring or the atoms forming a substituent are not counted as the quinazoline ring atoms. Accordingly, a quinazoline ring bonded to hydrogen atom(s) or a substituent(s) has 10 ring atoms.

Herein, “XX to YY carbon atoms” in the description of “substituted or unsubstituted ZZ group having XX to YY carbon atoms” represent carbon atoms of an unsubstituted ZZ group and do not include carbon atoms of a substituent(s) of the substituted ZZ group. Herein, “YY” is larger than “XX,” “XX” representing an integer of 1 or more and “YY” representing an integer of 2 or more.

Herein, “XX to YY atoms” in the description of “substituted or unsubstituted ZZ group having XX to W atoms” represent atoms of an unsubstituted ZZ group and does not include atoms of a substituent(s) of the substituted ZZ group. Herein, “YY” is larger than “XX,” “XX” representing an integer of 1 or more and “YY” representing an integer of 2 or more.

Herein, an unsubstituted ZZ group refers to an “unsubstituted ZZ group” in a “substituted or unsubstituted ZZ group,” and a substituted ZZ group refers to a “substituted ZZ group” in a “substituted or unsubstituted ZZ group.”

Herein, the term “unsubstituted” used in a “substituted or unsubstituted ZZ group” means that a hydrogen atom(s) in the ZZ group is not substituted with a substituent(s). The hydrogen atom(s) in the “unsubstituted ZZ group” is protium, deuterium, or tritium.

Herein, the term “substituted” used in a “substituted or unsubstituted ZZ group” means that at least one hydrogen atom in the ZZ group is substituted with a substituent. Similarly, the term “substituted” used in a “BB group substituted by AA group” means that at least one hydrogen atom in the BB group is substituted with the AA group.

### Substituent Mentioned Herein

Substituent mentioned herein will be described below.

An “unsubstituted aryl group” mentioned herein has, unless otherwise specified herein, 6 to 50, preferably 6 to 30, more preferably 6 to 18 ring carbon atoms.

An “unsubstituted heterocyclic group” mentioned herein has, unless otherwise specified herein, 5 to 50, preferably 5 to 30, more preferably 5 to 18 ring atoms.

An “unsubstituted alkyl group” mentioned herein has, unless otherwise specified herein, 1 to 50, preferably 1 to 20, more preferably 1 to 6 carbon atoms.

An “unsubstituted alkenyl group” mentioned herein has, unless otherwise specified herein, 2 to 50, preferably 2 to 20, more preferably 2 to 6 carbon atoms.

An “unsubstituted alkynyl group” mentioned herein has, unless otherwise specified herein, 2 to 50, preferably 2 to 20, more preferably 2 to 6 carbon atoms.

An “unsubstituted cycloalkyl group” mentioned herein has, unless otherwise specified herein, 3 to 50, preferably 3 to 20, more preferably 3 to 6 ring carbon atoms.

An “unsubstituted arylene group” mentioned herein has, unless otherwise specified herein, 6 to 50, preferably 6 to 30, more preferably 6 to 18 ring carbon atoms.

An “unsubstituted divalent heterocyclic group” mentioned herein has, unless otherwise specified herein, 5 to 50, preferably 5 to 30, more preferably 5 to 18 ring atoms.

An “unsubstituted alkylene group” mentioned herein has, unless otherwise specified herein, 1 to 50, preferably 1 to 20, more preferably 1 to 6 carbon atoms.

### Substituted or Unsubstituted Aryl Group

Specific examples (specific example group G1) of the “substituted or unsubstituted aryl group” mentioned herein include unsubstituted aryl groups (specific example group G1A) below and substituted aryl groups (specific example group G1B). (Herein, an unsubstituted aryl group refers to an “unsubstituted aryl group” in a “substituted or unsubstituted aryl group”, and a substituted aryl group refers to a “substituted aryl group” in a “substituted or unsubstituted aryl group.”) A simply termed “aryl group” herein includes both of an “unsubstituted aryl group” and a “substituted aryl group”.

The “substituted aryl group” refers to a group derived by substituting at least one hydrogen atom in an “unsubstituted aryl group” with a substituent. Examples of the “substituted aryl group” include a group derived by substituting at least one hydrogen atom in the “unsubstituted aryl group” in the specific example group G1A below with a substituent, and examples of the substituted aryl group in the specific example group G1B below. It should be noted that the examples of the “unsubstituted aryl group” and the “substituted aryl group” mentioned herein are merely exemplary, and the “substituted aryl group” mentioned herein includes a group derived by further substituting a hydrogen atom bonded to a carbon atom of a skeleton of a “substituted aryl group” in the specific example group G1B below, and a group derived by further substituting a hydrogen atom of a substituent of the “substituted aryl group” in the specific example group G1B below.

### Unsubstituted Aryl Group (Specific Example Group G1A):

a phenyl group, p-biphenyl group, m-biphenyl group, o-biphenyl group, p-terphenyl-4yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, benzanthryl group, phenanthryl group, benzophenanthryl group, phenalenyl group, pyrenyl group, chrysenyl group, benzochrysenyl group, triphenylenyl group, benzotriphenylenyl group, tetracenyl group, pentacenyl group, fluorenyl group, 9,9′-spirobifluorenyl group, benzofluorenyl group, dibenzofluorenyl group, fluoranthenyl group, benzofluoranthenyl group, perylenyl group, and monovalent aryl group derived by removing one hydrogen atom from cyclic structures represented by formulae (TEMP-1) to (TEMP-15) below.

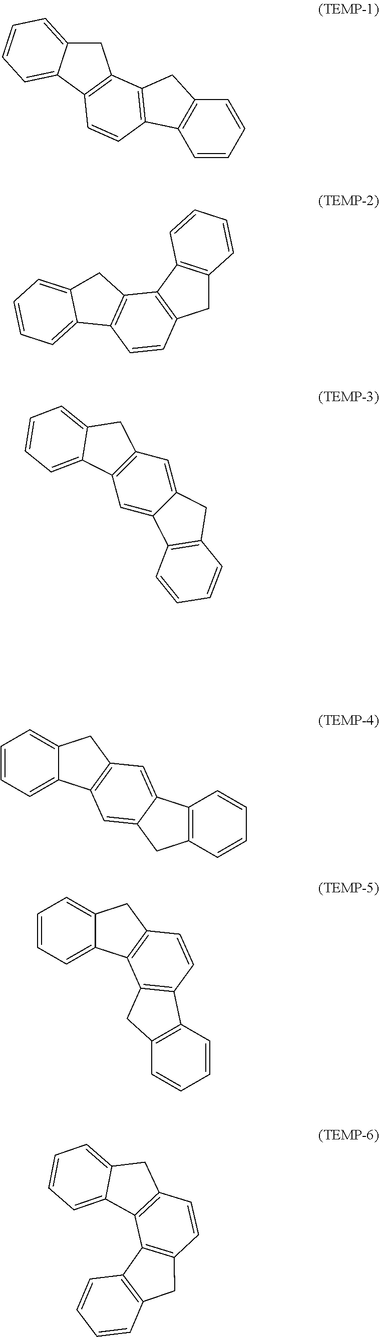

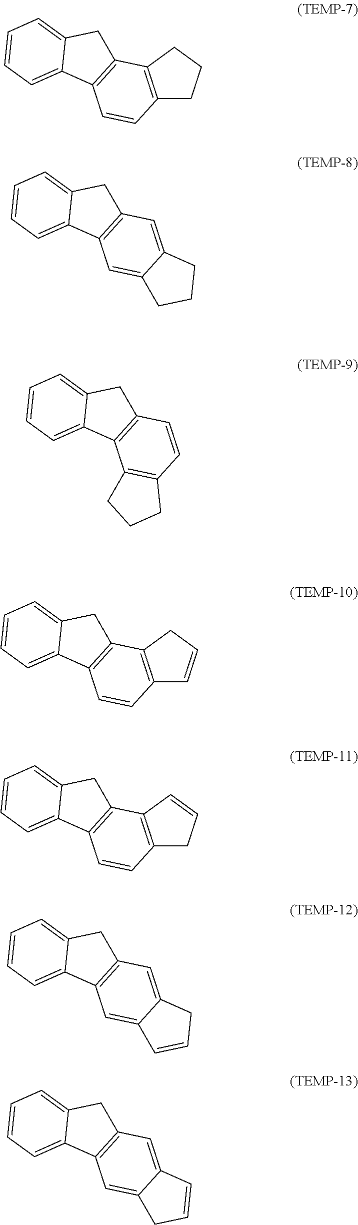

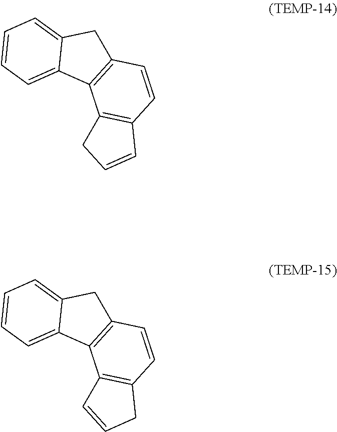

### Substituted Aryl Group (Specific Example Group G1B):

an o-tolyl group, m-tolyl group, p-tolyl group, para-xylyl group, meta-xylyl group, ortho-xylyl group, para-isopropylphenyl group, meta-isopropylphenyl group, ortho-isopropylphenyl group, para-t-butylphenyl group, meta-t-butylphenyl group, ortho-t-butylphenyl group, 3,4,5-trimethylphenyl group, 9,9-dimethyifluorenyl group, 9,9-diphenylfluorenyl group, 9,9-bis(4-methylphenyl)fluorenyl group, 9,9-bis(4-isopropylphenyl)fluorenyl group, 9,9-bis(4-t-butylphenyl)fluorenyl group, cyanophenyl group, triphenylsilylphenyl group, trimethylsilylphenyl group, phenylnaphthyl group, naphthylphenyl group, and group derived by substituting at least one hydrogen atom of a monovalent group derived from one of the cyclic structures represented by the formulae (TEMP-1) to (TEMP-15) with a substituent.

### Substituted or Unsubstituted Heterocyclic Group

The “heterocyclic group” mentioned herein refers to a cyclic group having at least one heteroatom in the ring atoms. Specific examples of the heteroatom include a nitrogen atom, oxygen atom, sulfur atom, silicon atom, phosphorus atom, and boron atom.

The “heterocyclic group” mentioned herein is a monocyclic group or a fused-ring group.

The “heterocyclic group” mentioned herein is an aromatic heterocyclic group or a non-aromatic heterocyclic group.

Specific examples (specific example group G2) of the “substituted or unsubstituted heterocyclic group” mentioned herein include unsubstituted heterocyclic groups (specific example group G2A) and substituted heterocyclic groups (specific example group G2B). (Herein, an unsubstituted heterocyclic group refers to an “unsubstituted heterocyclic group” in a “substituted or unsubstituted heterocyclic group,” and a substituted heterocyclic group refers to a “substituted heterocyclic group” in a “substituted or unsubstituted heterocyclic group.”) A simply termed “heterocyclic group” herein includes both of an “unsubstituted heterocyclic group” and a “substituted heterocyclic group.”

The “substituted heterocyclic group” refers to a group derived by substituting at least one hydrogen atom in an “unsubstituted heterocyclic group” with a substituent. Specific examples of the “substituted heterocyclic group” include a group derived by substituting at least one hydrogen atom in the “unsubstituted heterocyclic group” in the specific example group G2A below with a substituent, and examples of the substituted heterocyclic group in the specific example group G2B below. It should be noted that the examples of the “unsubstituted heterocyclic group” and the “substituted heterocyclic group” mentioned herein are merely exemplary, and the “substituted heterocyclic group” mentioned herein includes a group derived by further substituting a hydrogen atom bonded to a ring atom of a skeleton of a “substituted heterocyclic group” in the specific example group G2B below, and a group derived by further substituting a hydrogen atom of a substituent of the “substituted heterocyclic group” in the specific example group G2B below.

The specific example group G2A includes, for instance, unsubstituted heterocyclic groups including a nitrogen atom (specific example group G2A1) below, unsubstituted heterocyclic groups including an oxygen atom (specific example group G2A2) below, unsubstituted heterocyclic groups including a sulfur atom (specific example group G2A3) below, and monovalent heterocyclic groups (specific example group G2A4) derived by removing a hydrogen atom from cyclic structures represented by formulae (TEMP-16) to (TEMP-33) below.

The specific example group G2B includes, for instance, substituted heterocyclic groups including a nitrogen atom (specific example group G2B1) below, substituted heterocyclic groups including an oxygen atom (specific example group G2B2) below, substituted heterocyclic groups including a sulfur atom (specific example group G2B3) below, and groups derived by substituting at least one hydrogen atom of the monovalent heterocyclic groups (specific example group G2B4) derived from the cyclic structures represented by formulae (TEMP-16) to (TEMP-33) below.

### Unsubstituted Heterocyclic Groups Including Nitrogen Atom (Specific Example Group G2A1):

a pyrrolyl group, imidazolyl group, pyrazolyl group, triazolyl group, tetrazolyl group, oxazolyl group, isoxazolyl group, oxadiazolyl group, thiazolyl group, isothiazolyl group, thiadiazolyl group, pyridyl group, pyridazynyl group, pyrimidinyl group, pyrazinyl group, triazinyl group, indolyl group, isoindolyl group, indolizinyl group, quinolizinyl group, quinolyl group, isoquinolyl group, cinnolyl group, phthalazinyl group, quinazolinyl group, quinoxalinyl group, benzimidazolyl group, indazolyl group, phenanthrolinyl group, phenanthridinyl group, acridinyl group, phenazinyl group, carbazolyl group, benzocarbazolyl group, morpholino group, phenoxazinyl group, phenothiazinyl group, azacarbazolyl group, and diazacarbazolyl group.

### Unsubstituted Heterocyclic Groups Including Oxygen Atom (Specific Example Group G2A2):

a furyl group, oxazolyl group, isoxazolyl group, oxadiazolyl group, xanthenyl group, benzofuranyl group, isobenzofuranyl group, dibenzofuranyl group, naphthobenzofuranyl group, benzoxazolyl group, benzisoxazolyl group, phenoxazinyl group, morpholino group, dinaphthofuranyl group, azadibenzofuranyl group, diazadibenzofuranyl group, azanaphthobenzofuranyl group, and diazanaphthobenzofuranyl group.

### Unsubstituted Heterocyclic Groups Including Sulfur Atom (Specific Example Group G2A3):

a thienyl group, thiazolyl group, isothiazolyl group, thiadiazolyl group, benzothiophenyl group (benzothienyl group), isobenzothiophenyl group (isobenzothienyl group), dibenzothiophenyl group (dibenzothienyl group), naphthobenzothiophenyl group (nahthobenzothienyl group), benzothiazolyl group, benzisothiazolyl group, phenothiazinyl group, dinaphthothiophenyl group (dinaphthothienyl group), azadibenzothiophenyl group (azadibenzothienyl group), diazadibenzothiophenyl group (diazadibenzothienyl group), azanaphthobenzothiophenyl group (azanaphthobenzothienyl group), and diazanaphthobenzothiophenyl group (diazanaphthobenzothienyl group).
   Monovalent Heterocyclic Groups Derived by Removing One Hydrogen Atom from Cyclic Structures Represented by Formulae (TEMP-16) to (TEMP-33) (Specific Example Group G2A4):

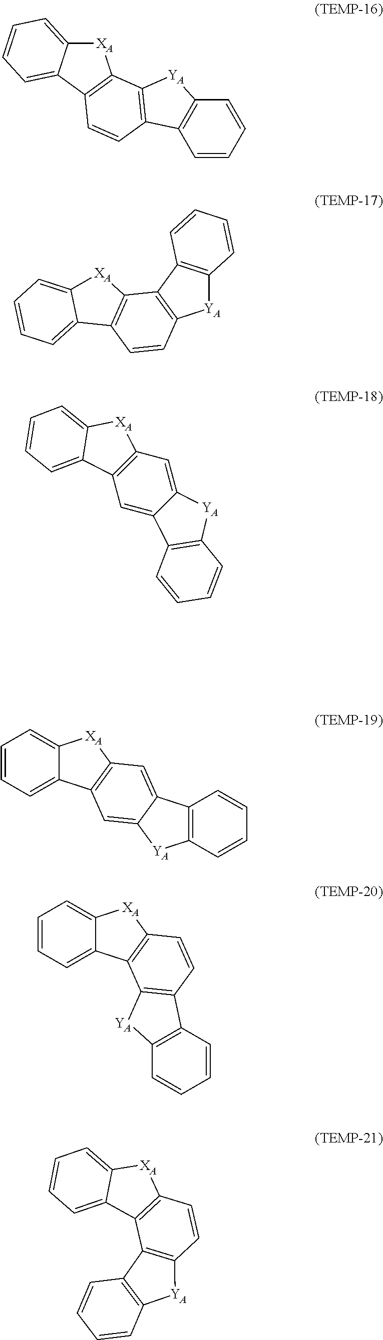

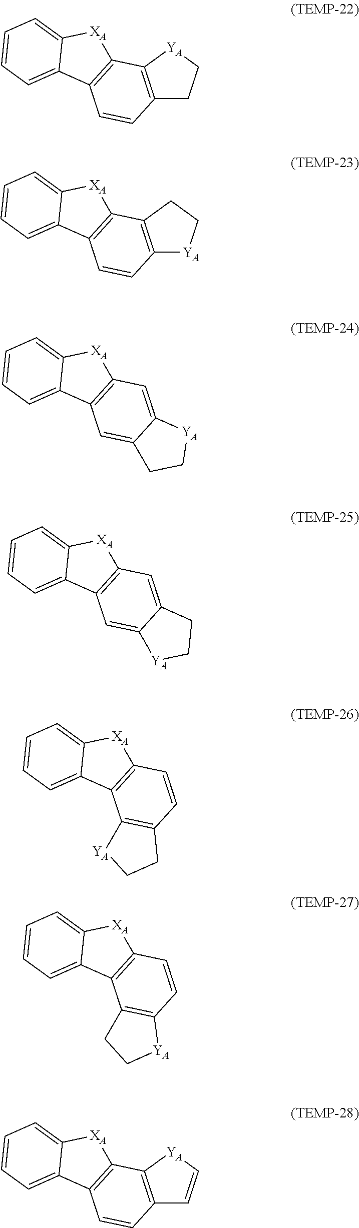

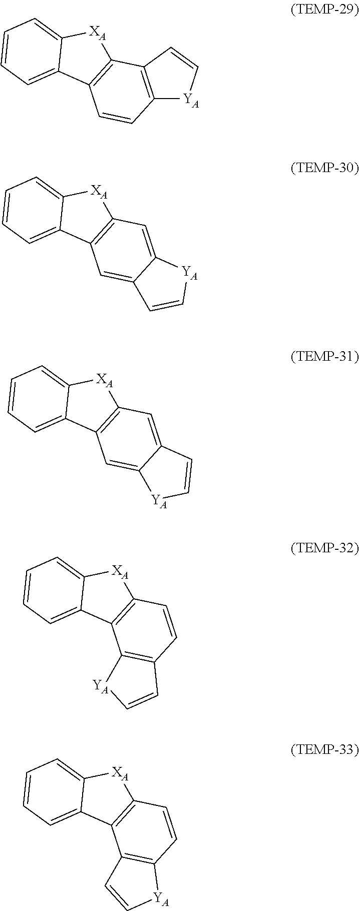

In the formulae (TEMP-16) to (TEMP-33), X_{A} and Y_{A} are each independently an oxygen atom, a sulfur atom, NH or CH₂, with a proviso that at least one of X_{A} or Y_{A} is an oxygen atom, a sulfur atom, or NH.

When at least one of X_{A} or Y_{A} in the formulae (TEMP-16) to (TEMP-33) is NH or CH₂, the monovalent heterocyclic groups derived from the cyclic structures represented by the formulae (TEMP-16) to (TEMP-33) include a monovalent group derived by removing one hydrogen atom from NH or CH₂.

### Substituted Heterocyclic Groups Including Nitrogen Atom (Specific Example Group G2B1):

a (9-phenyl)carbazolyl group, (9-biphenylyl)carbazolyl group, (9-phenyl)phenylcarbazolyl group, (9-naphthyl)carbazolyl group, diphenylcarbazole-9-yl group, phenylcarbazole-9-yl group, methylbenzimidazolyl group, ethylbenzimidazolyl group, phenyltriazinyl group, biphenylyltriazinyl group, diphenyltriazinyl group, phenylquinazolinyl group, and biphenyiquinazolinyl group.

### Substituted Heterocyclic Groups Including Oxygen Atom (Specific Example Group G2B2):

a phenyldibenzofuranyl group, methyldibenzofuranyl group, t-butyldibenzofuranyl group, and monovalent residue of spiro[9H-xanthene-9,9′-[9H]fluorene].

### Substituted Heterocyclic Groups Including Sulfur Atom (Specific Example Group G2B3):

a phenyldibenzothiophenyl group, methyldibenzothiophenyl group, t-butyldibenzothiophenyl group, and monovalent residue of spiro[9H-thioxanthene-9,9′-[9H]fluorene].
   Groups Obtained by Substituting at Least One Hydrogen Atom of Monovalent Heterocyclic Group Derived from Cyclic Structures Represented by Formulae (TEMP-16) to (TEMP-33) with Substituent (Specific Example Group G2B4):

The “at least one hydrogen atom of a monovalent heterocyclic group” means at least one hydrogen atom selected from a hydrogen atom bonded to a ring carbon atom of the monovalent heterocyclic group, a hydrogen atom bonded to a nitrogen atom of at least one of X_{A} or Y_{A} in a form of NH, and a hydrogen atom of one of X_{A} and Y_{A} in a form of a methylene group (CH₂).

### Substituted or Unsubstituted Alkyl Group

Specific examples (specific example group G3) of the “substituted or unsubstituted alkyl group” mentioned herein include unsubstituted alkyl groups (specific example group G3A) and substituted alkyl groups (specific example group G3B) below. (Herein, an unsubstituted alkyl group refers to an “unsubstituted alkyl group” in a “substituted or unsubstituted alkyl group,” and a substituted alkyl group refers to a “substituted alkyl group” in a “substituted or unsubstituted alkyl group.”) A simply termed “alkyl group” herein includes both of an “unsubstituted alkyl group” and a “substituted alkyl group”.

The “substituted alkyl group” refers to a group derived by substituting at least one hydrogen atom in an “unsubstituted alkyl group” with a substituent. Specific examples of the “substituted alkyl group” include a group derived by substituting at least one hydrogen atom of an “unsubstituted alkyl group” (specific example group G3A) below with a substituent, and examples of the substituted alkyl group (specific example group G3B) below. Herein, the alkyl group for the “unsubstituted alkyl group” refers to a chain alkyl group. Accordingly, the “unsubstituted alkyl group” include linear “unsubstituted alkyl group” and branched “unsubstituted alkyl group.” It should be noted that the examples of the “unsubstituted alkyl group” and the “substituted alkyl group” mentioned herein are merely exemplary, and the “substituted alkyl group” mentioned herein includes a group derived by further substituting a hydrogen atom of a skeleton of the “substituted alkyl group” in the specific example group G3B, and a group derived by further substituting a hydrogen atom of a substituent of the “substituted alkyl group” in the specific example group G3B.

### Unsubstituted Alkyl Group (Specific Example Group G3A):

a methyl group, ethyl group, n-propyl group, isopropyl group, n-butyl group, isobutyl group, s-butyl group, and t-butyl group.

### Substituted Alkyl Group (Specific Example Group G3B):

a heptafluoropropyl group (including isomer thereof), pentafluoroethyl group, 2,2,2-trifluoroethyl group, and trifluoromethyl group.

### Substituted or Unsubstituted Alkenyl Group

Specific examples (specific example group G4) of the “substituted or unsubstituted alkenyl group” mentioned herein include unsubstituted alkenyl groups (specific example group G4A) and substituted alkenyl groups (specific example group G4B). (Herein, an unsubstituted alkenyl group refers to an “unsubstituted alkenyl group” in a “substituted or unsubstituted alkenyl group,” and a substituted alkenyl group refers to a “substituted alkenyl group” in a “substituted or unsubstituted alkenyl group.”) A simply termed “alkenyl group” herein includes both of an “unsubstituted alkenyl group” and a “substituted alkenyl group”.

The “substituted alkenyl group” refers to a group derived by substituting at least one hydrogen atom in an “unsubstituted alkenyl group” with a substituent. Specific examples of the “substituted alkenyl group” include an “unsubstituted alkenyl group” (specific example group G4A) substituted by a substituent, and examples of the substituted alkenyl group (specific example group G4B) below. It should be noted that the examples of the “unsubstituted alkenyl group” and the “substituted alkenyl group” mentioned herein are merely exemplary, and the “substituted alkenyl group” mentioned herein includes a group derived by further substituting a hydrogen atom of a skeleton of the “substituted alkenyl group” in the specific example group G4B with a substituent, and a group derived by further substituting a hydrogen atom of a substituent of the “substituted alkenyl group” in the specific example group G4B with a substituent.

### Unsubstituted Alkenyl Group (Specific Example Group G4A):

a vinyl group, allyl group, 1-butenyl group, 2-butenyl group, and 3-butenyl group.

### Substituted Alkenyl Group (Specific Example Group G4B):

a 1,3-butanedienyl group, 1-methylvinyl group, 1-methylallyl group, 1,1-dimethylallyl group, 2-methylallyl group, and 1,2-dimethylallyl group.

### Substituted or Unsubstituted Alkynyl Group

Specific examples (specific example group G5) of the “substituted or unsubstituted alkynyl group” mentioned herein include unsubstituted alkynyl groups (specific example group G5A) below. (Herein, an unsubstituted alkynyl group refers to an “unsubstituted alkynyl group” in a “substituted or unsubstituted alkynyl group.”) A simply termed “alkynyl group” herein includes both of “unsubstituted alkynyl group” and “substituted alkynyl group”.

The “substituted alkynyl group” refers to a group derived by substituting at least one hydrogen atom in an “unsubstituted alkynyl group” with a substituent. Specific examples of the “substituted alkynyl group” include a group derived by substituting at least one hydrogen atom of the “unsubstituted alkynyl group” (specific example group G5A) below with a substituent.

### Unsubstituted Alkynyl Group (Specific Example Group G5A): An Ethynyl Group.

### Substituted or Unsubstituted Cycloalkyl Group

Specific examples (specific example group G6) of the “substituted or unsubstituted cycloalkyl group” mentioned herein include unsubstituted cycloalkyl groups (specific example group G6A) and substituted cycloalkyl groups (specific example group G6B). (Herein, an unsubstituted cycloalkyl group refers to an “unsubstituted cycloalkyl group” in a “substituted or unsubstituted cycloalkyl group,” and a substituted cycloalkyl group refers to a “substituted cycloalkyl group” in a “substituted or unsubstituted cycloalkyl group.”) A simply termed “cycloalkyl group” herein includes both of “unsubstituted cycloalkyl group” and “substituted cycloalkyl group”.

The “substituted cycloalkyl group” refers to a group derived by substituting at least one hydrogen atom of an “unsubstituted cycloalkyl group” with a substituent. Specific examples of the “substituted cycloalkyl group” include a group derived by substituting at least one hydrogen atom of the “unsubstituted cycloalkyl group” (specific example group G6A) below with a substituent, and examples of the substituted cycloalkyl group (specific example group G6B) below. It should be noted that the examples of the “unsubstituted cycloalkyl group” and the “substituted cycloalkyl group” mentioned herein are merely exemplary, and the “substituted cycloalkyl group” mentioned herein includes a group derived by substituting at least one hydrogen atom bonded to a carbon atom of a skeleton of the “substituted cycloalkyl group” in the specific example group G6B with a substituent, and a group derived by further substituting a hydrogen atom of a substituent of the “substituted cycloalkyl group” in the specific example group G6B with a substituent.

### Unsubstituted Cycloalkyl Group (Specific Example Group G6A):

a cyclopropyl group, cyclobutyl group, cyclopentyl group, cyclohexyl group, 1-adamantyl group, 2-adamantyl group, 1-norbornyl group, and 2-norbornyl group.

### Substituted Cycloalkyl Group (Specific Example Group G6B):

a 4-methylcyclohexyl group.
   Group Represented by —Si(R₉₀₁)(R₉₀₂)(R₉₀₃)

Specific examples (specific example group G7) of the group represented herein by —Si(R₉₀₁)(R₉₀₂)(R₉₀₃) include: —Si(G1)(G1)(G1); —Si(G1)(G2)(G2); —Si(G1)(G1)(G2); —Si(G2)(G2)(G2); —Si(G3)(G3)(G3); and —Si(G6)(G6)(G6); where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3;
G6 represents a “substituted or unsubstituted cycloalkyl group” in the specific example group G6.
a plurality of G1 in —Si(G1)(G1)(G1) are mutually the same or different;
a plurality of G2 in —Si(G1)(G2)(G2) are mutually the same or different;
a plurality of G1 in —Si(G1)(G1)(G2) are mutually the same or different;
a plurality of G2 in —Si(G2)(G2)(G2) are mutually the same or different;
a plurality of G3 in —Si(G3)(G3)(G3) are mutually the same or different; and
a plurality of G6 in —Si(G6)(G6)(G6) are mutually the same or different.

### Group Represented by —O—(R₉₀₄)

Specific examples (specific example group G8) of a group represented by —O—(R₉₀₄) herein include: —O(G1); —O(G2); —O(G3); and —O(G6);
where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6.

### Group Represented by —S—(R₉₀₅)

Specific examples (specific example group G9) of a group represented herein by —S—(R₉₀₅) include: —S(G1); —S(G2); —S(G3); and —S(G6);
where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6.
   Group Represented by —N(R₉₀₆)(R₉₀₇)

Specific examples (specific example group G10) of a group represented herein by —N(R₉₀₆)(R₉₀₇) include: —N(G1)(G1); —N(G2)(G2); —N(G1)(G2); —N(G3)(G3); and —N(G6)(G6);
where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3;
G6 represents a “substituted or unsubstituted cycloalkyl group” in the specific example group G6;
a plurality of G1 in —N(G1)(G1) are mutually the same or different;
a plurality of G2 in —N(G2)(G2) are mutually the same or different;
a plurality of G3 in —N(G3)(G3) are mutually the same or different; and
a plurality of G6 in —N(G6)(G6) are mutually the same or different.

### Halogen Atom

Specific examples (specific example group G11) of “halogen atom” mentioned herein include a fluorine atom, chlorine atom, bromine atom, and iodine atom.

Substituted or Unsubstituted Fluoroalkyl Group The “substituted or unsubstituted fluoroalkyl group” mentioned herein refers to a group derived by substituting at least one hydrogen atom bonded to at least one of carbon atoms forming an alkyl group in the “substituted or unsubstituted alkyl group” with a fluorine atom, and also includes a group (perfluoro group) derived by substituting all of hydrogen atoms bonded to carbon atoms forming the alkyl group in the “substituted or unsubstituted alkyl group” with fluorine atoms. An “unsubstituted fluoroalkyl group” has, unless otherwise specified herein, 1 to 50, preferably 1 to 30, more preferably 1 to 18 carbon atoms. The “substituted fluoroalkyl group” refers to a group derived by substituting at least one hydrogen atom in a “fluoroalkyl group” with a substituent. It should be noted that the examples of the “substituted fluoroalkyl group” mentioned herein include a group derived by further substituting at least one hydrogen atom bonded to a carbon atom of an alkyl chain of a “substituted fluoroalkyl group” with a substituent, and a group derived by further substituting at least one hydrogen atom of a substituent of the “substituted fluoroalkyl group” with a substituent. Specific examples of the “unsubstituted fluoroalkyl group” include a group derived by substituting at least one hydrogen atom of the “alkyl group” (specific example group G3) with a fluorine atom.

### Substituted or Unsubstituted Haloalkyl Group

The “substituted or unsubstituted haloalkyl group” mentioned herein refers to a group derived by substituting at least one hydrogen atom bonded to carbon atoms forming the alkyl group in the “substituted or unsubstituted alkyl group” with a halogen atom, and also includes a group derived by substituting all hydrogen atoms bonded to carbon atoms forming the alkyl group in the “substituted or unsubstituted alkyl group” with halogen atoms. An “unsubstituted haloalkyl group” has, unless otherwise specified herein, 1 to 50, preferably 1 to 30, and more preferably 1 to 18 carbon atoms. The “substituted haloalkyl group” refers to a group derived by substituting at least one hydrogen atom in a “haloalkyl group” with a substituent. It should be noted that the examples of the “substituted haloalkyl group” mentioned herein include a group derived by further substituting at least one hydrogen atom bonded to a carbon atom of an alkyl chain of a “substituted haloalkyl group” with a substituent, and a group derived by further substituting at least one hydrogen atom of a substituent of the “substituted haloalkyl group” with a substituent. Specific examples of the “unsubstituted haloalkyl group” include a group derived by substituting at least one hydrogen atom of the “alkyl group” (specific example group G3) with a halogen atom. The haloalkyl group is occasionally referred to as a halogenated alkyl group.

### Substituted or Unsubstituted Alkoxy Group

Specific examples of a “substituted or unsubstituted alkoxy group” mentioned herein include a group represented by —O(G3), G3 being the “substituted or unsubstituted alkyl group” in the specific example group G3. An “unsubstituted alkoxy group” has, unless otherwise specified herein, 1 to 50, preferably 1 to 30, more preferably 1 to 18 carbon atoms.

### Substituted or Unsubstituted Alkylthio Group

Specific examples of a “substituted or unsubstituted alkylthio group” mentioned herein include a group represented by —S(G3), G3 being the “substituted or unsubstituted alkyl group” in the specific example group G3. An “unsubstituted alkylthio group” has, unless otherwise specified herein, 1 to 50, preferably 1 to 30, more preferably 1 to 18 carbon atoms.

### Substituted or Unsubstituted Aryloxy Group

Specific examples of a “substituted or unsubstituted aryloxy group” mentioned herein include a group represented by —O(G1), G1 being the “substituted or unsubstituted aryl group” in the specific example group G1. An “unsubstituted aryloxy group” has, unless otherwise specified herein, 6 to 50, preferably 6 to 30, more preferably 6 to 18 ring carbon atoms.

### Substituted or Unsubstituted Arylthio Group

Specific examples of a “substituted or unsubstituted arylthio group” mentioned herein include a group represented by —S(G1), G1 being the “substituted or unsubstituted aryl group” in the specific example group G1. An “unsubstituted arylthio group” has, unless otherwise specified herein, 6 to 50, preferably 6 to 30, more preferably 6 to 18 ring carbon atoms.

### Substituted or Unsubstituted Trialkylsilyl Group

Specific examples of a “trialkylsilyl group” mentioned herein include a group represented by —Si(G3)(G3)(G3), G3 being the “substituted or unsubstituted alkyl group” in the specific example group G3. A plurality of G3 in —Si(G3)(G3)(G3) are mutually the same or different. Each of the alkyl groups in the “trialkylsilyl group” has, unless otherwise specified herein, 1 to 50, preferably 1 to 20, more preferably 1 to 6 carbon atoms.

### Substituted or Unsubstituted Aralkyl Group

Specific examples of a “substituted or unsubstituted aralkyl group” mentioned herein include a group represented by —(G3)-(G1), G3 being the “substituted or unsubstituted alkyl group” in the specific example group G3, G1 being the “substituted or unsubstituted aryl group” in the specific example group G1. Accordingly, the “aralkyl group” is a group derived by substituting a hydrogen atom of the “alkyl group” with a substituent in a form of the “aryl group,” which is an example of the “substituted alkyl group.” An “unsubstituted aralkyl group,” which is an “unsubstituted alkyl group” substituted by an “unsubstituted aryl group,” has, unless otherwise specified herein, 7 to 50 carbon atoms, preferably 7 to 30 carbon atoms, more preferably 7 to 18 carbon atoms.

Specific examples of the “substituted or unsubstituted aralkyl group” include a benzyl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, β-naphthylmethyl group, 1-2-β-naphthylethyl group, 2-β-naphthylethyl group, 1-β-naphthylisopropyl group, and 2-β-naphthylisopropyl group.

Preferable examples of the substituted or unsubstituted aryl group mentioned herein include, unless otherwise specified herein, a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-β-yl group, m-terphenyl-2-yl group, o-terphenyl-4-yl group, o-terphenyl-3-yl group, o-terphenyl-2-yl group, 1-naphthyl group, 2-naphthyl group, anthryl group, phenanthryl group, pyrenyl group, chrysenyl group, triphenylenyl group, fluorenyl group, 9,9′-spirobifluorenyl group, 9,9-dimethylfluorenyl group, and 9,9-diphenyifluorenyl group.

Preferable examples of the substituted or unsubstituted heterocyclic group mentioned herein include, unless otherwise specified herein, a pyridyl group, pyrimidinyl group, triazinyl group, quinolyl group, isoquinolyl group, quinazolinyl group, benzimidazolyl group, phenanthrolinyl group, carbazolyl group (1-carbazolyl group, 2-carbazolyl group, 3-carbazolyl group, 4-carbazolyl group, or 9-carbazolyl group), benzocarbazolyl group, azacarbazolyl group, diazacarbazolyl group, dibenzofuranyl group, naphthobenzofuranyl group, azadibenzofuranyl group, diazadibenzofuranyl group, dibenzothiophenyl group, naphthobenzothiophenyl group, azadibenzothiophenyl group, diazadibenzothiophenyl group, (9-phenyl)carbazolyl group ((9-phenyl)carbazole-1-yl group, (9-phenyl)carbazole-2-yl group, (9-phenyl)carbazole-3-yl group, or (9-phenyl)carbazole-4-yl group), (9-biphenylyl)carbazolyl group, (9-phenyl)phenylcarbazolyl group, diphenylcarbazole-9-yl group, phenylcarbazole-9-yl group, phenyltriazinyl group, biphenylyltriazinyl group, diphenyltriazinyl group, phenyldibenzofuranyl group, and phenyldibenzothiophenyl group.

The carbazolyl group mentioned herein is, unless otherwise specified herein, specifically a group represented by one of formulae below.

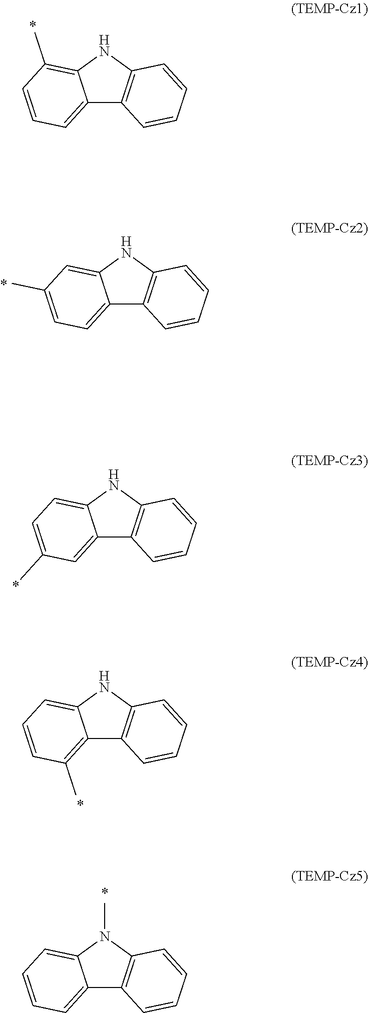

The (9-phenyl)carbazolyl group mentioned herein is, unless otherwise specified herein, specifically a group represented by one of formulae below.

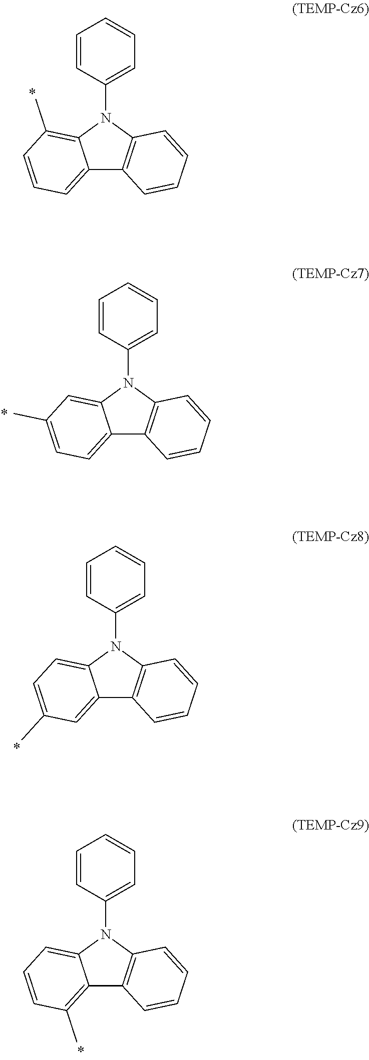 [SMILES: Cc1cccc2c3ccccc3n(-c3ccccc3)c12 ; Cc1ccc2c3ccccc3n(-c3ccccc3)c2c1 ; Cc1ccc2c(c1)c1ccccc1n2-c1ccccc1 ; Cc1cccc2c1c1ccccc1n2-c1ccccc1]

In the formulae (TEMP-Cz1) to (TEMP-Cz9), * represents a bonding position.

The dibenzofuranyl group and dibenzothiophenyl group mentioned herein are, unless otherwise specified herein, each specifically represented by one of formulae below.

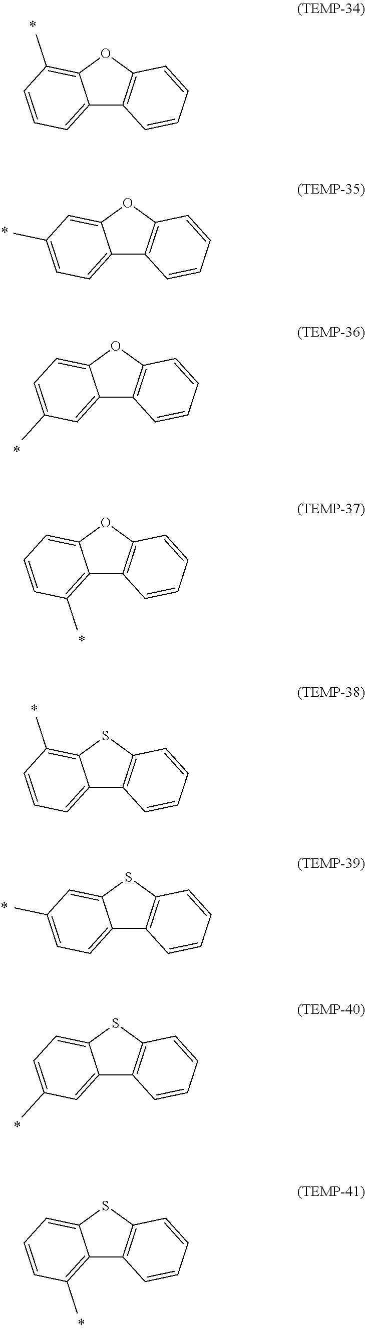

In the formulae (TEMP-34) to (TEMP-41), * represents a bonding position.

Preferable examples of the substituted or unsubstituted alkyl group mentioned herein include, unless otherwise specified herein, a methyl group, ethyl group, propyl group, isopropyl group, n-butyl group, isobutyl group, and t-butyl group.

### Substituted or Unsubstituted Arylene Group

The “substituted or unsubstituted arylene group” mentioned herein is, unless otherwise specified herein, a divalent group derived by removing one hydrogen atom on an aryl ring of the “substituted or unsubstituted aryl group.” Specific examples of the “substituted or unsubstituted arylene group” (specific example group G12) include a divalent group derived by removing one hydrogen atom on an aryl ring of the “substituted or unsubstituted aryl group” in the specific example group G1.

### Substituted or Unsubstituted Divalent Heterocyclic Group

The “substituted or unsubstituted divalent heterocyclic group” mentioned herein is, unless otherwise specified herein, a divalent group derived by removing one hydrogen atom on a heterocycle of the “substituted or unsubstituted heterocyclic group.” Specific examples of the “substituted or unsubstituted divalent heterocyclic group” (specific example group G13) include a divalent group derived by removing one hydrogen atom on a heterocyclic ring of the “substituted or unsubstituted heterocyclic group” in the specific example group G2.

### Substituted or Unsubstituted Alkylene Group

The “substituted or unsubstituted alkylene group” mentioned herein is, unless otherwise specified herein, a divalent group derived by removing one hydrogen atom on an alkyl chain of the “substituted or unsubstituted alkyl group.” Specific examples of the “substituted or unsubstituted alkylene group” (specific example group G14) include a divalent group derived by removing one hydrogen atom on an alkyl chain of the “substituted or unsubstituted alkyl group” in the specific example group G3.

The substituted or unsubstituted arylene group mentioned herein is, unless otherwise specified herein, preferably any one of groups represented by formulae (TEMP-42) to (TEMP-68) below.

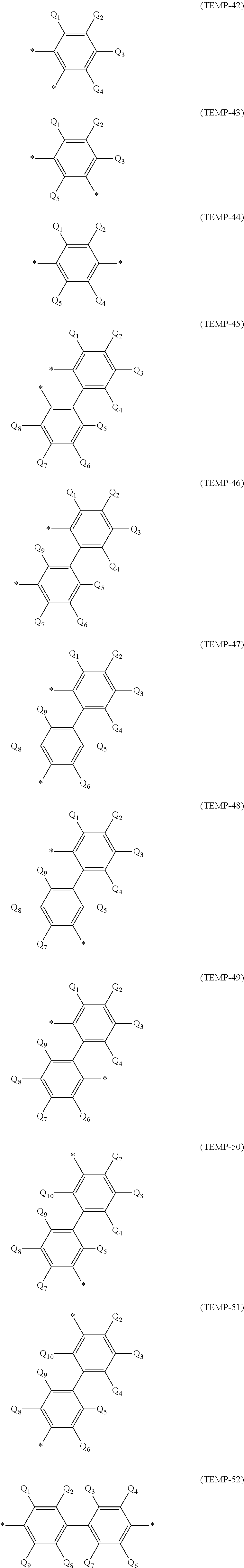

In the formulae (TEMP-42) to (TEMP-52), Q₁ to Q₁₀ are each independently a hydrogen atom or a substituent.

In the formulae (TEMP-42) to (TEMP-52), * represents a bonding position.

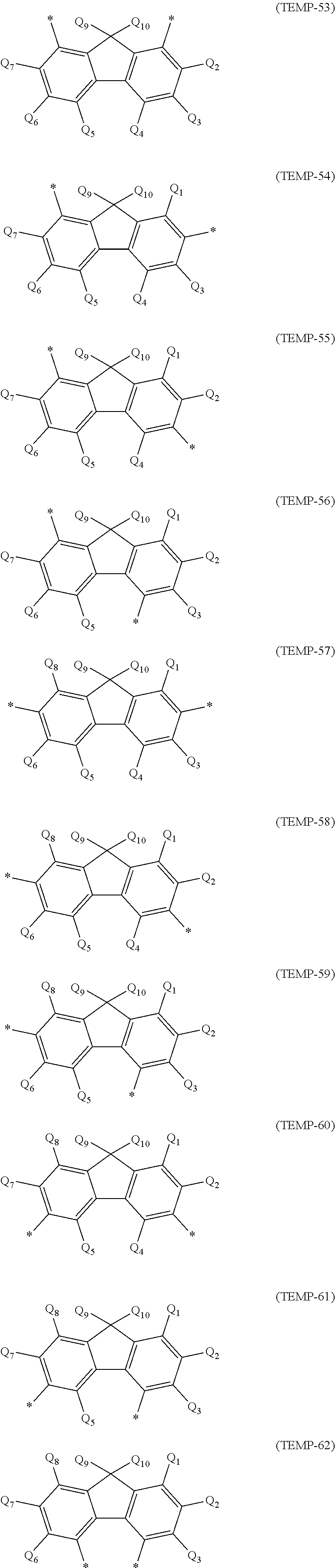

In the formulae (TEMP-53) to (TEMP-62), Q₁ to Q₁₀ are each independently a hydrogen atom or a substituent.

In the formulae, Q₉ and Q₁₀ may be mutually bonded through a single bond to form a ring.

In the formulae (TEMP-53) to (TEMP-62), * represents a bonding position.

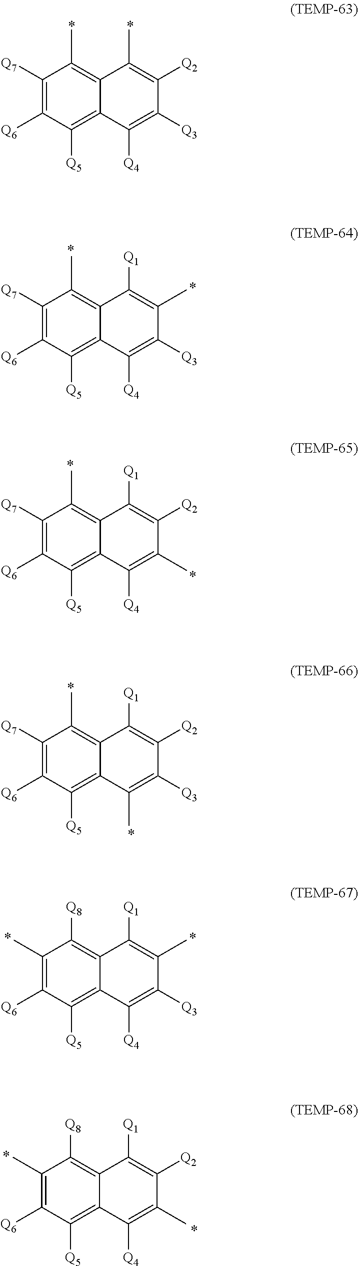

In the formulae (TEMP-63) to (TEMP-68), Q₁ to Q₈ are each independently a hydrogen atom or a substituent.

In the formulae (TEMP-63) to (TEMP-68), * represents a bonding position.

The substituted or unsubstituted divalent heterocyclic group mentioned herein is, unless otherwise specified herein, preferably a group represented by any one of formulae (TEMP-69) to (TEMP-102) below.

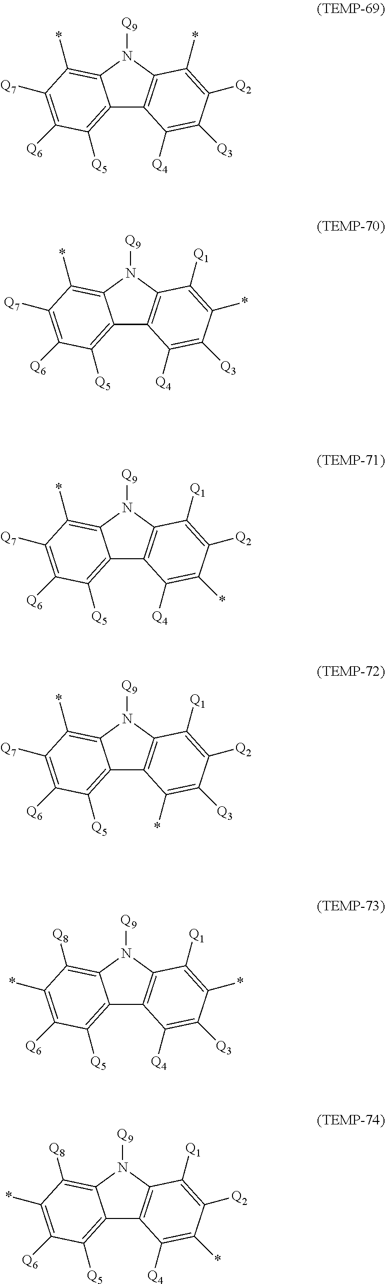

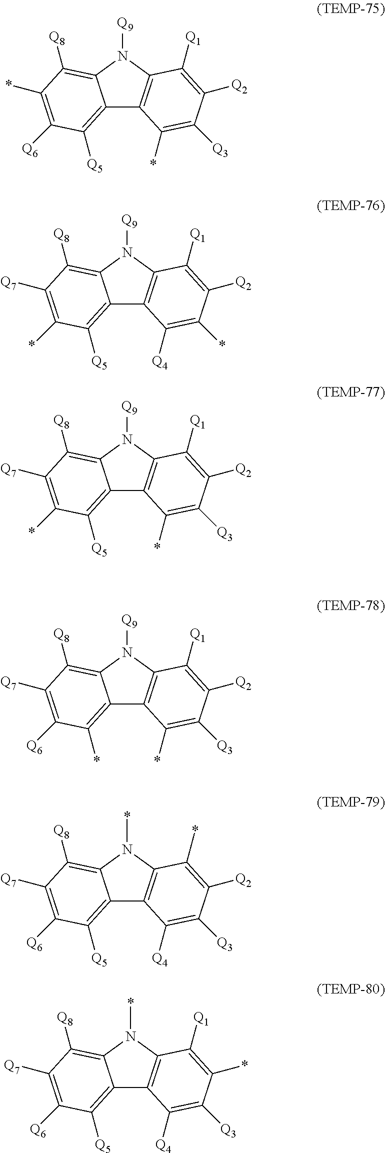

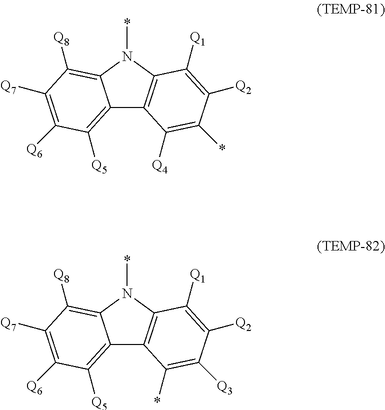

In the formulae (TEMP-69) to (TEMP-82), Q₁ to Q₉ are each independently a hydrogen atom or a substituent.

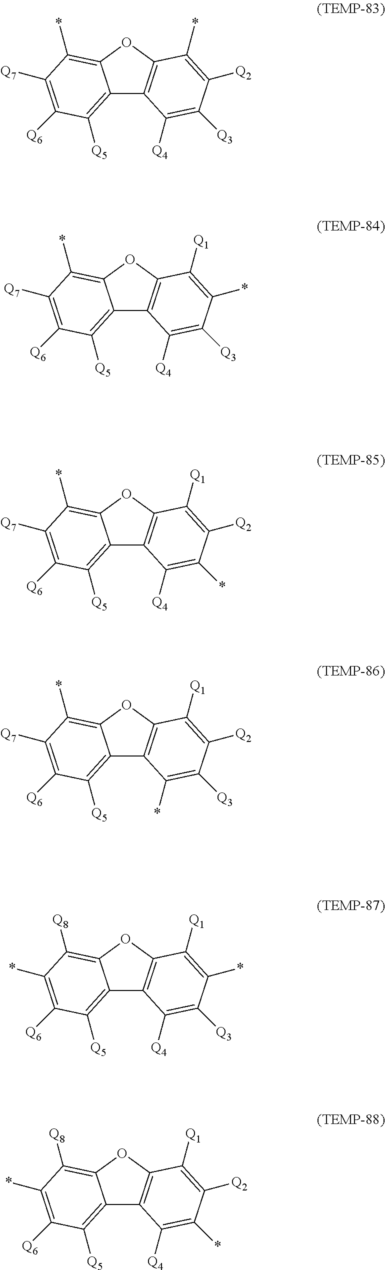

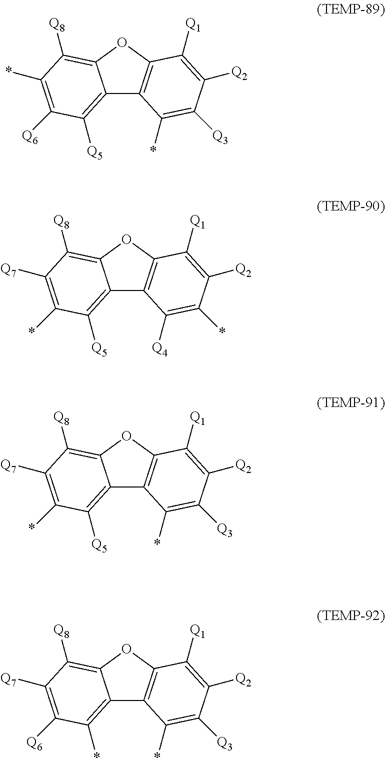

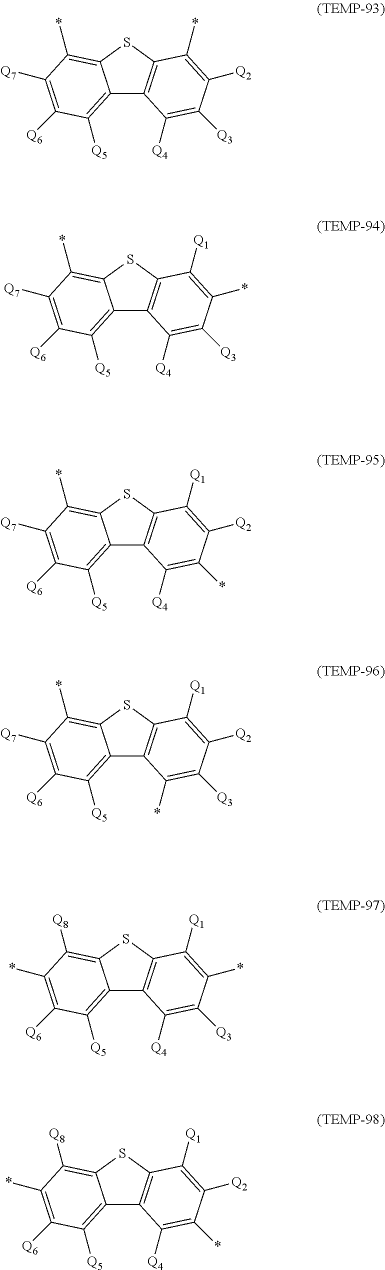

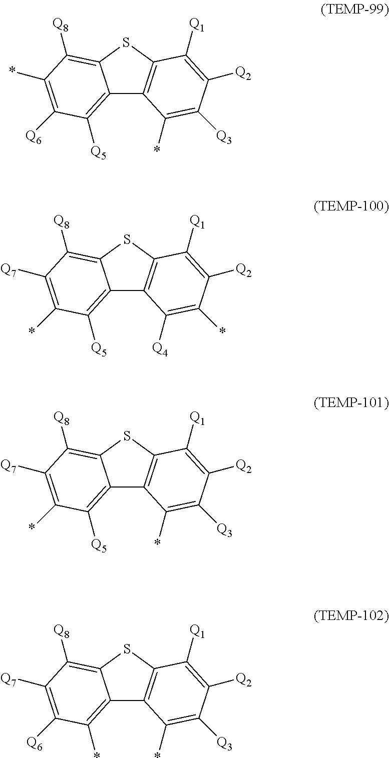

In the formulae (TEMP-83) to (TEMP-102), Q₁ to Q₈ are each independently a hydrogen atom or a substituent.

The substituent mentioned herein has been described above.

### Instance of “Bonded to Form Ring”

Instances where “at least one combination of adjacent two or more (of . . . ) are mutually bonded to form a substituted or unsubstituted monocyclic ring, mutually bonded to form a substituted or unsubstituted fused ring, or not mutually bonded” mentioned herein refer to instances where “at least one combination of adjacent two or more (of . . . ) are mutually bonded to form a substituted or unsubstituted monocyclic ring, “at least one combination of adjacent two or more (of . . . ) are mutually bonded to form a substituted or unsubstituted fused ring,” and “at least one combination of adjacent two or more (of . . . ) are not mutually bonded.”

Instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (these instances will be sometimes collectively referred to as an instance of “bonded to form a ring” hereinafter) will be described below. An anthracene compound having a basic skeleton in a form of an anthracene ring and represented by a formula (TEMP-103) below will be used as an example for the description.

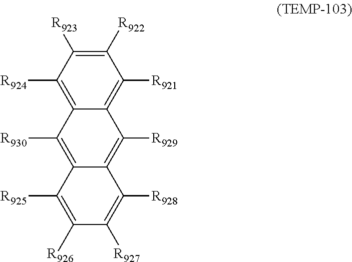

For instance, when “at least one combination of adjacent two or more of R₉₂₁ to R₉₃₀ are mutually bonded to form a ring,” the combination of adjacent ones of R₉₂₁ to R₉₃₀ (i.e. the combination at issue) is a combination of R₉₂₁ and R₉₂₂, a combination of R₉₂₂ and R₉₂₃, a combination of R₉₂₃ and R₉₂₄, a combination of R₉₂₄ and R₉₃₀, a combination of R₉₃₀ and R₉₂₅, a combination of R₉₂₅ and R₉₂₆, a combination of R₉₂₆ and R₉₂₇, a combination of R₉₂₇ and R₉₂₈, a combination of R₉₂₈ and R₉₂₉, or a combination of R₉₂₉ and R₉₂₁.

The term “at least one combination” means that two or more of the above combinations of adjacent two or more of R₉₂₁ to R₉₃₀ may simultaneously form rings. For instance, when R₉₂₁ and R₉₃₀ are mutually bonded to form a ring Q_{A} and R₉₂₅ and R₉₂₆ are simultaneously mutually bonded to form a ring Q_{B}, the anthracene compound represented by the formula (TEMP-103) is represented by a formula (TEMP-104) below.

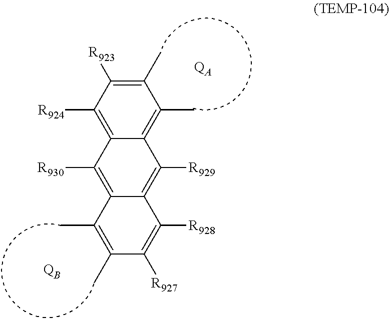

The instance where the “combination of adjacent two or more” form a ring means not only an instance where the “two” adjacent components are bonded but also an instance where adjacent “three or more” are bonded. For instance, R₉₂₁ and R₉₂₂ are mutually bonded to form a ring Q_{A} and R₉₂₂ and R₉₂₃ are mutually bonded to form a ring Q_{C}, and mutually adjacent three components (R₉₂₁, R₉₂₂ and R₉₂₃) are mutually bonded to form a ring fused to the anthracene basic skeleton. In this case, the anthracene compound represented by the formula (TEMP-103) is represented by a formula (TEMP-105) below. In the formula (TEMP-105) below, the ring Q_{A} and the ring Q_{C} share R₉₂₂.

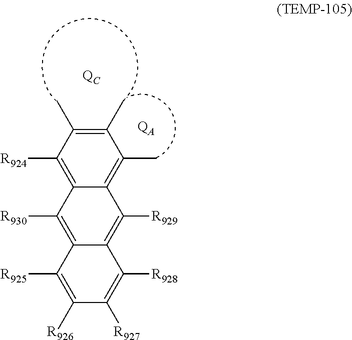

The formed “monocyclic ring” or “fused ring” may be, in terms of the formed ring in itself, a saturated ring or an unsaturated ring. When the “combination of adjacent two” form a “monocyclic ring” or a “fused ring,” the “monocyclic ring” or “fused ring” may be a saturated ring or an unsaturated ring. For instance, the ring Q_{A} and the ring Q_{B} formed in the formula (TEMP-104) are each independently a “monocyclic ring” or a “fused ring.” Further, the ring Q_{A} and the ring Q_{C} formed in the formula (TEMP-105) are each a “fused ring.” The ring Q_{A} and the ring Q_{C} in the formula (TEMP-105) are fused to form a fused ring. When the ring Q_{A} in the formula (TEMP-104) is a benzene ring, the ring Q_{A} is a monocyclic ring. When the ring Q_{A} in the formula (TEMP-104) is a naphthalene ring, the ring Q_{A} is a fused ring.

The “unsaturated ring” represents an aromatic hydrocarbon ring or an aromatic heterocycle. The “saturated ring” represents an aliphatic hydrocarbon ring or a non-aromatic heterocycle.

Specific examples of the aromatic hydrocarbon ring include a ring formed by terminating a bond of a group in the specific example of the specific example group G1 with a hydrogen atom.

Specific examples of the aromatic heterocycle include a ring formed by terminating a bond of an aromatic heterocyclic group in the specific example of the specific example group G2 with a hydrogen atom.

Specific examples of the aliphatic hydrocarbon ring include a ring formed by terminating a bond of a group in the specific example of the specific example group G6 with a hydrogen atom.

The phrase “to form a ring” herein means that a ring is formed only by a plurality of atoms of a basic skeleton, or by a combination of a plurality of atoms of the basic skeleton and one or more optional atoms. For instance, the ring Q_{A} formed by mutually bonding R₉₂₁ and R₉₂₂ shown in the formula (TEMP-104) is a ring formed by a carbon atom of the anthracene skeleton bonded to R₉₂₁, a carbon atom of the anthracene skeleton bonded to R₉₂₂, and one or more optional atoms. Specifically, when the ring Q_{A} is a monocyclic unsaturated ring formed by R₉₂₁ and R₉₂₂, the ring formed by a carbon atom of the anthracene skeleton bonded to R₉₂₁, a carbon atom of the anthracene skeleton bonded to R₉₂₂, and four carbon atoms is a benzene ring.

The “optional atom” is, unless otherwise specified herein, preferably at least one atom selected from the group consisting of a carbon atom, nitrogen atom, oxygen atom, and sulfur atom. A bond of the optional atom (e.g. a carbon atom and a nitrogen atom) not forming a ring may be terminated by a hydrogen atom or the like or may be substituted by an “optional substituent” described later. When the ring includes any other optional element than the carbon atom, the resultant ring is a heterocycle.

The number of “one or more optional atoms” forming the monocyclic ring or fused ring is, unless otherwise specified herein, preferably in a range from 2 to 15, more preferably in a range from 3 to 12, further preferably in a range from 3 to 5.

Unless otherwise specified herein, the ring, which may be a “monocyclic ring” or “fused ring,” is preferably a “monocyclic ring.”

Unless otherwise specified herein, the ring, which may be a “saturated ring” or “unsaturated ring,” is preferably an “unsaturated ring.”

Unless otherwise specified herein, the “monocyclic ring” is preferably a benzene ring.

Unless otherwise specified herein, the “unsaturated ring” is preferably a benzene ring.

When “at least one combination of adjacent two or more” (of . . . ) are “mutually bonded to form a substituted or unsubstituted monocyclic ring” or “mutually bonded to form a substituted or unsubstituted fused ring,” unless otherwise specified herein, at least one combination of adjacent two or more of components are preferably mutually bonded to form a substituted or unsubstituted “unsaturated ring” formed of a plurality of atoms of the basic skeleton, and 1 to 15 atoms of at least one element selected from the group consisting of carbon, nitrogen, oxygen and sulfur.

When the “monocyclic ring” or the “fused ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

When the “saturated ring” or the “unsaturated ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

The above is the description for the instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (sometimes referred to as an instance of “bonded to form a ring”).

### Substituent for Substituted or Unsubstituted Group

In an exemplary embodiment herein, the substituent for the substituted or unsubstituted group (hereinafter occasionally referred to as an “optional substituent”), is for instance, a group selected from the group consisting of an unsubstituted alkyl group having 1 to 50 carbon atoms, an unsubstituted alkenyl group having 2 to 50 carbon atoms, an unsubstituted alkynyl group having 2 to 50 carbon atoms, an unsubstituted cycloalkyl group having 3 to 50 ring carbon atoms, —Si(R₉₀₁)(R₉₀₂)(R₉₀₃), —O—(R₉₀₄₎, —S—(R₉₀₅), —N(R₉₀₆)(R₉₀₇), a halogen atom, a cyano group, a nitro group, an unsubstituted aryl group having 6 to 50 ring carbon atoms, and an unsubstituted heterocyclic group having 5 to 50 ring atoms;

R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when two or more R₉₀₁ are present, the two or more R₉₀₁ are mutually the same or different;
when two or more R₉₀₂ are present, the two or more R₉₀₂ are mutually the same or different;
when two or more R₉₀₃ are present, the two or more R₉₀₃ are mutually the same or different;
when two or more R₉₀₄ are present, the two or more R₉₀₄ are mutually the same or different;
when two or more R₉₀₅ are present, the two or more R₉₀₅ are mutually the same or different;
when two or more R₉₀₆ are present, the two or more R₉₀₆ are mutually the same or different; and
when two or more R₉₀₇ are present, the two or more R₉₀₇ are mutually the same or different.

In an exemplary embodiment, the substituent for the substituted or unsubstituted group is a group selected from the group consisting of an alkyl group having 1 to 50 carbon atoms, an aryl group having 6 to 50 ring carbon atoms, and a heterocyclic group having 5 to 50 ring atoms.

In an exemplary embodiment, the substituent for the substituted or unsubstituted group is a group selected from the group consisting of an alkyl group having 1 to 18 carbon atoms, an aryl group having 6 to 18 ring carbon atoms, and a heterocyclic group having 5 to 18 ring atoms.

Specific examples of the above optional substituent are the same as the specific examples of the substituent described in the above under the subtitle “Substituent Mentioned Herein.”

Unless otherwise specified herein, adjacent ones of the optional substituents may form a “saturated ring” or an “unsaturated ring,” preferably a substituted or unsubstituted saturated five-membered ring, a substituted or unsubstituted saturated six-membered ring, a substituted or unsubstituted unsaturated five-membered ring, or a substituted or unsubstituted unsaturated six-membered ring, more preferably a benzene ring.

Unless otherwise specified herein, the optional substituent may further include a substituent. Examples of the substituent for the optional substituent are the same as the examples of the optional substituent.

Herein, numerical ranges represented by “AA to BB” represent a range whose lower limit is the value (AA) recited before “to” and whose upper limit is the value (BB) recited after “to.”

Herein, a numerical formula represented by “A Z B” means that the value A is equal to the value B, or the value A is larger than the value B.

Herein, a numerical formula represented by “A s B” means that the value A is equal to the value B, or the value A is smaller than the value B.

### First Exemplary Embodiment

### Compounds

A compound according to a first exemplary embodiment is a compound represented by a formula (1) below.

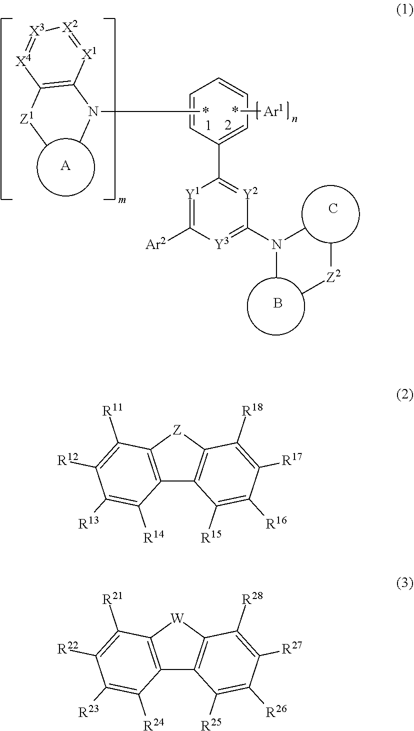

In the formula (1):
X¹ to X⁴ are each independently a nitrogen atom or CRx;
at least one of X¹ to X⁴ is a nitrogen atom;
when a plurality of Rx are present, the plurality of Rx are mutually the same or different;
Y¹ to Y³ are each independently a nitrogen atom or CH;
two or more of Y¹ to Y³ are each a nitrogen atom;
a ring A, a ring B, and a ring C are each independently a substituted or unsubstituted aromatic hydrocarbon ring having 6 to 60 ring carbon atoms, or a substituted or unsubstituted heterocycle having 5 to 60 ring atoms;
Z¹ and Z² are each independently a single bond, C(R₁)(R²), NR³, an oxygen atom, or a sulfur atom;
Ar¹ is a hydrogen atom, a substituted or unsubstituted aryl group having 6 to 50 ring carbon atoms, or a group represented by a formula (2) above;
Ar² is a substituted or unsubstituted aryl group having 6 to 50 ring carbon atoms, or a group represented by a formula (3) above;
m is 1, 2, 3, 4, or 5;
n is 0, 1, 2, 3, or 4;
m+n=5 is satisfied;
when m is 2, 3, 4, or 5,
a plurality of X¹ are mutually the same or different;
a plurality of X² are mutually the same or different;
a plurality of X³ are mutually the same or different;
a plurality of X⁴ are mutually the same or different;
a plurality of rings A are mutually the same or different; and
a plurality of Z¹ are mutually the same or different; and
when n is 2, 3, or 4,
a plurality of Ar¹ are mutually the same or different; and
*1 and *2 each represent a bonding position to a benzene ring, in the formula (2):
Z is C(R_{1A})(R^{2A}), NR^{3A}, an oxygen atom, or a sulfur atom;
at least one combination of adjacent two or more of R₁₁ to R₁₈ are mutually bonded to form a substituted or unsubstituted monocyclic ring, mutually bonded to form a substituted or unsubstituted fused ring, or not mutually bonded; and
when Ar¹ is a group represented by the formula (2), one of R₁₁ to R₁₈ and R^{3A} is a single bond with a benzene ring in the formula (1),
in the formula (3):
W is C(R^{21A})(R^{22A}), an oxygen atom, or a sulfur atom;
at least one combination of adjacent two or more of R²¹ to R²⁸ are mutually bonded to form a substituted or unsubstituted monocyclic ring, mutually bonded to form a substituted or unsubstituted fused ring, or not mutually bonded;
when Ar² is a group represented by the formula (3), one of R²¹ to R²⁸ is a single bond with a carbon atom between Y¹ and Y³ in the formula (1); and
Rx, R₁, R², R³, R_{1A}, R^{2A}, R^{21A}, R^{22A}, and R^{3A}, R₁₁ to R₁₈ and R²¹ to R²⁸ not being the single bond with the benzene ring, not being the single bond with the carbon atom between Y¹ and Y³, and forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms, and
in the compound represented by the formula (1), R₉₀₆ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

The compound of the exemplary embodiment (the compound represented by the formula (1)) has a structure in which at least (i) an azine ring having a donor group such as a carbazolyl group and (ii) a donor group having a nitrogen-containing six-membered cyclic structure such as an azacarbazolyl group are bonded to a benzene ring. The compound of the exemplary embodiment including (i) and (ii) obtains the following effects.

In the compound of the exemplary embodiment, the azacarbazolyl group as the donor group having the nitrogen-containing six-membered cyclic structure is lower in electron donating property but higher in lowest triplet energy than the carbazolyl group having no nitrogen-containing six-membered cyclic structure. The compound of the exemplary embodiment thus shortens a maximum fluorescence peak wavelength λ_{FL} (PL peak) while maintaining TADF.

Further, since the maximum fluorescence peak wavelength λ_{FL} is shortened by using the compound of the exemplary embodiment, blue chromaticity of the organic EL device is improved. As a result, it is possible to improve BI (Blue Index), which is a luminous efficiency index that takes into account the chromaticity.

Herein, a value of L/J/CIEy is referred to as Blue Index (BI). A chromaticity CIEy and a current efficiency L/J (unit: cd/A) are calculated by a method described in Examples.

For instance, a compound TADF1 below has a structure in which an azine ring having a donor group (a carbazolyl group) and a donor group having a nitrogen-containing six-membered cyclic structure (an azacarbazolyl group) are bonded to a benzene ring. A compound Ref-1 below is a compound in which the azacarbazolyl group in the compound TADF1 is substituted with the carbazolyl group.

As below, it is found out that the maximum fluorescence peak wavelength λ_{FL} of the compound TADF1 is shorter than that of the compound Ref-1. The maximum fluorescence peak wavelength λ_{FL} is measured by a method described in Examples.

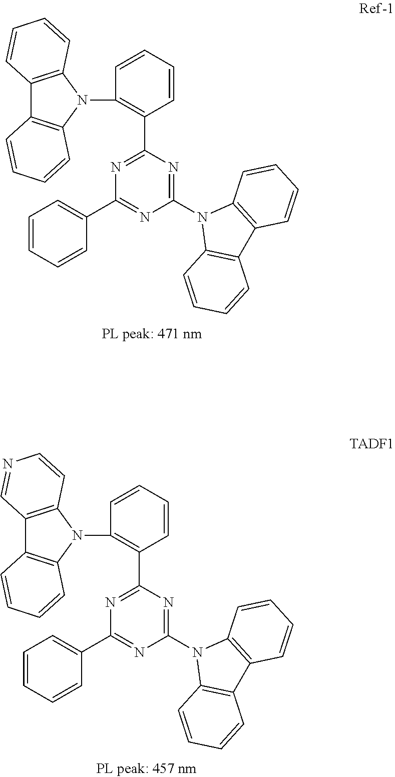 [SMILES: c1ccc(-c2nc(-c3ccccc3-n3c4ccccc4c4ccccc43)nc(-n3c4ccccc4c4ccccc43)n2)cc1 ; c1ccc(-c2nc(-c3ccccc3-n3c4ccccc4c4cnccc43)nc(-n3c4ccccc4c4ccccc43)n2)cc1]

In the compound of the exemplary embodiment, Z¹ and Z² are each preferably a single bond.

In the compound of the exemplary embodiment, the ring A, the ring B, and the ring C are preferably each independently a substituted or unsubstituted benzene ring, or a substituted or unsubstituted dibenzothiophene ring.

In the compound of the exemplary embodiment, it is preferable that: both of the rings B and C are each a substituted or unsubstituted benzene ring; or one of the rings B and C is a substituted or unsubstituted benzene ring and the other is a substituted or unsubstituted dibenzothiophene ring.

In the compound of the exemplary embodiment, it is preferable that one of X¹ to X³ is a nitrogen atom and X¹ to X⁴ not being the nitrogen atom are each CRx.

In the compound of the exemplary embodiment, it is preferable that X² or X⁴ is a nitrogen atom and X¹ to X⁴ not being the nitrogen atom are each CRx.

In a compound of an exemplary embodiment, X¹ is a nitrogen atom and X² to X⁴ are each CRx. In a compound of an exemplary embodiment, X² is a nitrogen atom, and X¹, X³, and X⁴ are each CRx. In a compound of an exemplary embodiment, X³ is a nitrogen atom, and X¹, X², and X⁴ are each CRx. In a compound of an exemplary embodiment, X⁴ is a nitrogen atom, and X¹, X², and X³ are each CRx.

In the compound of the exemplary embodiment, it is preferable that: Y² and Y³ are each a nitrogen atom and Y¹ is CH; or Y¹ to Y³ are each a nitrogen atom.

In the compound of the exemplary embodiment, Y¹ to Y³ are each preferably a nitrogen atom.

In the compound of the exemplary embodiment, Ar¹ is preferably a hydrogen atom, a substituted or unsubstituted phenyl group, a substituted or unsubstituted biphenyl group, a substituted or unsubstituted fluorenyl group, a substituted or unsubstituted carbazolyl group, a substituted or unsubstituted dibenzofuranyl group, or a substituted or unsubstituted dibenzothiophenyl group.

In the compound of the exemplary embodiment, Ar² is preferably a substituted or unsubstituted phenyl group, a substituted or unsubstituted biphenyl group, a substituted or unsubstituted fluorenyl group, a substituted or unsubstituted dibenzofuranyl group, or a substituted or unsubstituted dibenzothiophenyl group.

In the compound represented by the formula (1) of the exemplary embodiment, a partial structure represented by a formula (1A) below is preferably a group represented by one of formulae (11A) to (14A) below. When a plurality of partial structures represented by the formula (1A) are present in the compound represented by the formula (1), the partial structures represented by the formula (1A) are mutually the same or different.

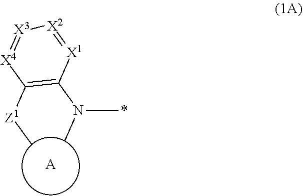

In the formula (1A):
a ring A, X¹ to X⁴, and Z¹ respectively represent the same as the ring A, X¹ to X⁴, and Z¹ in the formula (1);
when a plurality of partial structures represented by the formula (1A) are present, the partial structures represented by the formula (1A) are mutually the same or different; and
*1 represents the same as *1 in the formula (1).

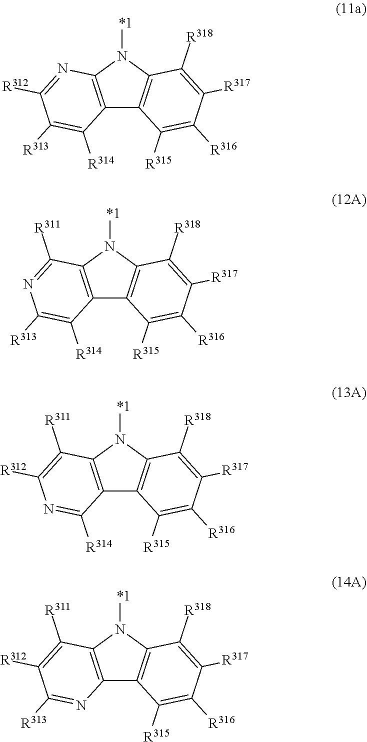

In the formulae (11A) to (14A):
at least one combination of adjacent two or more of R₃₁₁ to R₃₁₈ are mutually bonded to form a substituted or unsubstituted monocyclic ring, mutually bonded to form a substituted or unsubstituted fused ring, or not mutually bonded;
R₃₁₁ to R₃₁₈ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;

R₉₀₁ to R₉₀₇ in the formulae (11A) to (14A) respectively represent the same as R₉₀₆ to R₉₀₇ in the formula (1); and
*1 represents the same as *1 in the formula (1A).

In the formulae (11A) to (14A), it is preferable that no combination of adjacent two or more of R³¹¹ to R³¹⁸ are mutually bonded.

In the compound represented by the formula (1) of the exemplary embodiment, the partial structure represented by the formula (1A) is also preferably a group represented by one of formulae (15A) to (18A) below.

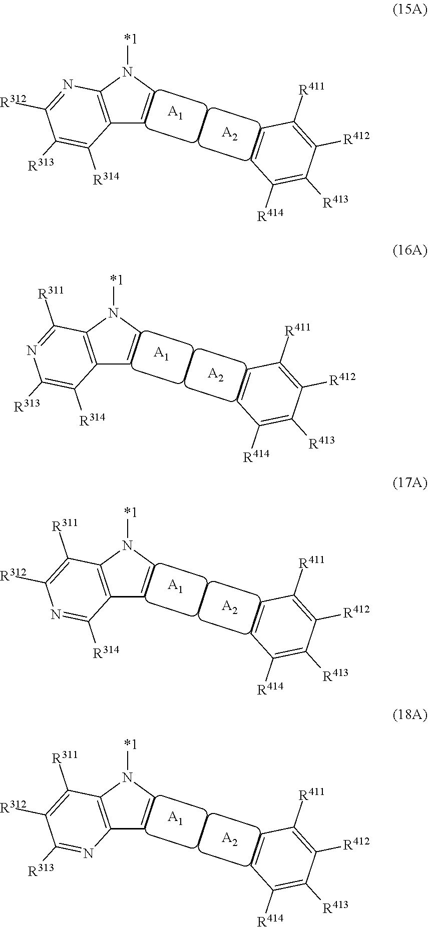

In the formulae (15A) to (18A):
a ring A₁ is a cyclic structure represented by a formula (19A) below;
a ring A₂ is a cyclic structure represented by a formula (20A) below;
the ring A₁ and the ring A₂ are fused with adjacent rings at any positions;
at least one combination of adjacent two or more of R₃₁₁ to R₃₁₄ are mutually bonded to form a substituted or unsubstituted monocyclic ring, mutually bonded to form a substituted or unsubstituted fused ring, or not mutually bonded; and
at least one combination of adjacent two or more of R₄₁₁ to R₄₁₄ are mutually bonded to form a substituted or unsubstituted monocyclic ring, mutually bonded to form a substituted or unsubstituted fused ring, or not mutually bonded;
in the formulae (15A) to (18A):
R³¹¹ to R³¹⁴ and R⁴¹¹ to R⁴¹⁴ forming neither the substituted or unsubstituted monocyclic ring nor the substituted or unsubstituted fused ring each independently represent the same as R³¹¹ to R³¹⁸ forming neither the substituted or unsubstituted monocyclic ring nor the substituted or unsubstituted fused ring in the formulae (11A) to (14A); and
*1 represents the same as *1 in the formula (1A).

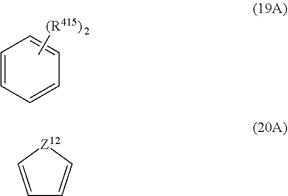

In the formula (19A):
a combination of a plurality of R⁴¹⁵ are mutually bonded to form a substituted or unsubstituted monocyclic ring, mutually bonded to form a substituted or unsubstituted fused ring, or not mutually bonded;
R⁴¹⁵ forming neither the substituted or unsubstituted monocyclic ring nor the substituted or unsubstituted fused ring represents the same as R³¹¹ to R₃₁₈ forming neither the substituted or unsubstituted monocyclic ring nor the substituted or unsubstituted fused ring in the formulae (11A) to (14A); and
a plurality of R⁴¹⁵ are mutually the same or different, and
in the formula (20A):
Z¹² is C(R_{1B})(R^{2B}), NR^{3B}, an oxygen atom, or a sulfur atom; and
R_{1B}, R^{2B}, and R^{3B} each independently represent the same as R³¹¹ to R³¹⁸ forming neither the substituted or unsubstituted monocyclic ring nor the substituted or unsubstituted fused ring in the formulae (11A) to (14A).

In the formulae (15A) to (18A), it is preferable that no combination of adjacent two or more of R³¹¹ to R³¹⁴ and R⁴¹¹ to R⁴¹⁴ are mutually bonded.

In the formula (19A), it is preferable that no combination of a plurality of R⁴¹⁵ are mutually bonded.

In the formula (19A), R⁴¹⁵, R^{1B}, R^{2B}, and R^{3B} are preferably each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In the formula (20A), Z¹² is preferably a sulfur atom.

In the compound represented by the formula (1) of the exemplary embodiment, a partial structure represented by a formula (1B) below is preferably a group represented by one of formulae (11B) to (13B) below.

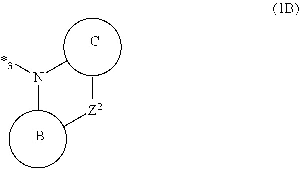

In the formula (1B), a ring B, a ring C, and Z² respectively represent the same as the ring B, the ring C, and Z² in the formula (1), and *3 represents a bonding position to a carbon atom between Y² and Y³ in the formula (1).

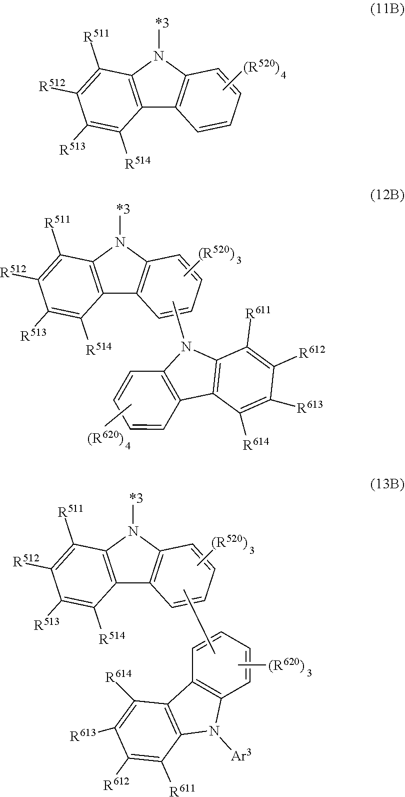

In the formulae (11B) to (13B):
at least one combination of adjacent two or more of R⁵¹¹ to R⁵¹⁴ and R⁵²⁰ are mutually bonded to form a substituted or unsubstituted monocyclic ring, mutually bonded to form a substituted or unsubstituted fused ring, or not mutually bonded;
at least one combination of adjacent two or more of R⁶¹¹ to R⁶¹⁴ and R⁶²⁰ are mutually bonded to form a substituted or unsubstituted monocyclic ring, mutually bonded to form a substituted or unsubstituted fused ring, or not mutually bonded;
a plurality of R⁵²⁰ are mutually the same or different;
a plurality of R⁶²⁰ are mutually the same or different;
Ar³ is a substituted or unsubstituted aryl group having 6 to 50 ring carbon atoms, or a substituted or unsubstituted heterocyclic group having 5 to 50 ring atoms;
R⁵¹¹ to R⁵¹⁴, R⁵²⁰, R⁶¹¹ to R⁶¹⁴, and R⁶²⁰ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄₎, a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
R₉₀₁ to R₉₀₇ in the formulae (11B) to (13B) respectively represent the same as R₉₀₆ to R₉₀₇ in the formula (1); and
3 represents the same as *3 in the formula (1B).

In the compound of the exemplary embodiment, m is preferably 1.

In the formulae (11B) to (13B), it is preferable that no combination of adjacent two or more of R⁵¹¹ to R⁵¹⁴, R⁵²⁰, R⁶¹¹ to R⁶¹⁴ and R⁶²⁰ are mutually bonded.

In the compound represented by the formula (1) of the exemplary embodiment, the partial structure represented by the formula (1B) is also preferably a group represented by a formula (14B) or a formula (15B) below.

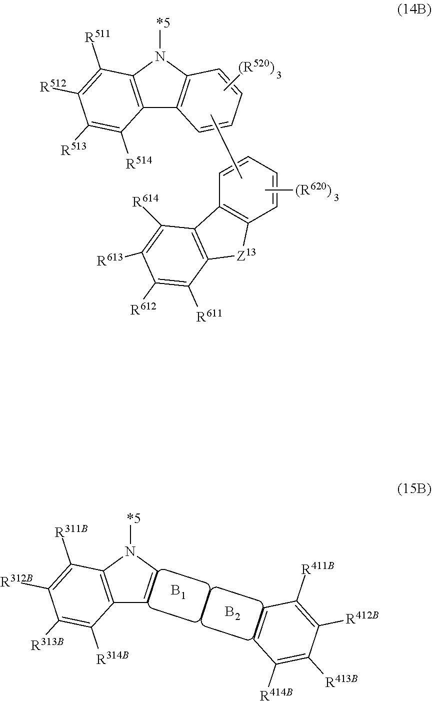

In the formula (14B):
Z¹³ is C(R^{1C})(R^{2C}), an oxygen atom, or a sulfur atom;
R^{1C} and R^{2C} are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms;
R⁵¹¹ to R⁵¹⁴, R⁵²⁰, R⁶¹¹ to R⁶¹⁴ and R⁶²⁰ respectively represent the same as R⁵¹¹ to R⁵¹⁴, R⁵²⁰, R⁶¹¹ to R⁶¹⁴ and R⁶²⁰ in the formula (13B); and
*5 represents the same as *3 in the formula (1B).

In the formula (15B):
a ring B₁, a ring B₂, R^{31B} to R_{314B}, and R^{411B} to R_{414B} respectively represent the same as the ring A₁, the ring A₂, R³¹¹ to R³¹⁴, and R⁴¹¹ to R⁴¹⁴ in the formulae (15A) to (18A); and
*5 represents the same as *3 in the formula (1B).

In the formula (14B), it is preferable that no combination of adjacent two or more of R⁵¹¹ to R⁵¹⁴, R⁵²⁰, R⁶¹¹ to R⁶¹⁴, and R⁶²⁰ are mutually bonded.

In the formula (15B), it is preferable that no combination of adjacent two or more of R^{31B} to R_{314B} and R^{411B} to R_{414B} are mutually bonded.

In the formula (15B), Z¹² in the formula (20A) representing the ring B₂ is preferably a sulfur atom.

In an exemplary embodiment, m is 1, 2, 3, or 4. In an exemplary embodiment, m is 1, 2, or 3. In an exemplary embodiment, m is 1 or 2. In an exemplary embodiment, m is 1.

In the exemplary embodiment, the compound represented by the formula (1) is preferably a compound represented by one of formulae (110) to (117) below.

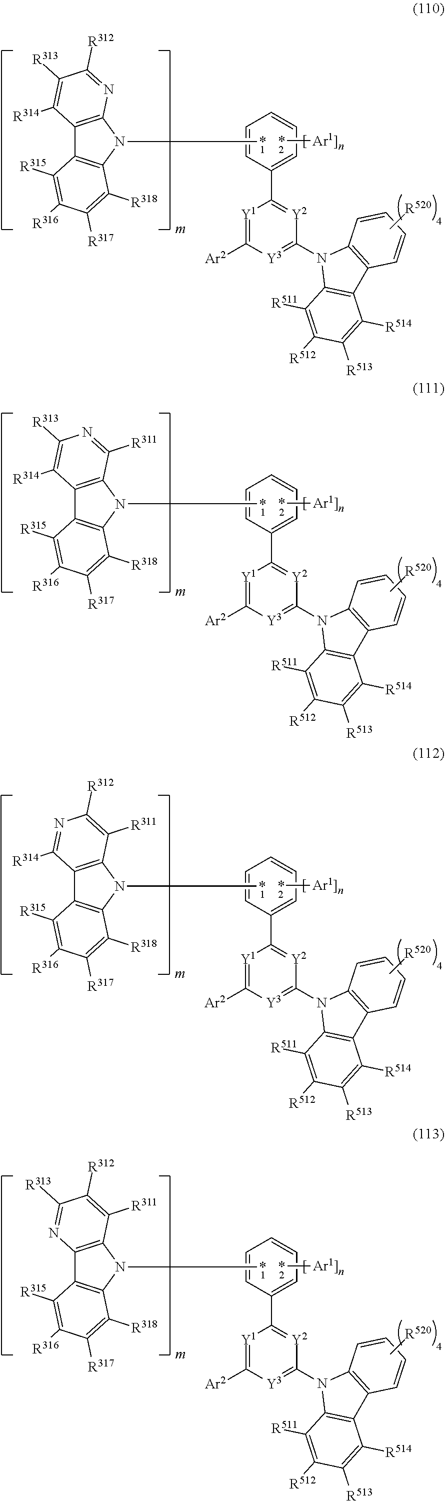

In the formulae (110) to (113):
R³¹¹ to R³¹⁸ respectively represent the same as R³¹¹ to R³¹⁸ in the formulae (11A) to (14A);
Y¹ to Y³, Ar¹, Ar², m and n respectively represent the same as Y¹ to Y³, Ar¹, Ar², m and n in the formula (1);
*1 and *2 respectively represent the same as *1 and *2 in the formula (1); and R⁵¹¹ to R⁵¹⁴ and R⁵²⁰ respectively represent the same as R⁵¹¹ to R⁵¹⁴ and R⁵²⁰ in the formula (11B).

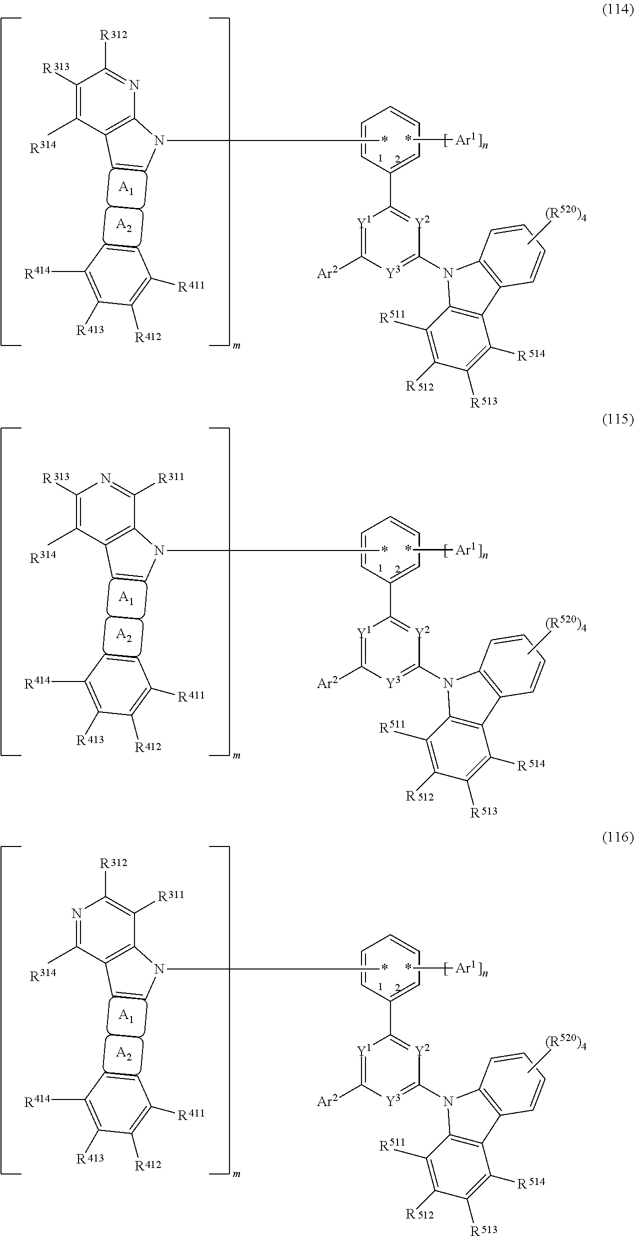

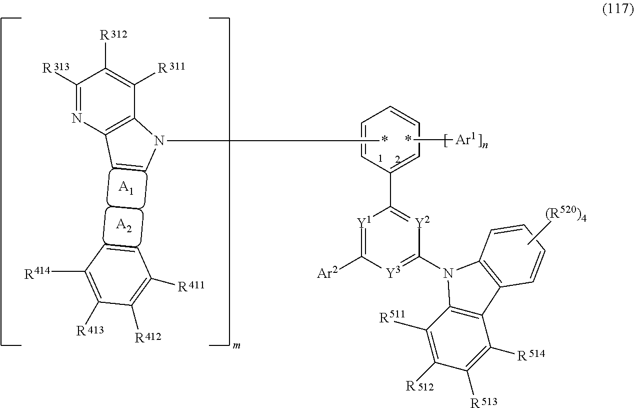

In the formulae (114) to (117):
a ring A₁, a ring A₂, R³¹¹ to R³¹⁴, and R⁴¹¹ to R⁴¹⁴ respectively represent the same as the ring A₁, the ring A₂, R³¹¹ to R³¹⁴, and R⁴¹¹ to R⁴¹⁴ in the formulae (15A) to (18A);
Y¹ to Y³, Ar¹, Ar², m and n respectively represent the same as Y¹ to Y³, Ar¹, Ar², m and n in the formula (1);
*1 and *2 respectively represent the same as *1 and *2 in the formula (1); and
R⁵¹¹ to R⁵¹⁴ and R⁵²⁰ respectively represent the same as R⁵¹¹ to R⁵¹⁴ and R⁵²⁰ in the formula (11B).

In the formulae (110) to (117), it is preferable that no combination of adjacent two or more of R⁵¹¹ to R⁵¹⁴ and R⁵²⁰ are mutually bonded.

In the compound of the exemplary embodiment, Rx, R₁, R², R³, R_{1A}, R^{2A}, R^{3A}, R^{21A}, R^{22A}, R₁₁ to R₁₈, and R²¹ to R²⁸ are preferably each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In the compound of the exemplary embodiment, R³¹¹ to R³¹⁸, R⁴¹¹ to R₄₁₄, R⁴¹⁵, R⁵¹¹ to R⁵¹⁴, R⁵²⁰, R⁶¹¹ to R⁶¹⁴, R⁶²⁰, R^{311B} to R_{314B}, and R^{411B} to R^{414B} are preferably each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In an exemplary embodiment, the compound represented by the formula (1) contains at least one deuterium atom.

In an exemplary embodiment, the compound represented by the formula (1) contains no deuterium atom.

In the compound of the exemplary embodiment, R₉₀₁, R₉₀₂, R₉₀₃, R₉₀₄, R₉₀₅, R₉₀₆, and R₉₀₇ are preferably each independently a hydrogen atom, an unsubstituted alkyl group having 1 to 25 carbon atoms, an unsubstituted aryl group having 6 to 25 ring carbon atoms, or an unsubstituted heterocyclic group having 5 to 25 ring atoms.

In the compound of the exemplary embodiment, the substituent for the “substituted or unsubstituted” group is preferably a halogen atom, an unsubstituted alkyl group having 1 to 25 carbon atoms, an unsubstituted aryl group having 6 to 25 ring carbon atoms, or an unsubstituted heterocyclic group having 5 to 25 ring atoms.

In the compound of the exemplary embodiment, the substituent for the “substituted or unsubstituted” group is more preferably an unsubstituted alkyl group having 1 to 10 carbon atoms, an unsubstituted aryl group having 6 to 12 ring carbon atoms, or an unsubstituted heterocyclic group having 5 to 12 ring atoms.

In the compound of the exemplary embodiment, the groups specified to be “substituted or unsubstituted” are each also preferably an “unsubstituted” group.

The compound of the exemplary embodiment is preferably a material to be used for an emitting layer.

The compound of the exemplary embodiment is preferably a host material.

The compound of the exemplary embodiment is preferably a sensitizing material.

The compound of the exemplary embodiment is preferably a compound exhibiting thermally activated delayed fluorescence.

The compound of the exemplary embodiment is preferably a sensitizing material and a compound exhibiting thermally activated delayed fluorescence.

### Thermally Activated Delayed Fluorescence

Herein, thermally activated delayed fluorescence is occasionally referred to as delayed fluorescence.

Delayed fluorescence is explained in “Yuki Hando-tai no Debaisu Bussei (Device Physics of Organic Semiconductors)” (edited by ADACHI, Chihaya, published by Kodansha, on pages 261-268). This document describes that, if an energy difference ΔE₁₃ of a fluorescent material between an excited singlet state and an excited triplet state is reducible, a reverse energy transfer from the excited triplet state to the excited singlet state, which usually occurs at a low transition probability, would occur at a high efficiency to express thermally activated delayed fluorescence (TADF). Further, a generation mechanism of delayed fluorescence is explained in FIG. 10.38 in the document. The TADF mechanism uses a phenomenon in which inverse intersystem crossing from triplet excitons to singlet excitons thermally occurs when a material having a small energy difference (ΔST) between singlet energy level and triplet energy level is used. As a compound exhibiting TADF properties (hereinafter also referred to as a TADF compound), for instance, a compound in which a donor moiety and an acceptor moiety are bonded in a molecule is known.

In general, the emission of delayed fluorescence can be confirmed by measuring the transient PL (photoluminescence).

The behavior of delayed fluorescence can also be analyzed based on the decay curve obtained from the transient PL measurement. The transient PL measurement is a method of irradiating a sample with a pulse laser to excite the sample, and measuring the decay behavior (transient characteristics) of PL emission after the irradiation is stopped. PL emission in TADF compounds is classified into a light emission component from a singlet exciton generated by the first PL excitation and a light emission component from a singlet exciton generated via a triplet exciton. The lifetime of the singlet exciton generated by the first PL excitation is on the order of nanoseconds and is very short. Therefore, light emission from the singlet exciton rapidly attenuates after irradiation with the pulse laser.

On the other hand, the delayed fluorescence is gradually attenuated due to light emission from a singlet exciton generated via a triplet exciton having a long lifetime. As described above, there is a large temporal difference between the light emission from the singlet exciton generated by the first PL excitation and the light emission from the singlet exciton generated via the triplet exciton. Therefore, the luminous intensity derived from delayed fluorescence can be determined.

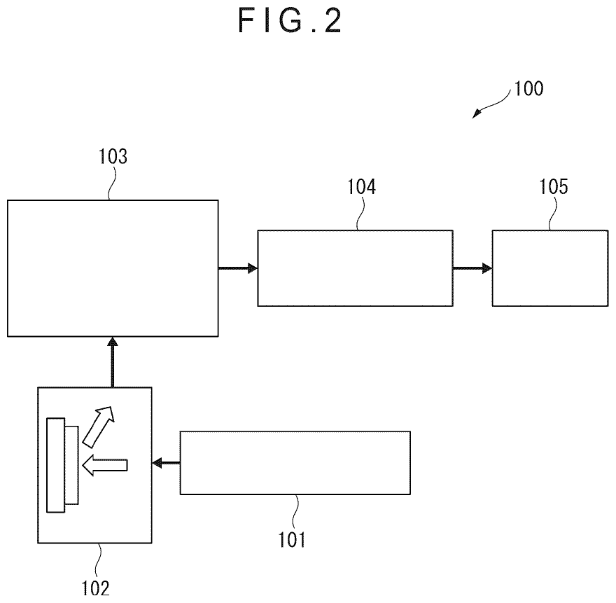

FIG. 2 schematically illustrates an exemplary apparatus for measuring the transient PL. An example of a measurement method of the transient PL using FIG. 2 and an example of behavior analysis of delayed fluorescence will be described.

A transient PL measuring apparatus 100 in FIG. 2 includes: a pulse laser 101 capable of radiating light having a predetermined wavelength; a sample chamber 102 configured to house a measurement sample; a spectrometer 103 configured to divide light radiated from the measurement sample; a streak camera 104 configured to provide a two-dimensional image; and a personal computer 105 configured to import and analyze the two-dimensional image. The transient PL may be measured by any other apparatus than the apparatus illustrated in FIG. 2.

The sample housed in the sample chamber 102 is obtained by forming a thin film, in which a matrix material is doped with a doping material at a concentration of 12 mass %, on a quartz substrate.

The thin film sample housed in the sample chamber 102 is irradiated with the pulse laser from the pulse laser 101 to excite the doping material. Emission is extracted in a direction of 90 degrees with respect to a radiation direction of the excited light. The extracted emission is divided by the spectrometer 103 to form a two-dimensional image in the streak camera 104. As a result, the two-dimensional image is obtainable in which the ordinate axis represents the time, the abscissa axis represents the wavelength, and the bright spot represents the luminous intensity. When this two-dimensional image is taken out at a predetermined time axis, an emission spectrum in which the ordinate axis represents the luminous intensity and the abscissa axis represents the wavelength is obtainable. Moreover, when this two-dimensional image is taken out at a wavelength axis, a decay curve (transient PL) in which the ordinate axis represents the logarithm of the luminous intensity and the abscissa axis represents the time is obtainable.

For instance, a thin film sample A was prepared as described above from a compound HX1 below as the matrix material and a compound DX1 below as the doping material, and was measured in terms of the transient PL.

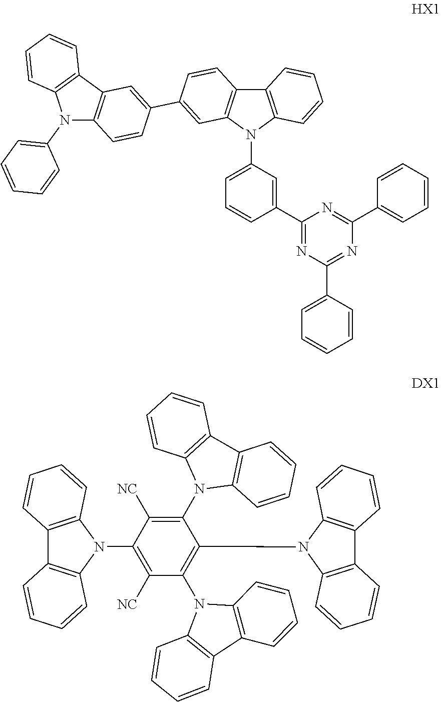 [SMILES: c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1 ; N#Cc1c(-n2c3ccccc3c3ccccc32)c(C#N)c(-n2c3ccccc3c3ccccc32)c(-n2c3ccccc3c3ccccc32)c1-n1c2ccccc2c2ccccc21]

The decay curve was analyzed using the thin film sample A and a thin film sample B. The thin film sample B was produced as above from a compound HX2 below as the matrix material and the compound DX1 as the doping material.

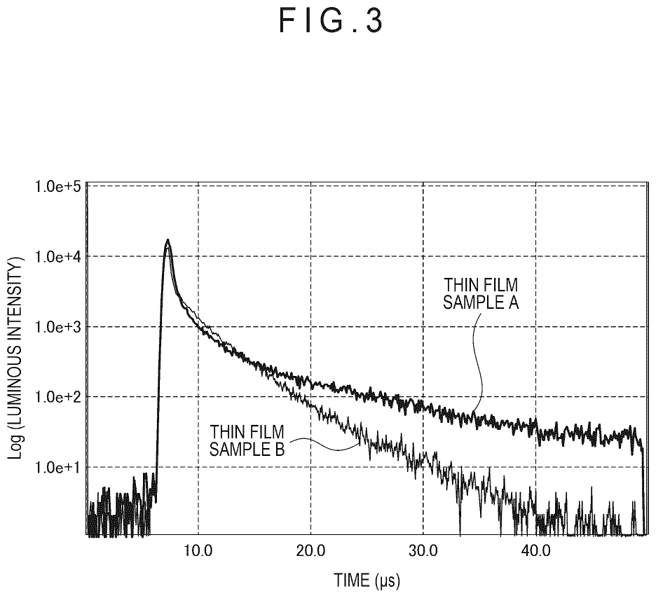

FIG. 3 illustrates decay curves obtained from the transient PL obtained by measuring the thin film samples A and B.

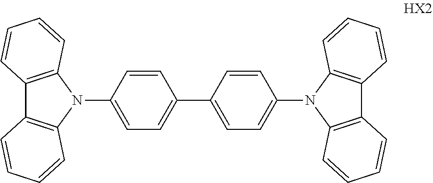 [SMILES: c1ccc2c(c1)c1ccccc1n2-c1ccc(-c2ccc(-n3c4ccccc4c4ccccc43)cc2)cc1]

As described above, an emission decay curve in which the ordinate axis represents the luminous intensity and the abscissa axis represents the time can be obtained by the transient PL measurement. Based on the emission decay curve, a fluorescence intensity ratio between fluorescence emitted from a singlet state generated by photo-excitation and delayed fluorescence emitted from a singlet state generated by reverse energy transfer via a triplet state can be estimated. In a delayed fluorescent material, a ratio of the intensity of the slowly decaying delayed fluorescence to the intensity of the promptly decaying fluorescence is relatively large.

Specifically, Prompt emission and Delay emission are present as emission from the delayed fluorescent material. Prompt emission is observed promptly when the excited state is achieved by exciting the compound of the exemplary embodiment with a pulse beam (i.e., a beam emitted from a pulse laser) having a wavelength absorbable by the delayed fluorescent material. Delay emission is observed not promptly when the excited state is achieved but after the excited state is achieved.

An amount of Prompt emission, an amount of Delay emission and a ratio between their amounts can be obtained according to the method as described in “Nature 492, 234 to 238, 2012” (Reference Document 1). The amount of Prompt emission and the amount of Delay emission may be calculated using an apparatus different from one described in Reference Document 1 or one depicted in FIG. 2.

A sample produced by the following method is used for measuring delayed fluorescence of the delayed fluorescent compound according to the exemplary embodiment. For instance, the delayed fluorescent compound according to the exemplary embodiment is dissolved in toluene to prepare a dilute solution with an absorbance of 0.05 or less at the excitation wavelength to eliminate the contribution of self-absorption. In order to prevent quenching due to oxygen, the sample solution is frozen and degassed and then sealed in a cell with a lid under an argon atmosphere to obtain an oxygen-free sample solution saturated with argon.

The fluorescence spectrum of the sample solution is measured with a spectrofluorometer FP-8600 (produced by JASCO Corporation), and the fluorescence spectrum of a 9,10-diphenylanthracene ethanol solution is measured under the same conditions. Using the fluorescence area intensities of both spectra, the total fluorescence quantum yield is calculated by Equation (1) in Morris et al. J. Phys. Chem. 80 (1976) 969.

In the exemplary embodiment, provided that an amount of Prompt emission of a measurement target compound is denoted by X_{P} and an amount of Delay emission is denoted by X_{D}, a value of X_{D}/X_{P} is preferably 0.05 or more.

The amounts of Prompt emission and Delay emission and the ratio between their amounts in compounds other than the delayed fluorescent compound herein are measured in the same manner as those of the delayed fluorescent compound according to the exemplary embodiment.

### ΔST

In the exemplary embodiment, a difference (S₁− T_{77K}) between a lowest singlet energy S₁ and an energy gap T_{77K} at 77 K is defined as ΔST.

A difference ΔST(M2) between a lowest singlet energy S₁(M2) of the compound of the exemplary embodiment (preferably the delayed fluorescent compound) and an energy gap T_{77K}(M2) at 77 K of the compound of the exemplary embodiment is preferably less than 0.5 eV, more preferably less than 0.3 eV, still more preferably less than 0.2 eV, still further more preferably less than 0.1 eV, and yet still further preferably less than 0.01 eV. That is, ΔST(M2) preferably satisfies a relationship of a numerical formula (Numerical Formula 10, Numerical Formula 1, Numerical Formula 1A, Numerical Formula 1B, or Numerical Formula 1C) below.
ΔST(M2)=S₁(M2)−T_{77K}(M2)<0.5 eV . . . (Numerical Formula 10)
ΔST(M2)=S₁(M2)−T_{77K}(M2)<0.3 eV . . . (Numerical Formula 1)
ΔST(M2)=S₁(M2)−T_{77K}(M2)<0.2 eV . . . (Numerical Formula 1A)
ΔST(M2)=S₁(M2)−T_{77K}(M2)<0.1 eV . . . (Numerical Formula 1B)
ΔST(M2)=S₁(M2)−T_{77K}(M2)<0.01 eV . . . (Numerical Formula 1C)
   Relationship between Triplet Energy and Energy Gap at 77 K

Here, a relationship between a triplet energy and an energy gap at 77 K will be described. In the exemplary embodiment, the energy gap at 77 K is different from a typical triplet energy in some aspects.

The triplet energy is measured as follows. First, a solution in which a compound (measurement target) is dissolved in an appropriate solvent is encapsulated in a quartz glass tube to prepare a sample. A phosphorescent spectrum (ordinate axis: phosphorescent luminous intensity, abscissa axis: wavelength) of the sample is measured at a low temperature (77 K). A tangent is drawn to the rise of the phosphorescent spectrum close to the short-wavelength region. The triplet energy is calculated by a predetermined conversion equation based on a wavelength value at an intersection of the tangent and the abscissa axis.

Here, the thermally activated delayed fluorescent compound among the compounds according to the exemplary embodiment is preferably a compound having a small ΔST. When ΔST is small, intersystem crossing and inverse intersystem crossing are likely to occur even at a low temperature (77 K), so that the singlet state and the triplet state coexist. As a result, the spectrum to be measured as above includes emission from both the singlet state and the triplet state. Although it is difficult to distinguish the emission from the singlet state from the emission from the triplet state, the value of the triplet energy is basically considered dominant.

Accordingly, in the exemplary embodiment, the triplet energy is measured by the same method as a typical triplet energy T, but a value measured in the following manner is referred to as an energy gap T_{77K} in order to differentiate the measured energy from the typical triplet energy in a strict meaning. A measurement target compound is dissolved in EPA (diethylether:isopentane:ethanol=5:52 in volume ratio) at a concentration of 10 μmol/L, and the obtained solution was put in a quartz cell to provide a measurement sample. A phosphorescent spectrum (ordinate axis: phosphorescent luminous intensity, abscissa axis: wavelength) of the measurement sample is measured at a low temperature (77 K). A tangent is drawn to the rise of the phosphorescent spectrum close to the short-wavelength region. An energy amount is calculated by a conversion equation (F1) below based on a wavelength value λ_{edge}[nm] at an intersection of the tangent and the abscissa axis and is defined as an energy gap T_{77K} at 77 K.
Conversion Equation (F1): T_{77K}[eV]=1239.85/λ_{edge}

The tangent to the rise of the phosphorescence spectrum close to the short-wavelength region is drawn as follows. While moving on a curve of the phosphorescence spectrum from the short-wavelength region to the local maximum value closest to the short-wavelength region among the local maximum values of the phosphorescence spectrum, a tangent is checked at each point on the curve toward the long-wavelength of the phosphorescence spectrum. An inclination of the tangent is increased along the rise of the curve (i.e., a value of the ordinate axis is increased). A tangent drawn at a point of the local maximum inclination (i.e., a tangent at an inflection point) is defined as the tangent to the rise of the phosphorescence spectrum close to the short-wavelength region.

A local maximum point where a peak intensity is 15% or less of the maximum peak intensity of the spectrum is not counted as the above-mentioned local maximum peak intensity closest to the short-wavelength region. The tangent drawn at a point that is closest to the local maximum peak intensity closest to the short-wavelength region and where the inclination of the curve is the local maximum is defined as a tangent to the rise of the phosphorescence spectrum close to the short-wavelength region.

For phosphorescence measurement, a spectrophotofluorometer body F-4500 (produced by Hitachi High-Technologies Corporation) is usable. The measurement apparatus is not limited thereto. A combination of a cooling unit, a low temperature container, an excitation light source and a light-receiving unit may be used for measurement.

### Lowest Singlet Energy S₁

A method for measuring the lowest singlet energy S₁ with the use of a solution (occasionally referred to as a solution method) is exemplified by a method below.

A toluene solution of a measurement target compound at a concentration of 10 μmol/L is prepared and put in a quartz cell. An absorption spectrum (ordinate axis: absorption intensity, abscissa axis: wavelength) of the thus-obtained sample is measured at a normal temperature (300 K). A tangent is drawn to the fall of the absorption spectrum close to the long-wavelength region, and a wavelength value kedge (nm) at an intersection of the tangent and the abscissa axis is assigned to a conversion equation (F2) below to calculate the lowest singlet energy.
Conversion Equation (F2): S₁[eV]=1239.85/λ_{edge}

Any apparatus for measuring the absorption spectrum is usable. For instance, a spectrophotometer (U3310 produced by Hitachi, Ltd.) is usable.

The tangent to the fall of the absorption spectrum close to the long-wavelength region is drawn as follows. While moving on a curve of the absorption spectrum from the local maximum value closest to the long-wavelength region, among the local maximum values of the absorption spectrum, in a long-wavelength direction, a tangent at each point on the curve is checked. An inclination of the tangent is decreased and increased in a repeated manner as the curve falls (i.e., a value of the ordinate axis is decreased). A tangent drawn at a point where the inclination of the curve is the local minimum closest to the long-wavelength region (except when absorbance is 0.1 or less) is defined as the tangent to the fall of the absorption spectrum close to the long-wavelength region.

The local maximum absorbance of 0.2 or less is not counted as the above-mentioned local maximum absorbance closest to the long-wavelength region.

### Method for Producing Compound of the Exemplary Embodiment

The compound of the exemplary embodiment (the compound represented by the formula (1)) can be produced by application of known substitution reactions and materials tailored for the target compound, according to a synthesis method described later in Examples or in a similar manner as the synthesis method.

### Specific Examples of Compound of the Exemplary Embodiment

Specific examples of the compound of the exemplary embodiment include compounds below. The invention, however, is not limited to the specific examples.

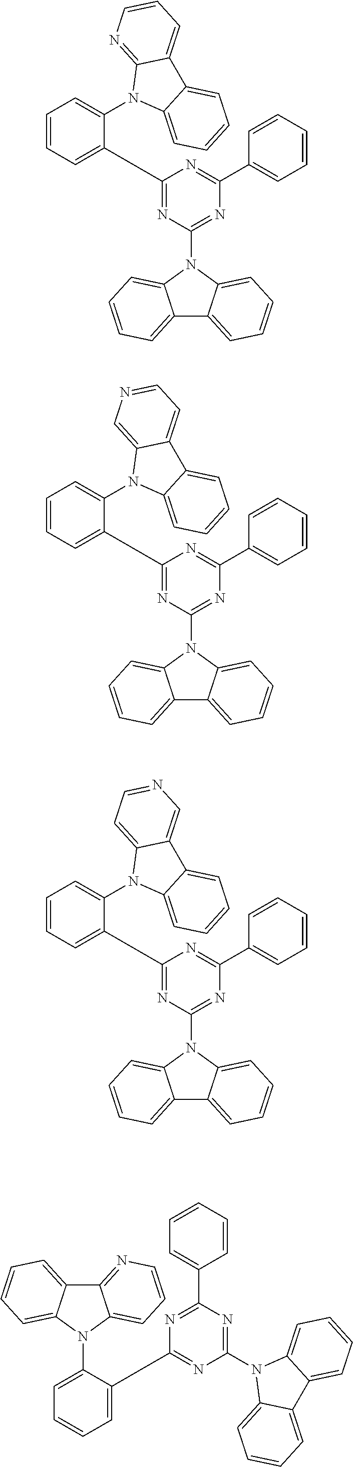

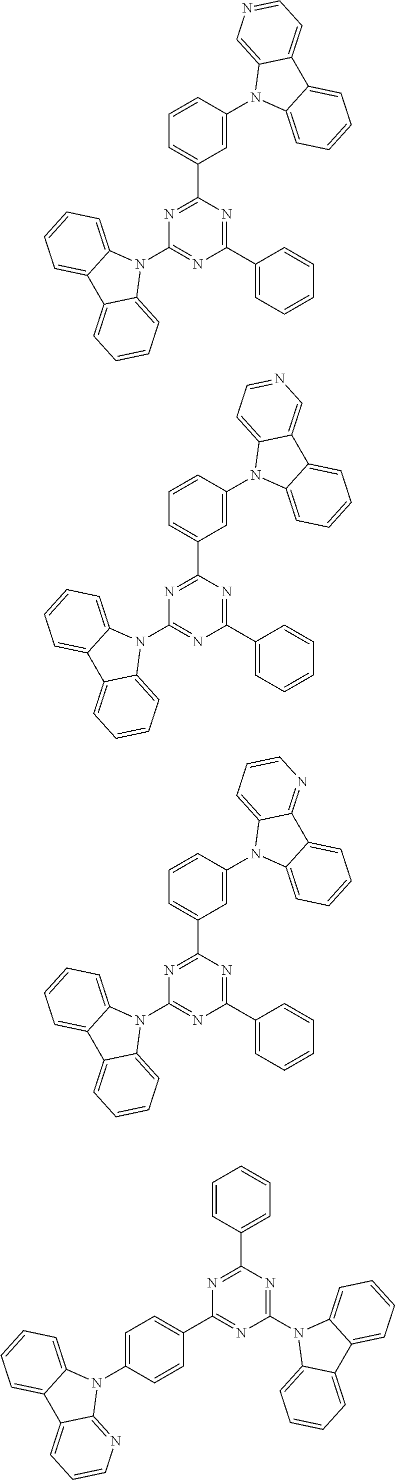 [SMILES: c1ccc(-c2nc(-c3cccc(-n4c5ccccc5c5ccncc54)c3)nc(-n3c4ccccc4c4ccccc43)n2)cc1 ; c1ccc(-c2nc(-c3cccc(-n4c5ccccc5c5cnccc54)c3)nc(-n3c4ccccc4c4ccccc43)n2)cc1 ; c1ccc(-c2nc(-c3cccc(-n4c5ccccc5c5ncccc54)c3)nc(-n3c4ccccc4c4ccccc43)n2)cc1 ; c1ccc(-c2nc(-c3ccc(-n4c5ccccc5c5cccnc54)cc3)nc(-n3c4ccccc4c4ccccc43)n2)cc1]

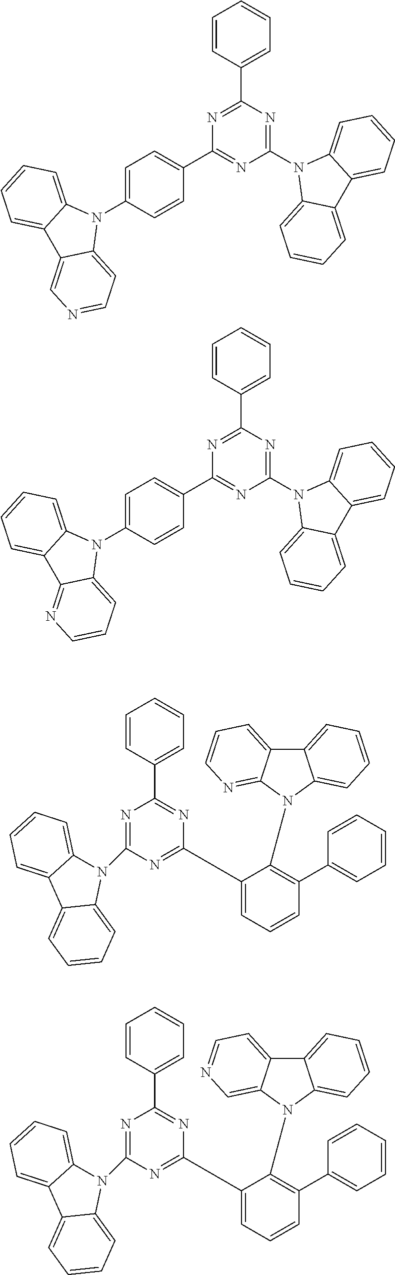 [SMILES: c1ccc(-c2nc(-c3ccc(-n4c5ccccc5c5cnccc54)cc3)nc(-n3c4ccccc4c4ccccc43)n2)cc1 ; c1ccc(-c2nc(-c3ccc(-n4c5ccccc5c5ncccc54)cc3)nc(-n3c4ccccc4c4ccccc43)n2)cc1 ; c1ccc(-c2nc(-c3cccc(-c4ccccc4)c3-n3c4ccccc4c4cccnc43)nc(-n3c4ccccc4c4ccccc43)n2)cc1 ; c1ccc(-c2nc(-c3cccc(-c4ccccc4)c3-n3c4ccccc4c4ccncc43)nc(-n3c4ccccc4c4ccccc43)n2)cc1]

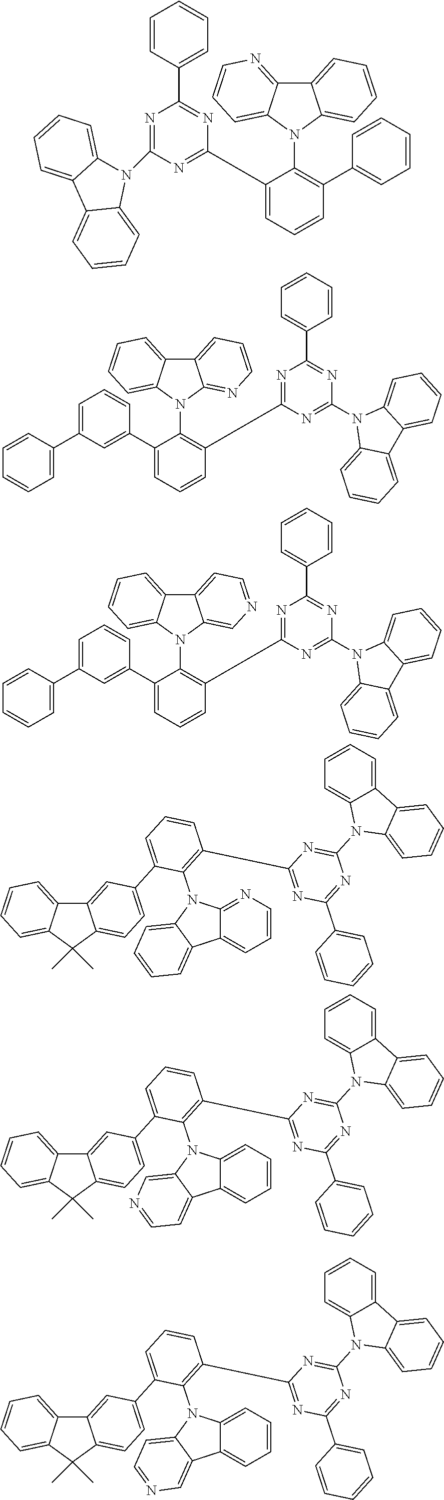

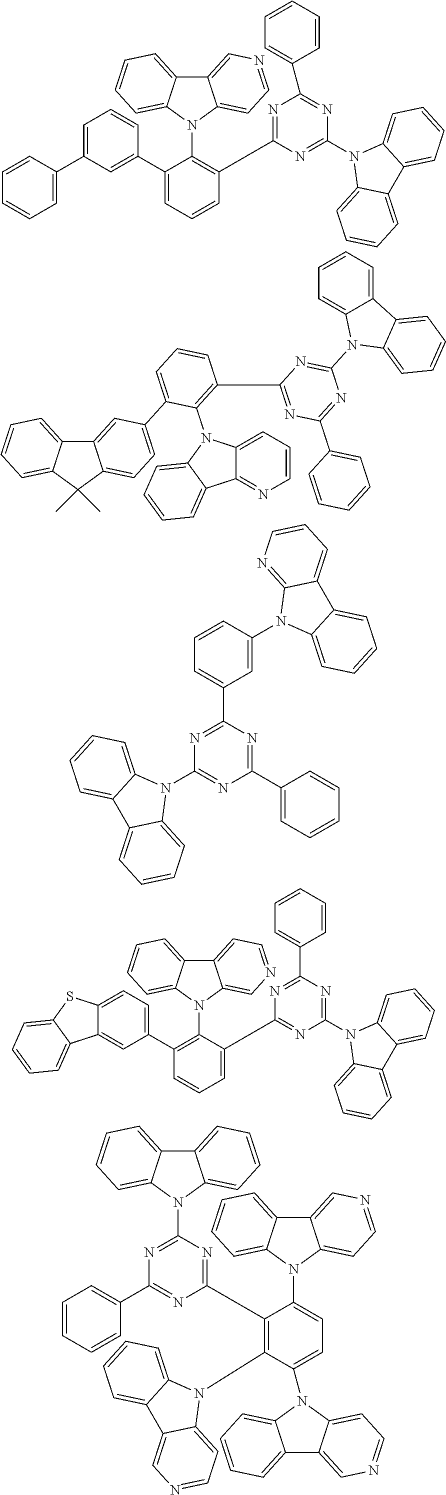

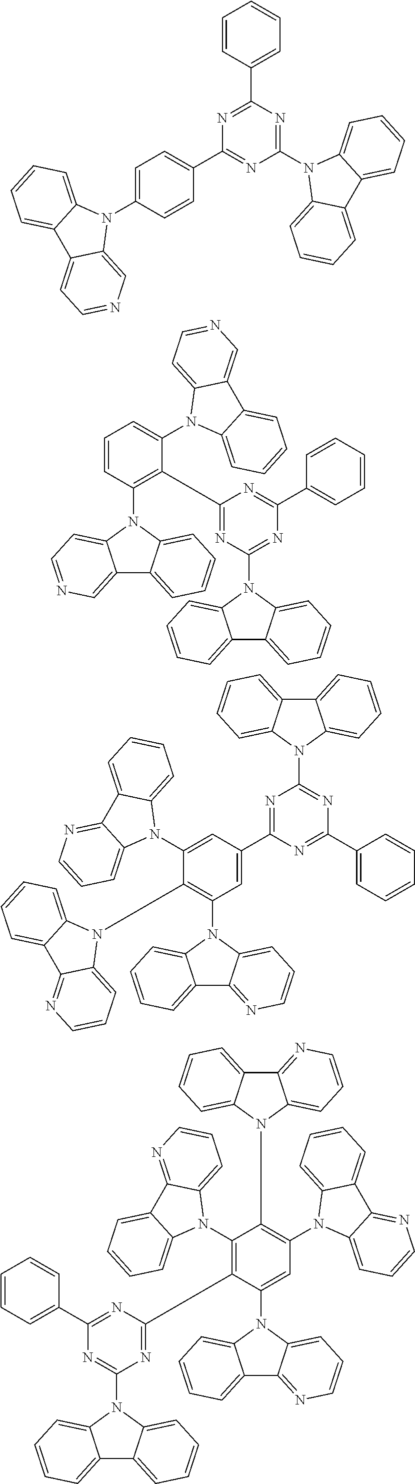

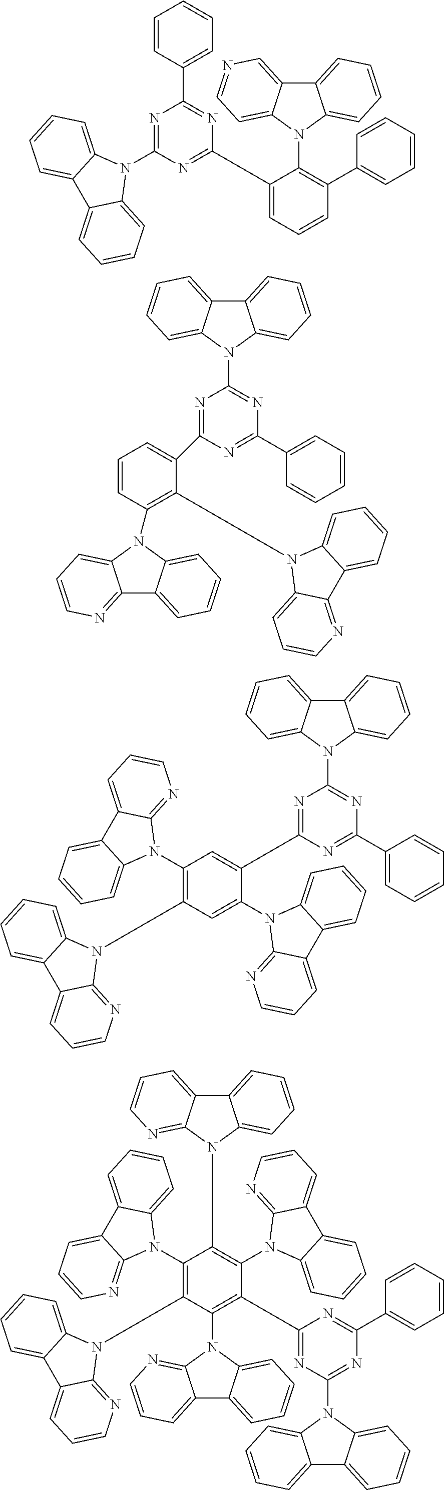

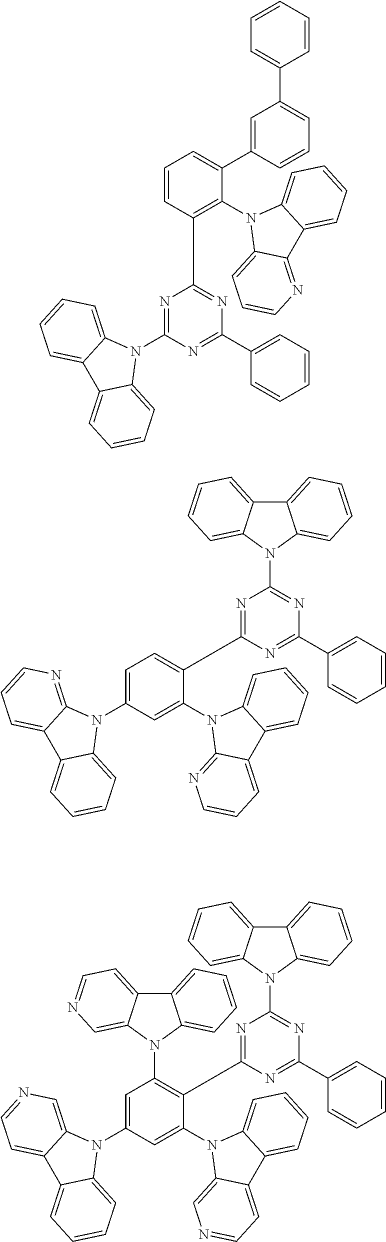 [SMILES: c1ccc(-c2cccc(-c3cccc(-c4nc(-c5ccccc5)nc(-n5c6ccccc6c6ccccc65)n4)c3-n3c4ccccc4c4ncccc43)c2)cc1 ; c1ccc(-c2nc(-c3ccc(-n4c5ccccc5c5cccnc54)cc3-n3c4ccccc4c4cccnc43)nc(-n3c4ccccc4c4ccccc43)n2)cc1 ; c1ccc(-c2nc(-c3c(-n4c5ccccc5c5ccncc54)cc(-n4c5ccccc5c5ccncc54)cc3-n3c4ccccc4c4ccncc43)nc(-n3c4ccccc4c4ccccc43)n2)cc1]

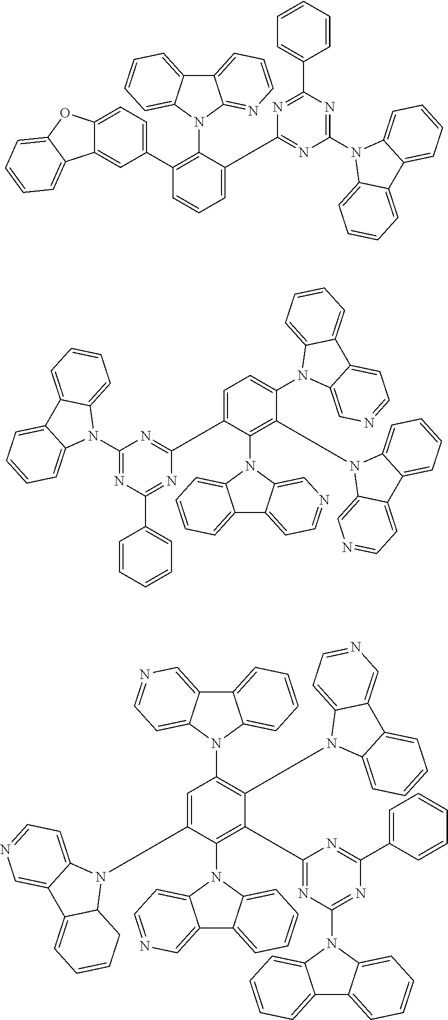 [SMILES: c1ccc(-c2nc(-c3cccc(-c4ccc5oc6ccccc6c5c4)c3-n3c4ccccc4c4cccnc43)nc(-n3c4ccccc4c4ccccc43)n2)cc1 ; c1ccc(-c2nc(-c3ccc(-n4c5ccccc5c5ccncc54)c(-n4c5ccccc5c5ccncc54)c3-n3c4ccccc4c4ccncc43)nc(-n3c4ccccc4c4ccccc43)n2)cc1 ; C1=CCC2C(=C1)c1cnccc1N2c1cc(-n2c3ccccc3c3cnccc32)c(-n2c3ccccc3c3cnccc32)c(-c2nc(-c3ccccc3)nc(-n3c4ccccc4c4ccccc43)n2)c1-n1c2ccccc2c2cnccc21]

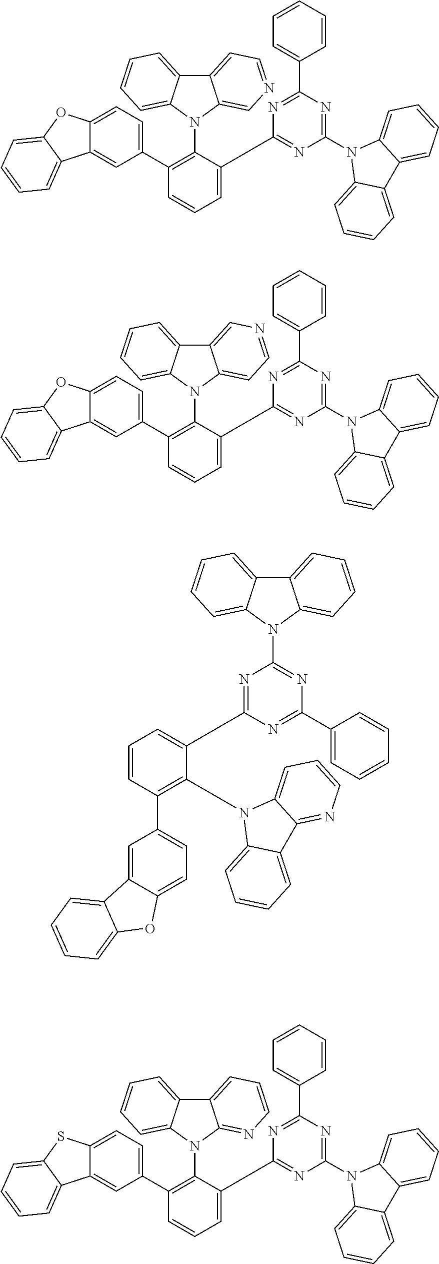 [SMILES: c1ccc(-c2nc(Cc3cccc(-c4ccc5oc6ccccc6c5c4)c3-n3c4ccccc4c4ccncc43)nc(-n3c4ccccc4c4ccccc43)n2)cc1 ; c1ccc(-c2nc(Cc3cccc(-c4ccc5oc6ccccc6c5c4)c3-n3c4ccccc4c4cnccc43)nc(-n3c4ccccc4c4ccccc43)n2)cc1 ; c1ccc(-c2nc(-c3cccc(-c4ccc5oc6ccccc6c5c4)c3-n3c4ccccc4c4ncccc43)nc(-n3c4ccccc4c4ccccc43)n2)cc1 ; c1ccc(-c2nc(Cc3cccc(-c4ccc5sc6ccccc6c5c4)c3-n3c4ccccc4c4cccnc43)nc(-n3c4ccccc4c4ccccc43)n2)cc1]

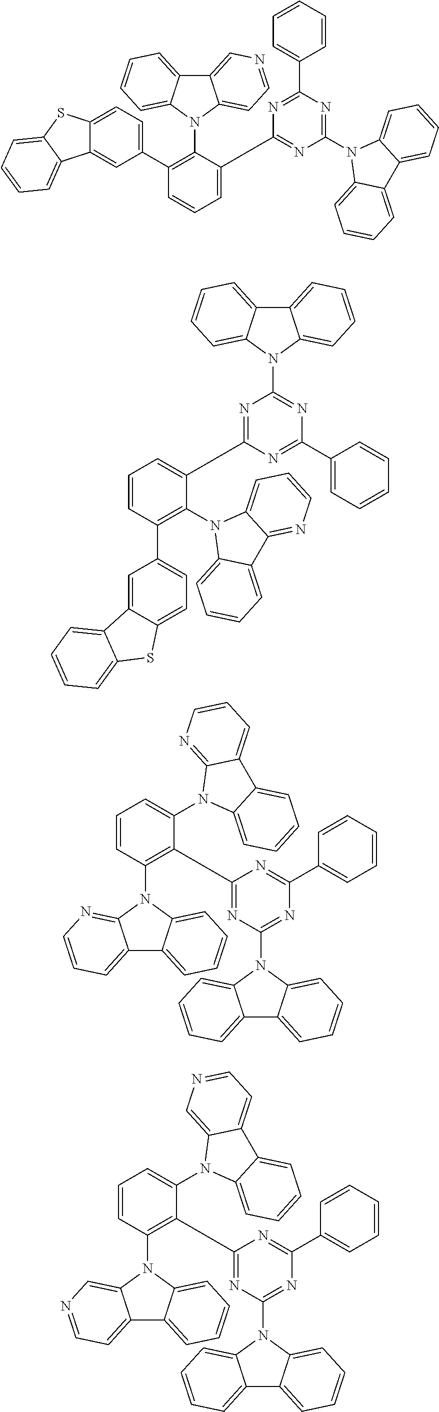

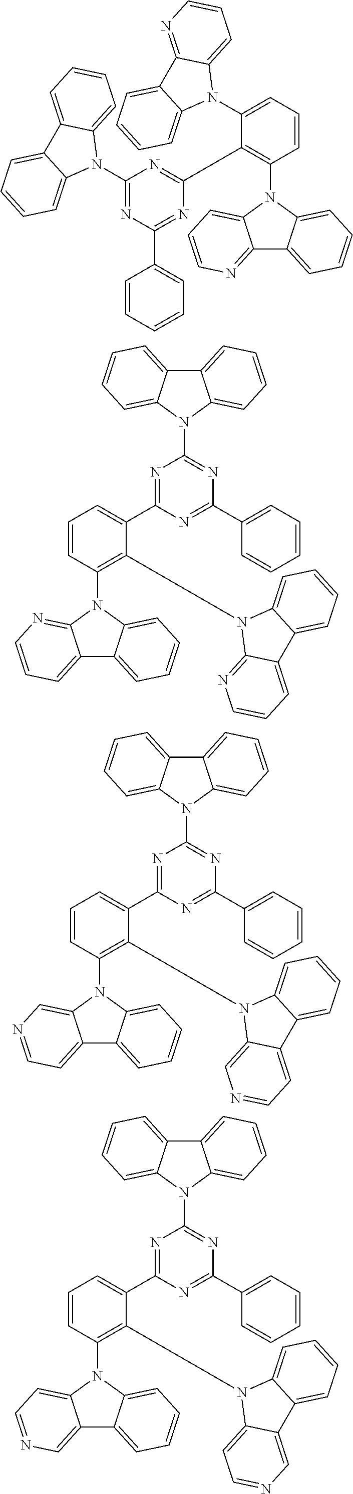

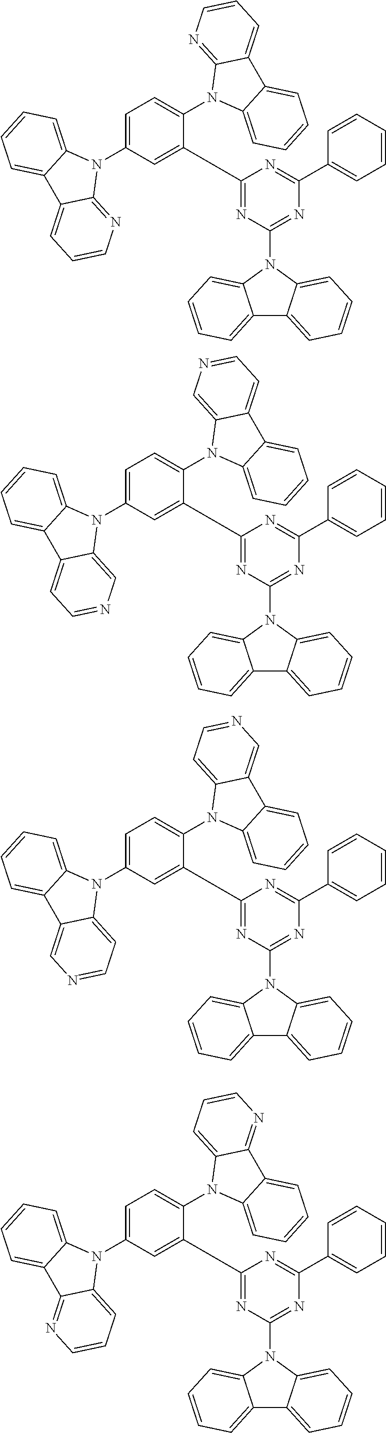

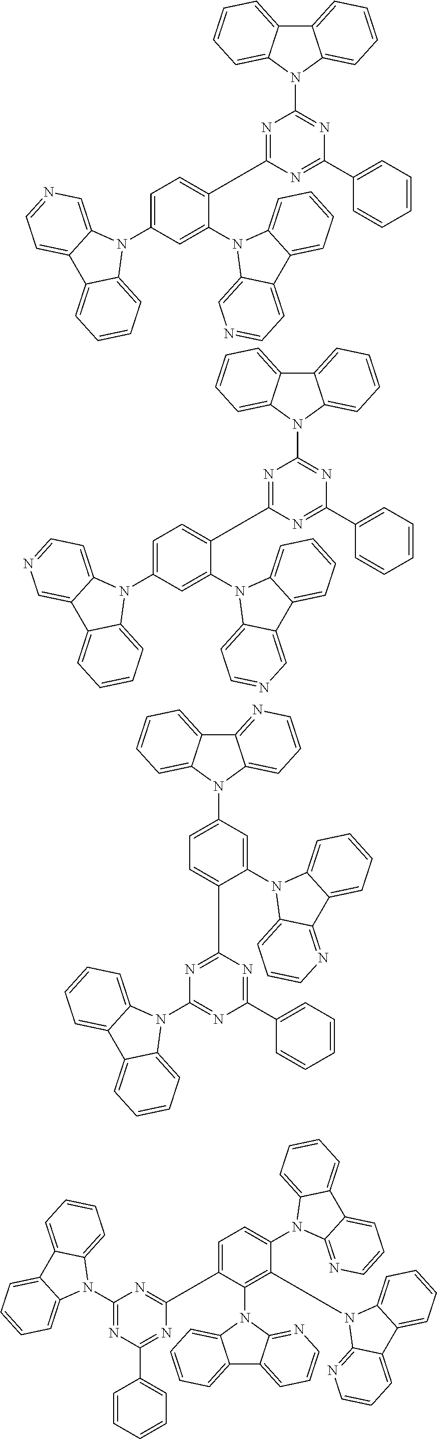

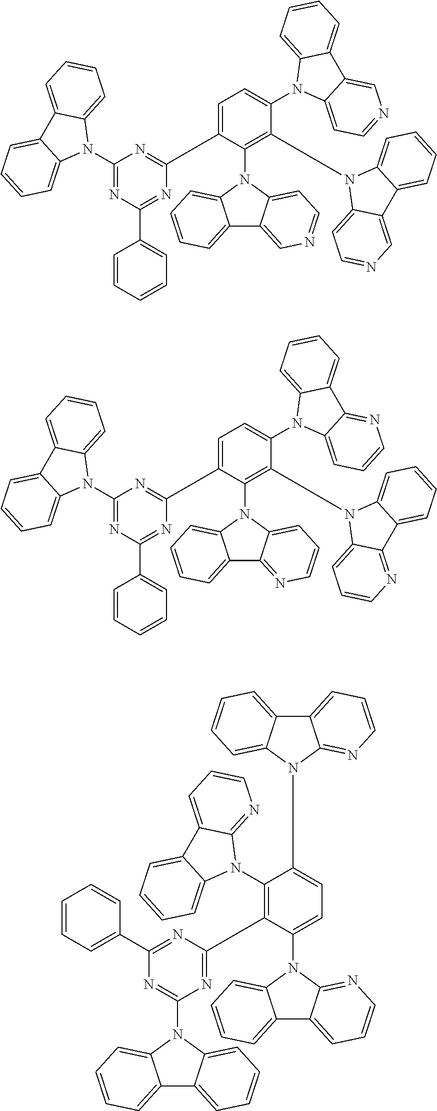 [SMILES: c1ccc(-c2nc(-c3ccc(-n4c5ccccc5c5cnccc54)c(CCn4c5ccccc5c5cnccc54)c3-n3c4ccccc4c4cnccc43)nc(-n3c4ccccc4c4ccccc43)n2)cc1 ; c1ccc(-c2nc(-c3ccc(-n4c5ccccc5c5ncccc54)c(CCn4c5ccccc5c5ncccc54)c3-n3c4ccccc4c4ncccc43)nc(-n3c4ccccc4c4ccccc43)n2)cc1 ; c1ccc(-c2nc(-c3c(-n4c5ccccc5c5cccnc54)ccc(-n4c5ccccc5c5cccnc54)c3-n3c4ccccc4c4cccnc43)nc(-n3c4ccccc4c4ccccc43)n2)cc1]

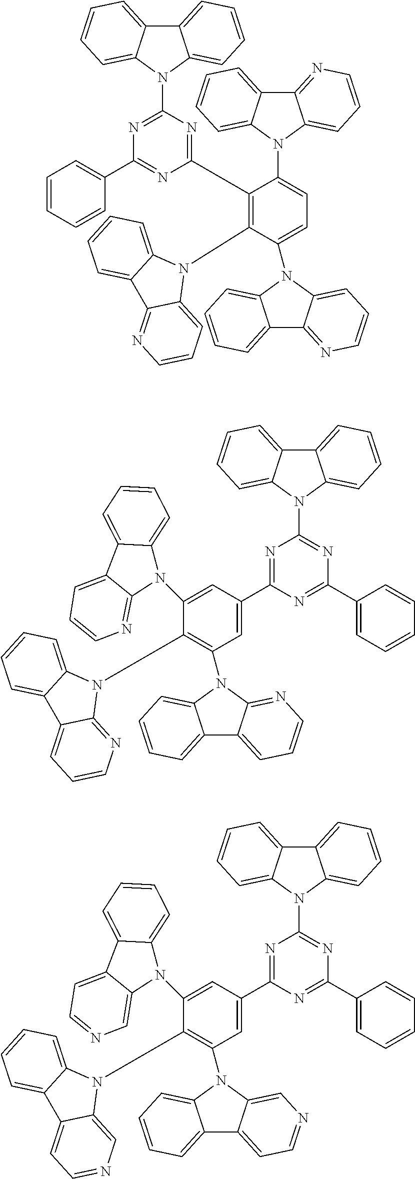 [SMILES: c1ccc(-c2nc(-c3c(-n4c5ccccc5c5ncccc54)ccc(-n4c5ccccc5c5ncccc54)c3-n3c4ccccc4c4ncccc43)nc(-n3c4ccccc4c4ccccc43)n2)cc1 ; c1ccc(-c2nc(-c3cc(-n4c5ccccc5c5cccnc54)c(CCn4c5ccccc5c5cccnc54)c(-n4c5ccccc5c5cccnc54)c3)nc(-n3c4ccccc4c4ccccc43)n2)cc1 ; c1ccc(-c2nc(-c3cc(-n4c5ccccc5c5ccncc54)c(CCn4c5ccccc5c5ccncc54)c(-n4c5ccccc5c5ccncc54)c3)nc(-n3c4ccccc4c4ccccc43)n2)cc1]

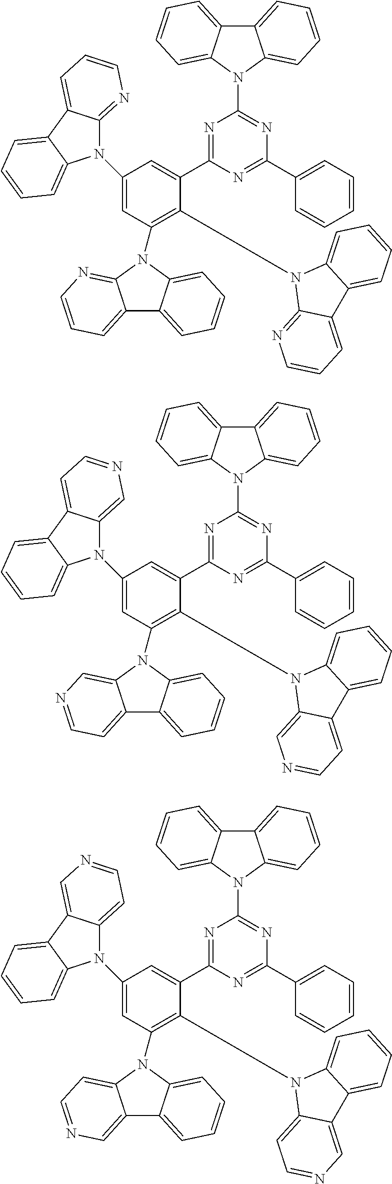

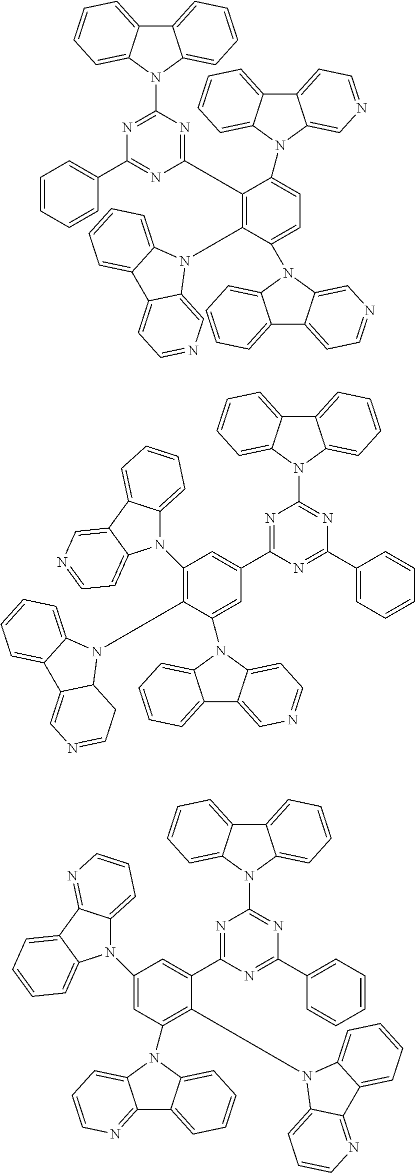

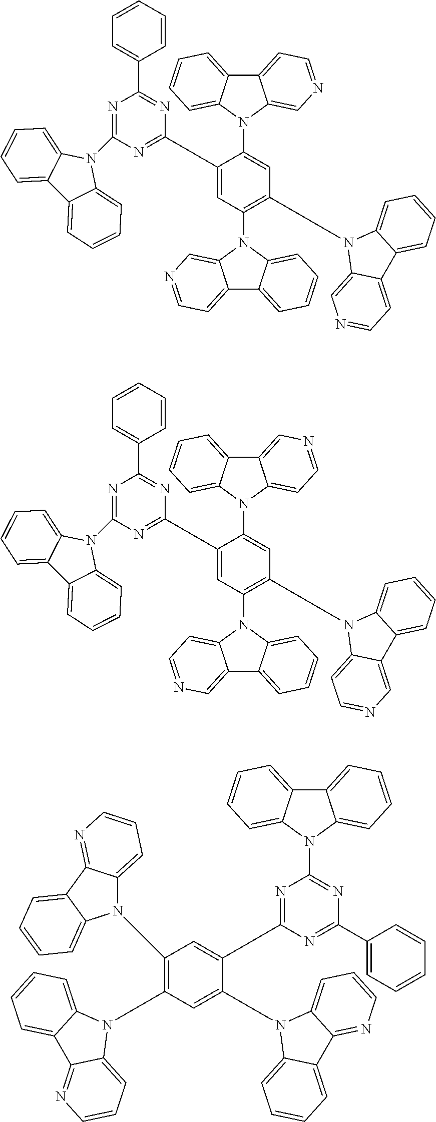 [SMILES: c1ccc(-c2nc(-c3cc(-n4c5ccccc5c5ccncc54)c(-n4c5ccccc5c5ccncc54)cc3-n3c4ccccc4c4ccncc43)nc(-n3c4ccccc4c4ccccc43)n2)cc1 ; c1ccc(-c2nc(-c3cc(-n4c5ccccc5c5cnccc54)c(-n4c5ccccc5c5cnccc54)cc3-n3c4ccccc4c4cnccc43)nc(-n3c4ccccc4c4ccccc43)n2)cc1 ; c1ccc(-c2nc(-c3cc(-n4c5ccccc5c5ncccc54)c(-n4c5ccccc5c5ncccc54)cc3-n3c4ccccc4c4ncccc43)nc(-n3c4ccccc4c4ccccc43)n2)cc1]

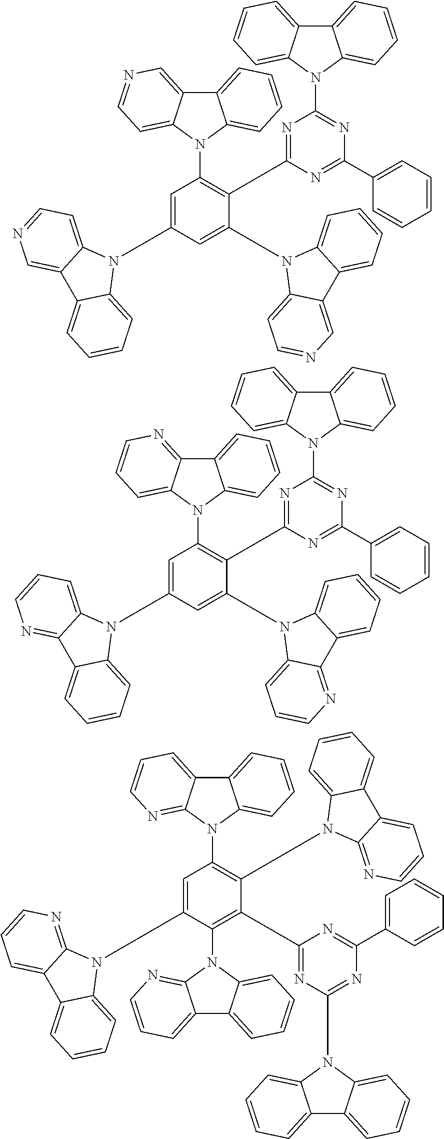

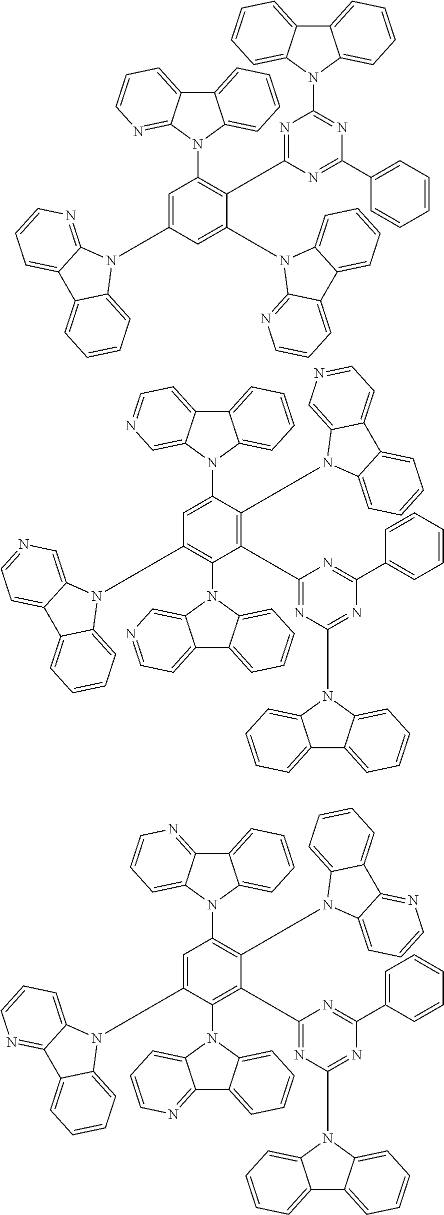

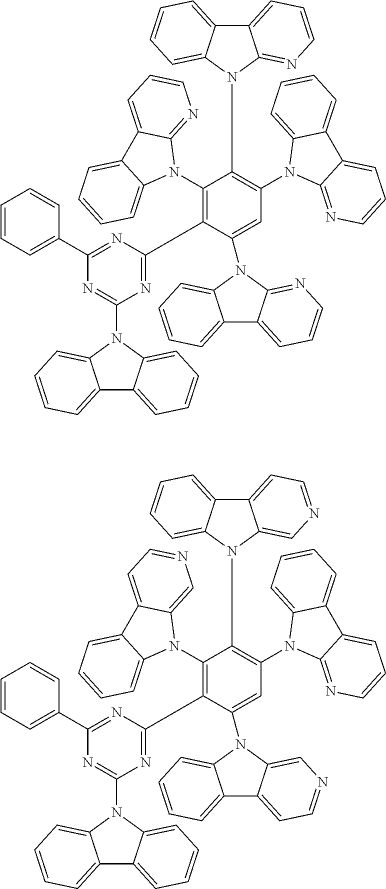 [SMILES: c1ccc(-c2nc(-c3c(-n4c5ccccc5c5cccnc54)cc(-n4c5ccccc5c5cccnc54)c(-n4c5ccccc5c5cccnc54)c3-n3c4ccccc4c4cccnc43)nc(-n3c4ccccc4c4ccccc43)n2)cc1 ; c1ccc(-c2nc(-c3c(-n4c5ccccc5c5ccncc54)cc(-n4c5ccccc5c5cccnc54)c(-n4c5ccccc5c5ccncc54)c3-n3c4ccccc4c4ccncc43)nc(-n3c4ccccc4c4ccccc43)n2)cc1]

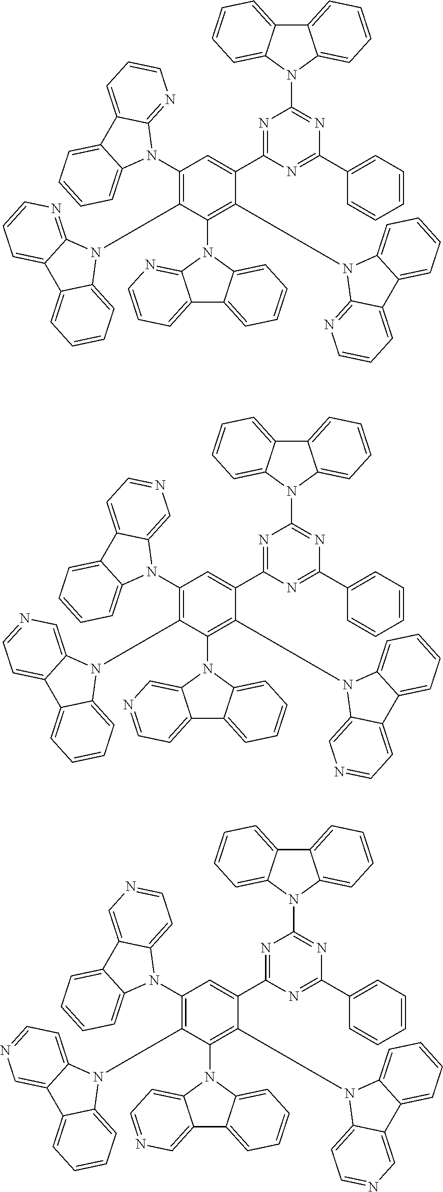 [SMILES: c1ccc(-c2nc(-c3cc(-n4c5ccccc5c5cccnc54)c(CCn4c5ccccc5c5cccnc54)c(-n4c5ccccc5c5cccnc54)c3-n3c4ccccc4c4cccnc43)nc(-n3c4ccccc4c4ccccc43)n2)cc1 ; c1ccc(-c2nc(-c3cc(-n4c5ccccc5c5ccncc54)c(CCn4c5ccccc5c5ccncc54)c(-n4c5ccccc5c5ccncc54)c3-n3c4ccccc4c4ccncc43)nc(-n3c4ccccc4c4ccccc43)n2)cc1 ; c1ccc(-c2nc(-c3cc(-n4c5ccccc5c5cnccc54)c(CCn4c5ccccc5c5cnccc54)c(-n4c5ccccc5c5cnccc54)c3-n3c4ccccc4c4cnccc43)nc(-n3c4ccccc4c4ccccc43)n2)cc1]

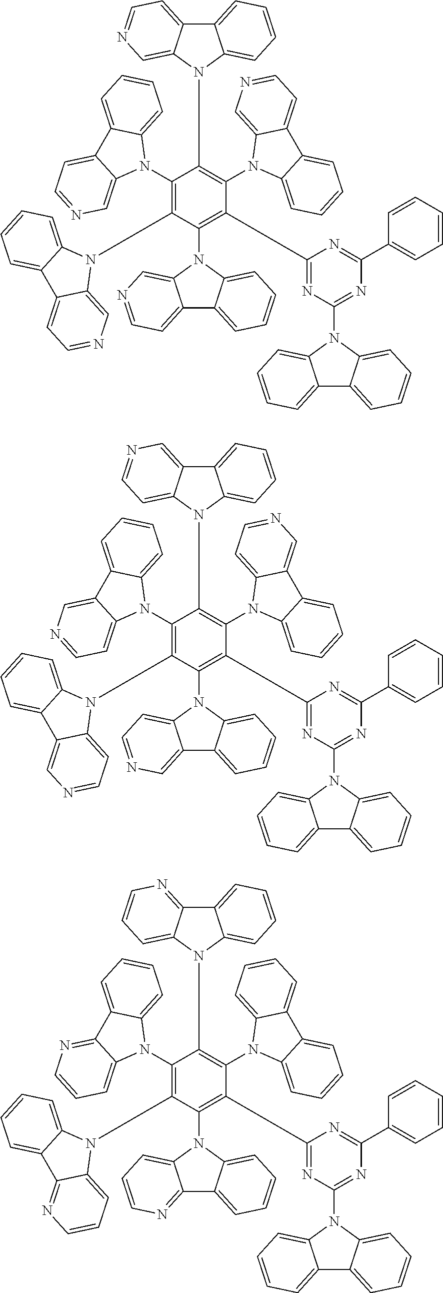 [SMILES: c1ccc(-c2nc(-c3c(-n4c5ccccc5c5ccncc54)c(-n4c5ccccc5c5ccncc54)c(-n4c5ccccc5c5ccncc54)c(-n4c5ccccc5c5ccncc54)c3-n3c4ccccc4c4ccncc43)nc(-n3c4ccccc4c4ccccc43)n2)cc1 ; c1ccc(-c2nc(-c3c(-n4c5ccccc5c5cnccc54)c(-n4c5ccccc5c5cnccc54)c(-n4c5ccccc5c5cnccc54)c(-n4c5ccccc5c5cnccc54)c3-n3c4ccccc4c4cnccc43)nc(-n3c4ccccc4c4ccccc43)n2)cc1 ; c1ccc(-c2nc(-c3c(-n4c5ccccc5c5ccccc54)c(-n4c5ccccc5c5ncccc54)c(-n4c5ccccc5c5ncccc54)c(-n4c5ccccc5c5ncccc54)c3-n3c4ccccc4c4ncccc43)nc(-n3c4ccccc4c4ccccc43)n2)cc1]

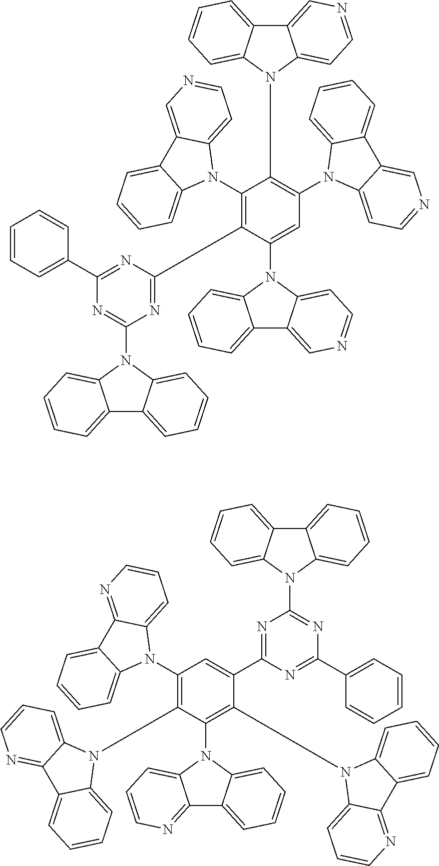 [SMILES: c1ccc(-c2nc(-c3c(-n4c5ccccc5c5cnccc54)cc(-n4c5ccccc5c5cnccc54)c(-n4c5ccccc5c5cnccc54)c3-n3c4ccccc4c4cnccc43)nc(-n3c4ccccc4c4ccccc43)n2)cc1 ; c1ccc(-c2nc(-c3cc(-n4c5ccccc5c5ncccc54)c(-n4c5ccccc5c5ncccc54)c(-n4c5ccccc5c5ncccc54)c3-n3c4ccccc4c4ncccc43)nc(-n3c4ccccc4c4ccccc43)n2)cc1]

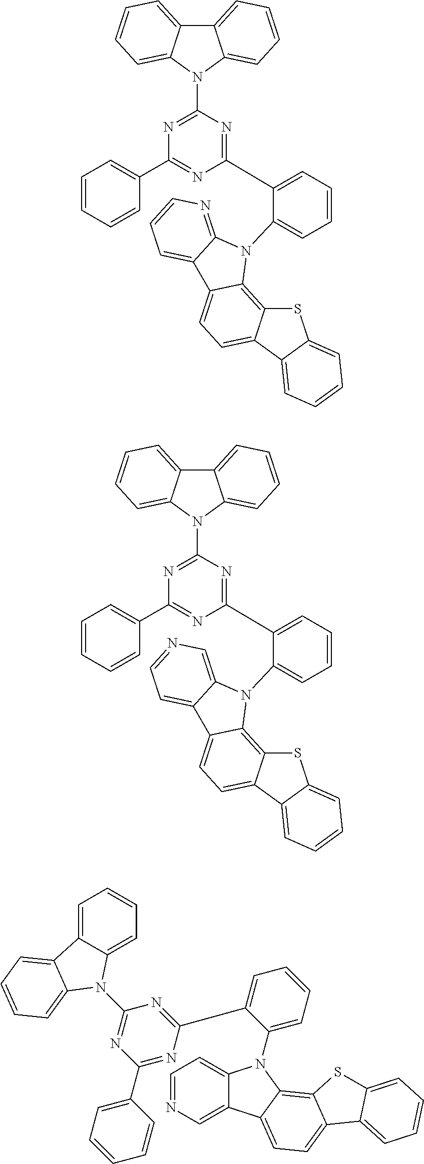 [SMILES: c1ccc(-c2nc(-c3ccccc3-n3c4ncccc4c4ccc5c6ccccc6sc5c43)nc(-n3c4ccccc4c4ccccc43)n2)cc1 ; c1ccc(-c2nc(-c3ccccc3-n3c4cnccc4c4ccc5c6ccccc6sc5c43)nc(-n3c4ccccc4c4ccccc43)n2)cc1 ; c1ccc(-c2nc(-c3ccccc3-n3c4ccncc4c4ccc5c6ccccc6sc5c43)nc(-n3c4ccccc4c4ccccc43)n2)cc1]

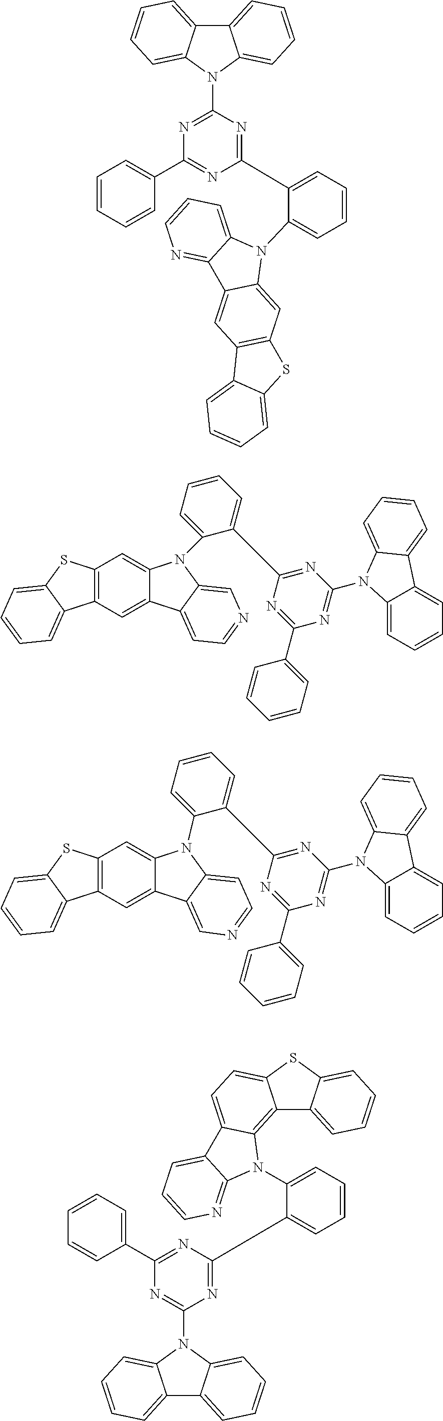

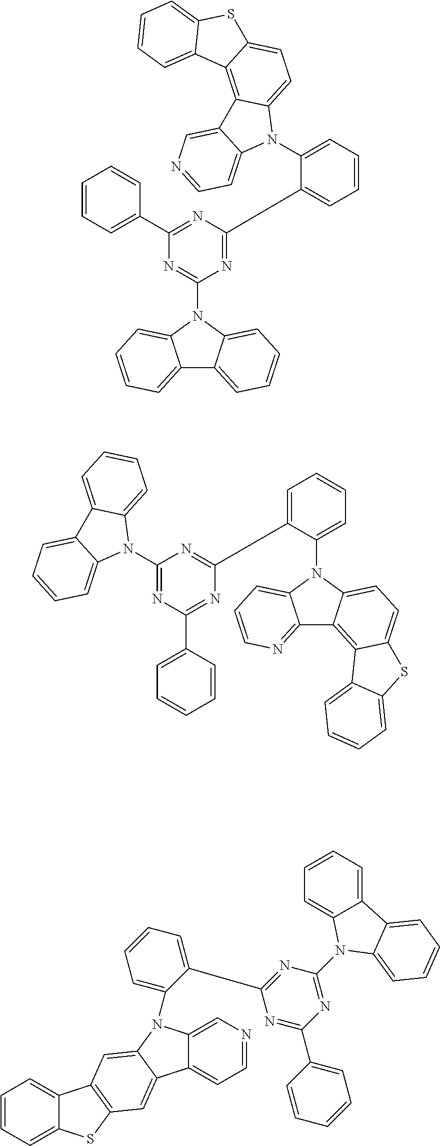 [SMILES: c1ccc(-c2nc(-c3ccccc3-n3c4ccncc4c4c5c(ccc43)sc3ccccc35)nc(-n3c4ccccc4c4ccccc43)n2)cc1 ; c1ccc(-c2nc(-c3ccccc3-n3c4cccnc4c4c5c(ccc43)sc3ccccc35)nc(-n3c4ccccc4c4ccccc43)n2)cc1 ; c1ccc(-c2nc(-c3ccccc3-n3c4cnccc4c4cc5sc6ccccc6c5cc43)nc(-n3c4ccccc4c4ccccc43)n2)cc1]

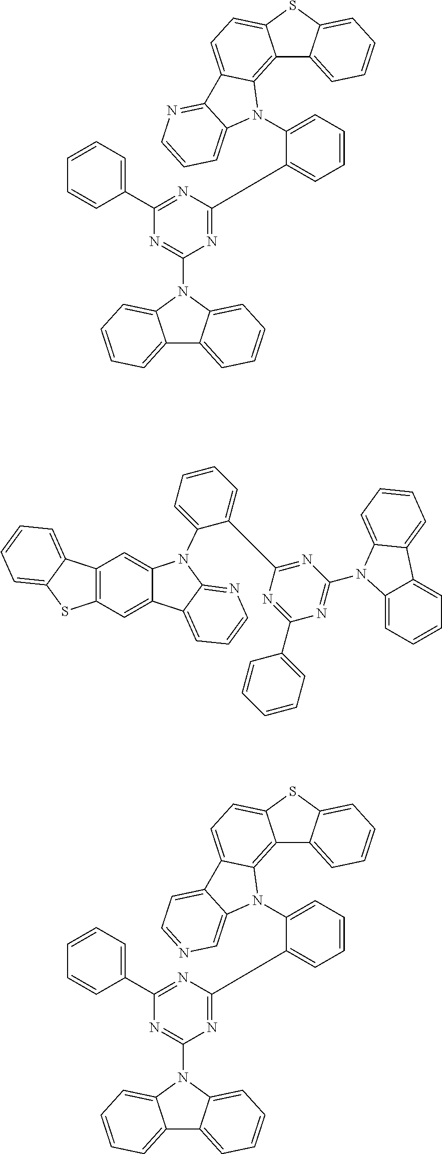 [SMILES: c1ccc(-c2nc(-c3ccccc3-n3c4cccnc4c4ccc5sc6ccccc6c5c43)nc(-n3c4ccccc4c4ccccc43)n2)cc1 ; c1ccc(-c2nc(-c3ccccc3-n3c4cc5c(cc4c4cccnc43)sc3ccccc35)nc(-n3c4ccccc4c4ccccc43)n2)cc1 ; c1ccc(-c2nc(-c3ccccc3-n3c4cnccc4c4ccc5sc6ccccc6c5c43)nc(-n3c4ccccc4c4ccccc43)n2)cc1]

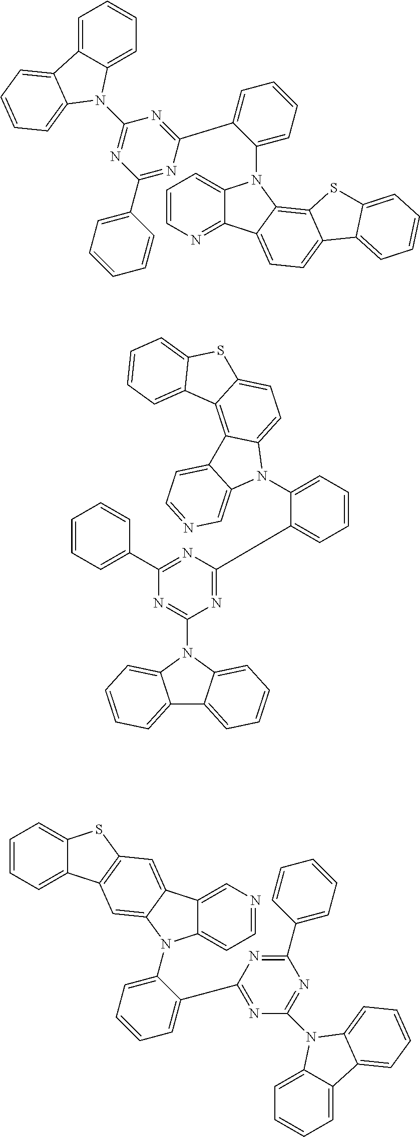 [SMILES: c1ccc(-c2nc(-c3ccccc3-n3c4cccnc4c4ccc5c6ccccc6sc5c43)nc(-n3c4ccccc4c4ccccc43)n2)cc1 ; c1ccc(-c2nc(-c3ccccc3-n3c4cnccc4c4c5c(ccc43)sc3ccccc35)nc(-n3c4ccccc4c4ccccc43)n2)cc1 ; c1ccc(-c2nc(-c3ccccc3-n3c4ccncc4c4cc5sc6ccccc6c5cc43)nc(-n3c4ccccc4c4ccccc43)n2)cc1]

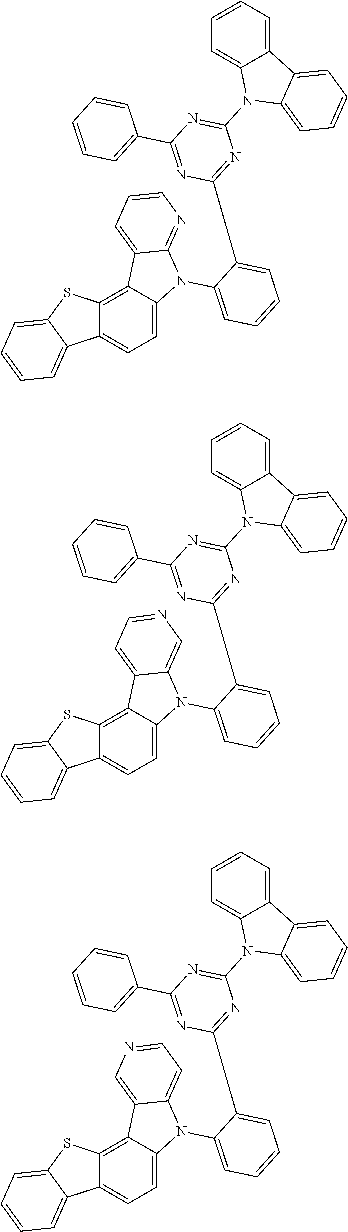 [SMILES: c1ccc(-c2nc(-c3ccccc3-n3c4ccc5c6ccccc6sc5c4c4cccnc43)nc(-n3c4ccccc4c4ccccc43)n2)cc1 ; c1ccc(-c2nc(-c3ccccc3-n3c4cnccc4c4c5sc6ccccc6c5ccc43)nc(-n3c4ccccc4c4ccccc43)n2)cc1 ; c1ccc(-c2nc(-c3ccccc3-n3c4ccncc4c4c5sc6ccccc6c5ccc43)nc(-n3c4ccccc4c4ccccc43)n2)cc1]

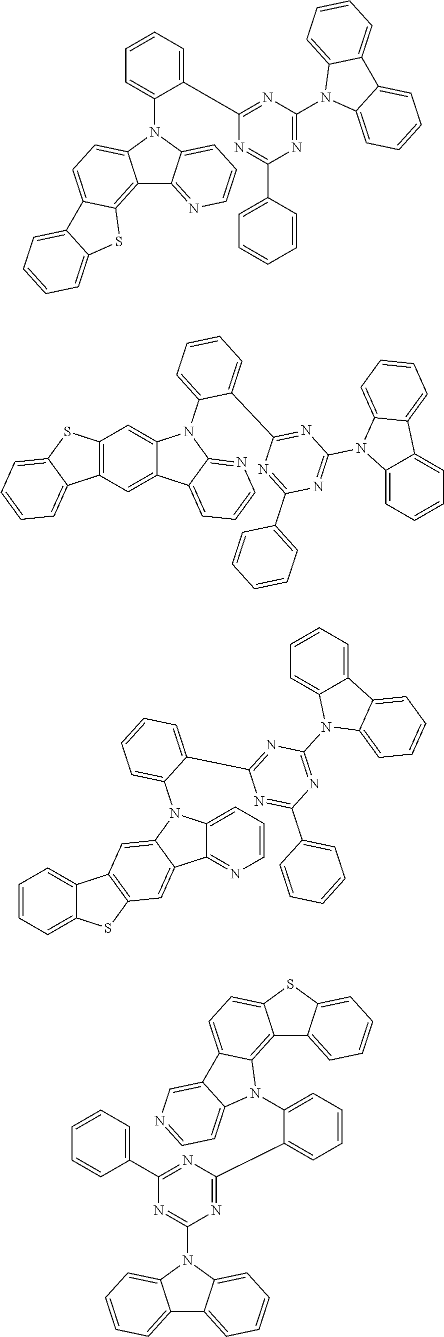 [SMILES: c1ccc(-c2nc(-c3ccccc3-n3c4cccnc4c4c5sc6ccccc6c5ccc43)nc(-n3c4ccccc4c4ccccc43)n2)cc1 ; c1ccc(-c2nc(-c3ccccc3-n3c4cc5sc6ccccc6c5cc4c4cccnc43)nc(-n3c4ccccc4c4ccccc43)n2)cc1 ; c1ccc(-c2nc(-c3ccccc3-n3c4cc5c(cc4c4ncccc43)sc3ccccc35)nc(-n3c4ccccc4c4ccccc43)n2)cc1 ; c1ccc(-c2nc(-c3ccccc3-n3c4ccncc4c4ccc5sc6ccccc6c5c43)nc(-n3c4ccccc4c4ccccc43)n2)cc1]

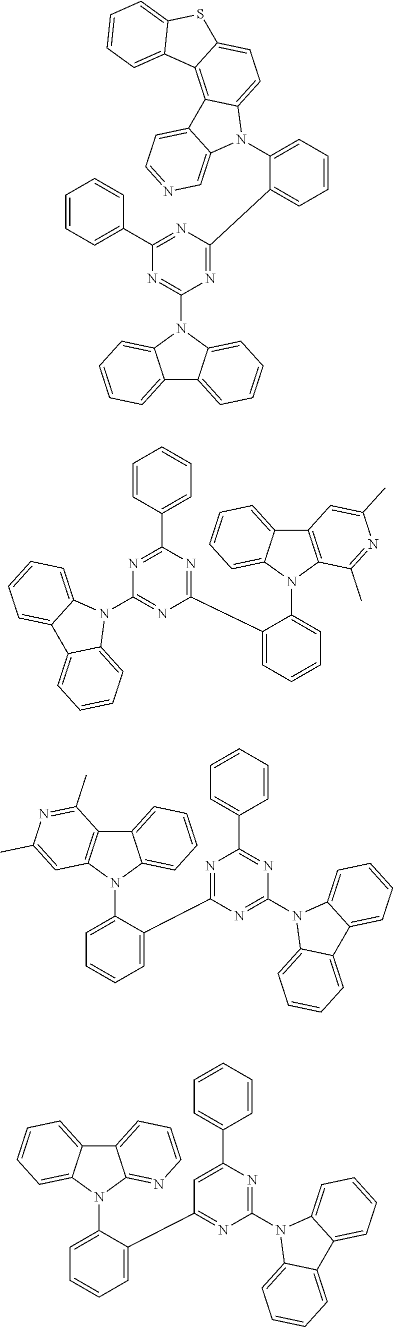

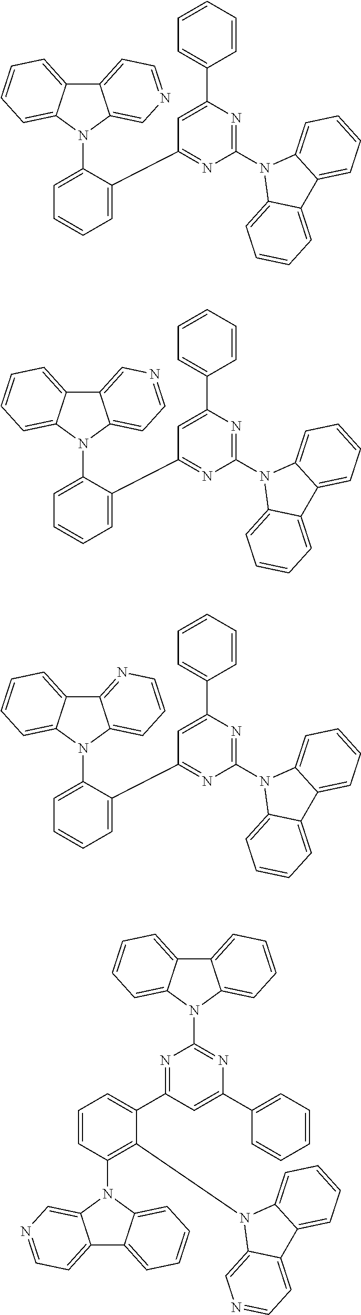 [SMILES: c1ccc(-c2cc(-c3ccccc3-n3c4ccccc4c4ccncc43)nc(-n3c4ccccc4c4ccccc43)n2)cc1 ; c1ccc(-c2cc(-c3ccccc3-n3c4ccccc4c4cnccc43)nc(-n3c4ccccc4c4ccccc43)n2)cc1 ; c1ccc(-c2cc(-c3ccccc3-n3c4ccccc4c4ncccc43)nc(-n3c4ccccc4c4ccccc43)n2)cc1 ; c1ccc(-c2cc(-c3cccc(-n4c5ccccc5c5ccncc54)c3-n3c4ccccc4c4ccncc43)nc(-n3c4ccccc4c4ccccc43)n2)cc1]

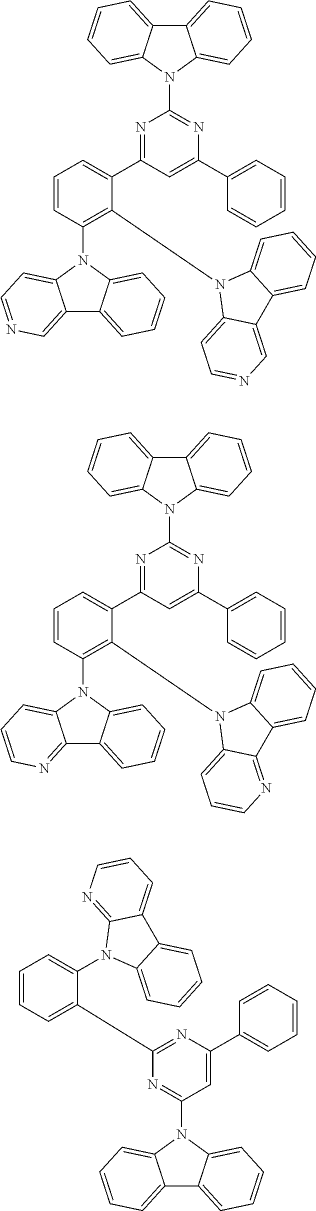 [SMILES: c1ccc(-c2cc(-c3cccc(-n4c5ccccc5c5cnccc54)c3-n3c4ccccc4c4cnccc43)nc(-n3c4ccccc4c4ccccc43)n2)cc1 ; c1ccc(-c2cc(-c3cccc(-n4c5ccccc5c5ncccc54)c3-n3c4ccccc4c4ncccc43)nc(-n3c4ccccc4c4ccccc43)n2)cc1 ; c1ccc(-c2cc(-n3c4ccccc4c4ccccc43)nc(-c3ccccc3-n3c4ccccc4c4cccnc43)n2)cc1]

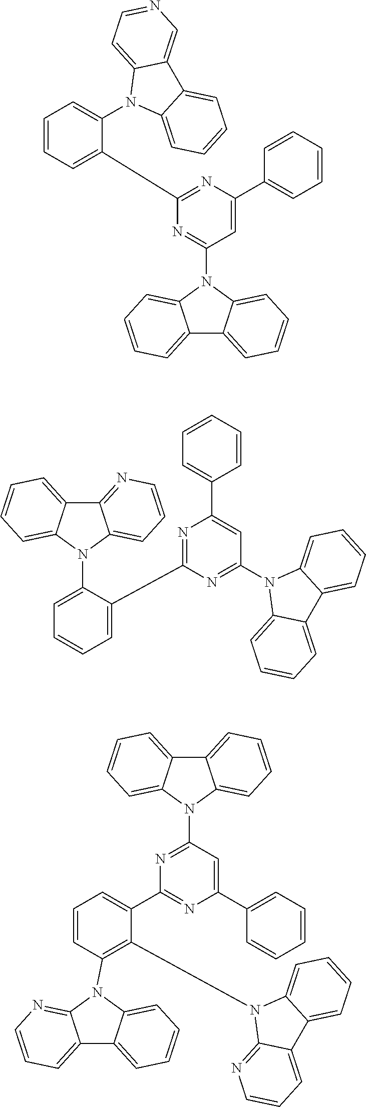 [SMILES: c1ccc(-c2cc(-n3c4ccccc4c4ccccc43)nc(-c3ccccc3-n3c4ccccc4c4cnccc43)n2)cc1 ; c1ccc(-c2cc(-n3c4ccccc4c4ccccc43)nc(-c3ccccc3-n3c4ccccc4c4ncccc43)n2)cc1 ; c1ccc(-c2cc(-n3c4ccccc4c4ccccc43)nc(-c3cccc(-n4c5ccccc5c5cccnc54)c3-n3c4ccccc4c4cccnc43)n2)cc1]

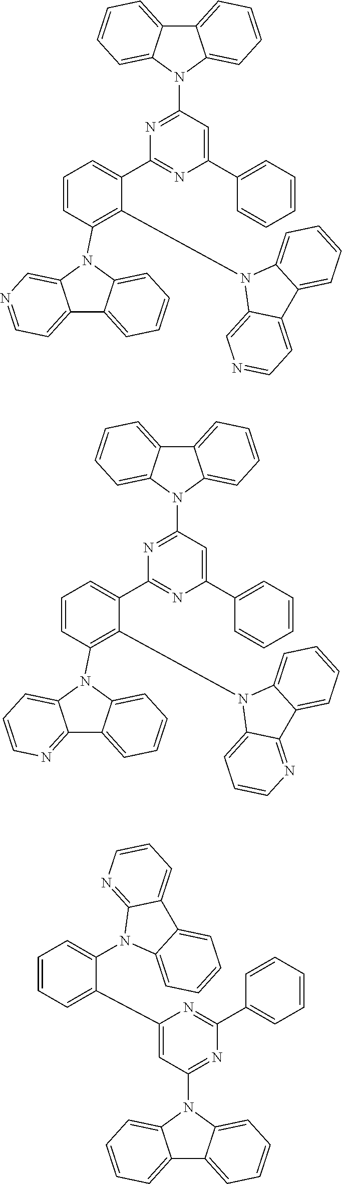 [SMILES: c1ccc(-c2cc(-n3c4ccccc4c4ccccc43)nc(-c3cccc(-n4c5ccccc5c5ccncc54)c3-n3c4ccccc4c4ccncc43)n2)cc1 ; c1ccc(-c2cc(-n3c4ccccc4c4ccccc43)nc(-c3cccc(-n4c5ccccc5c5ncccc54)c3-n3c4ccccc4c4ncccc43)n2)cc1 ; c1ccc(-c2nc(-c3ccccc3-n3c4ccccc4c4cccnc43)cc(-n3c4ccccc4c4ccccc43)n2)cc1]

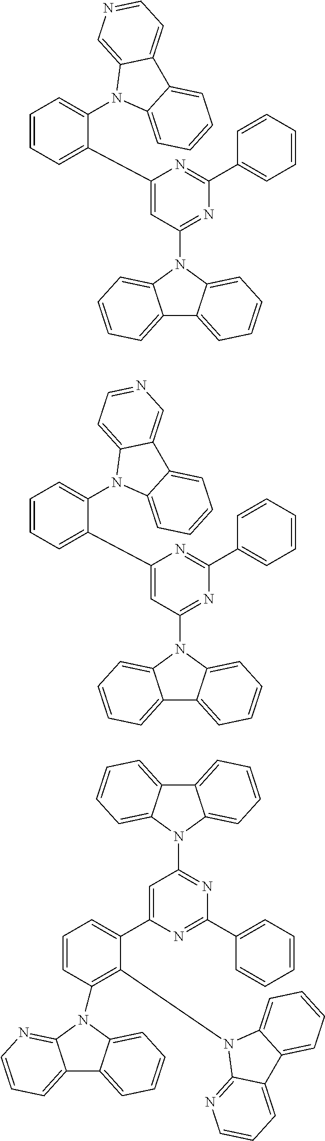

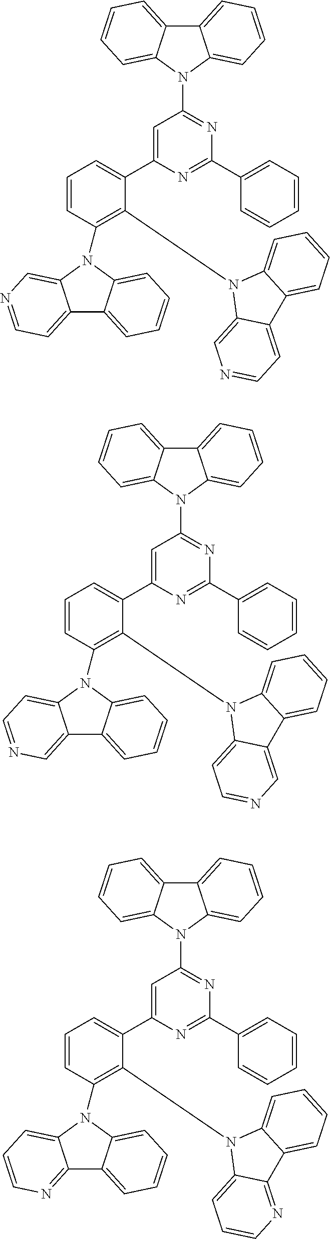 [SMILES: c1ccc(-c2nc(-c3cccc(-n4c5ccccc5c5ccncc54)c3-n3c4ccccc4c4ccncc43)cc(-n3c4ccccc4c4ccccc43)n2)cc1 ; c1ccc(-c2nc(-c3cccc(-n4c5ccccc5c5cnccc54)c3-n3c4ccccc4c4cnccc43)cc(-n3c4ccccc4c4ccccc43)n2)cc1 ; c1ccc(-c2nc(-c3cccc(-n4c5ccccc5c5ncccc54)c3-n3c4ccccc4c4ncccc43)cc(-n3c4ccccc4c4ccccc43)n2)cc1]

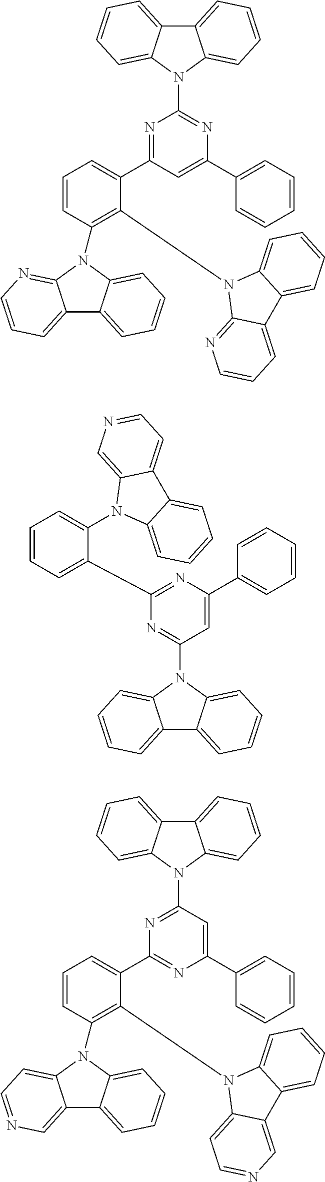

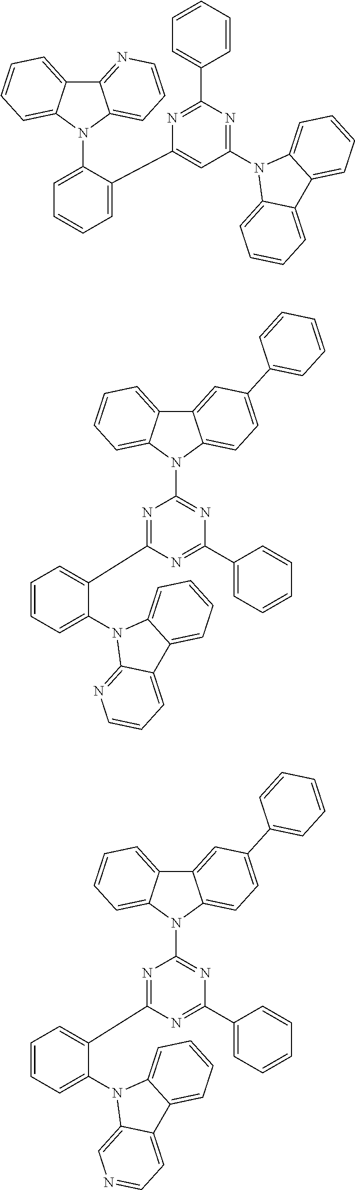 [SMILES: c1ccc(-c2nc(-c3ccccc3-n3c4ccccc4c4ncccc43)cc(-n3c4ccccc4c4ccccc43)n2)cc1 ; c1ccc(-c2ccc3c(c2)c2ccccc2n3-c2nc(-c3ccccc3)nc(-c3ccccc3-n3c4ccccc4c4cccnc43)n2)cc1 ; c1ccc(-c2ccc3c(c2)c2ccccc2n3-c2nc(-c3ccccc3)nc(-c3ccccc3-n3c4ccccc4c4ccncc43)n2)cc1]

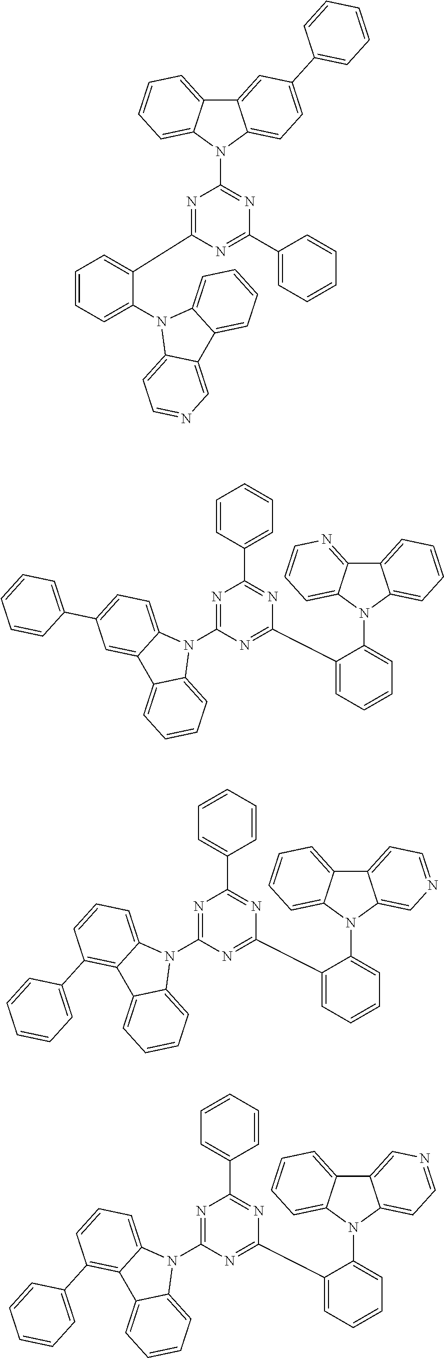 [SMILES: c1ccc(-c2ccc3c(c2)c2ccccc2n3-c2nc(-c3ccccc3)nc(-c3ccccc3-n3c4ccccc4c4cnccc43)n2)cc1 ; c1ccc(-c2ccc3c(c2)c2ccccc2n3-c2nc(-c3ccccc3)nc(-c3ccccc3-n3c4ccccc4c4ncccc43)n2)cc1 ; c1ccc(-c2nc(-c3ccccc3-n3c4ccccc4c4ccncc43)nc(-n3c4ccccc4c4c(-c5ccccc5)cccc43)n2)cc1 ; c1ccc(-c2nc(-c3ccccc3-n3c4ccccc4c4cnccc43)nc(-n3c4ccccc4c4c(-c5ccccc5)cccc43)n2)cc1]

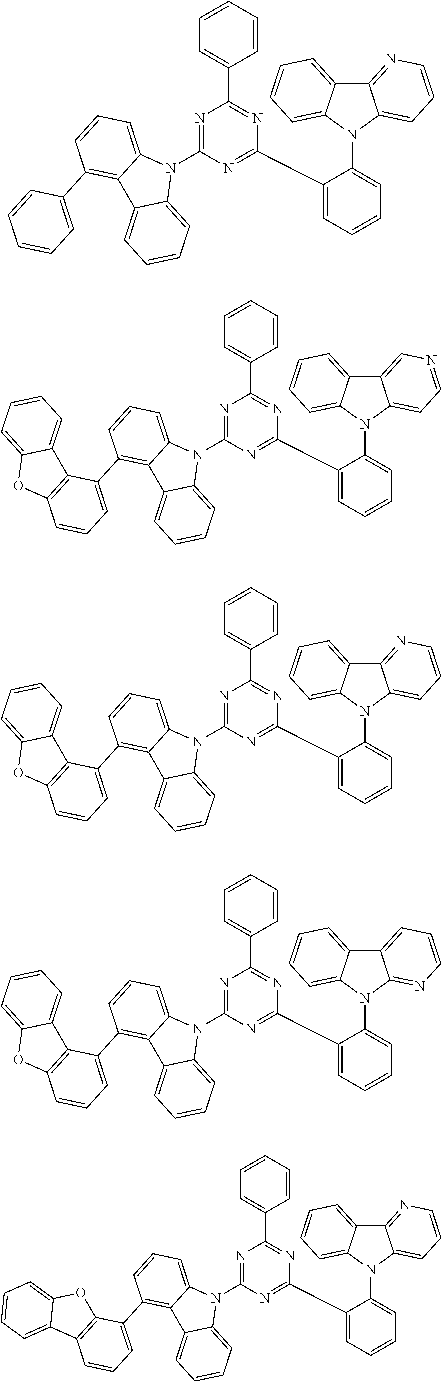 [SMILES: c1ccc(-c2nc(-c3ccccc3-n3c4ccccc4c4ncccc43)nc(-n3c4ccccc4c4c(-c5ccccc5)cccc43)n2)cc1 ; c1ccc(-c2nc(-c3ccccc3-n3c4ccccc4c4cnccc43)nc(-n3c4ccccc4c4c(-c5cccc6oc7ccccc7c56)cccc43)n2)cc1 ; c1ccc(-c2nc(-c3ccccc3-n3c4ccccc4c4ncccc43)nc(-n3c4ccccc4c4c(-c5cccc6oc7ccccc7c56)cccc43)n2)cc1 ; c1ccc(-c2nc(-c3ccccc3-n3c4ccccc4c4cccnc43)nc(-n3c4ccccc4c4c(-c5cccc6oc7ccccc7c56)cccc43)n2)cc1 ; c1ccc(-c2nc(-c3ccccc3-n3c4ccccc4c4ncccc43)nc(-n3c4ccccc4c4c(-c5cccc6c5oc5ccccc56)cccc43)n2)cc1]

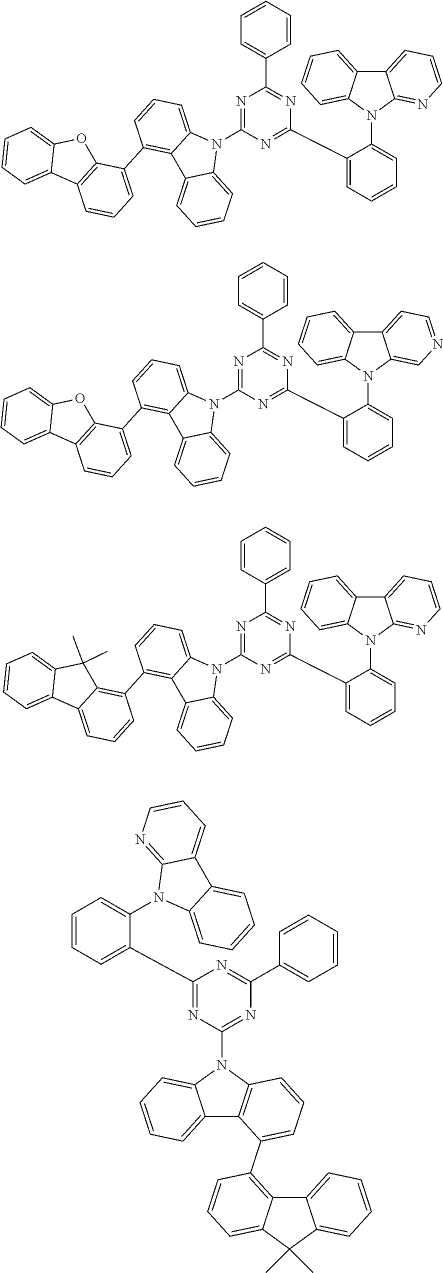 [SMILES: c1ccc(-c2nc(-c3ccccc3-n3c4ccccc4c4cccnc43)nc(-n3c4ccccc4c4c(-c5cccc6c5oc5ccccc56)cccc43)n2)cc1 ; c1ccc(-c2nc(-c3ccccc3-n3c4ccccc4c4ccncc43)nc(-n3c4ccccc4c4c(-c5cccc6c5oc5ccccc56)cccc43)n2)cc1 ; CC1(C)c2ccccc2-c2cccc(-c3cccc4c3c3ccccc3n4-c3nc(-c4ccccc4)nc(-c4ccccc4-n4c5ccccc5c5cccnc54)n3)c21 ; CC1(C)c2ccccc2-c2c(-c3cccc4c3c3ccccc3n4-c3nc(-c4ccccc4)nc(-c4ccccc4-n4c5ccccc5c5cccnc54)n3)cccc21]

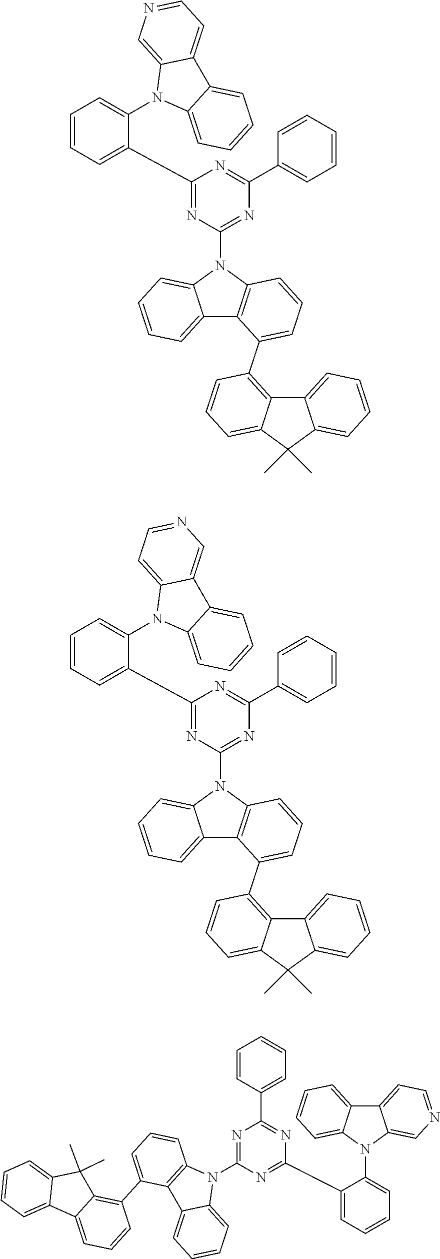

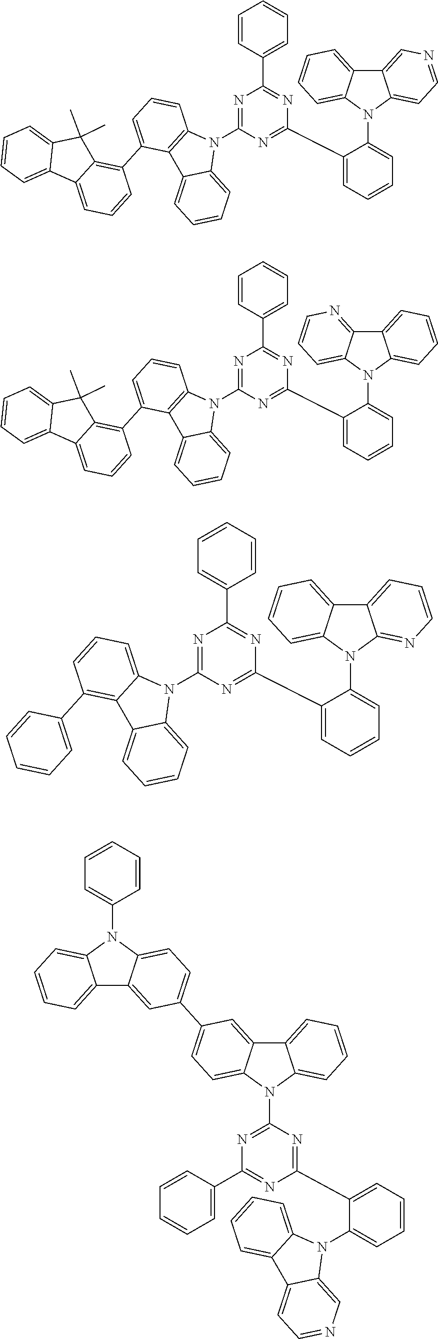 [SMILES: CC1(C)c2ccccc2-c2cccc(-c3cccc4c3c3ccccc3n4-c3nc(-c4ccccc4)nc(-c4ccccc4-n4c5ccccc5c5cnccc54)n3)c21 ; CC1(C)c2ccccc2-c2cccc(-c3cccc4c3c3ccccc3n4-c3nc(-c4ccccc4)nc(-c4ccccc4-n4c5ccccc5c5ncccc54)n3)c21 ; c1ccc(-c2nc(-c3ccccc3-n3c4ccccc4c4cccnc43)nc(-n3c4ccccc4c4c(-c5ccccc5)cccc43)n2)cc1 ; c1ccc(-c2nc(-c3ccccc3-n3c4ccccc4c4ccncc43)nc(-n3c4ccccc4c4cc(-c5ccc6c(c5)c5ccccc5n6-c5ccccc5)ccc43)n2)cc1]

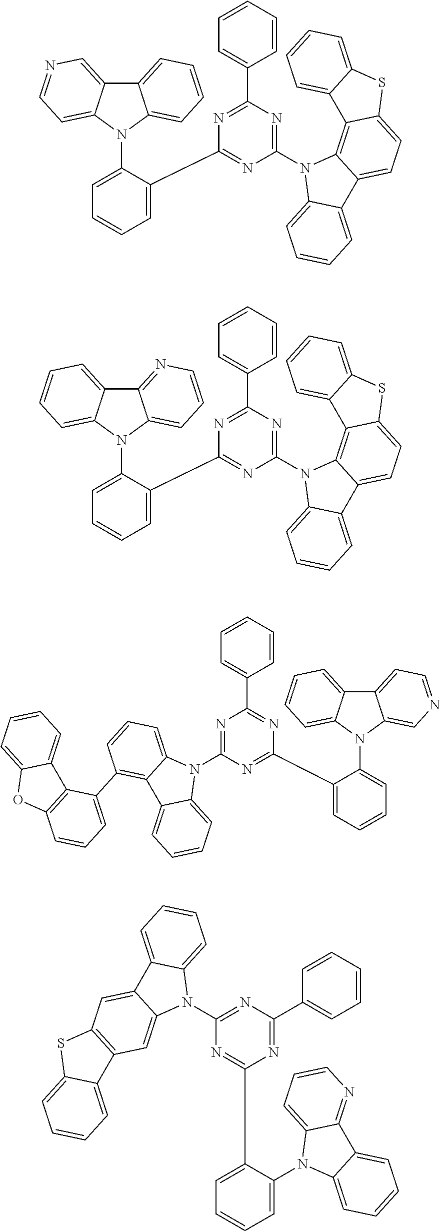 [SMILES: c1ccc(-c2nc(-c3ccccc3-n3c4ccccc4c4cnccc43)nc(-n3c4ccccc4c4ccc5sc6ccccc6c5c43)n2)cc1 ; c1ccc(-c2nc(-c3ccccc3-n3c4ccccc4c4ncccc43)nc(-n3c4ccccc4c4ccc5sc6ccccc6c5c43)n2)cc1 ; c1ccc(-c2nc(-c3ccccc3-n3c4ccccc4c4ccncc43)nc(-n3c4ccccc4c4c(-c5cccc6oc7ccccc7c56)cccc43)n2)cc1 ; c1ccc(-c2nc(-c3ccccc3-n3c4ccccc4c4ncccc43)nc(-n3c4ccccc4c4cc5sc6ccccc6c5cc43)n2)cc1]

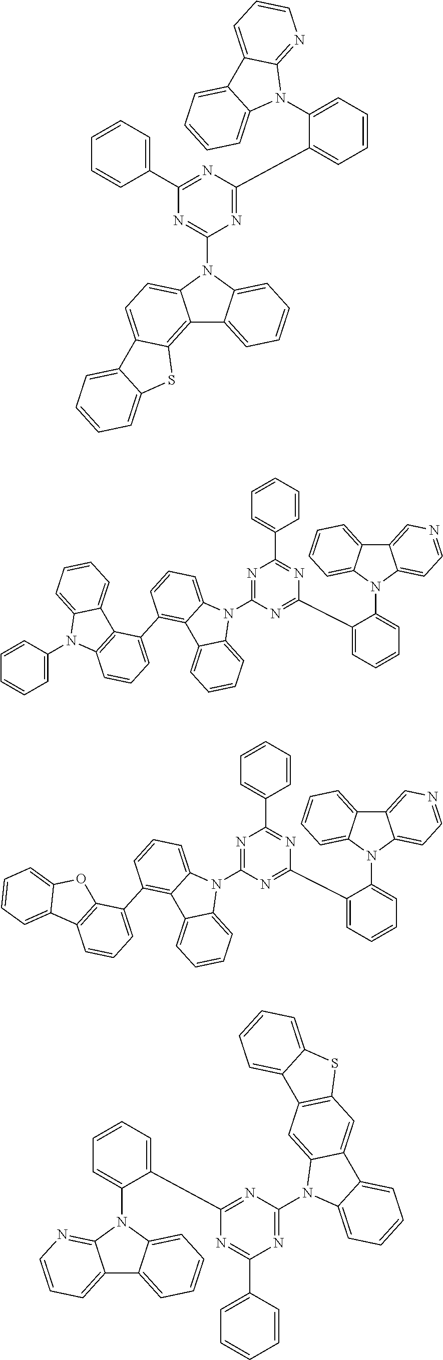 [SMILES: c1ccc(-c2nc(-c3ccccc3-n3c4ccccc4c4cccnc43)nc(-n3c4ccccc4c4c5sc6ccccc6c5ccc43)n2)cc1 ; c1ccc(-c2nc(-c3ccccc3-n3c4ccccc4c4cnccc43)nc(-n3c4ccccc4c4c(-c5cccc6c5c5ccccc5n6-c5ccccc5)cccc43)n2)cc1 ; c1ccc(-c2nc(-c3ccccc3-n3c4ccccc4c4cnccc43)nc(-n3c4ccccc4c4c(-c5cccc6c5oc5ccccc56)cccc43)n2)cc1 ; c1ccc(-c2nc(-c3ccccc3-n3c4ccccc4c4cccnc43)nc(-n3c4ccccc4c4cc5sc6ccccc6c5cc43)n2)cc1]

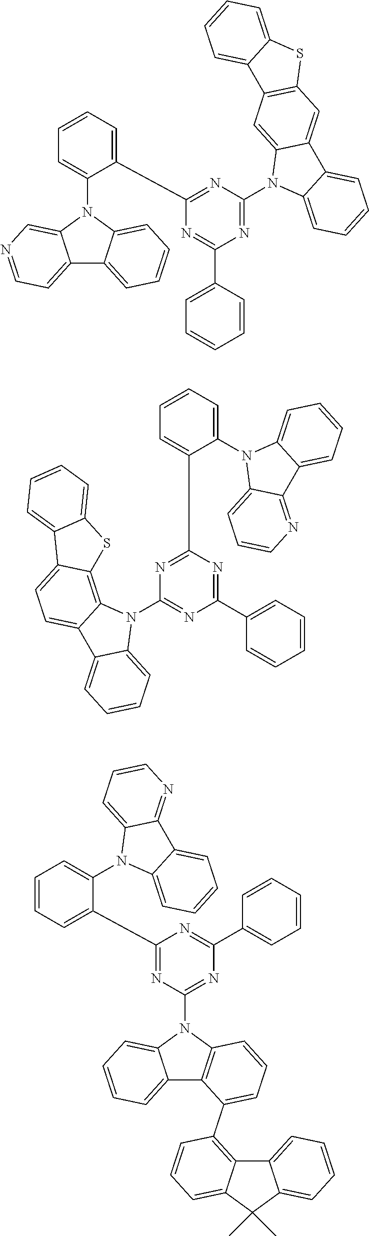 [SMILES: c1ccc(-c2nc(-c3ccccc3-n3c4ccccc4c4ccncc43)nc(-n3c4ccccc4c4cc5sc6ccccc6c5cc43)n2)cc1 ; c1ccc(-c2nc(-c3ccccc3-n3c4ccccc4c4ncccc43)nc(-n3c4ccccc4c4ccc5c6ccccc6sc5c43)n2)cc1 ; CC1(C)c2ccccc2-c2c(-c3cccc4c3c3ccccc3n4-c3nc(-c4ccccc4)nc(-c4ccccc4-n4c5ccccc5c5ncccc54)n3)cccc21]

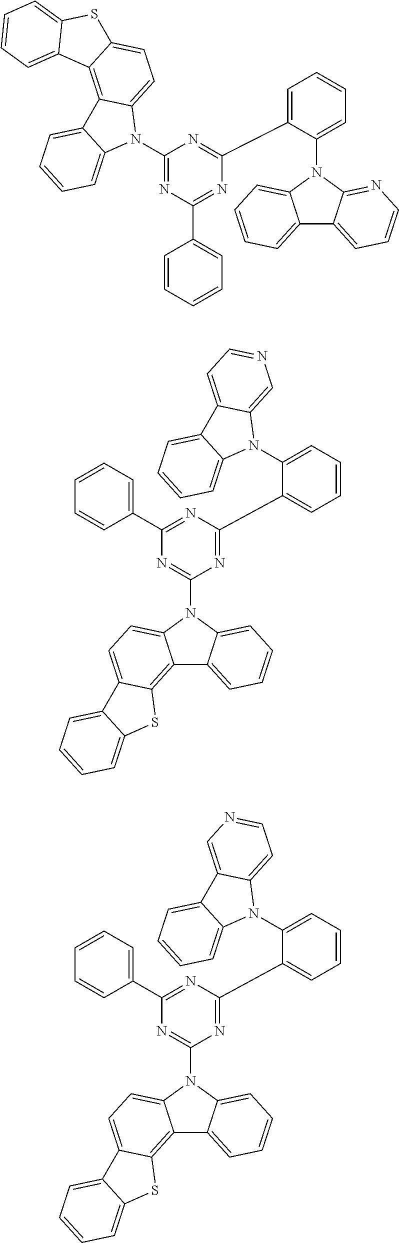 [SMILES: c1ccc(-c2nc(-c3ccccc3-n3c4ccccc4c4cccnc43)nc(-n3c4ccccc4c4c5c(ccc43)sc3ccccc35)n2)cc1 ; c1ccc(-c2nc(-c3ccccc3-n3c4ccccc4c4ccncc43)nc(-n3c4ccccc4c4c5sc6ccccc6c5ccc43)n2)cc1 ; c1ccc(-c2nc(-c3ccccc3-n3c4ccccc4c4cnccc43)nc(-n3c4ccccc4c4c5sc6ccccc6c5ccc43)n2)cc1]

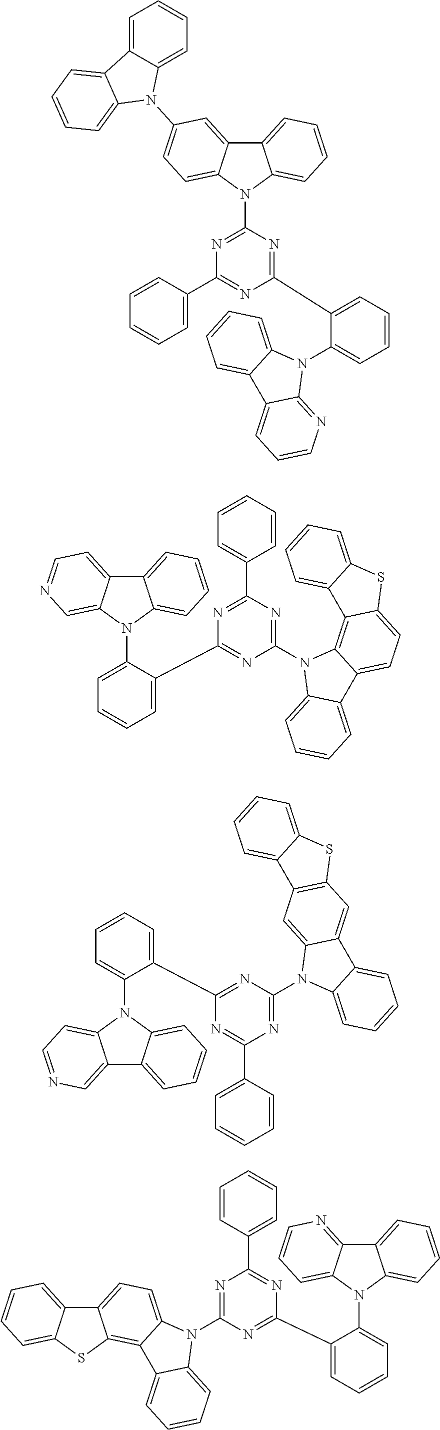

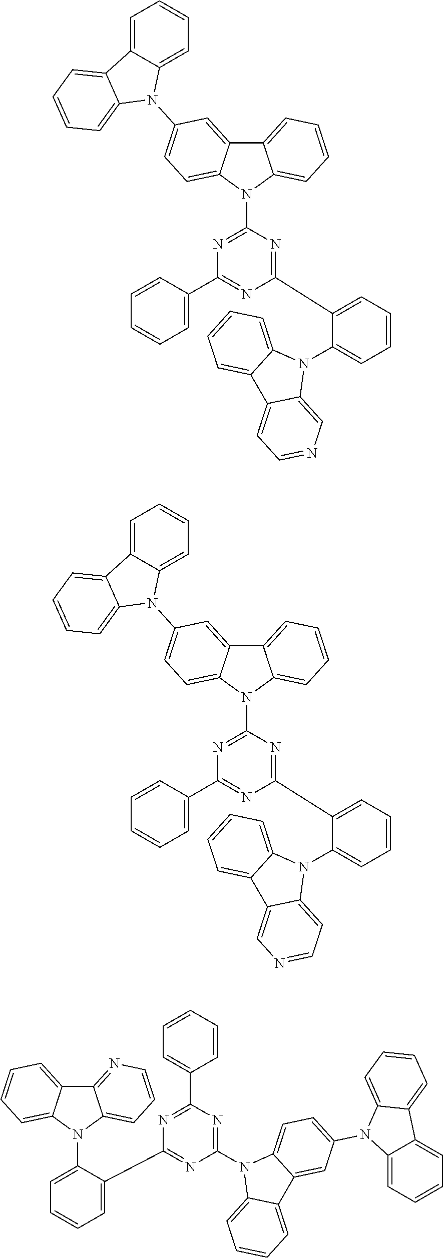 [SMILES: c1ccc(-c2nc(-c3ccccc3-n3c4ccccc4c4ccncc43)nc(-n3c4ccccc4c4cc(-n5c6ccccc6c6ccccc65)ccc43)n2)cc1 ; c1ccc(-c2nc(-c3ccccc3-n3c4ccccc4c4cnccc43)nc(-n3c4ccccc4c4cc(-n5c6ccccc6c6ccccc65)ccc43)n2)cc1 ; c1ccc(-c2nc(-c3ccccc3-n3c4ccccc4c4ncccc43)nc(-n3c4ccccc4c4cc(-n5c6ccccc6c6ccccc65)ccc43)n2)cc1]

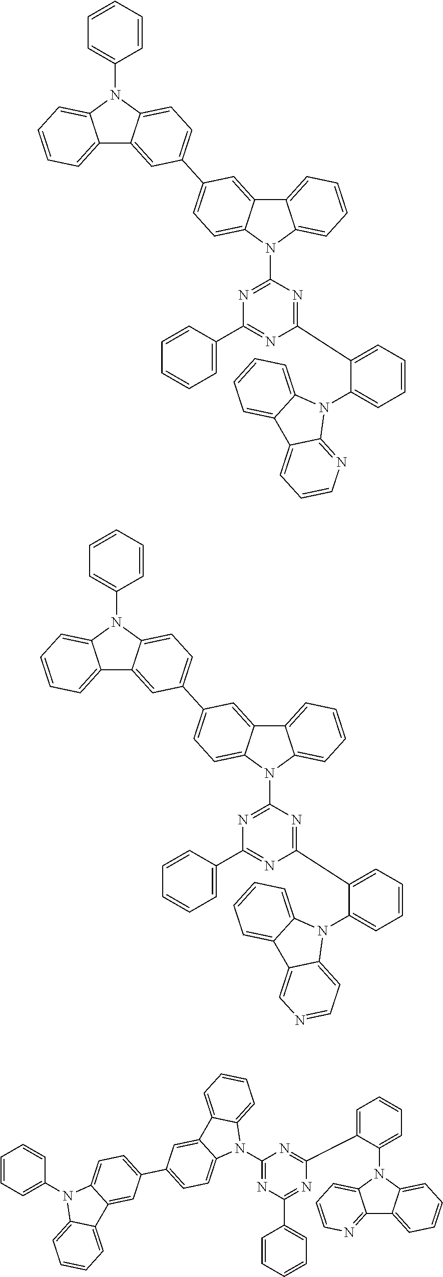 [SMILES: c1ccc(-c2nc(-c3ccccc3-n3c4ccccc4c4cccnc43)nc(-n3c4ccccc4c4cc(-c5ccc6c(c5)c5ccccc5n6-c5ccccc5)ccc43)n2)cc1 ; c1ccc(-c2nc(-c3ccccc3-n3c4ccccc4c4cnccc43)nc(-n3c4ccccc4c4cc(-c5ccc6c(c5)c5ccccc5n6-c5ccccc5)ccc43)n2)cc1 ; c1ccc(-c2nc(-c3ccccc3-n3c4ccccc4c4ncccc43)nc(-n3c4ccccc4c4cc(-c5ccc6c(c5)c5ccccc5n6-c5ccccc5)ccc43)n2)cc1]

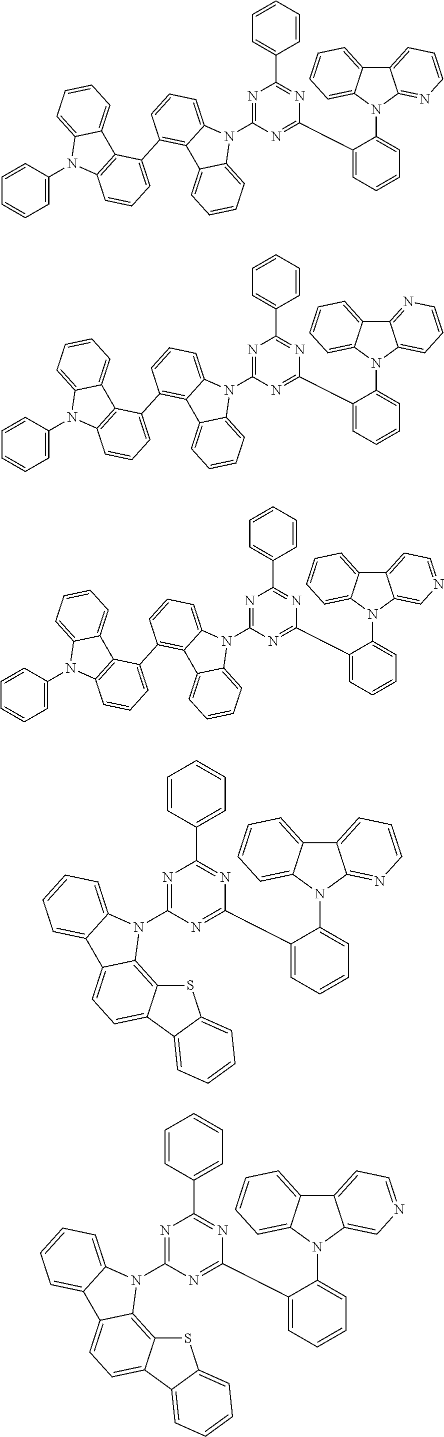

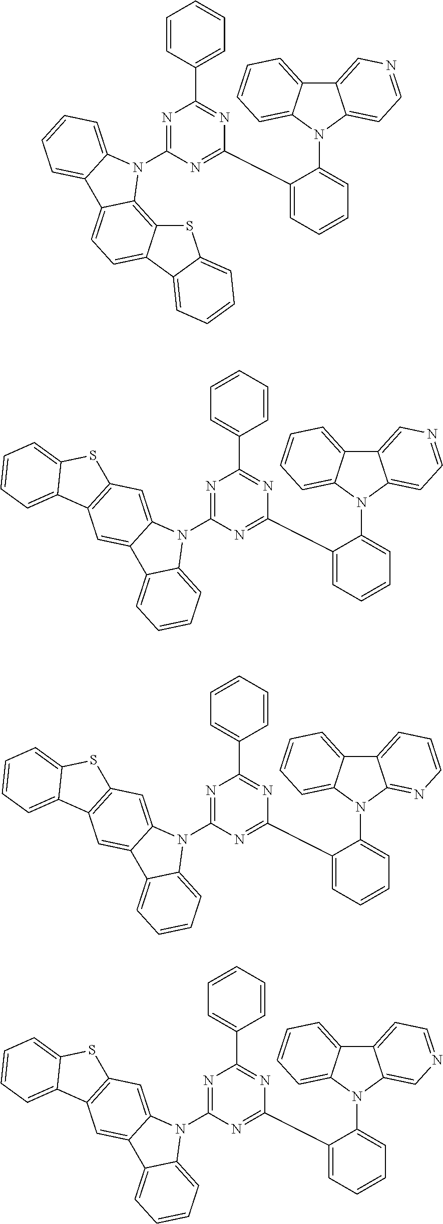 [SMILES: c1ccc(-c2nc(-c3ccccc3-n3c4ccccc4c4cnccc43)nc(-n3c4ccccc4c4ccc5c6ccccc6sc5c43)n2)cc1 ; c1ccc(-c2nc(-c3ccccc3-n3c4ccccc4c4cnccc43)nc(-n3c4ccccc4c4cc5c(cc43)sc3ccccc35)n2)cc1 ; c1ccc(-c2nc(-c3ccccc3-n3c4ccccc4c4cccnc43)nc(-n3c4ccccc4c4cc5c(cc43)sc3ccccc35)n2)cc1 ; c1ccc(-c2nc(-c3ccccc3-n3c4ccccc4c4ccncc43)nc(-n3c4ccccc4c4cc5c(cc43)sc3ccccc35)n2)cc1]

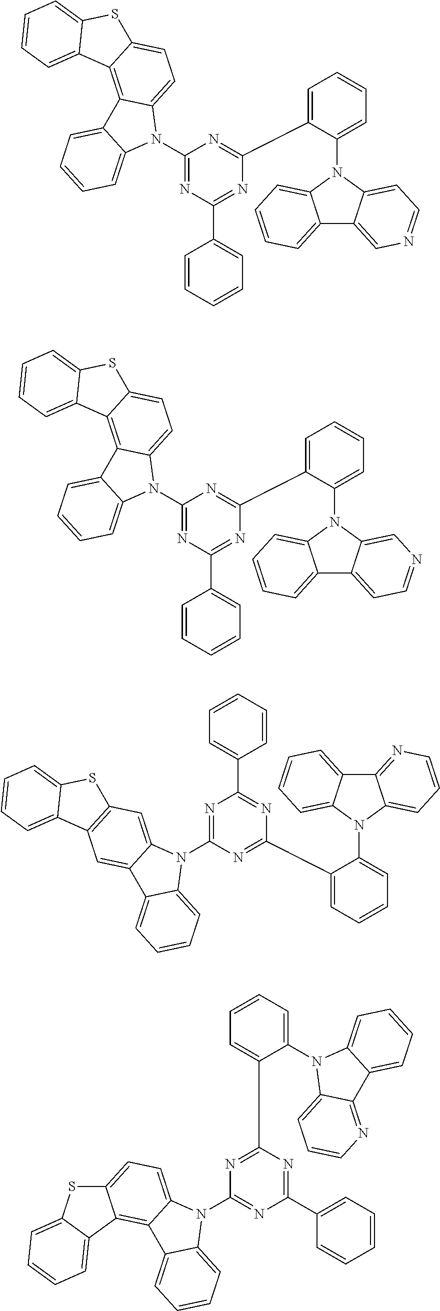 [SMILES: c1ccc(-c2nc(-c3ccccc3-n3c4ccccc4c4cnccc43)nc(-n3c4ccccc4c4c5c(ccc43)sc3ccccc35)n2)cc1 ; c1ccc(-c2nc(-c3ccccc3-n3c4ccccc4c4ccncc43)nc(-n3c4ccccc4c4c5c(ccc43)sc3ccccc35)n2)cc1 ; c1ccc(-c2nc(-c3ccccc3-n3c4ccccc4c4ncccc43)nc(-n3c4ccccc4c4cc5c(cc43)sc3ccccc35)n2)cc1 ; c1ccc(-c2nc(-c3ccccc3-n3c4ccccc4c4ncccc43)nc(-n3c4ccccc4c4c5c(ccc43)sc3ccccc35)n2)cc1]

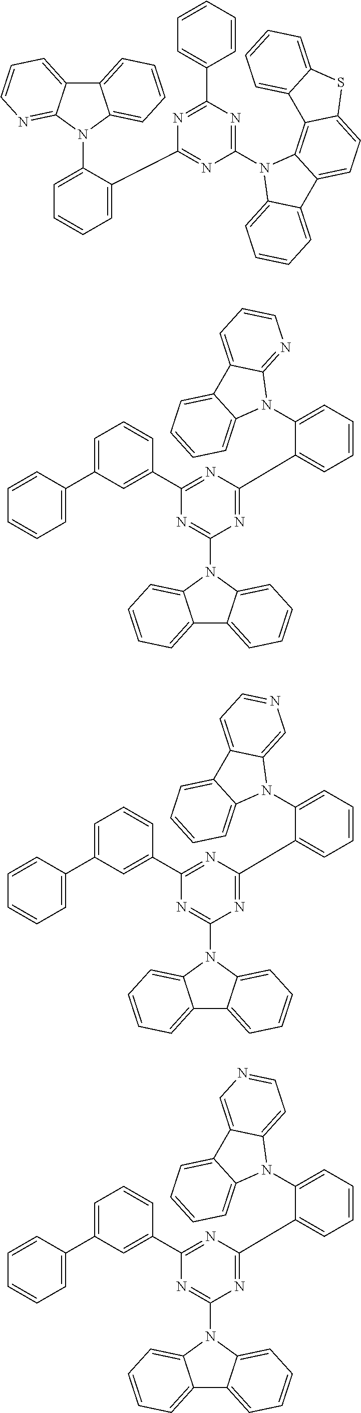

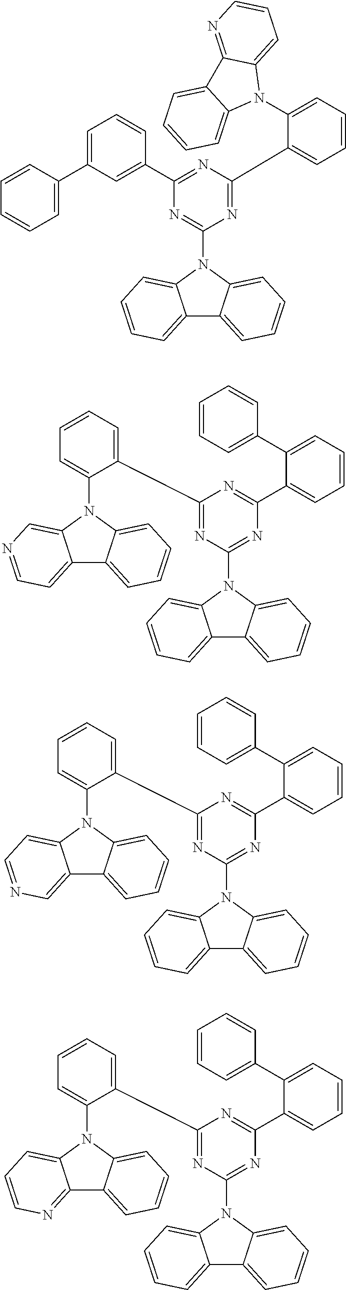 [SMILES: c1ccc(-c2cccc(-c3nc(-c4ccccc4-n4c5ccccc5c5ncccc54)nc(-n4c5ccccc5c5ccccc54)n3)c2)cc1 ; c1ccc(-c2ccccc2-c2nc(-c3ccccc3-n3c4ccccc4c4ccncc43)nc(-n3c4ccccc4c4ccccc43)n2)cc1 ; c1ccc(-c2ccccc2-c2nc(-c3ccccc3-n3c4ccccc4c4cnccc43)nc(-n3c4ccccc4c4ccccc43)n2)cc1 ; c1ccc(-c2ccccc2-c2nc(-c3ccccc3-n3c4ccccc4c4ncccc43)nc(-n3c4ccccc4c4ccccc43)n2)cc1]

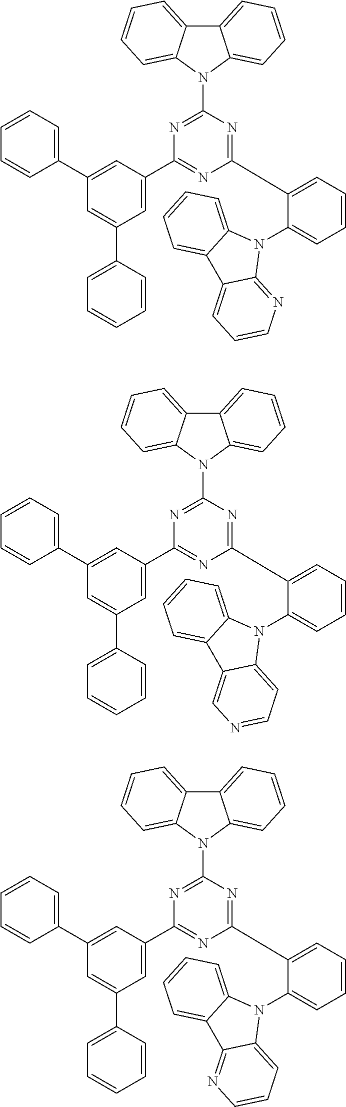

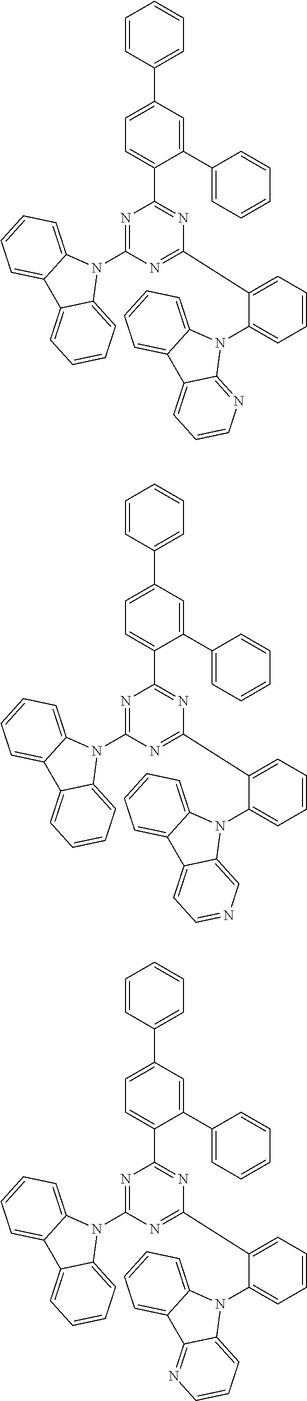 [SMILES: c1ccc(-c2ccc(-c3nc(-c4ccccc4-n4c5ccccc5c5cccnc54)nc(-n4c5ccccc5c5ccccc54)n3)c(-c3ccccc3)c2)cc1 ; c1ccc(-c2ccc(-c3nc(-c4ccccc4-n4c5ccccc5c5ccncc54)nc(-n4c5ccccc5c5ccccc54)n3)c(-c3ccccc3)c2)cc1 ; c1ccc(-c2ccc(-c3nc(-c4ccccc4-n4c5ccccc5c5ncccc54)nc(-n4c5ccccc5c5ccccc54)n3)c(-c3ccccc3)c2)cc1]

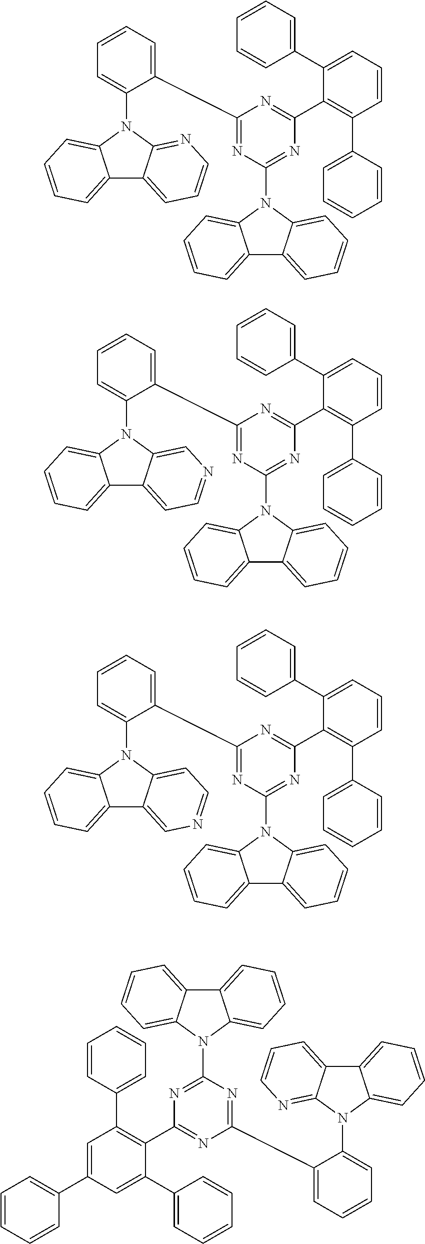 [SMILES: c1ccc(-c2cccc(-c3ccccc3)c2-c2nc(-c3ccccc3-n3c4ccccc4c4cccnc43)nc(-n3c4ccccc4c4ccccc43)n2)cc1 ; c1ccc(-c2cccc(-c3ccccc3)c2-c2nc(-c3ccccc3-n3c4ccccc4c4ccncc43)nc(-n3c4ccccc4c4ccccc43)n2)cc1 ; c1ccc(-c2cccc(-c3ccccc3)c2-c2nc(-c3ccccc3-n3c4ccccc4c4cnccc43)nc(-n3c4ccccc4c4ccccc43)n2)cc1 ; c1ccc(-c2cc(-c3ccccc3)c(-c3nc(-c4ccccc4-n4c5ccccc5c5cccnc54)nc(-n4c5ccccc5c5ccccc54)n3)c(-c3ccccc3)c2)cc1]

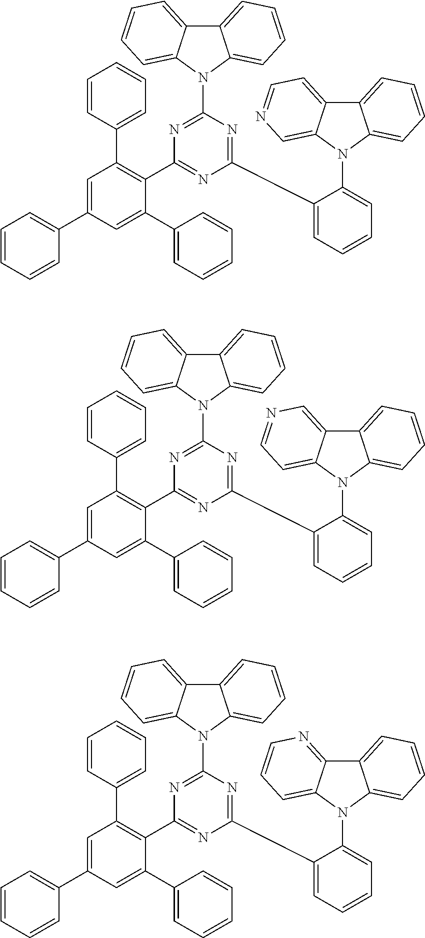 [SMILES: c1ccc(-c2cc(-c3ccccc3)c(-c3nc(-c4ccccc4-n4c5ccccc5c5ccncc54)nc(-n4c5ccccc5c5ccccc54)n3)c(-c3ccccc3)c2)cc1 ; c1ccc(-c2cc(-c3ccccc3)c(-c3nc(-c4ccccc4-n4c5ccccc5c5cnccc54)nc(-n4c5ccccc5c5ccccc54)n3)c(-c3ccccc3)c2)cc1 ; c1ccc(-c2cc(-c3ccccc3)c(-c3nc(-c4ccccc4-n4c5ccccc5c5ncccc54)nc(-n4c5ccccc5c5ccccc54)n3)c(-c3ccccc3)c2)cc1]

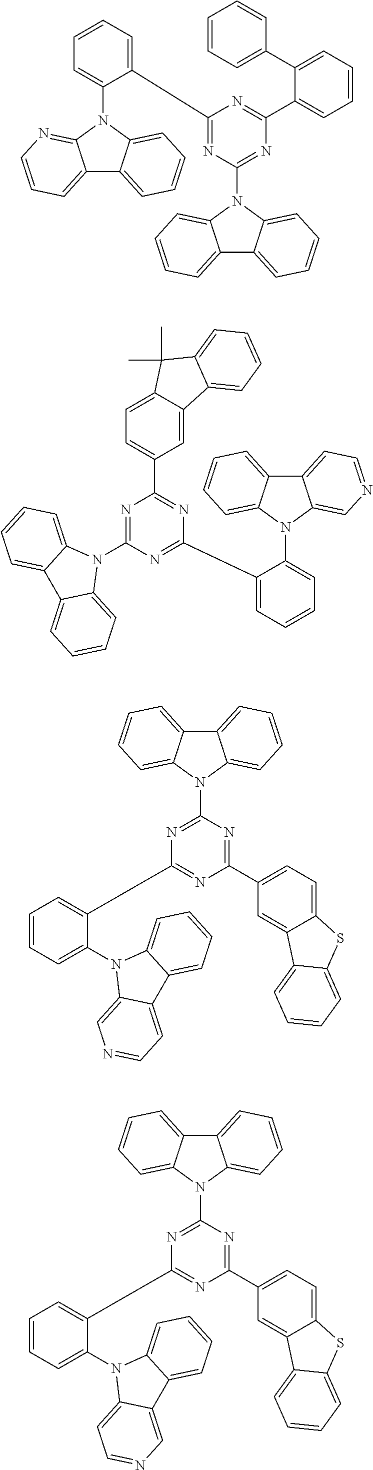 [SMILES: c1ccc(-c2ccccc2-c2nc(-c3ccccc3-n3c4ccccc4c4cccnc43)nc(-n3c4ccccc4c4ccccc43)n2)cc1 ; CC1(C)c2ccccc2-c2cc(-c3nc(-c4ccccc4-n4c5ccccc5c5ccncc54)nc(-n4c5ccccc5c5ccccc54)n3)ccc21 ; c1ccc(-n2c3ccccc3c3ccncc32)c(-c2nc(-c3ccc4sc5ccccc5c4c3)nc(-n3c4ccccc4c4ccccc43)n2)c1 ; c1ccc(-n2c3ccccc3c3cnccc32)c(-c2nc(-c3ccc4sc5ccccc5c4c3)nc(-n3c4ccccc4c4ccccc43)n2)c1]

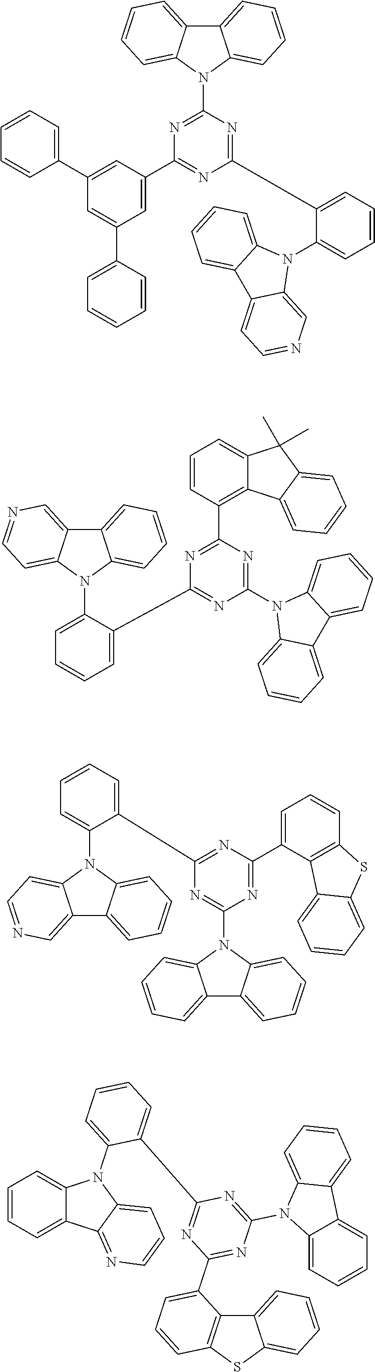 [SMILES: c1ccc(-c2cc(-c3ccccc3)cc(-c3nc(-c4ccccc4-n4c5ccccc5c5ccncc54)nc(-n4c5ccccc5c5ccccc54)n3)c2)cc1 ; CC1(C)c2ccccc2-c2c(-c3nc(-c4ccccc4-n4c5ccccc5c5cnccc54)nc(-n4c5ccccc5c5ccccc54)n3)cccc21 ; c1ccc(-n2c3ccccc3c3cnccc32)c(-c2nc(-c3cccc4sc5ccccc5c34)nc(-n3c4ccccc4c4ccccc43)n2)c1 ; c1ccc(-n2c3ccccc3c3ncccc32)c(-c2nc(-c3cccc4sc5ccccc5c34)nc(-n3c4ccccc4c4ccccc43)n2)c1]

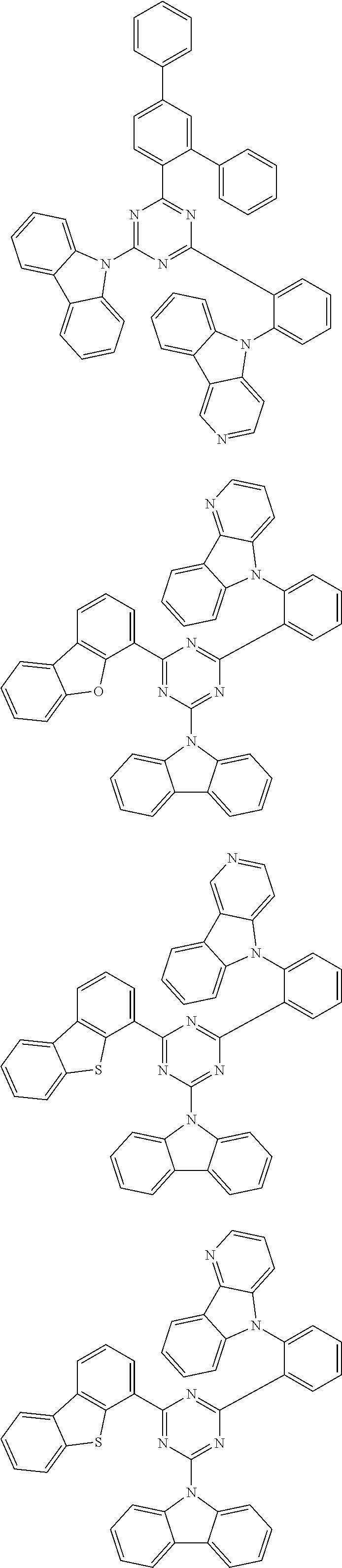

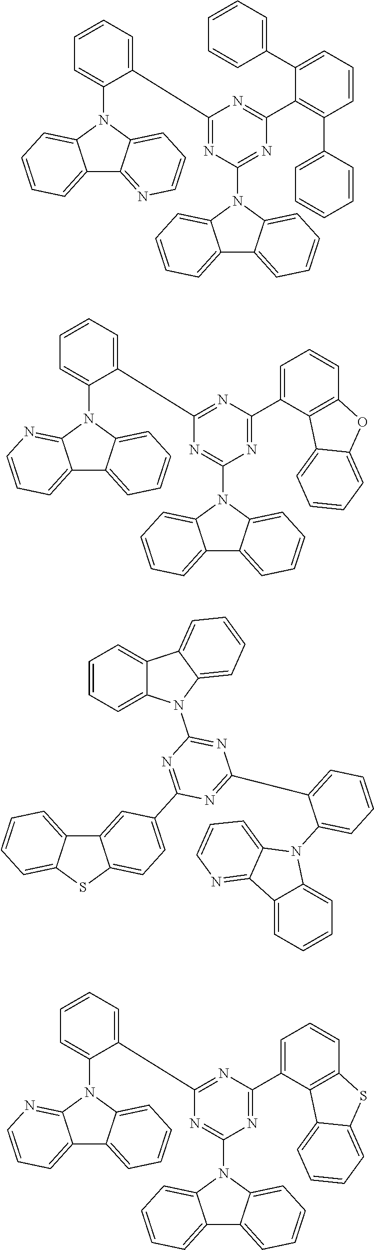

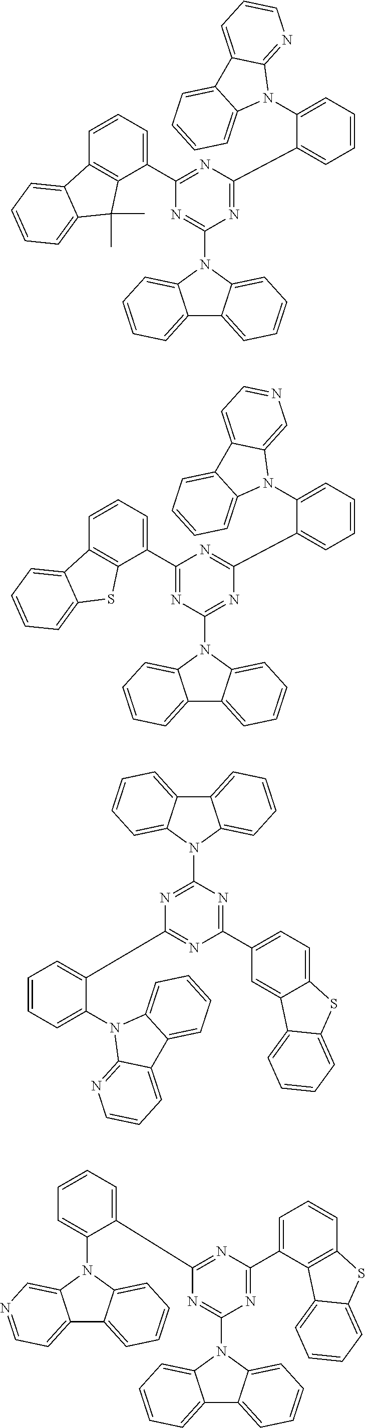

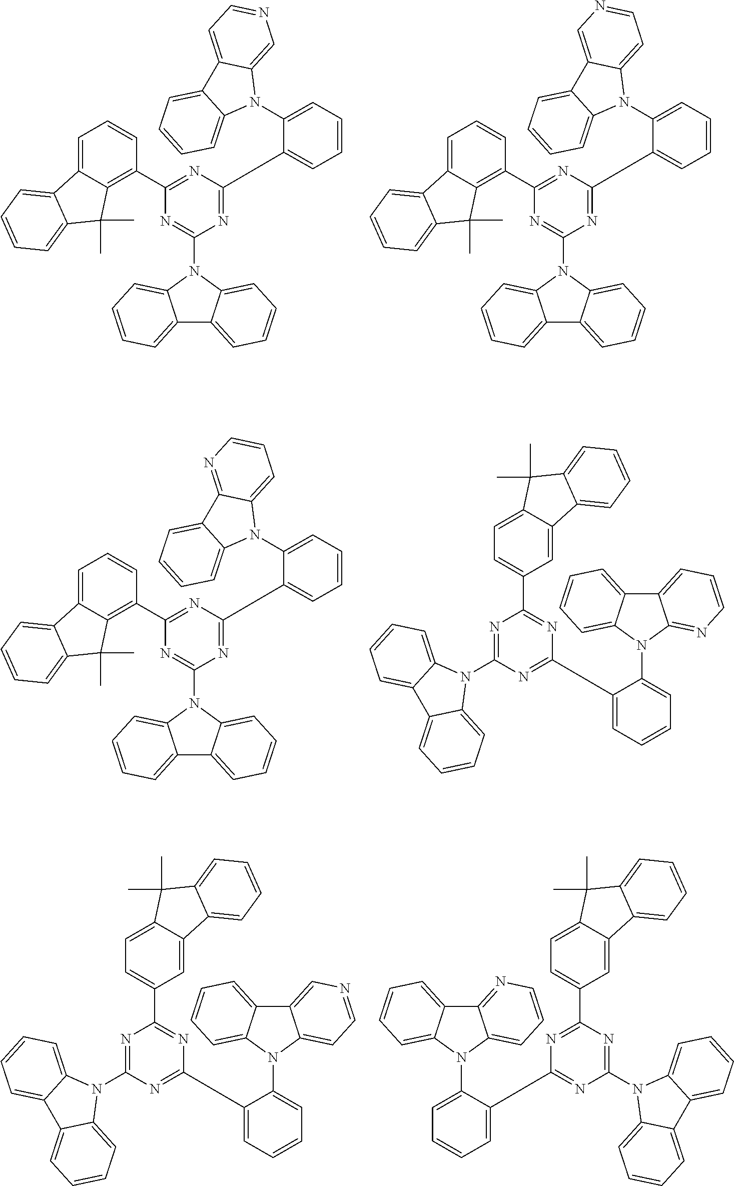

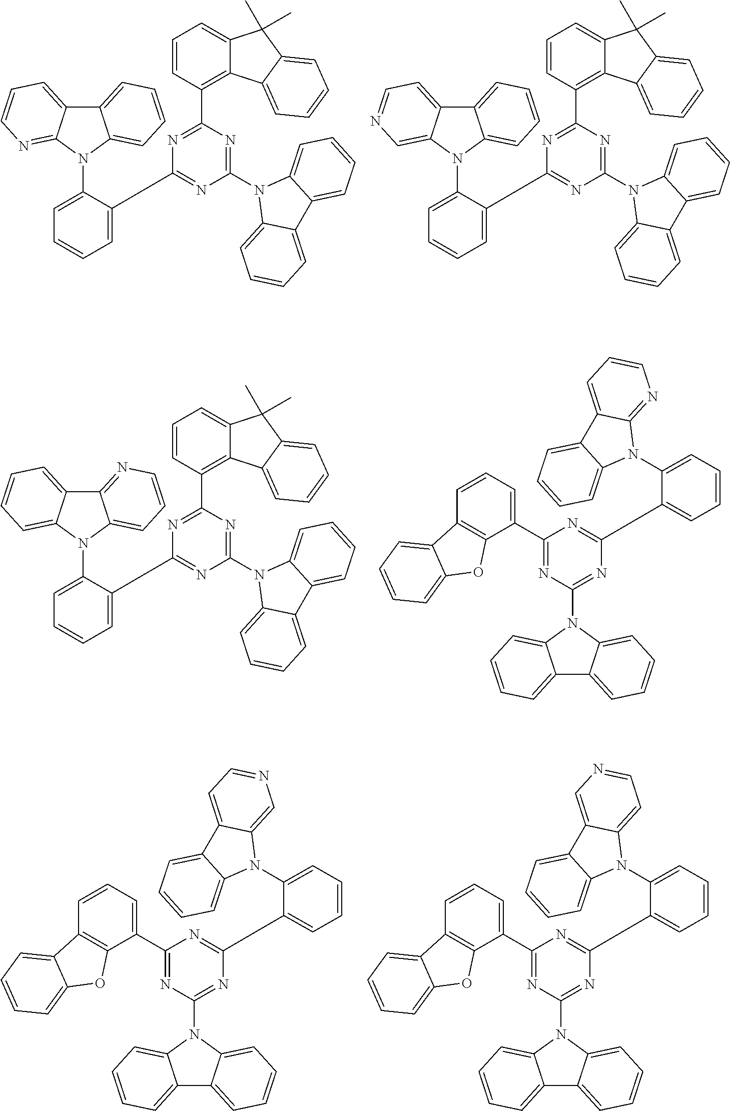

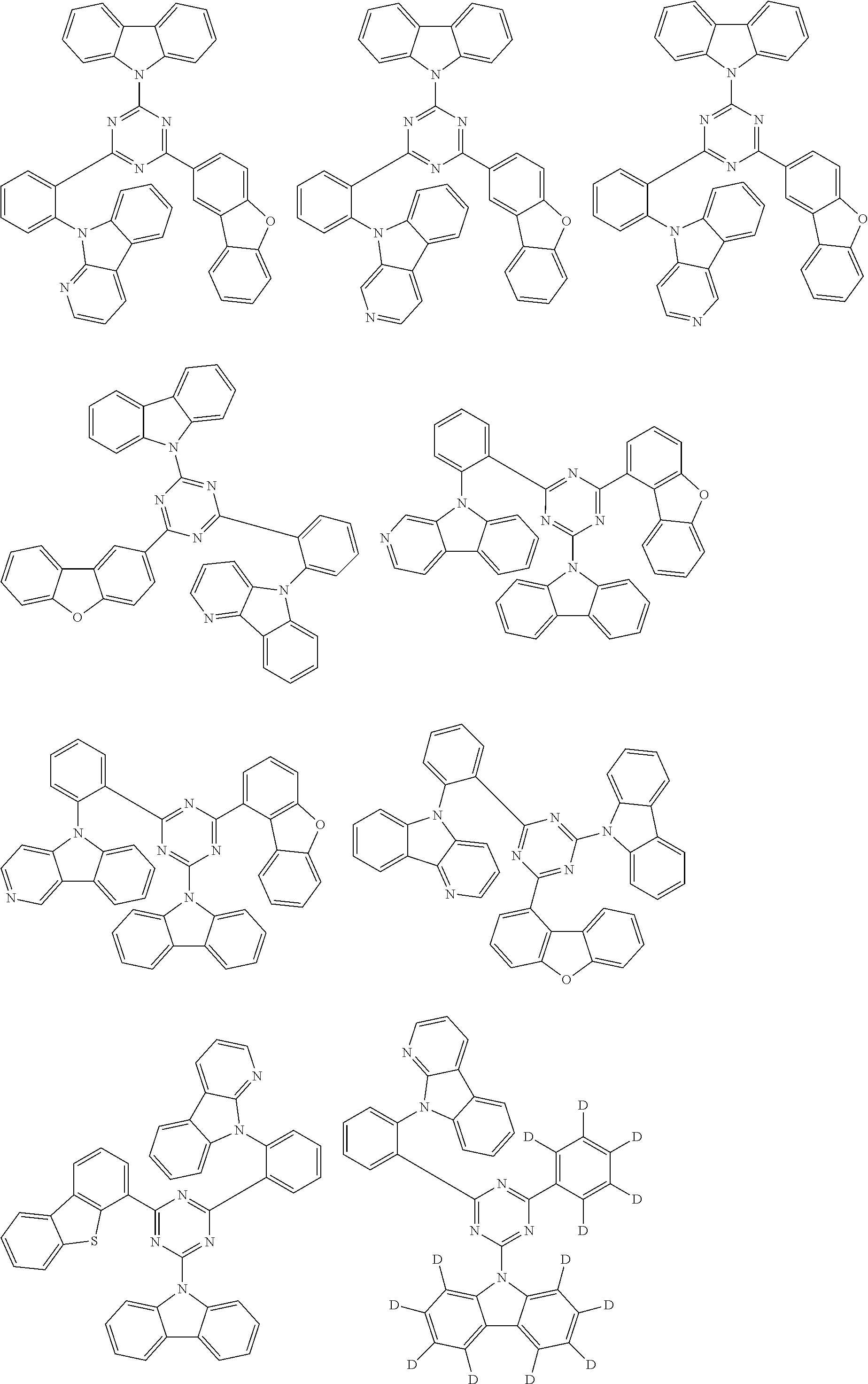

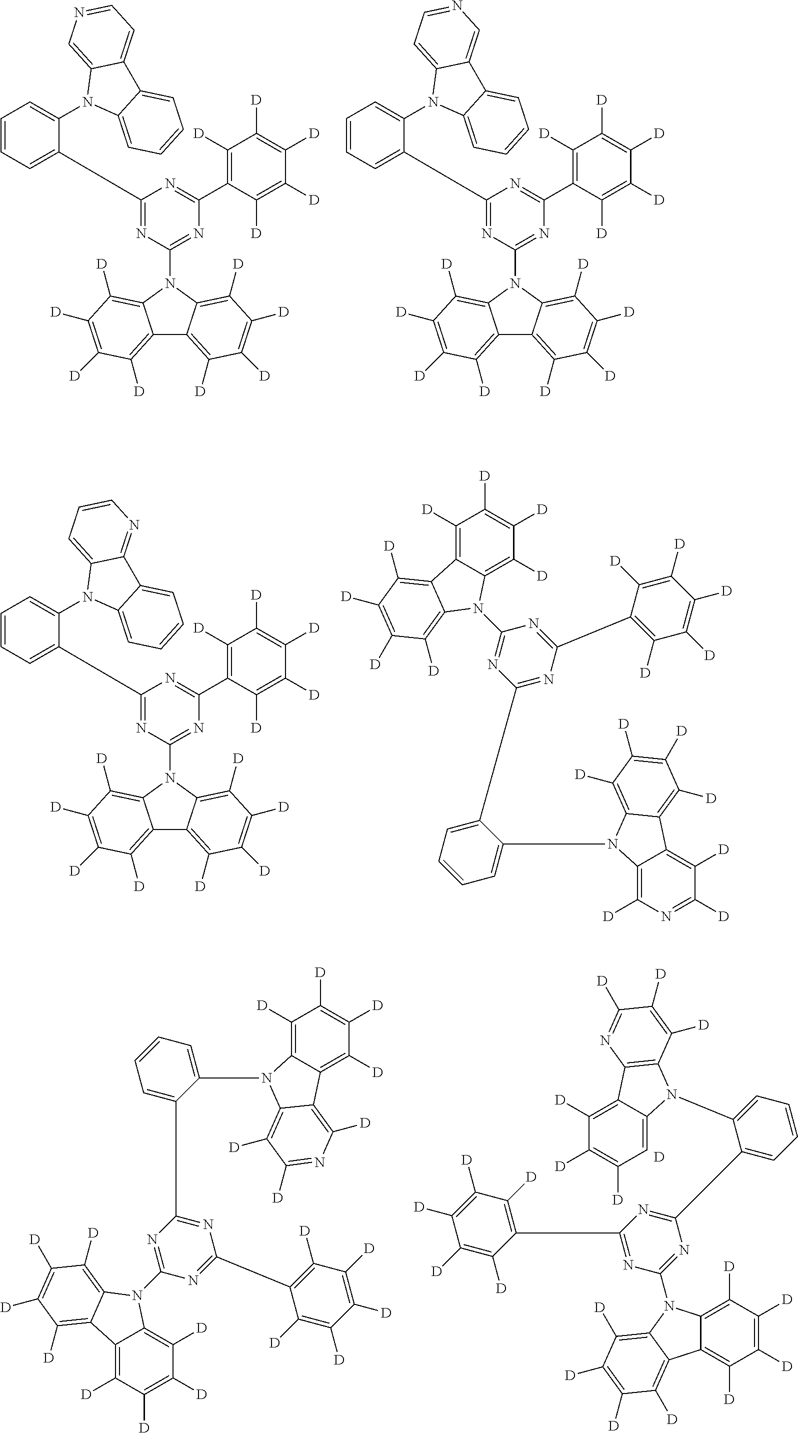

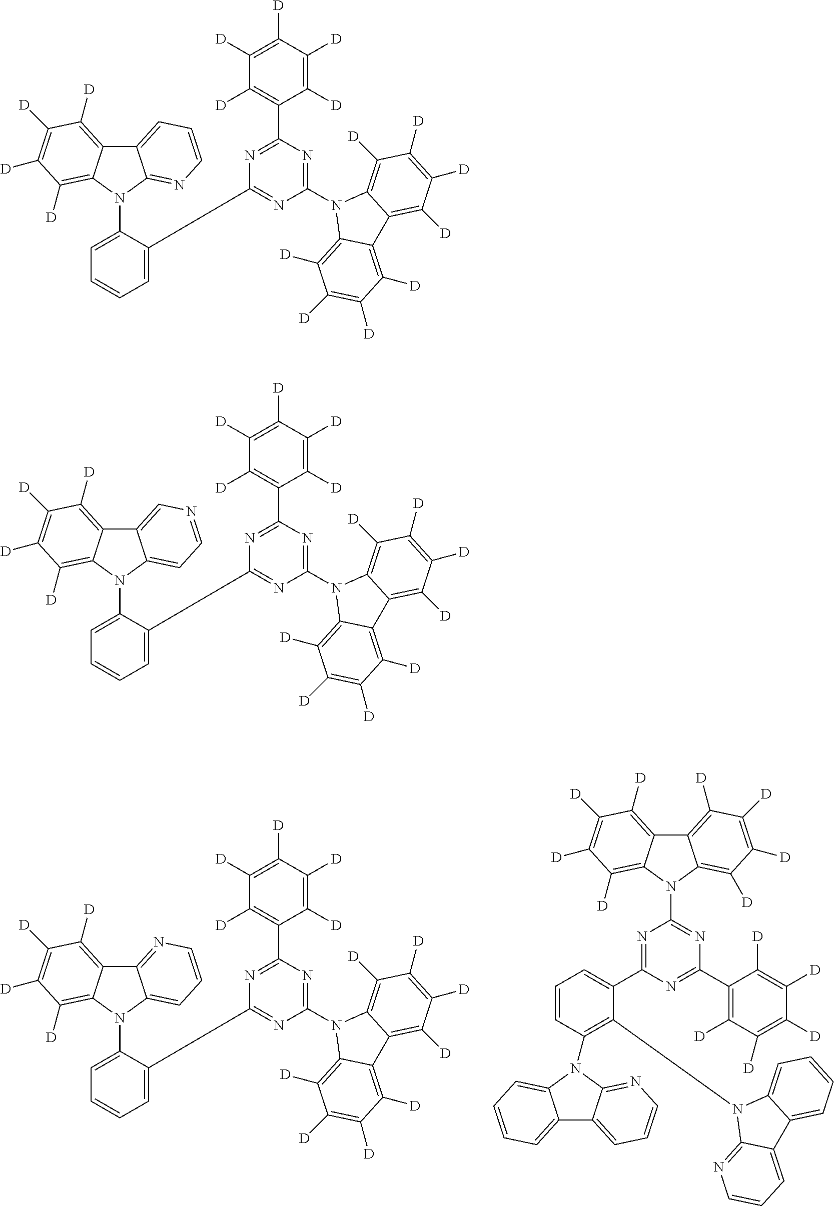

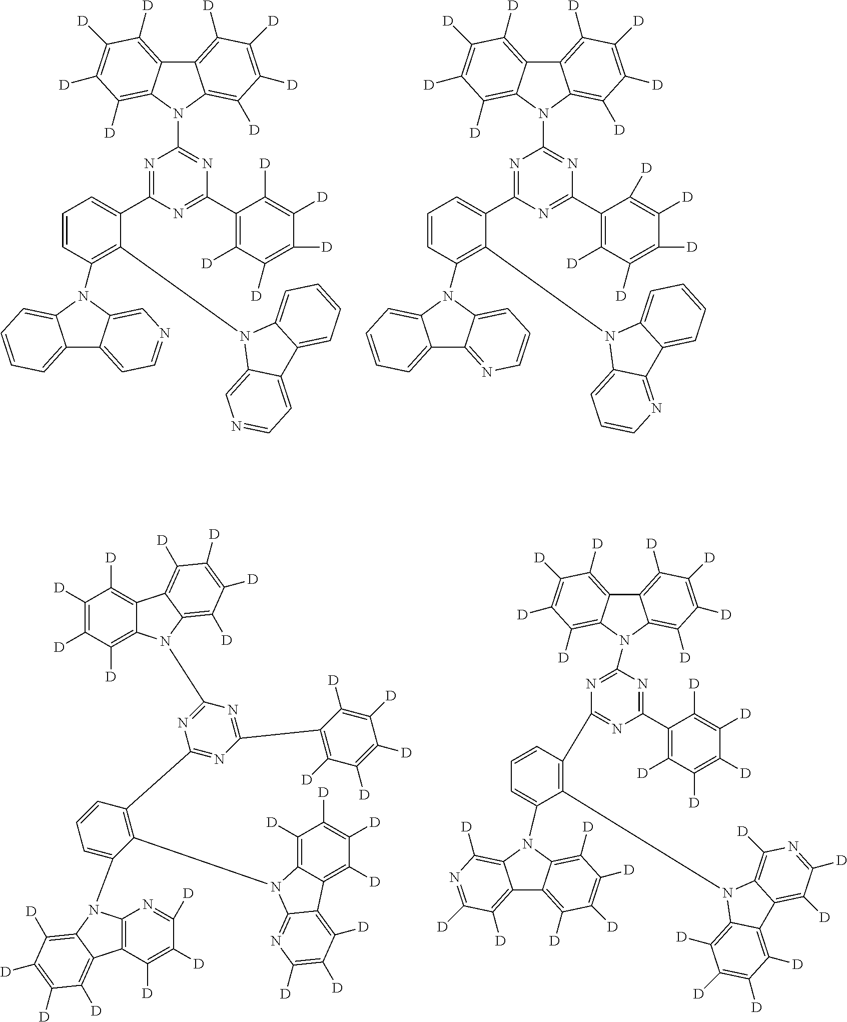

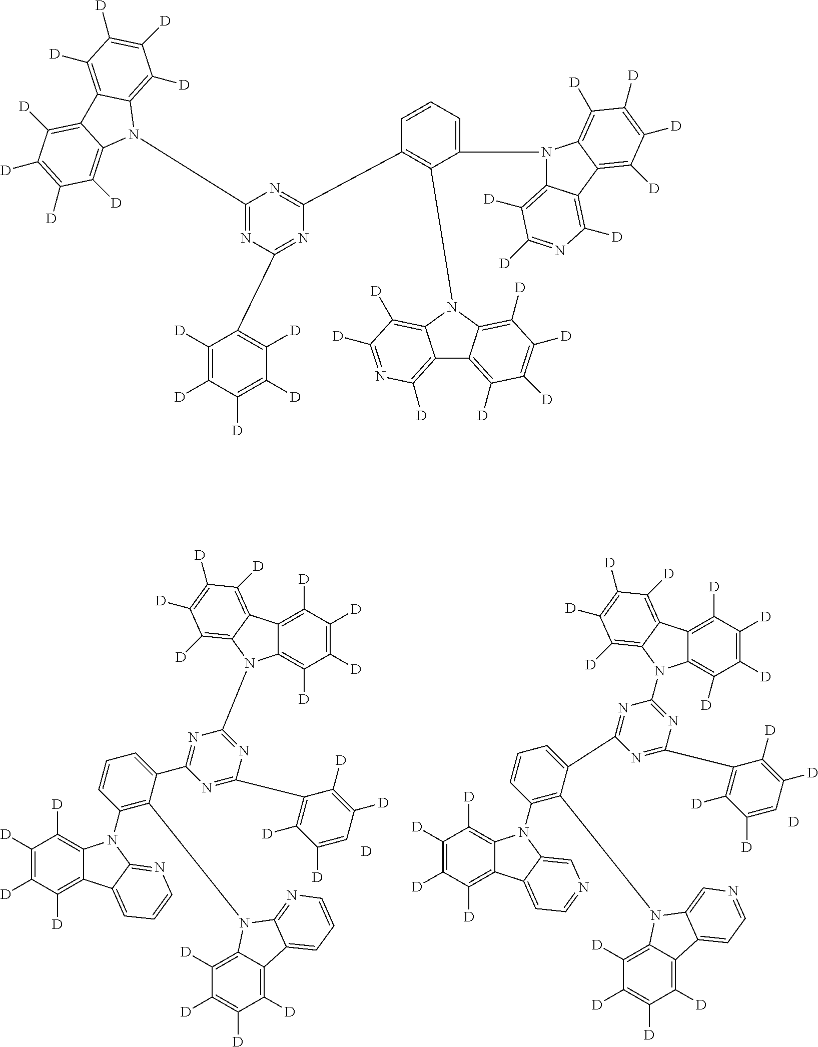

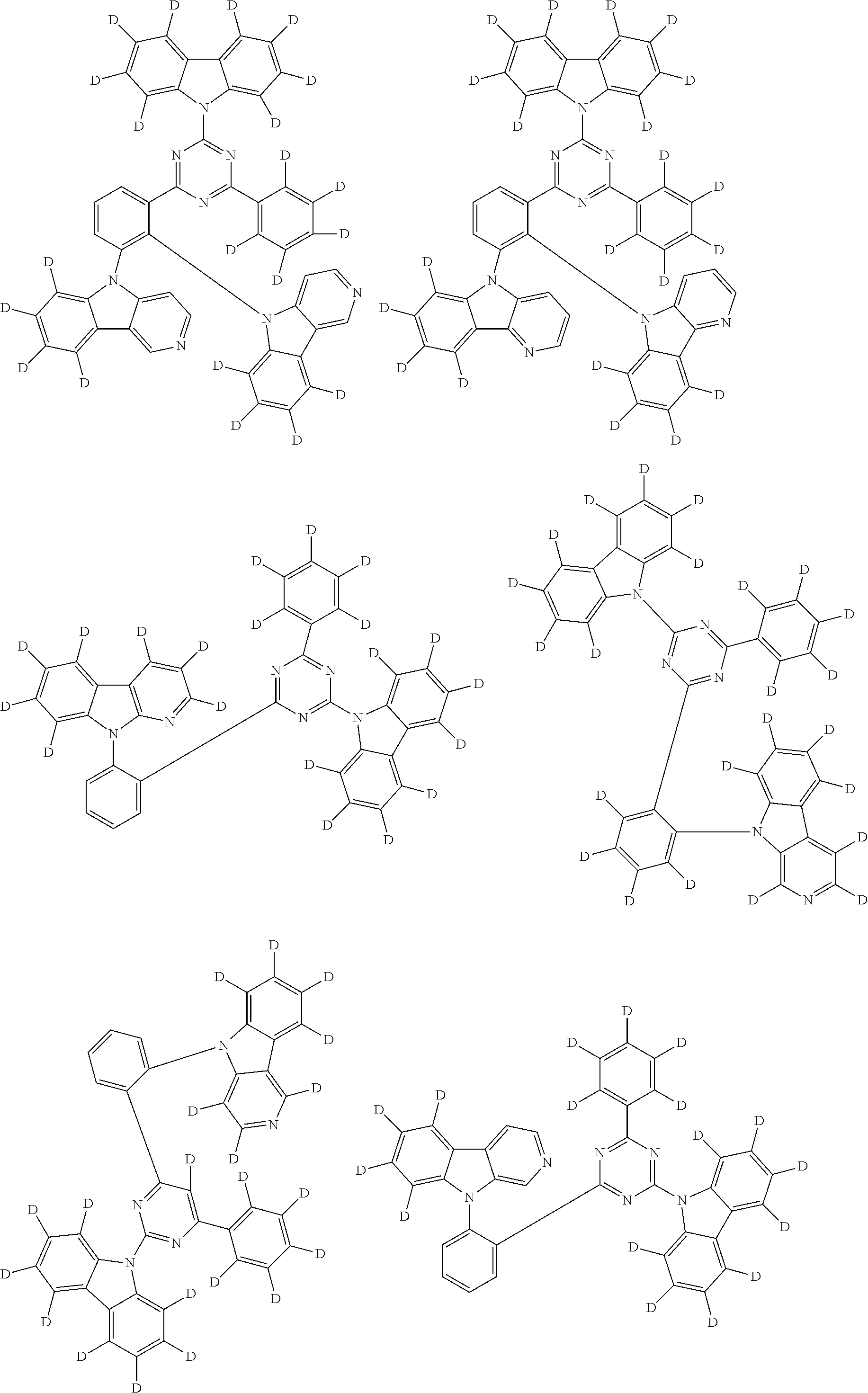

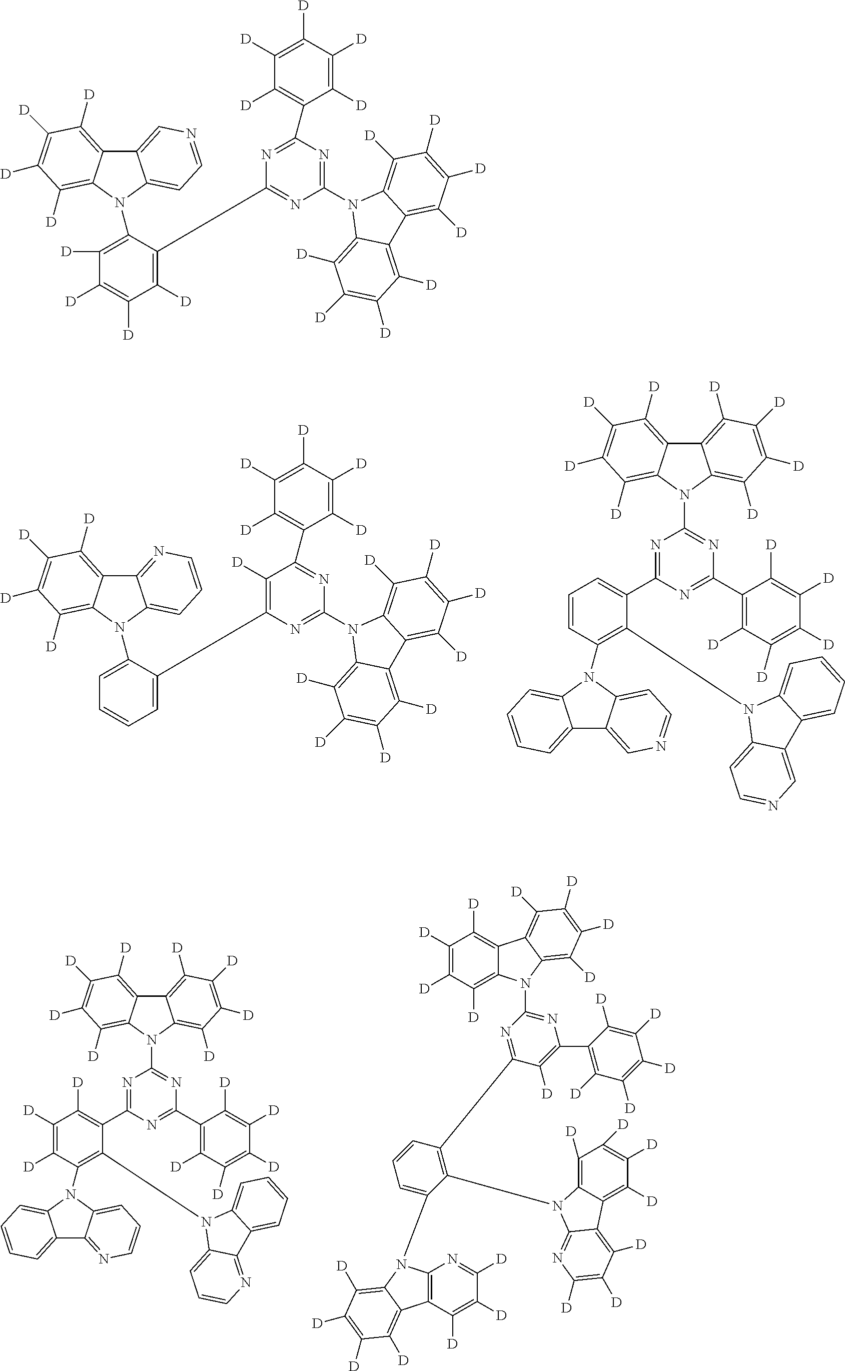

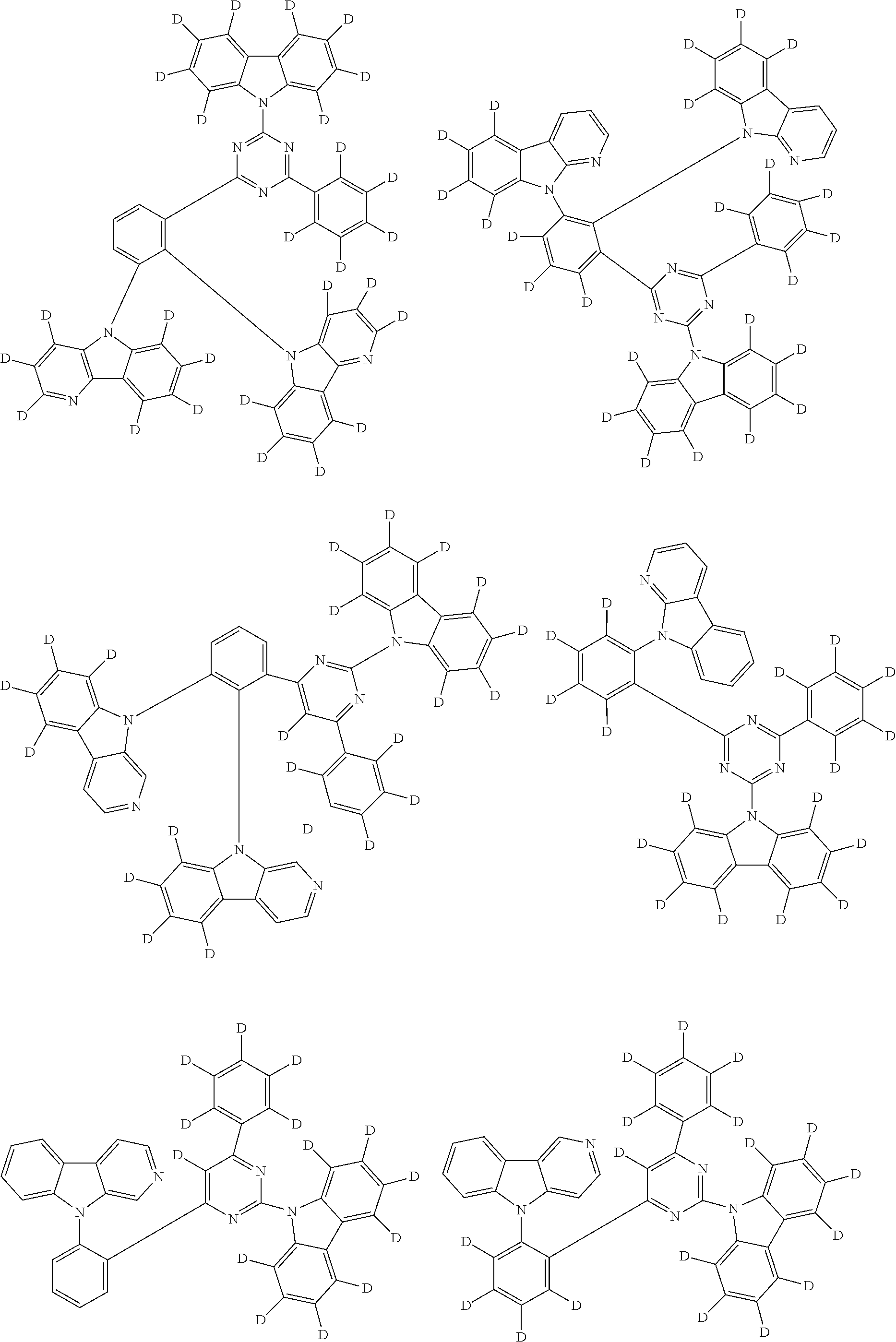

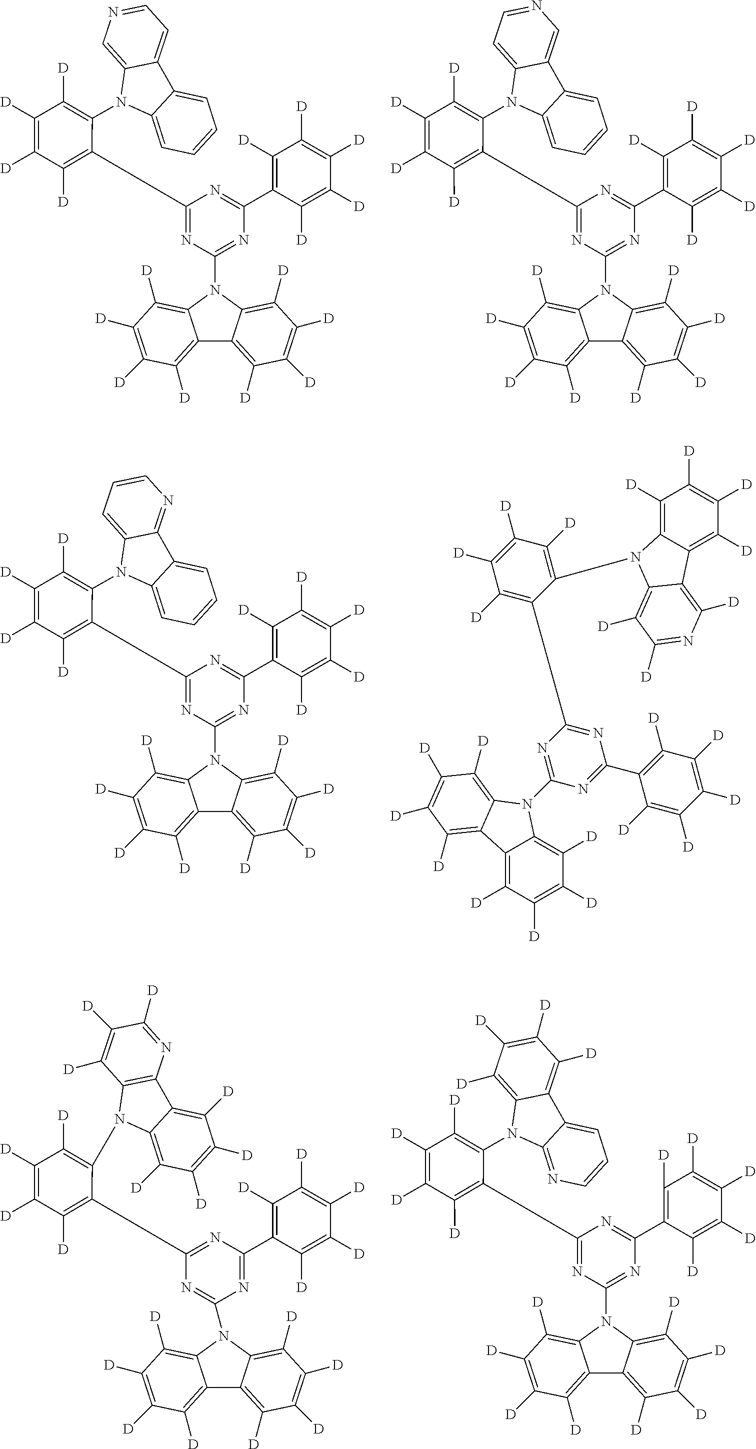

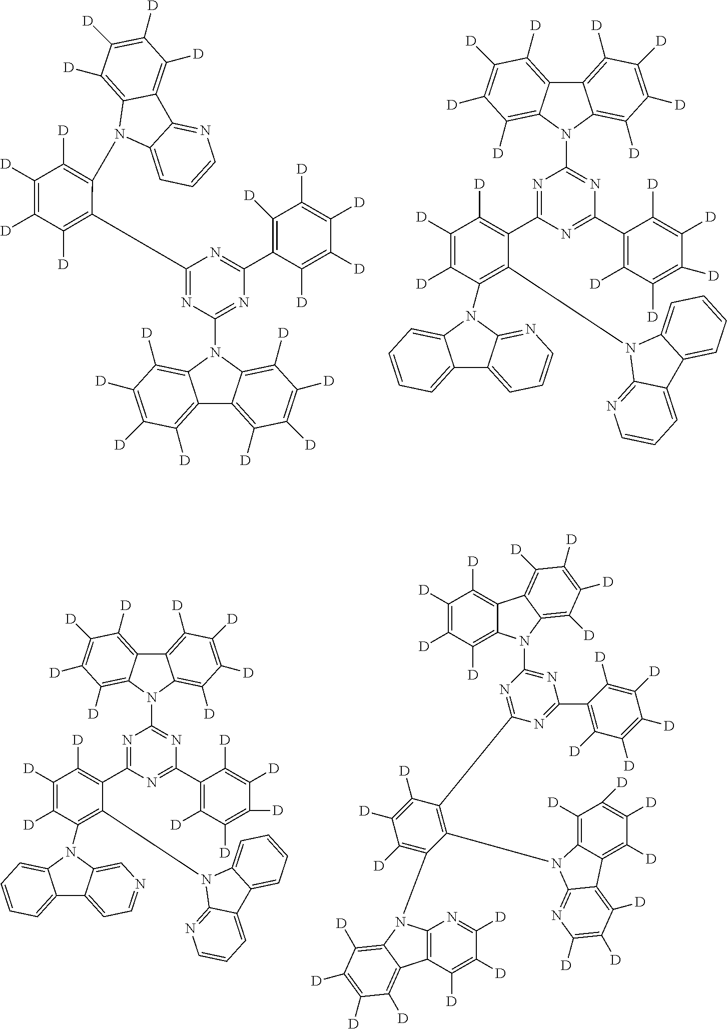

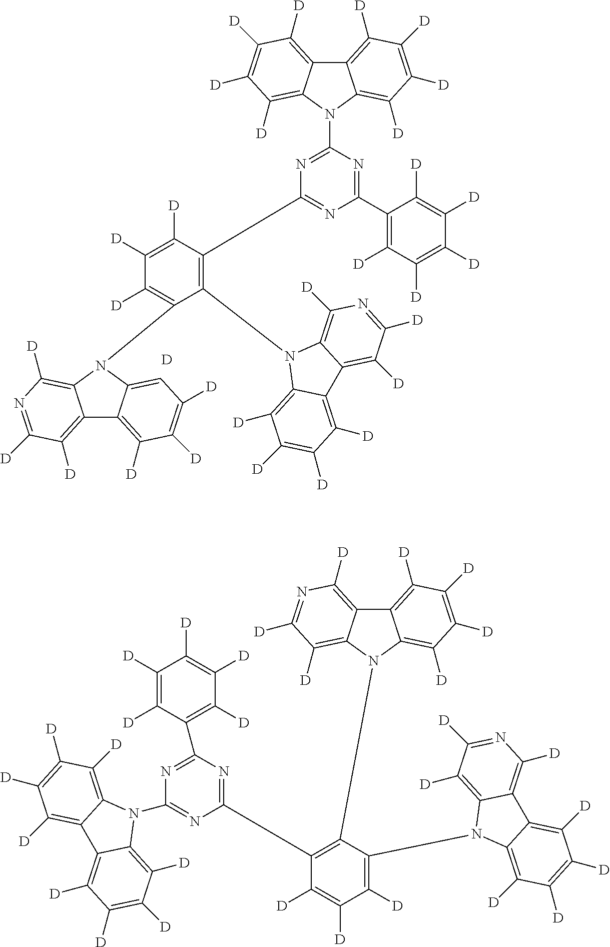

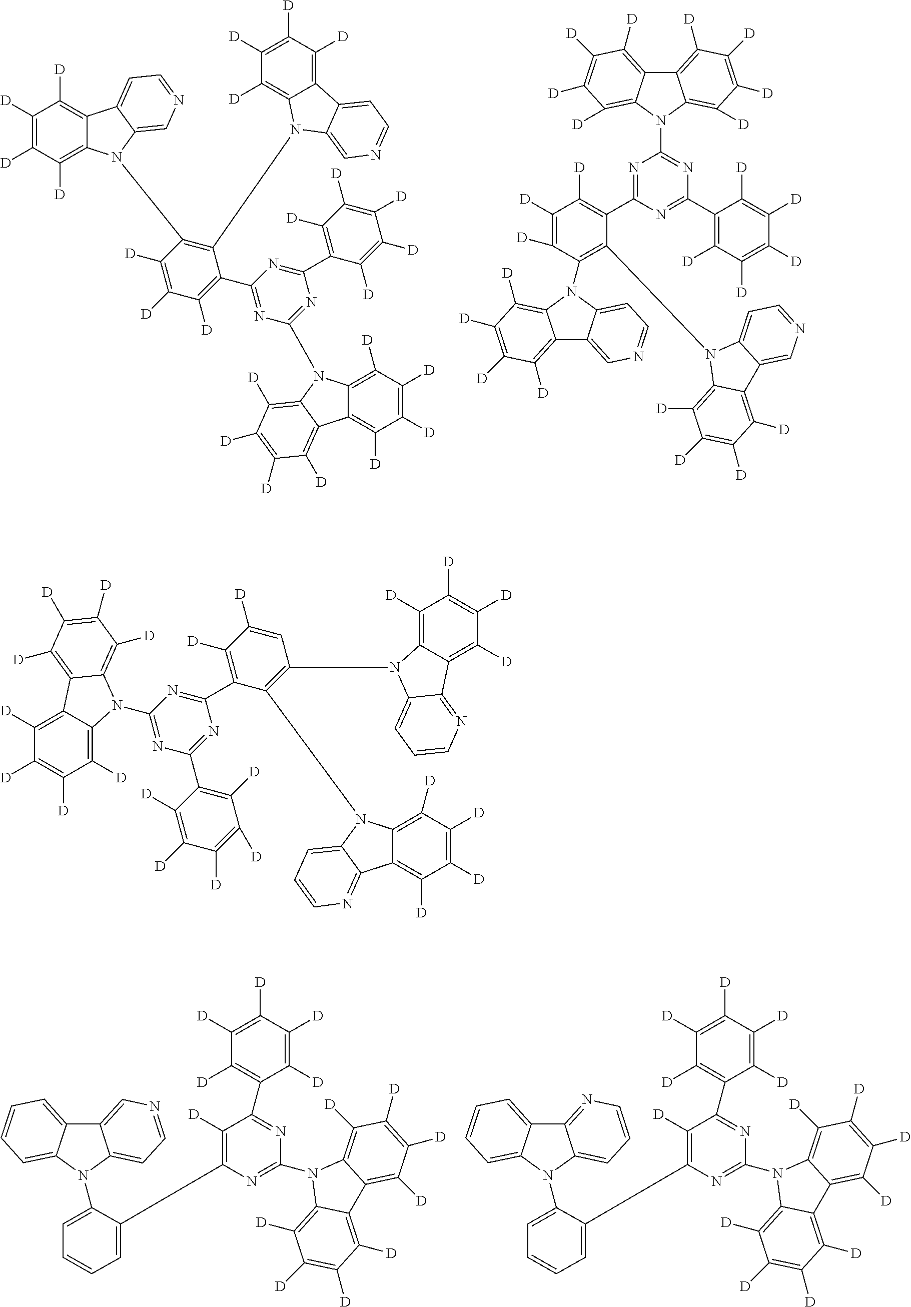

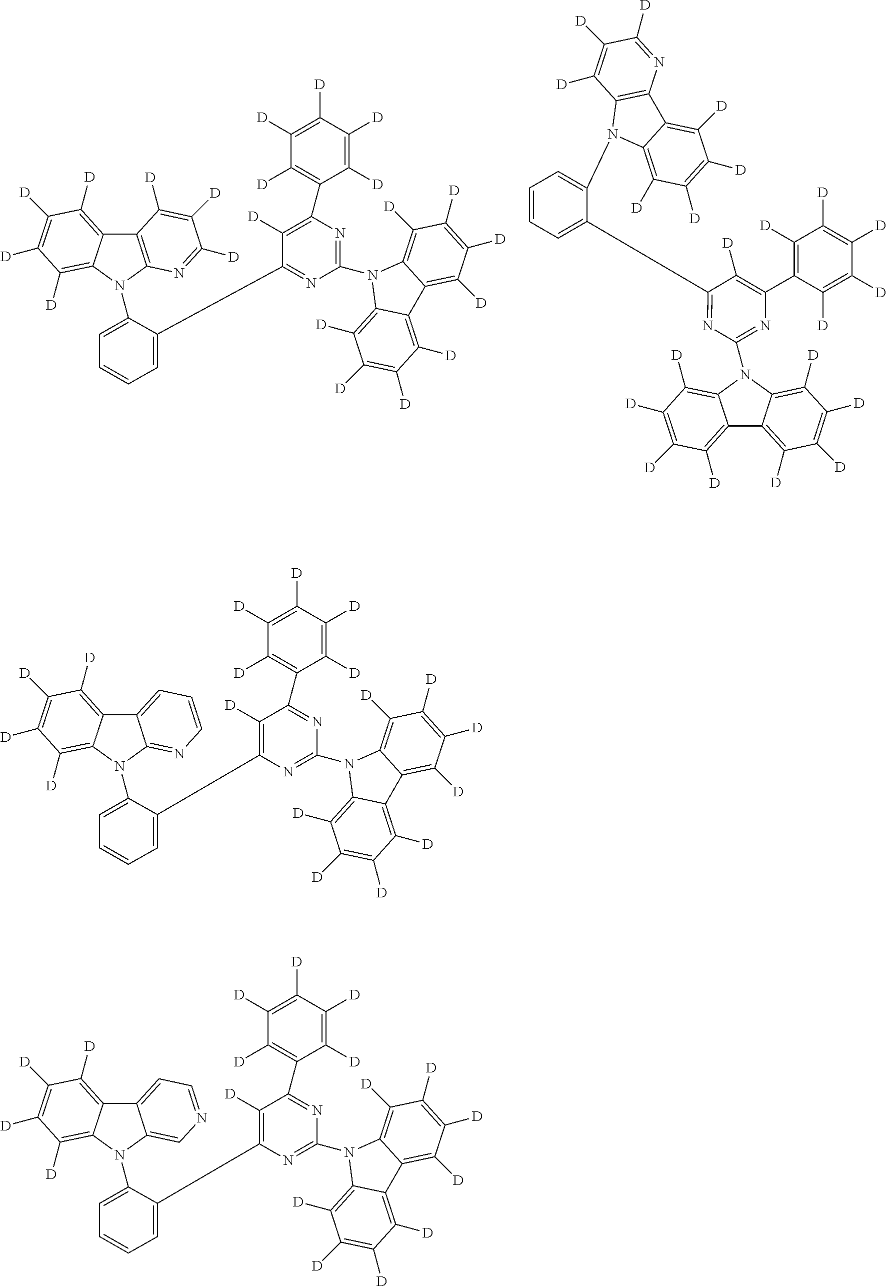

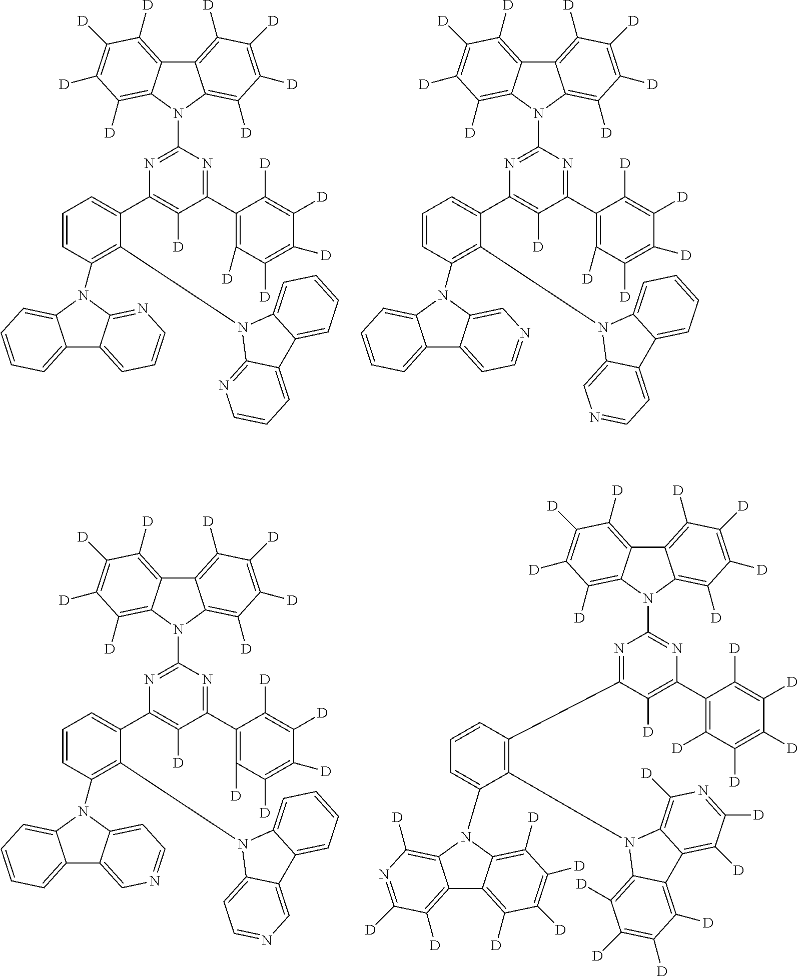

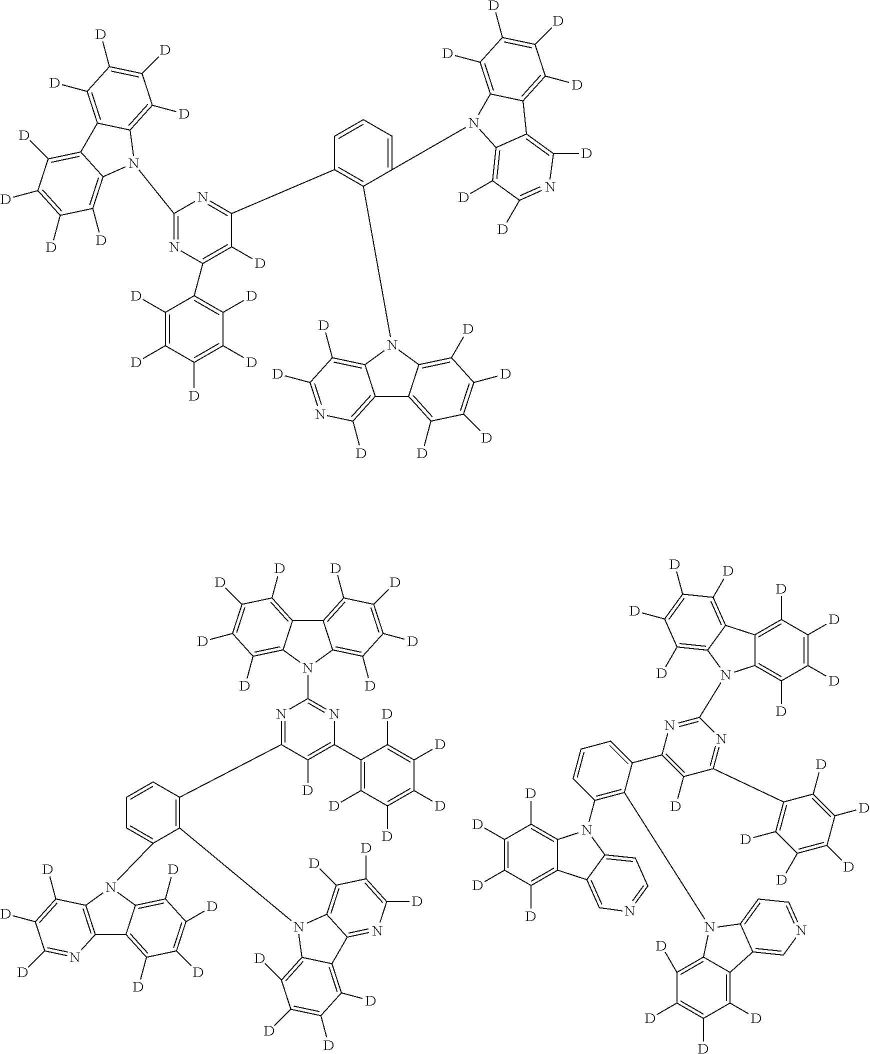

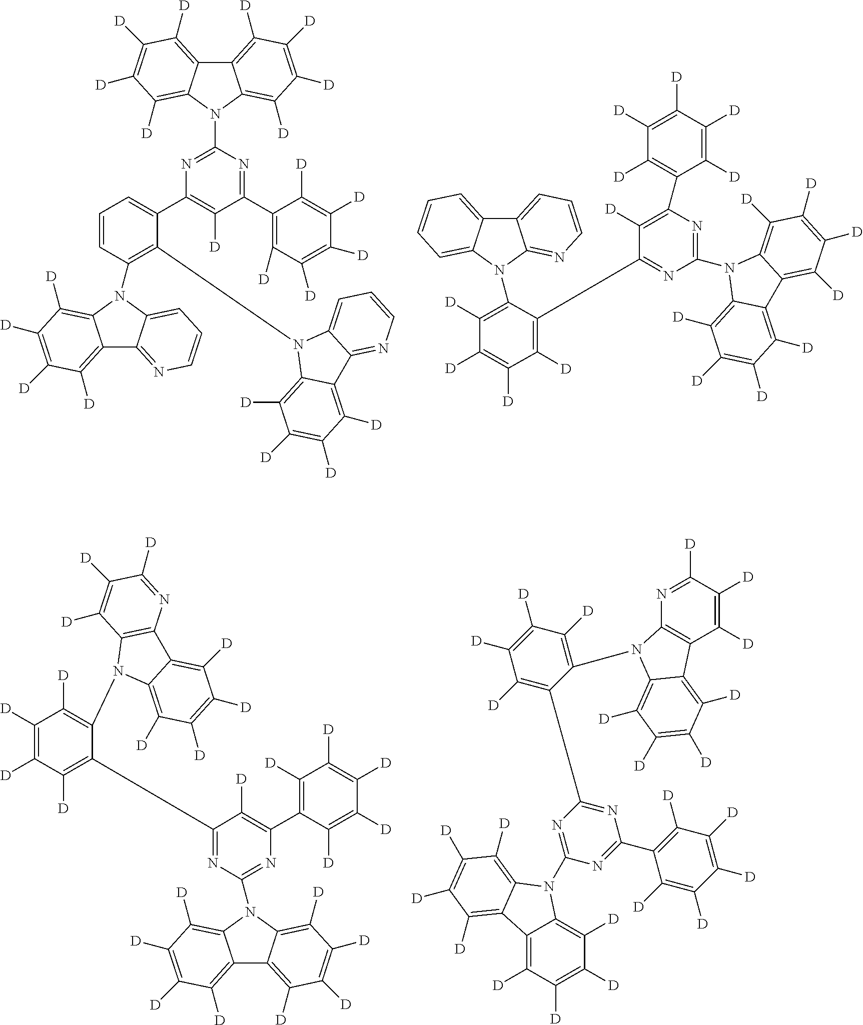

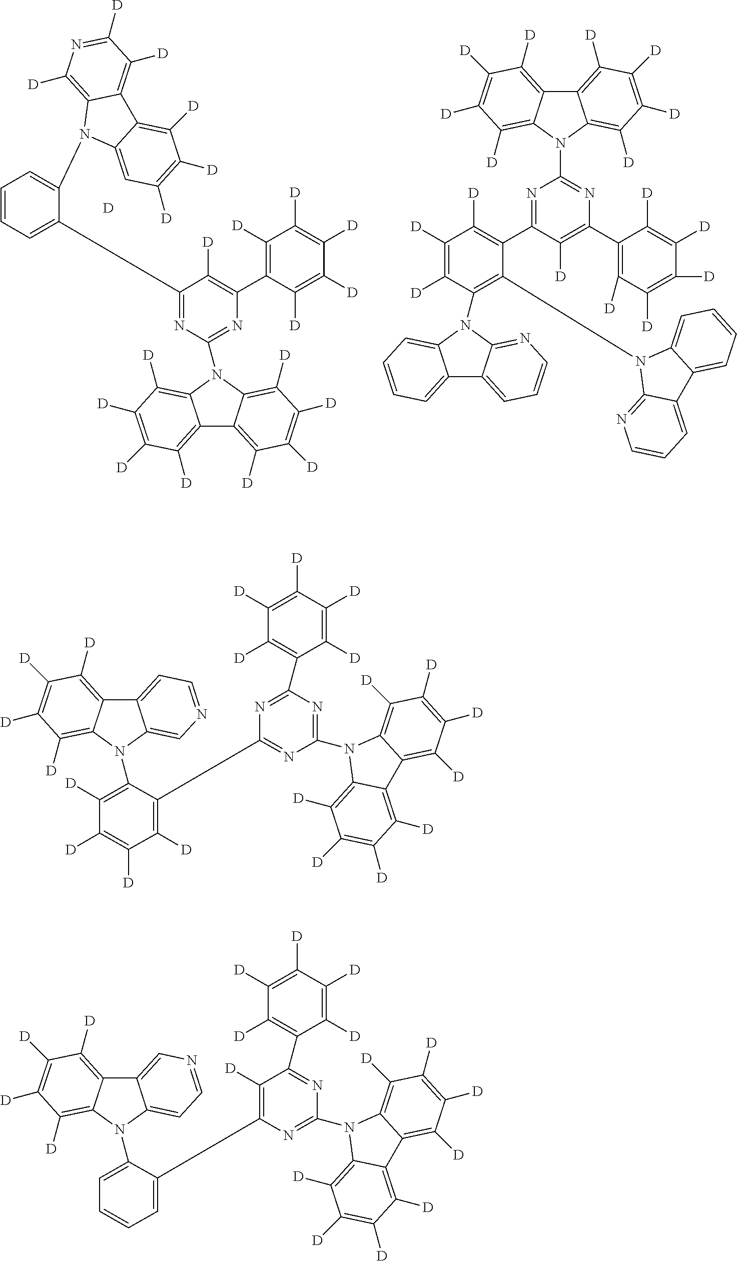

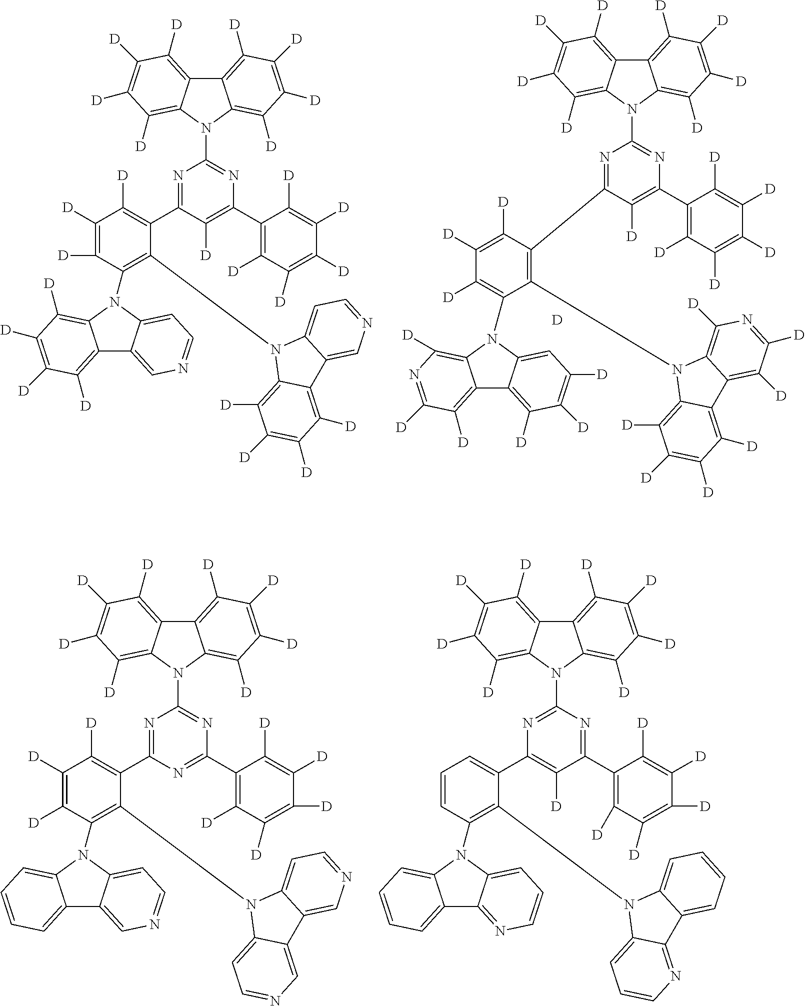

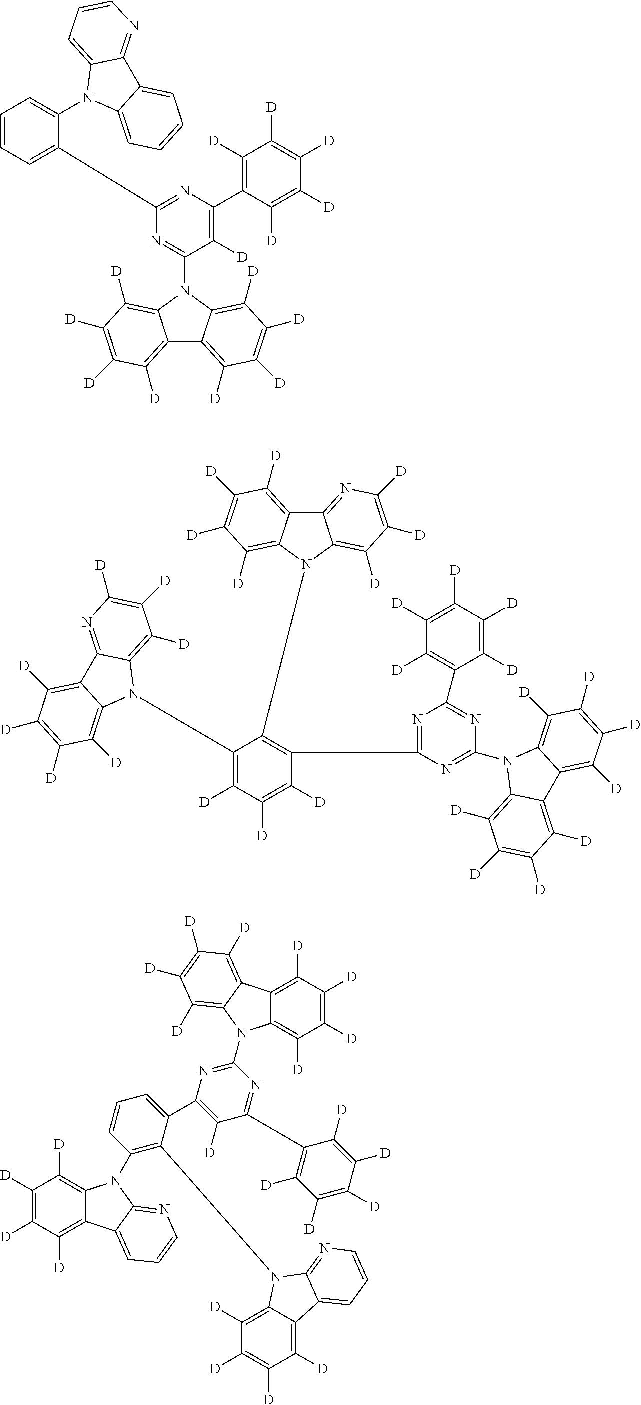

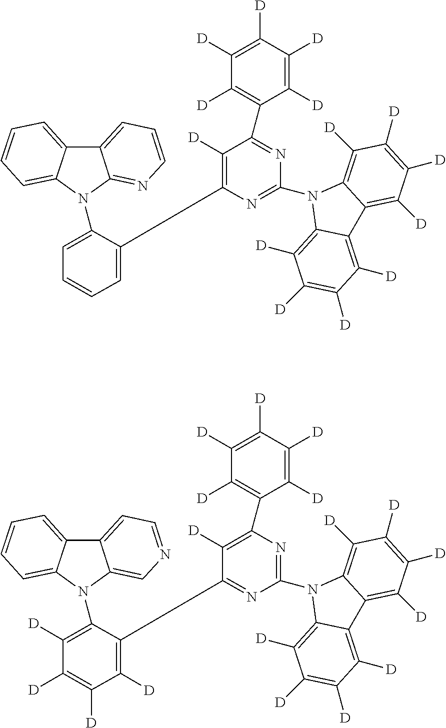 [SMILES: [2H]c1c(-c2ccccc2-n2c3ccccc3c3cccnc32)nc(-n2c3c([2H])c([2H])c([2H])c([2H])c3c3c([2H])c([2H])c([2H])c([2H])c32)nc1-c1c([2H])c([2H])c([2H])c([2H])c1[2H] ; [2H]c1c(-c2c([2H])c([2H])c([2H])c([2H])c2[2H])nc(-n2c3c([2H])c([2H])c([2H])c([2H])c3c3c([2H])c([2H])c([2H])c([2H])c32)nc1-c1c([2H])c([2H])c([2H])c([2H])c1-n1c2ccccc2c2ccncc21]

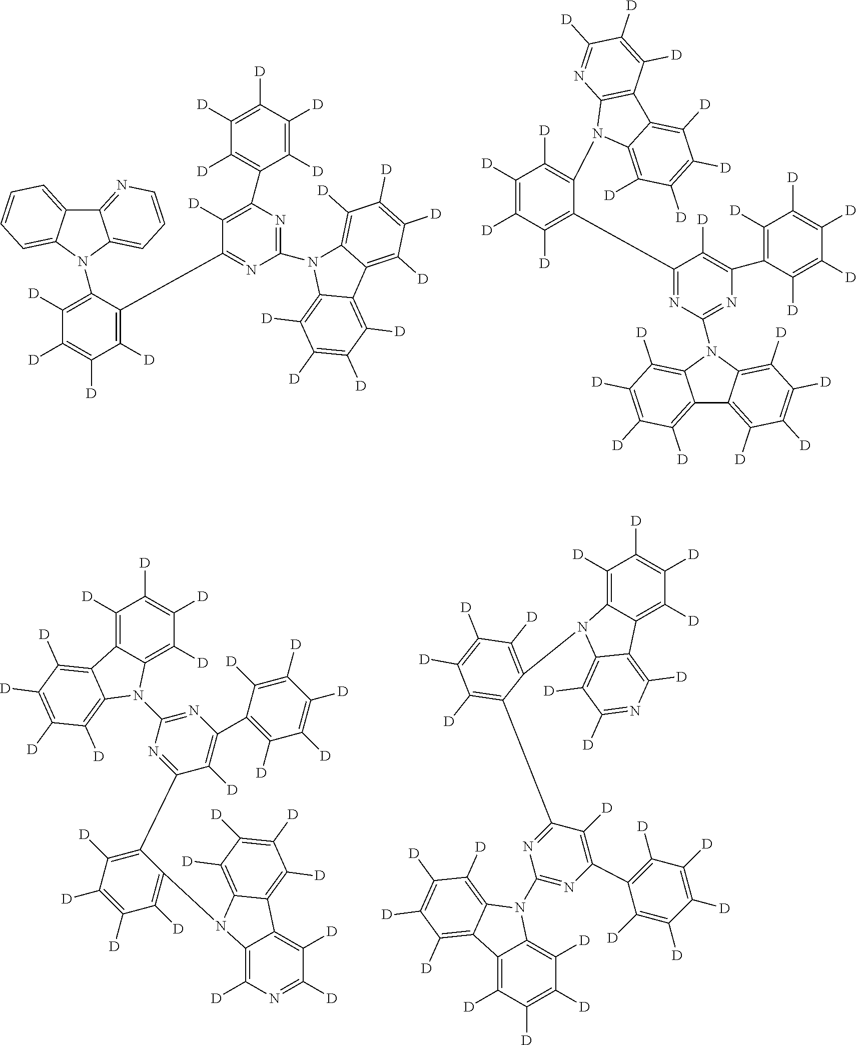

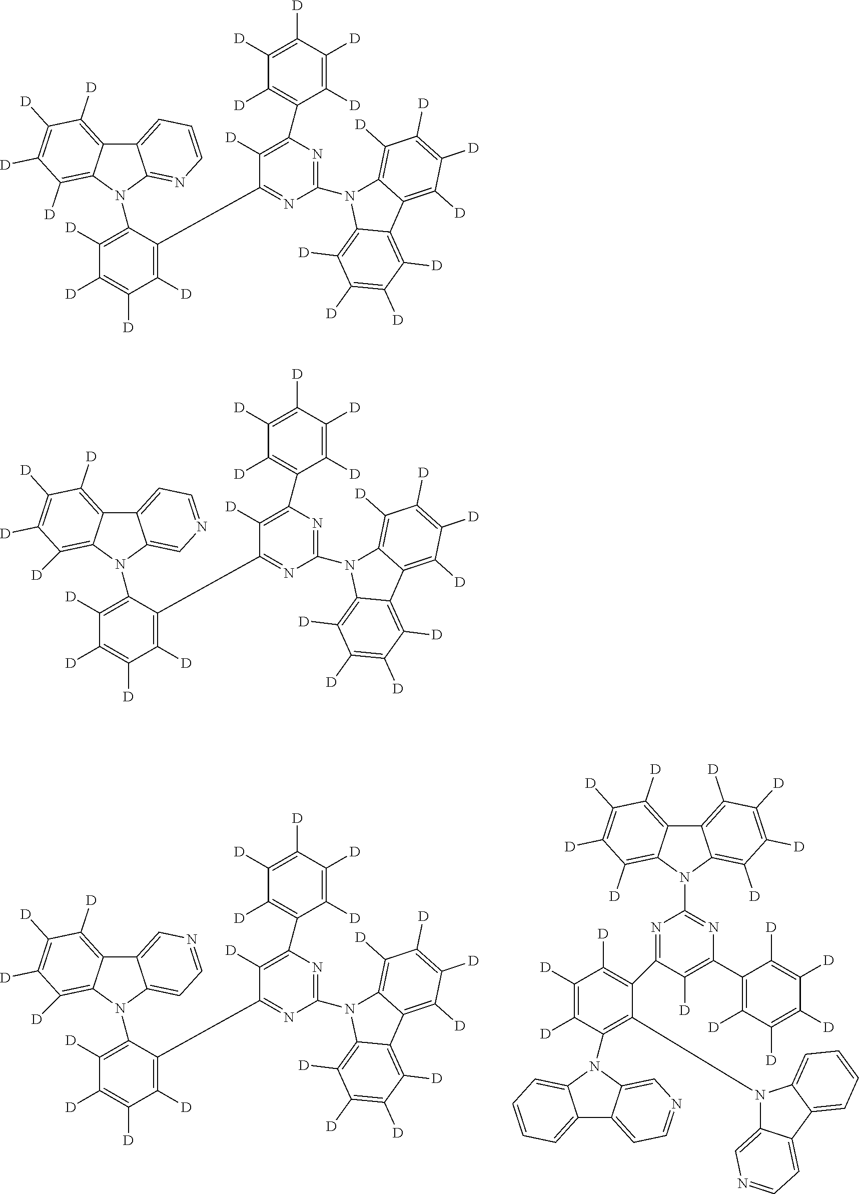

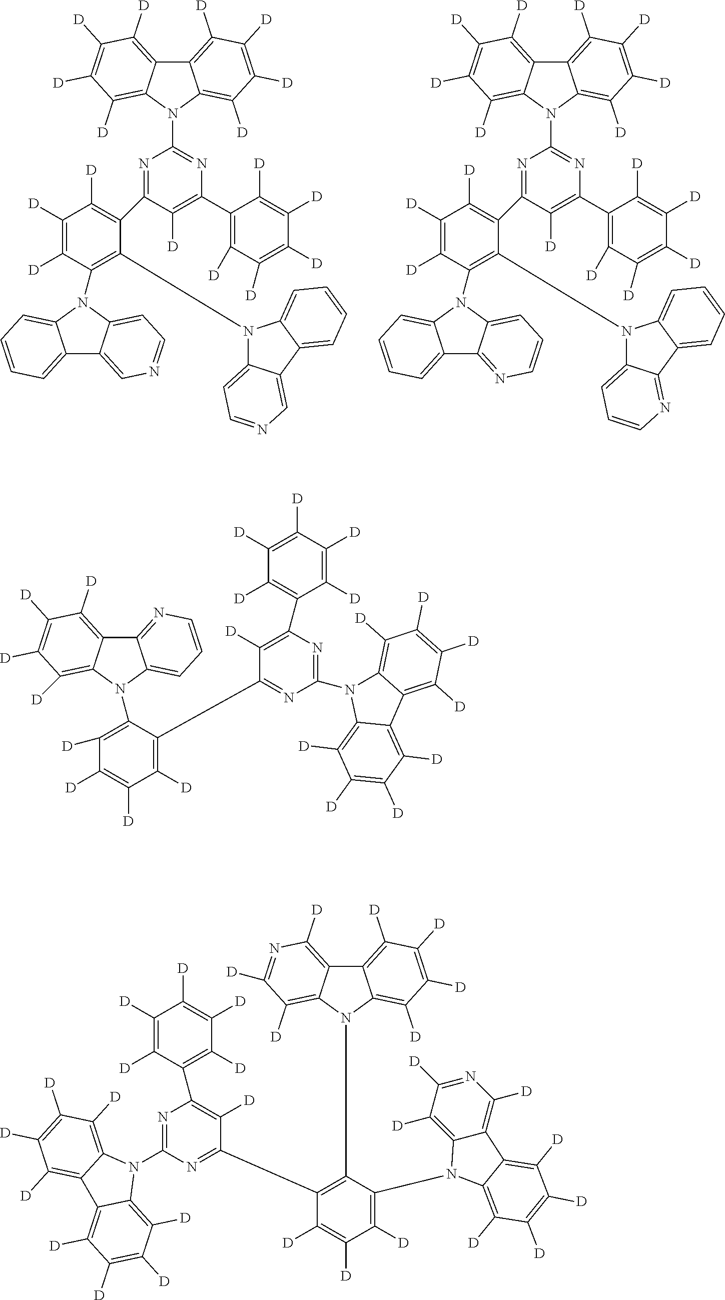

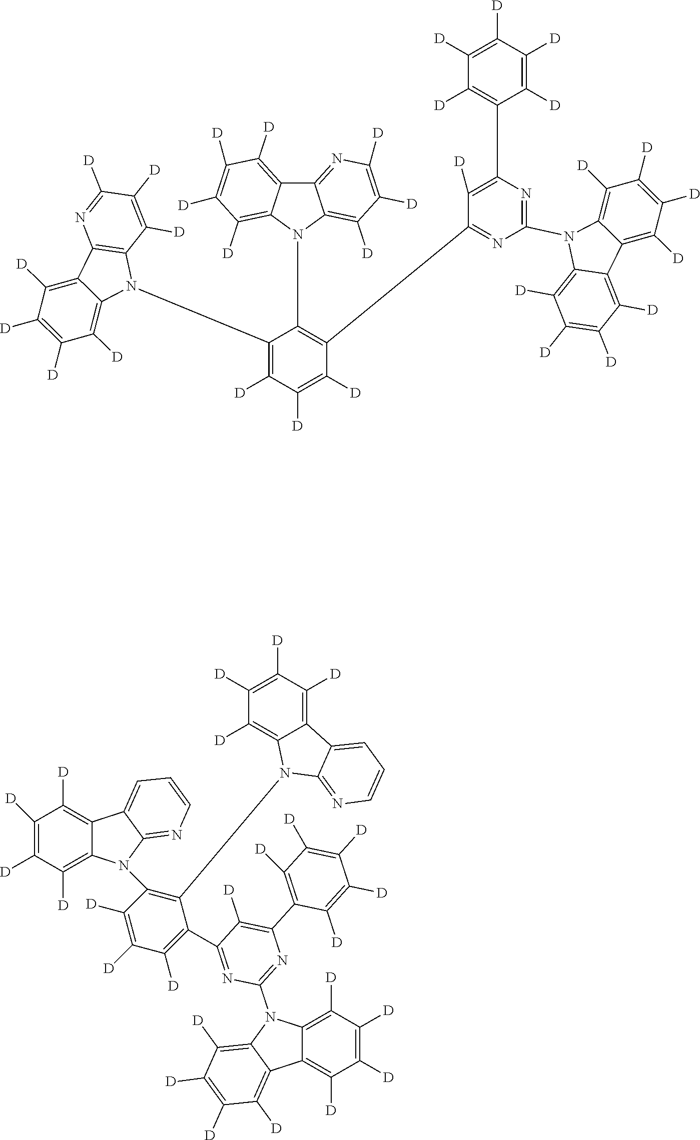

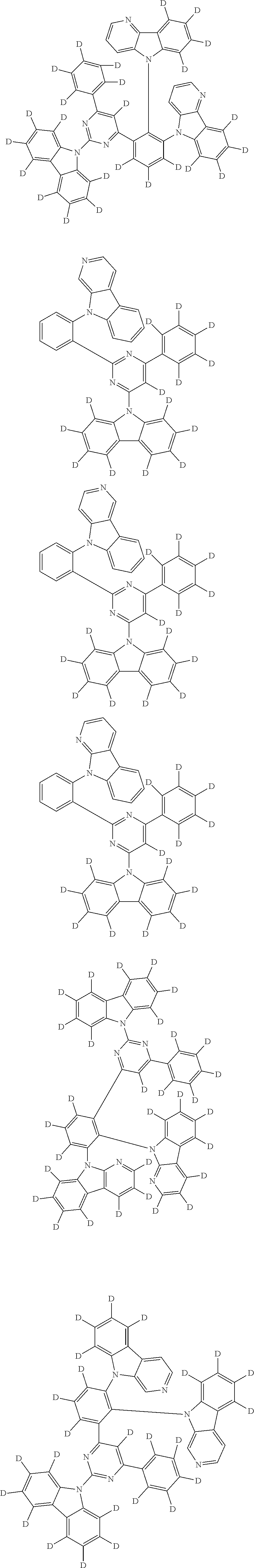

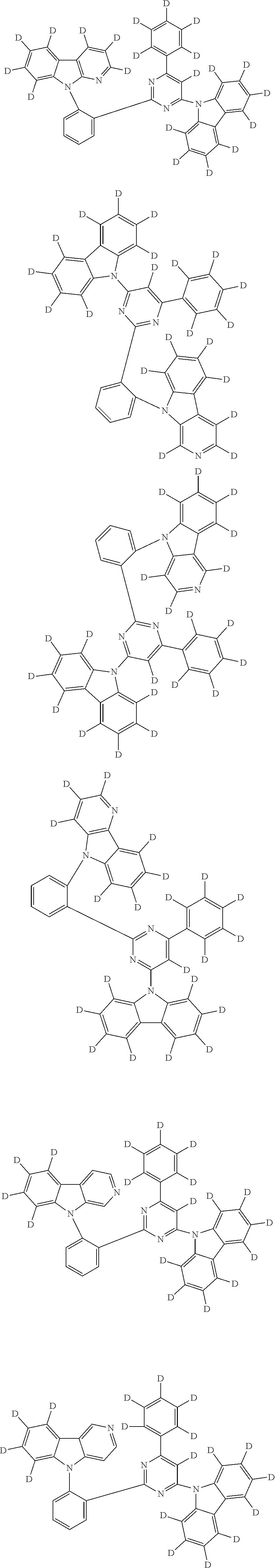

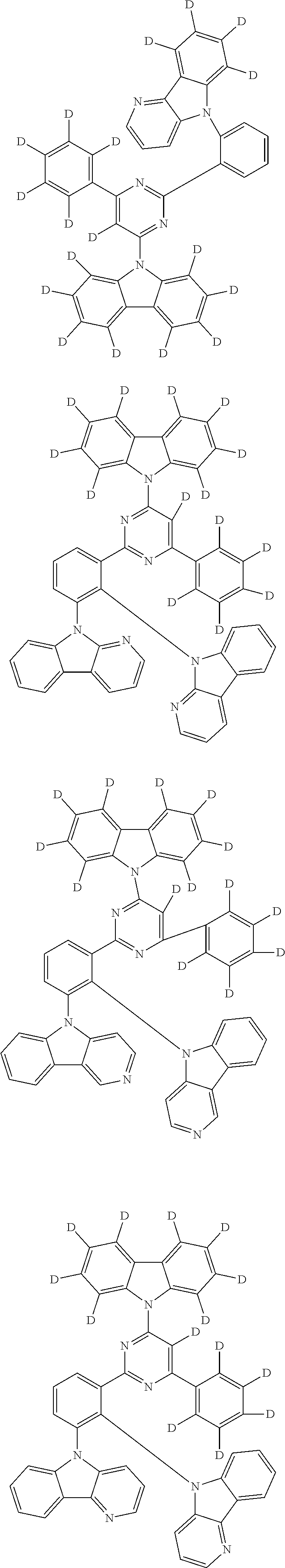

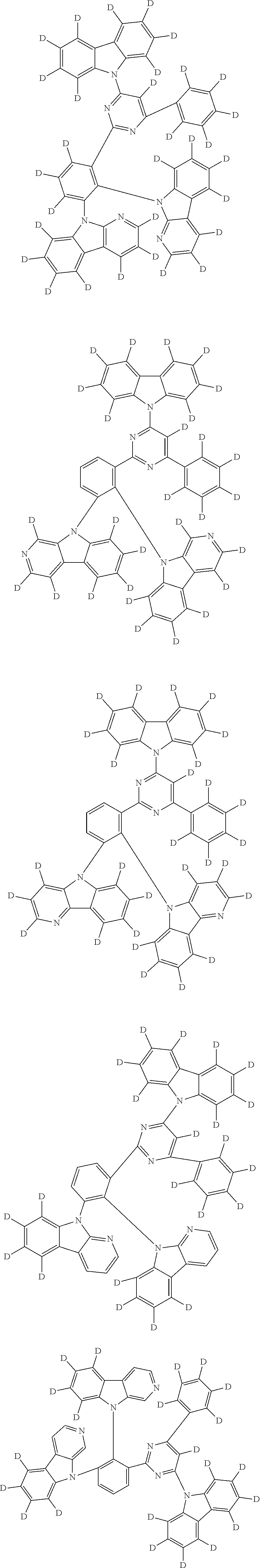

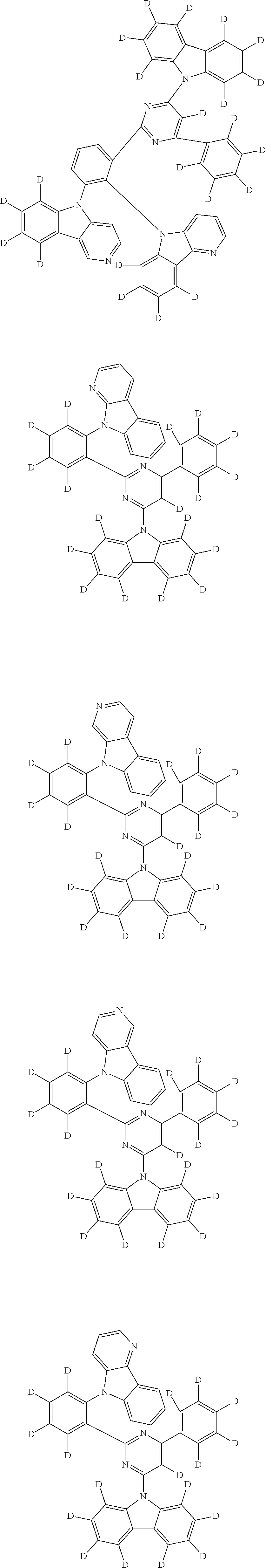

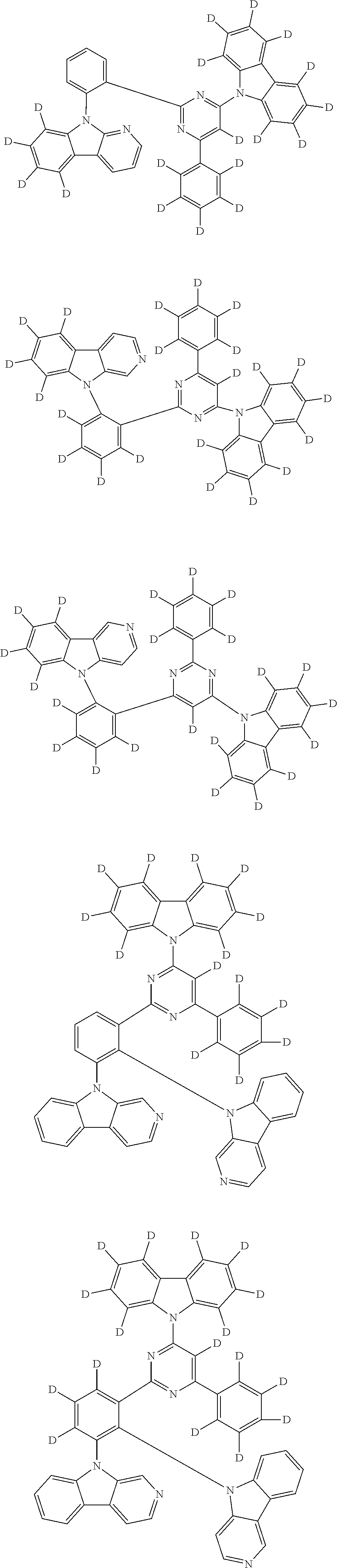

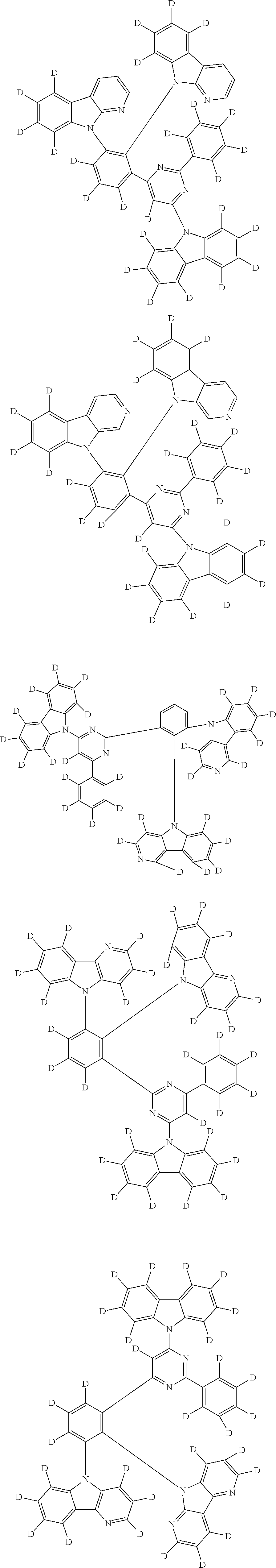

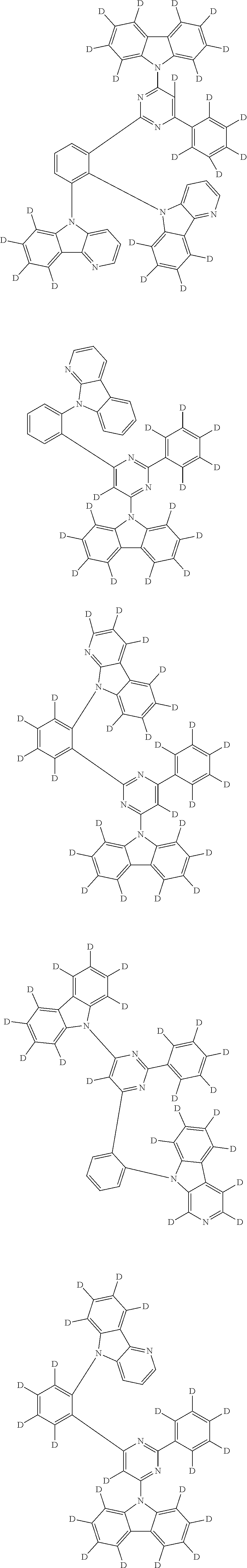

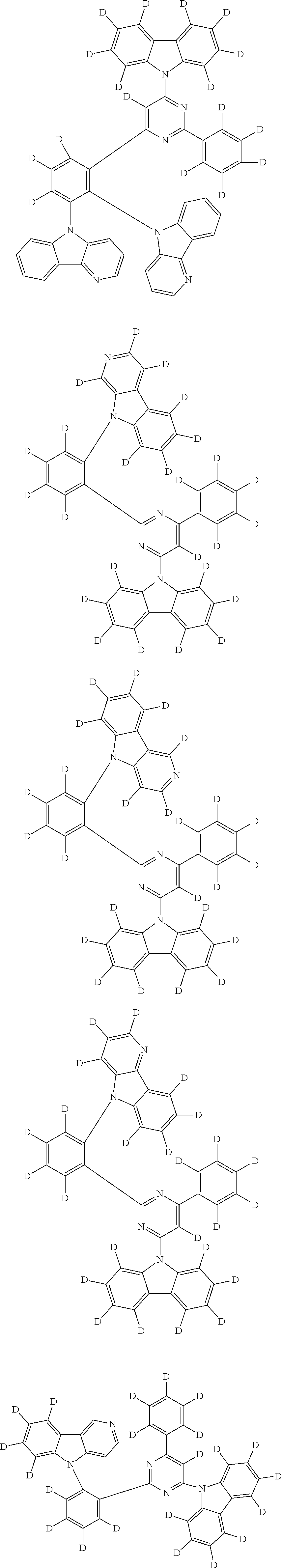

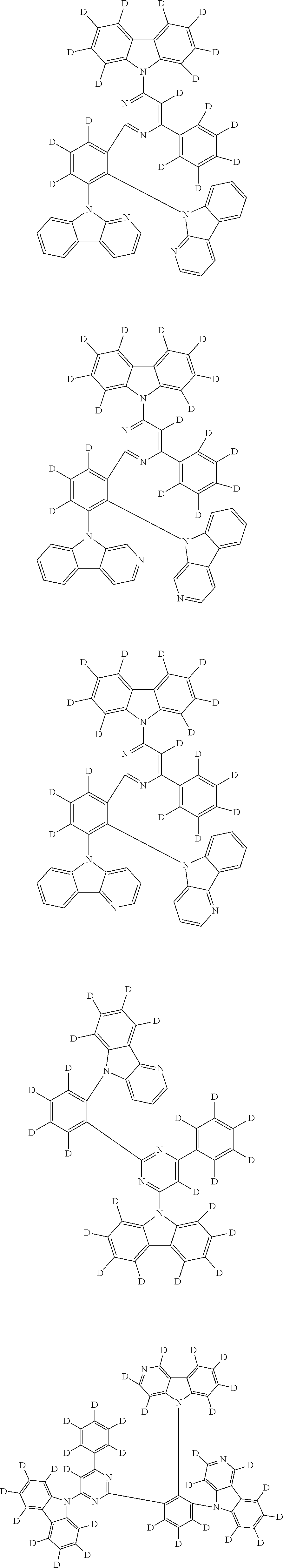

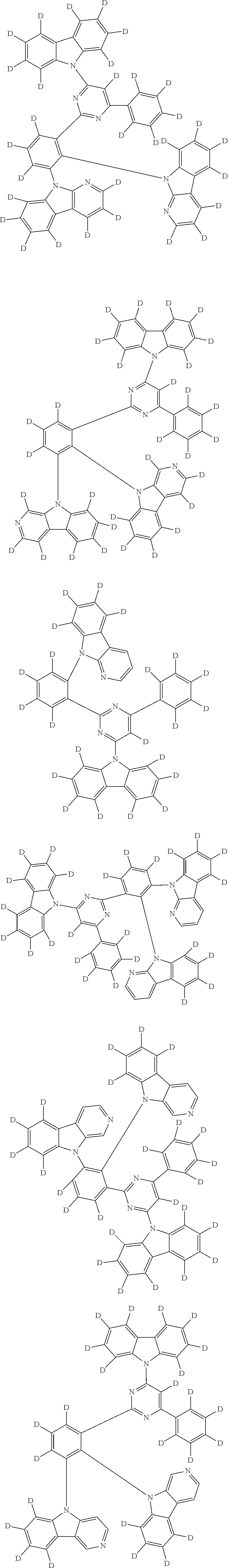

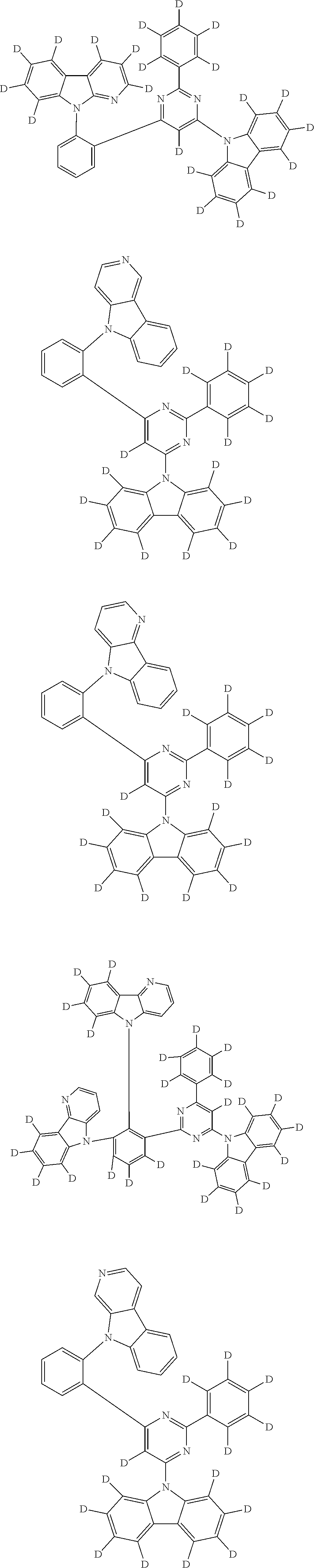

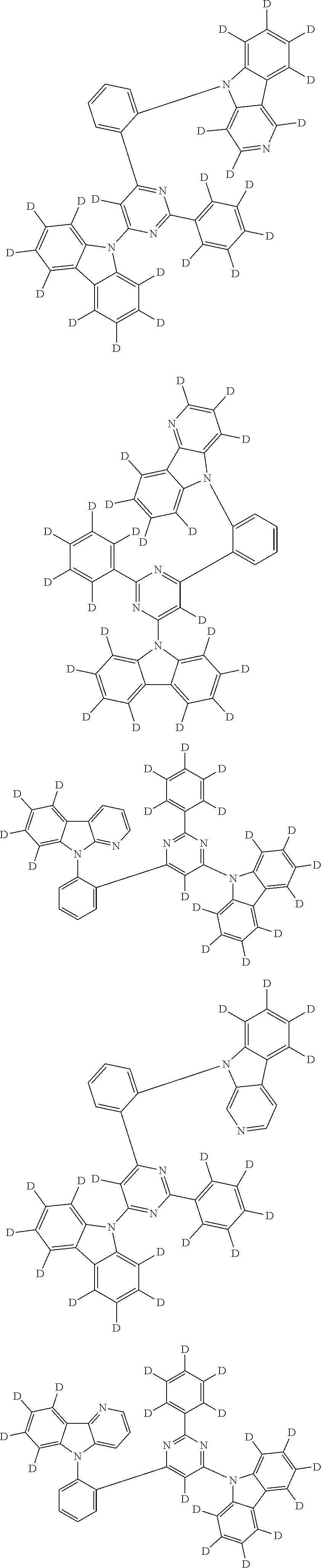

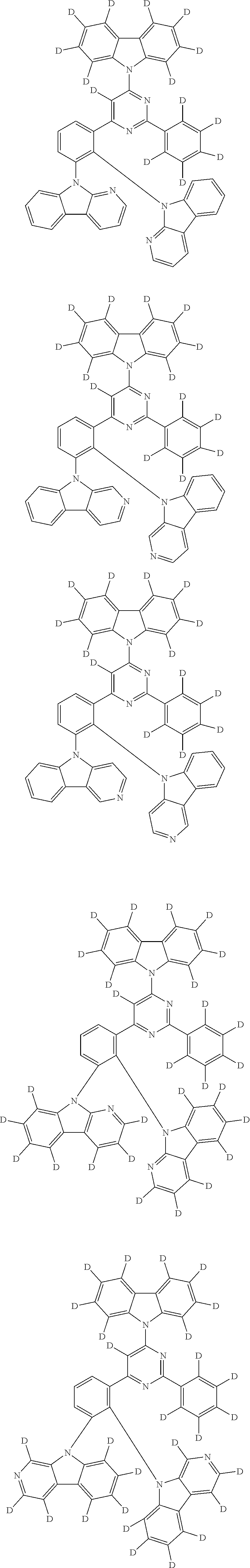

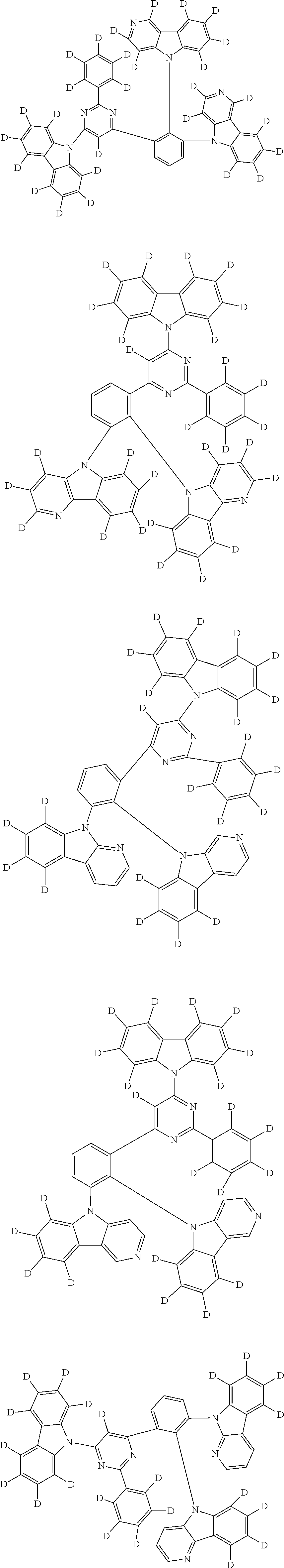

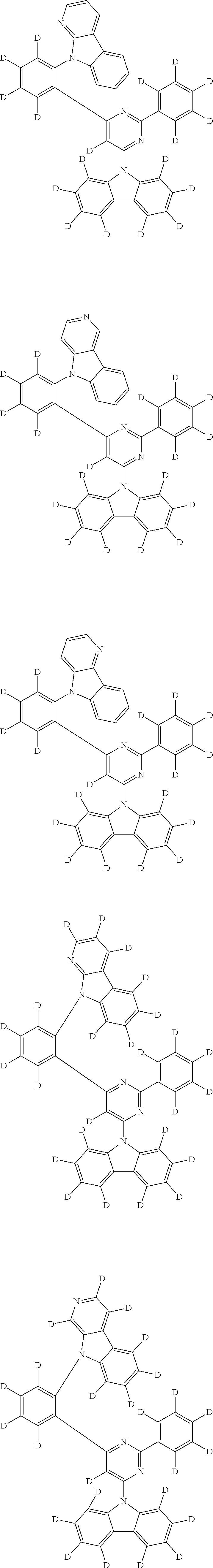

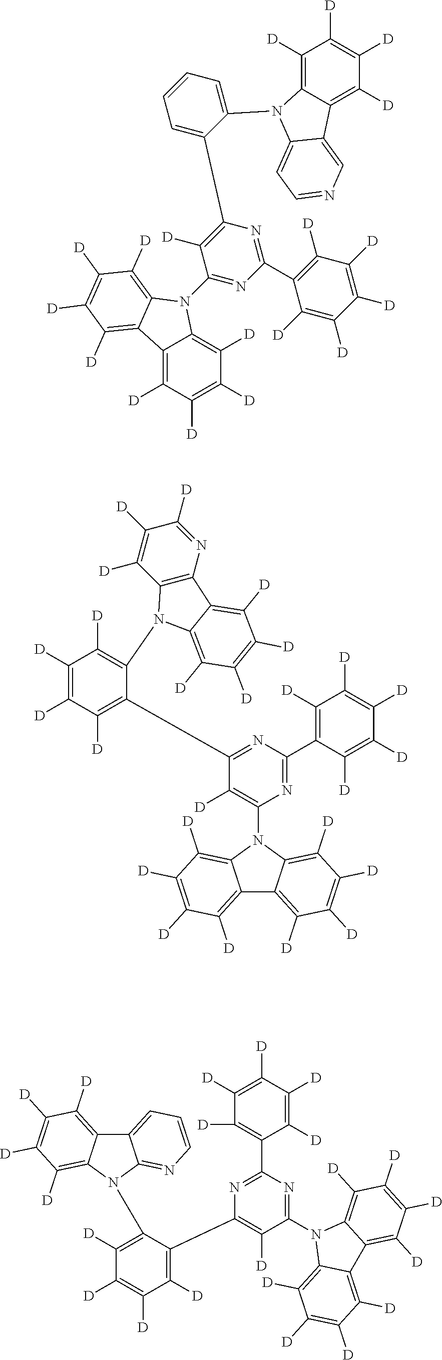

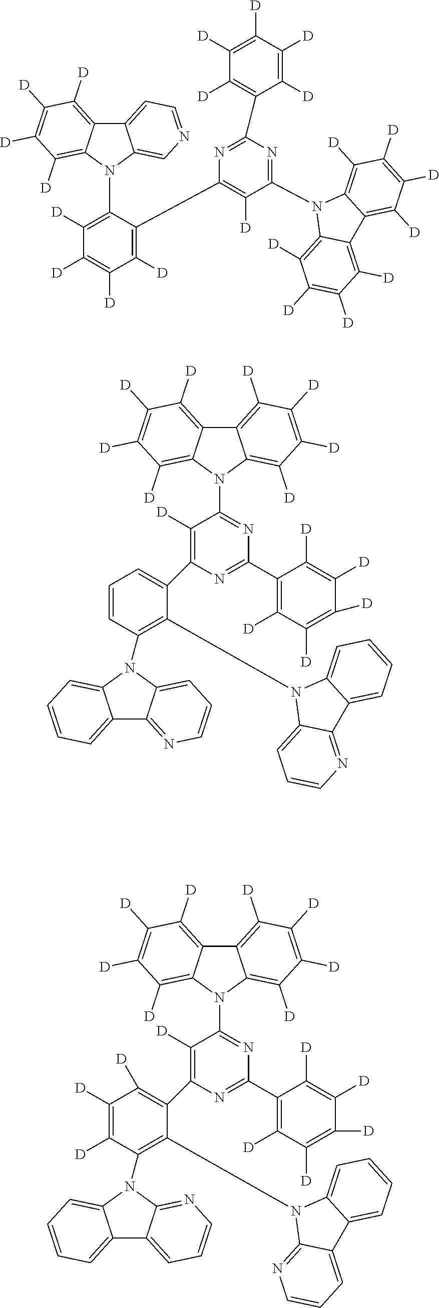

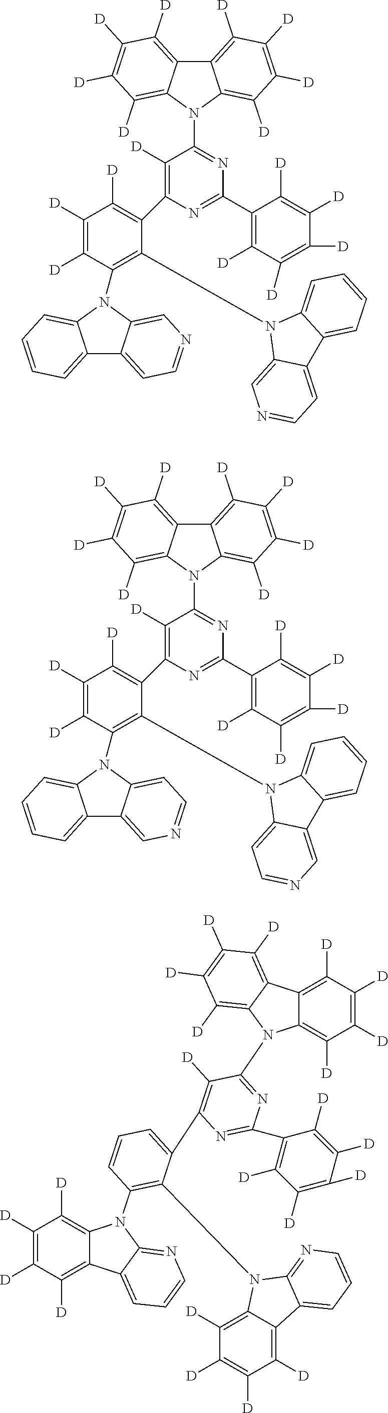

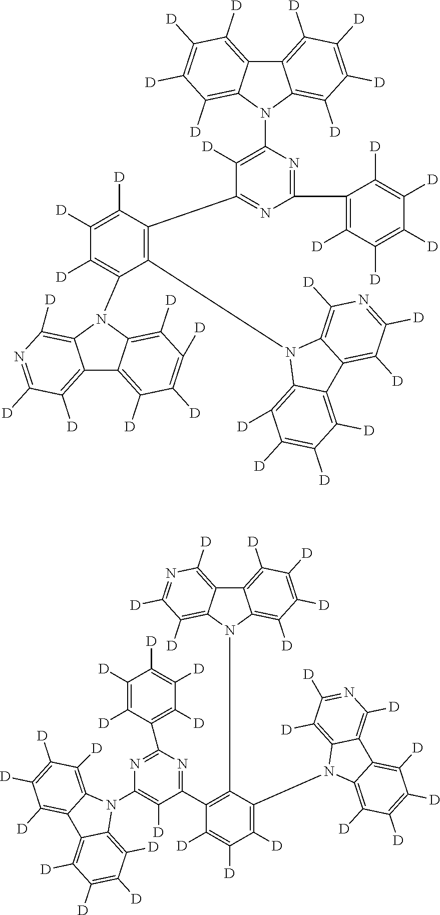

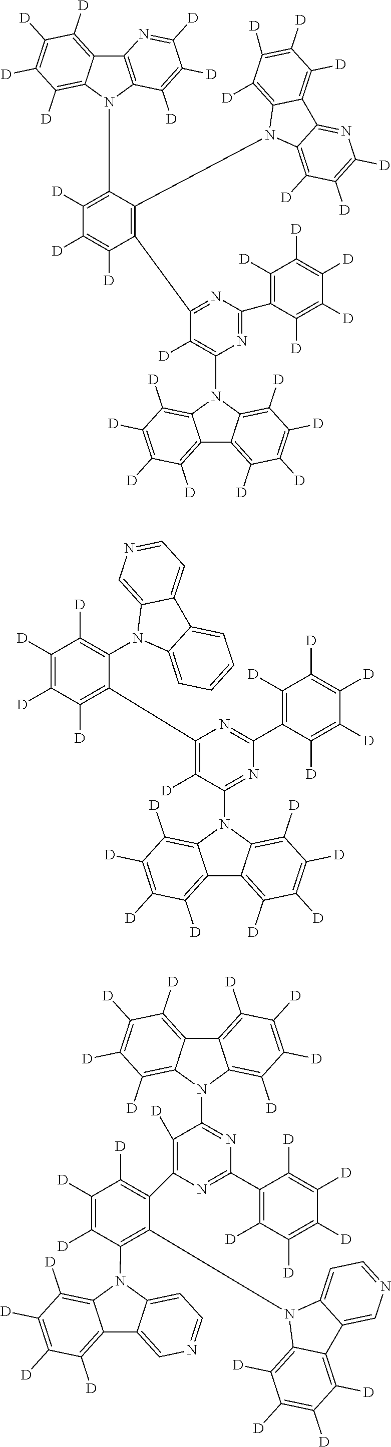

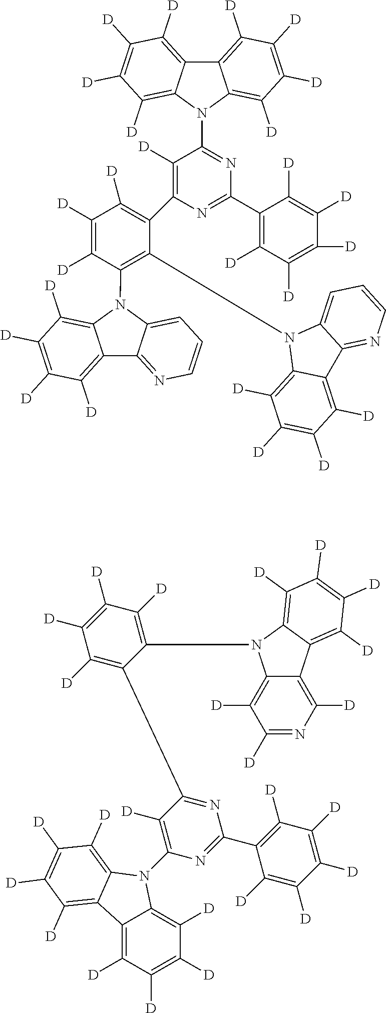 [SMILES: [2H]c1c(-c2c([2H])c([2H])c([2H])c(-n3c4cccnc4c4c([2H])c([2H])c([2H])c([2H])c43)c2-n2c3cccnc3c3c([2H])c([2H])c([2H])c([2H])c32)nc(-c2c([2H])c([2H])c([2H])c([2H])c2[2H])nc1-n1c2c([2H])c([2H])c([2H])c([2H])c2c2c([2H])c([2H])c([2H])c([2H])c21 ; [2H]c1nc([2H])c2c3c([2H])c([2H])c([2H])c([2H])c3n(-c3c([2H])c([2H])c([2H])c([2H])c3-c3nc(-c4c([2H])c([2H])c([2H])c([2H])c4[2H])nc(-n4c5c([2H])c([2H])c([2H])c([2H])c5c5c([2H])c([2H])c([2H])c([2H])c54)c3[2H])c2c1[2H]]

### Second Exemplary Embodiment

### Organic-Electroluminescence-Device Material

An organic-electroluminescence-device material according to a second exemplary embodiment contains a compound according to the first exemplary embodiment (a compound represented by the formula (1)). As an exemplary arrangement, an organic-electroluminescence-device material only containing a compound according to the first exemplary embodiment is exemplarily given. As another exemplary arrangement, an organic-electroluminescence-device material containing a compound according to the first exemplary embodiment and a compound different from the compound according to the first exemplary embodiment is exemplarily given.

In the organic-electroluminescence-device material according to the exemplary embodiment, the compound according to the first exemplary embodiment is preferably a host material.

Herein, when the compound according to the first exemplary embodiment is a host material, the host material is referred to as a first host material.

An organic-electroluminescence-device material according to an exemplary arrangement may contain the first host material (a compound according to the first exemplary embodiment) and another compound such as a dopant material. An organic-electroluminescence-device material according to an exemplary arrangement may contain the first host material (a compound according to the first exemplary embodiment), a second host material different from the first host material, and a dopant material.

In an organic-electroluminescence-device material according to an exemplary arrangement, the first host material is preferably a sensitizing material. In an organic-electroluminescence-device material according to an exemplary arrangement, the first host material is more preferably a sensitizing material and a delayed fluorescent compound.

### Third Exemplary Embodiment

### Organic Electroluminescence Device

An organic EL device according to a third exemplary embodiment will be described.

The organic EL device according to the third exemplary embodiment includes an organic layer between an anode and a cathode. The organic layer includes at least one layer formed from an organic compound. Alternatively, the organic layer includes a plurality of layers formed from an organic compound. The organic layer may further contain an inorganic compound. At least one layer of the organic layer contains, as a compound M2, a compound according to the first exemplary embodiment (a compound represented by the formula (1)).

### Organic Layer

In the organic EL device of the exemplary embodiment, the organic layer may consist of a single emitting layer, or may further include a layer that may be employed in the organic EL device. The layer that may be employed in the organic EL device, which is not particularly limited, is exemplified by at least one layer selected from the group consisting of a hole injecting layer, a hole transporting layer, an electron blocking layer, a hole blocking layer, an electron transporting layer, and an electron injecting layer.

In an exemplary arrangement of the organic EL device of the exemplary embodiment, the organic layer includes at least one emitting layer that contains the compound M2.

In an exemplary arrangement of the organic EL device of the exemplary embodiment, the emitting layer contains the compound M2 as the first host material. In this case, the first host material is preferably a delayed fluorescent compound.

In an exemplary embodiment, the emitting layer contains no metal complex.

In an exemplary embodiment, the emitting layer contains no phosphorescent material.

In the organic EL device of the exemplary embodiment, the hole transporting layer may be disposed between the anode and the emitting layer.

In the organic EL device of the exemplary embodiment, the electron transporting layer may be disposed between the cathode and the emitting layer.

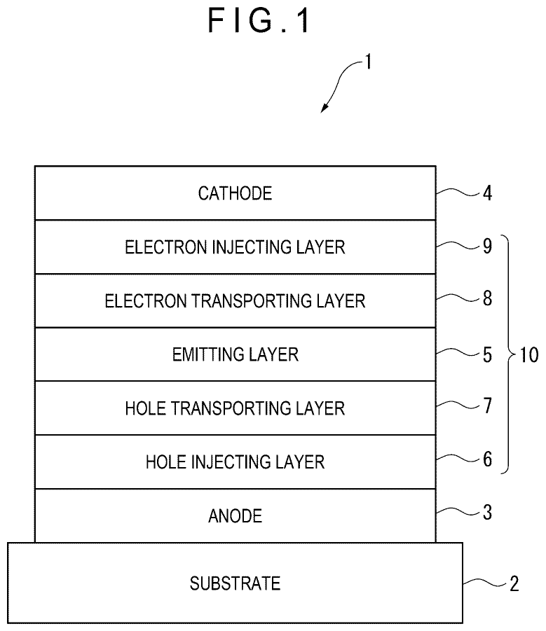

FIG. 1 schematically depicts an exemplary arrangement of the organic EL device according to the exemplary embodiment.

An organic EL device 1 includes a substrate 2, an anode 3, a cathode 4, and an organic layer 10 disposed between the anode 3 and the cathode 4. The organic layer 10 is configured including a hole injecting layer 6, a hole transporting layer 7, an emitting layer 5, an electron transporting layer 8, and an electron injecting layer 9 that are layered on the anode 3 in this order. The invention is not limited to the arrangement of the organic EL device depicted in FIG. 1.

### Emitting Layer

In an exemplary embodiment, a single layer contains the first host material (compound M2) and a fluorescent material. For instance, when the organic EL device includes a single emitting layer, the single emitting layer contains the first host material and the fluorescent material, and when the organic EL device includes a plurality of emitting layers, one of the plurality of emitting layers contains the first host material and the fluorescent material.

### First Host Material (Compound M2)

In an exemplary embodiment, the first host material (compound M2) is a sensitizing material.

In an exemplary embodiment, the emitting layer contains the compound M2 as the sensitizing material.

In an exemplary embodiment, the sensitizing material is a delayed fluorescent compound.

In an exemplary embodiment, the emitting layer contains the compound M2 as the sensitizing material and a fluorescent material.

In an exemplary embodiment, when the emitting layer contains a delayed fluorescent compound as the sensitizing material, the emitting layer contains no phosphorescent metal complex.

### Fluorescent Material

In an exemplary arrangement of the exemplary embodiment, the fluorescent material is a compound exhibiting no thermally activated delayed fluorescence. In an exemplary arrangement of the exemplary embodiment, the fluorescent material is a compound exhibiting thermally activated delayed fluorescence. In an exemplary arrangement of the exemplary embodiment, the fluorescent material is not a phosphorescent metal complex. In an exemplary arrangement of the exemplary embodiment, the fluorescent material is preferably not a metal complex.

Herein, the compound used as the fluorescent material is occasionally referred to as a compound M3.

Specific examples of the compound M3 (fluorescent material) in the third exemplary embodiment include a bisarylaminonaphthalene derivative, aryl-substituted naphthalene derivative, bisarylaminoanthracene derivative, aryl-substituted anthracene derivative, bisarylaminopyrene derivative, aryl-substituted pyrene derivative, bisarylamino chrysene derivative, aryl-substituted chrysene derivative, bisarylaminofluoranthene derivative, aryl-substituted fluoranthene derivative, indenoperylene derivative, acenaphthofluoranthene derivative, compound including a boron atom, pyromethene boron complex compound, compound having a pyromethene skeleton, metal complex of the compound having a pyrromethene skeleton, diketopyrrolopyrrole derivative, perylene derivative, and naphthacene derivative.

In the exemplary embodiment, the fluorescent material (compound M3) is preferably a compound represented by a formula (41) below.

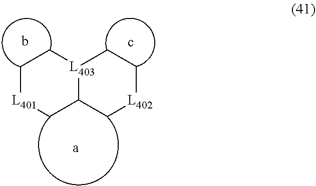

In the formula (41):
a ring a, a ring b, and a ring c are each independently a substituted or unsubstituted aromatic hydrocarbon ring having 6 to 50 ring carbon atoms, or a substituted or unsubstituted heterocycle having 5 to 50 ring atoms;
L₄₀₁ and L₄₀₂ are each independently O, S, Se, NR₄₀, C(R₄₁)(R₄₂), or Si(R₄₃)(R₄₄);
L₄₀₃ is B, P, or P═O;
R₄₀ to R₄₄ are each independently bonded with the ring a, ring b, or ring c to form a substituted or unsubstituted monocyclic ring, bonded with the ring a, ring b, or ring c to form a substituted or unsubstituted fused ring, or bonded neither with the ring a, ring b, nor ring c;
R₄₁ and R₄₂ are mutually bonded to form a substituted or unsubstituted monocyclic ring, mutually bonded to form a substituted or unsubstituted fused ring, or not mutually bonded;
R₄₃ and R₄₄ are mutually bonded to form a substituted or unsubstituted monocyclic ring, mutually bonded to form a substituted or unsubstituted fused ring, or not mutually bonded;
R₄₀ to R₄₄ forming neither the substituted or unsubstituted monocyclic ring nor the substituted or unsubstituted fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, an iminyl group represented by —CR₄₅═N, a substituted or unsubstituted aryl group having 6 to 50 ring carbon atoms, or a substituted or unsubstituted heterocyclic group having 5 to 50 ring atoms;
R₄₅ is a substituted or unsubstituted aryl group having 6 to 60 ring carbon atoms, a substituted or unsubstituted heterocyclic group having 5 to 60 ring atoms, a substituted or unsubstituted alkyl group having 1 to 20 carbon atoms, or a substituted or unsubstituted cycloalkyl group having 3 to 20 ring carbon atoms; when a plurality of Rao are present, the plurality of Rao are mutually the same or different;
when a plurality of R₄₁ are present, the plurality of R₄₁ are mutually the same or different;
when a plurality of R₄₂ are present, the plurality of R₄₂ are mutually the same or different;
when a plurality of R₄₃ are present, the plurality of R₄₃ are mutually the same or different;
when a plurality of R₄₄ are present, the plurality of R₄₄ are mutually the same or different; and
when a plurality of R₄₅ are present, the plurality of R₄₅ are mutually the same or different.

In the exemplary embodiment, the compound represented by the formula (41) is preferably a compound represented by a formula (410) below.

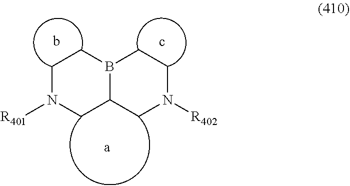

In the formula (410):
a ring a, a ring b, and a ring c are each independently a substituted or unsubstituted aromatic hydrocarbon ring having 6 to 50 ring carbon atoms, or a substituted or unsubstituted heterocycle having 5 to 50 ring atoms;
R₄₀₁ and R₄₀₂ are each independently bonded with the ring a, ring b, or ring c to form a substituted or unsubstituted monocyclic ring, bonded with the ring a, ring b, or ring c to form a substituted or unsubstituted fused ring, or bonded neither with the ring a, ring b, nor ring c; and
R₄₀₁ and R₄₀₂ forming neither the substituted or unsubstituted monocyclic ring nor the substituted or unsubstituted fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, an iminyl group represented by —CR₄₅═N, a substituted or unsubstituted aryl group having 6 to 50 ring carbon atoms, or a substituted or unsubstituted heterocyclic group having 5 to 50 ring atoms.

In the exemplary embodiment, the compound represented by the formula (41) is preferably a compound selected from the group consisting of compounds represented by formulae (41-1) to (41-6) below.

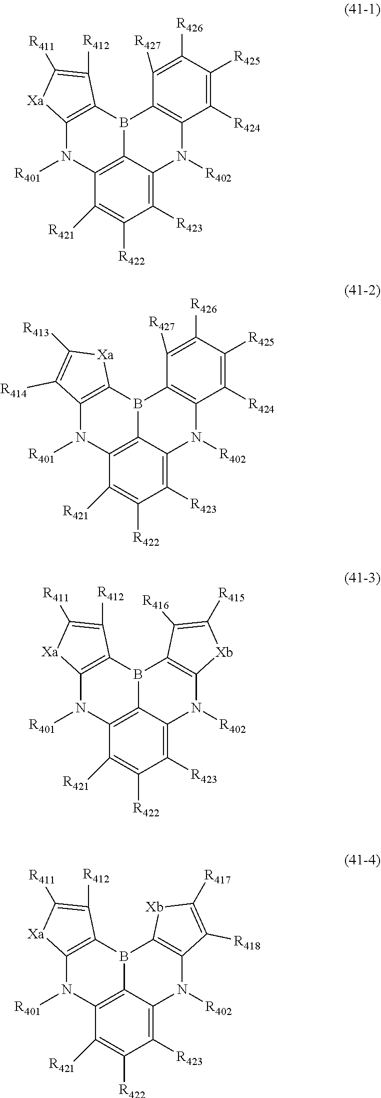

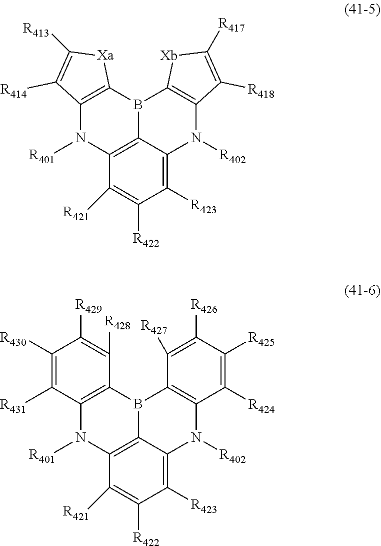

In the formula (41-1):
Xa is O, S, Se, C(R₄₀₃)(R₄₀₄), or NR_{405;}
at least one combination selected from the group consisting of a combination of R₄₀₁ and R₄₂₁, a combination of adjacent two or more of R₄₂₁ to R₄₂₃, a combination of R₄₂₃ and R₄₀₂, a combination of R₄₀₂ and R₄₂₄, a combination of adjacent two or more of R₄₂₄ to R₄₂₇, a combination of R₄₂₇ and R₄₁₂, and a combination of R₄₁₂ and R₄₁₁ are mutually bonded to form a substituted or unsubstituted monocyclic ring, mutually bonded to form a substituted or unsubstituted fused ring, or not mutually bonded;
R₄₀₁ and R₄₀₂ forming neither the substituted or unsubstituted monocyclic ring nor the substituted or unsubstituted fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, an iminyl group represented by —CR₄₅═N, a substituted or unsubstituted aryl group having 6 to 50 ring carbon atoms, or a substituted or unsubstituted heterocyclic group having 5 to 50 ring atoms;
R₄₀₃ to R₄₀₅, and R₄₁₁, R₄₁₂, and R₄₂₁ to R₄₂₇ forming neither the substituted or unsubstituted monocyclic ring nor the substituted or unsubstituted fused ring are each independently a hydrogen atom or a substituent Rx;
each substituent Rx is independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms; and
when a plurality of R₉₀₁ are present, the plurality of R₉₀₁ are mutually the same or different; when a plurality of R₉₀₂ are present, the plurality of R₉₀₂ are mutually the same or different; when a plurality of R₉₀₃ are present, the plurality of R₉₀₃ are mutually the same or different; when a plurality of R₉₀₄ are present, the plurality of R₉₀₄ are mutually the same or different; when a plurality of R₉₀₅ are present, the plurality of R₉₀₆ are mutually the same or different; when a plurality of R₉₀₆ are present, the plurality of R₉₀₆ are mutually the same or different; and when a plurality of R₉₀₇ are present, the plurality of R₉₀₇ are mutually the same or different.

In the formula (41-2):
Xa is O, S, Se, C(R₄₀₃)(R₄₀₄), or NR_{405;}
at least one combination selected from the group consisting of a combination of R₄₀₁ and R₄₂₁, a combination of adjacent two or more of R₄₂₁ to R₄₂₃, a combination of R₄₂₃ and R₄₀₂, a combination of R₄₀₂ and R₄₂₄, a combination of adjacent two or more of R₄₂₄ to R₄₂₇, a combination of R₄₁₃ and R₄₁₄, and a combination of R₄₁₄ and R₄₀₁ are mutually bonded to form a substituted or unsubstituted monocyclic ring, mutually bonded to form a substituted or unsubstituted fused ring, or not mutually bonded;
R₄₀₁ and R₄₀₂ forming neither the substituted or unsubstituted monocyclic ring nor the substituted or unsubstituted fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, an iminyl group represented by —CR₄₅═N, a substituted or unsubstituted aryl group having 6 to 50 ring carbon atoms, or a substituted or unsubstituted heterocyclic group having 5 to 50 ring atoms; and
R₄₀₃ to R₄₀₅, and R₄₁₃, R₄₁₄, and R₄₂₁ to R₄₂₇ forming neither the substituted or unsubstituted monocyclic ring nor the substituted or unsubstituted fused ring are each independently a hydrogen atom or a substituent Rx, and the substituent Rx represents the same as the substituent Rx in the formula (41-1).

In the formula (41-3):
Xa and Xb are each independently O, S, Se, C(R₄₀₃)(R₄₀₄), or NR_{405;}
at least one combination selected from the group consisting of a combination of R₄₀₁ and R₄₂₁, a combination of adjacent two or more of R₄₂₁ to R₄₂₃, a combination of R₄₂₃ and R₄₀₂, a combination of R₄₁₃ and R₄₁₆, a combination of R₄₁₆ and R₄₁₂, and a combination of R₄₁₂ and R₄₁₁ are mutually bonded to form a substituted or unsubstituted monocyclic ring, mutually bonded to form a substituted or unsubstituted fused ring, or not mutually bonded;
R₄₀₁ and R₄₀₂ forming neither the substituted or unsubstituted monocyclic ring nor the substituted or unsubstituted fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, an iminyl group represented by —CR₄₅═N, a substituted or unsubstituted aryl group having 6 to 50 ring carbon atoms, or a substituted or unsubstituted heterocyclic group having 5 to 50 ring atoms;
R₄₀₃ to R₄₀₅, and R₄₁₁, R₄₁₂, R₄₁₅, R₄₁₆, and R₄₂₁ to R₄₂₃ forming neither the substituted or unsubstituted monocyclic ring nor the substituted or unsubstituted fused ring are each independently a hydrogen atom or a substituent Rx, and the substituent Rx represents the same as the substituent Rx in the formula (41-1);
when a plurality of R₄₀₃ are present, the plurality of R₄₀₃ are mutually the same or different;
when a plurality of R₄₀₄ are present, the plurality of R₄₀₄ are mutually the same or different; and
when a plurality of R₄₀₅ are present, the plurality of R₄₀₅ are mutually the same or different.

In the formula (41-4):
Xa and Xb are each independently O, S, Se, C(R₄₀₃)(R₄₀₄), or NR_{405;}
at least one combination selected from the group consisting of a combination of R₄₀₁ and R₄₂₁, a combination of adjacent two or more of R₄₂₁ to R₄₂₃, a combination of R₄₂₃ and R₄₀₂, a combination of R₄₀₂ and R₄₁₈, a combination of R₄₁₈ and R₄₁₇, and a combination of R₄₁₂ and R₄₁₁ are mutually bonded to form a substituted or unsubstituted monocyclic ring, mutually bonded to form a substituted or unsubstituted fused ring, or not mutually bonded;
R₄₀₁ and R₄₀₂ forming neither the substituted or unsubstituted monocyclic ring nor the substituted or unsubstituted fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, an iminyl group represented by —CR₄₅═N, a substituted or unsubstituted aryl group having 6 to 50 ring carbon atoms, or a substituted or unsubstituted heterocyclic group having 5 to 50 ring atoms;
R₄₀₃ to R₄₀₅, and R₄₁₁, R₄₁₂, R₄₁₇, R₄₁₈, and R₄₂₁ to R₄₂₃ forming neither the substituted or unsubstituted monocyclic ring nor the substituted or unsubstituted fused ring are each independently a hydrogen atom or a substituent Rx, and the substituent Rx represents the same as the substituent Rx in the formula (41-1);
when a plurality of R₄₀₃ are present, the plurality of R₄₀₃ are mutually the same or different;
when a plurality of R₄₀₄ are present, the plurality of R₄₀₄ are mutually the same or different; and
when a plurality of R₄₀₅ are present, the plurality of R₄₀₅ are mutually the same or different.

In the formula (41-5):
Xa and Xb are each independently O, S, Se, C(R₄₀₃)(R₄₀₄), or NR_{405;}
at least one combination selected from the group consisting of a combination of R₄₀₁ and R₄₂₁, a combination of adjacent two or more of R₄₂₁ to R₄₂₃, a combination of R₄₂₃ and R₄₀₂, a combination of R₄₀₂ and R₄₁₈, a combination of R₄₁₈ to R₄₁₇, a combination of R₄₁₃ and R₄₁₄, and a combination of R₄₁₄ and R₄₀₁ are mutually bonded to form a substituted or unsubstituted monocyclic ring, mutually bonded to form a substituted or unsubstituted fused ring, or not mutually bonded;
R₄₀₁ and R₄₀₂ forming neither the substituted or unsubstituted monocyclic ring nor the substituted or unsubstituted fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, an iminyl group represented by —CR₄₅═N, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
R₄₀₃ to R₄₀₅, and R₄₁₃, R₄₁₄, R₄₁₇, R₄₁₈, and R₄₂₁ to R₄₂₃ forming neither the substituted or unsubstituted monocyclic ring nor the substituted or unsubstituted fused ring are each independently a hydrogen atom or a substituent Rx, and the substituent Rx represents the same as the substituent Rx in the formula (41-1);
when a plurality of R₄₀₃ are present, the plurality of R₄₀₃ are mutually the same or different;
when a plurality of R₄₀₄ are present, the plurality of R₄₀₄ are mutually the same or different; and
when a plurality of R₄₀₅ are present, the plurality of R₄₀₅ are mutually the same or different.

In the formula (41-6):
at least one combination selected from the group consisting of a combination of R₄₀₁ and R₄₂₁, a combination of adjacent two or more of R₄₂₁ to R₄₂₃, a combination of R₄₂₃ and R₄₀₂, a combination of R₄₀₂ and R₄₂₄, a combination of adjacent two or more of R₄₂₄ to R₄₂₇, a combination of R₄₂₇ and R₄₂₈, a combination of adjacent two or more of R₄₂₄ to R₄₃₁, and a combination of R₄₃₁ and R₄₀₁ are mutually bonded to form a substituted or unsubstituted monocyclic ring, mutually bonded to form a substituted or unsubstituted fused ring, or not mutually bonded;
R₄₀₁ and R₄₀₂ forming neither the substituted or unsubstituted monocyclic ring nor the substituted or unsubstituted fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, an iminyl group represented by —CR₄₅═N, a substituted or unsubstituted aryl group having 6 to 50 ring carbon atoms, or a substituted or unsubstituted heterocyclic group having 5 to 50 ring atoms; and
R₄₂₁ to R₄₃₁ forming neither the substituted or unsubstituted monocyclic ring nor the substituted or unsubstituted fused ring are each independently a hydrogen atom or a substituent Rx, and the substituent Rx represents the same as the substituent Rx in the formula (41-1).

In the compounds represented by the formulae (41-1) to (41-5), also preferably, at least one combination selected from the group consisting of a combination of R₄₁₂ and R₄₁₁, a combination of R₄₁₃ and R₄₁₄, a combination of R₄₁₅ and R₄₁₆, and a combination of R₄₁₇ and R₄₁₈ are mutually bonded to form a substituted or unsubstituted monocyclic ring or mutually bonded to form a substituted or unsubstituted fused ring.

In the exemplary embodiment, the compound represented by the formula (41) is also preferably a compound represented by a formula (41-7) below.

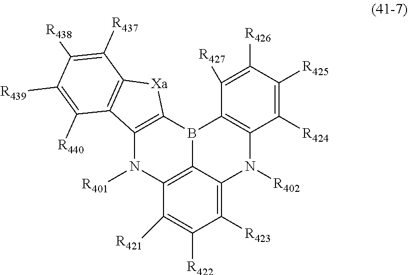

In the formula (41-7):
Xa is O, S, Se, C(R₄₀₃)(R₄₀₄), or NR_{405;}
at least one combination selected from the group consisting of a combination of R₄₀₁ and R₄₂₁, a combination of adjacent two or more of R₄₂₁ to R₄₂₃, a combination of R₄₂₃ and R₄₀₂, a combination of R₄₀₂ and R₄₂₄, a combination of adjacent two or more of R₄₂₄ to R₄₂₇, and a combination of adjacent two or more of R₄₃₇ to R₄₄₀ are mutually bonded to form a substituted or unsubstituted monocyclic ring, mutually bonded to form a substituted or unsubstituted fused ring, or not mutually bonded;
R₄₀₁ and R₄₀₂ forming neither the substituted or unsubstituted monocyclic ring nor the substituted or unsubstituted fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms; and
R₄₀₃ to R₄₀₅, and R₄₂₁ to R₄₂₇ and R₄₃₇ to R₄₄₀ forming neither the substituted or unsubstituted monocyclic ring nor the substituted or unsubstituted fused ring are each independently a hydrogen atom or a substituent Rx, and the substituent Rx represents the same as the substituent Rx in the formula (41-1).

In the exemplary embodiment, R₄₀₁ and R₄₀₂ are each independently preferably a substituted or unsubstituted aryl group having 6 to 50 ring carbon atoms, or a substituted or unsubstituted heterocyclic group having 5 to 50 ring atoms, more preferably a substituted or unsubstituted aryl group having 6 to 50 ring carbon atoms, and still more preferably a group represented by a formula (42) below.

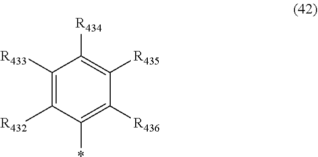

In the formula (42):
at least one combination of adjacent two or more of R₄₃₂ to R₄₃₆ are mutually bonded to form a substituted or unsubstituted monocyclic ring, mutually bonded to form a substituted or unsubstituted fused ring, or not mutually bonded;
R₄₃₂ to R₄₃₆ forming neither the substituted or unsubstituted monocyclic ring nor the substituted or unsubstituted fused ring are each independently a hydrogen atom or a substituent Rx, and the substituent Rx represents the same as the substituent Rx in the formula (41-1);
when a plurality of R₄₃₂ are present, the plurality of R₄₃₂ are mutually the same or different;
when a plurality of R₄₃₃ are present, the plurality of R₄₃₃ are mutually the same or different;
when a plurality of R₄₃₄ are present, the plurality of R₄₃₄ are mutually the same or different;
when a plurality of R₄₃₅ are present, the plurality of R₄₃₅ are mutually the same or different;
when a plurality of R₄₃₆ are present, the plurality of R₄₃₆ are mutually the same or different; and
* represents a bonding position.

In the exemplary embodiment, the compound represented by the formula (41) is also preferably a compound represented by a formula (42-1) below.

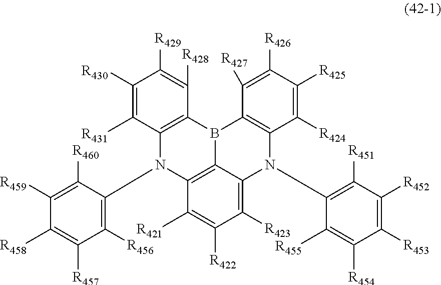

In the formula (42-1):
R₄₂₁ to R₄₃₁ respectively represent the same as R₄₂₁ to R₄₃₁ in the formula (41-6);
at least one combination of adjacent two or more of R₄₅₁ to R₄₅₅ are mutually bonded to form a substituted or unsubstituted monocyclic ring, mutually bonded to form a substituted or unsubstituted fused ring, or not mutually bonded;
at least one combination of adjacent two or more of R₄₅₆ to R₄₆₀ are mutually bonded to form a substituted or unsubstituted monocyclic ring, mutually bonded to form a substituted or unsubstituted fused ring, or not mutually bonded; and
R₄₅₁ to R₄₅₅ and R₄₅₆ to R₄₆₀ forming neither the substituted or unsubstituted monocyclic ring nor the substituted or unsubstituted fused ring are each independently a hydrogen atom or a substituent Rx, and the substituent Rx represents the same as the substituent Rx in the formula (41-1).

In the exemplary embodiment, the compound represented by the formula (41) is also preferably a compound represented by a formula (42-2) below.

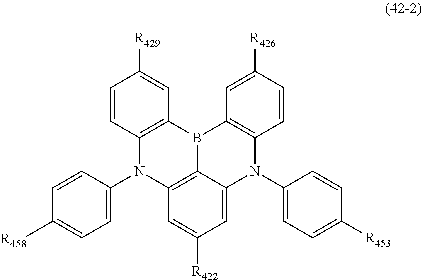

In the formula (42-2), R₄₂₂, R₄₂₆, R₄₂₉, R₄₅₃, and R₄₅₈ are each independently a hydrogen atom or a substituent Rx, and the substituent Rx represents the same as the substituent Rx in the formula (41-1).

In the exemplary embodiment, the compound represented by the formula (41) is also preferably a compound represented by a formula (42-3) below.

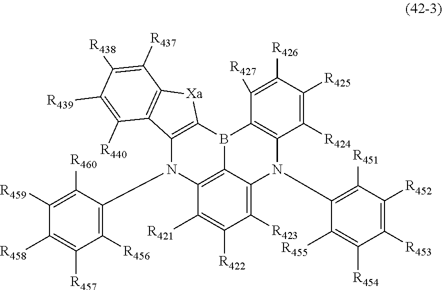

In the formula (42-3):
R₄₂₁ to R₄₂₇, R₄₃₇ to R₄₄₀ and Xa respectively represent the same as R₄₂₁ to R₄₂₇, R₄₃₇ to R₄₄₀ and Xa in the formula (41-7);
at least one combination of adjacent two or more of R₄₅₁ to Row are mutually bonded to form a substituted or unsubstituted monocyclic ring, mutually bonded to form a substituted or unsubstituted fused ring, or not mutually bonded;
at least one combination of adjacent two or more of R₄₅₆ to R₄₆₀ are mutually bonded to form a substituted or unsubstituted monocyclic ring, mutually bonded to form a substituted or unsubstituted fused ring, or not mutually bonded; and
R₄₅₁ to R₄₅₅ and R₄₅₆ to R₄₆₀ forming neither the substituted or unsubstituted monocyclic ring nor the substituted or unsubstituted fused ring are each independently a hydrogen atom or a substituent Rx, and the substituent Rx represents the same as the substituent Rx in the formula (41-1).

In the exemplary embodiment, the compound represented by the formula (41) is also preferably a compound represented by a formula (42-4) below.

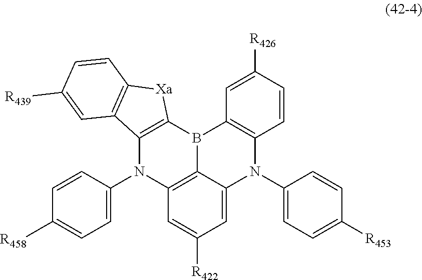

In the formula (42-4):
Xa represents the same as Xa in the formula (41-7);
R₄₂₂, R₄₂₆, R₄₂₉, R₄₃₉, R₄₅₃, and R₄₅₈ are each independently a hydrogen atom or a substituent Rx; and
the substituent Rx represents the same as the substituent Rx in the formula (41-1).

In the exemplary embodiment, R₄₂₂, R₄₂₆, R₄₂₉, R₄₃₉, R₄₅₃, and R₄₅₈ in the compound M3 are, each independently, preferably a substituted or unsubstituted alkyl group having 1 to 50 carbon atoms, more preferably a substituted or unsubstituted alkyl group having 1 to 20 carbon atoms, and still more preferably a substituted or unsubstituted alkyl group having 1 to 6 carbon atoms.

In the exemplary embodiment, Xa and Xb in the compound M3 are preferably each independently O or S.

### Method for Producing Compound Represented by Formula (41)

The compound represented by the formula (41) can be produced by a known method. Further, the compound represented by the formula (41) can be produced by application of known substitution reactions and materials tailored for the target compound, in accordance with the known method.

### Specific Examples of Compound Represented by Formula (41)

Specific examples of the compound represented by the formula (41) include compounds as below. In the specific examples below, Me represents a methyl group, tBu represents a tertiary butyl group, and Ph represents a phenyl group.

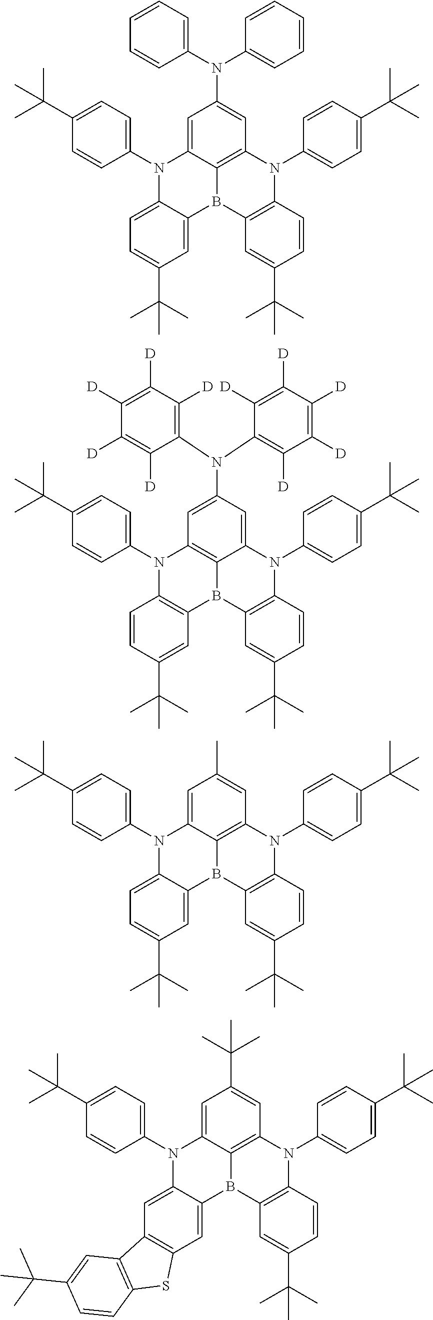

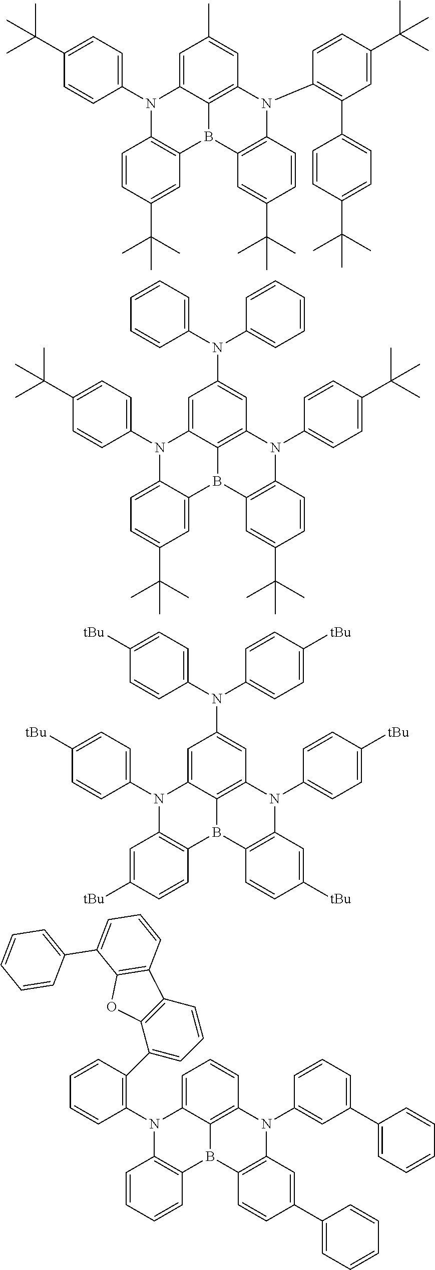

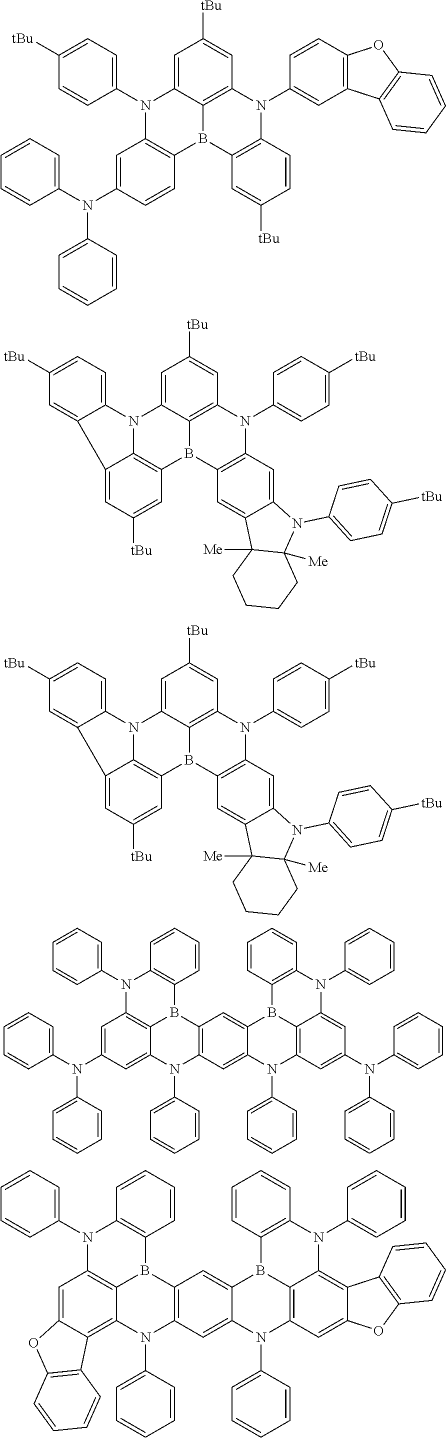

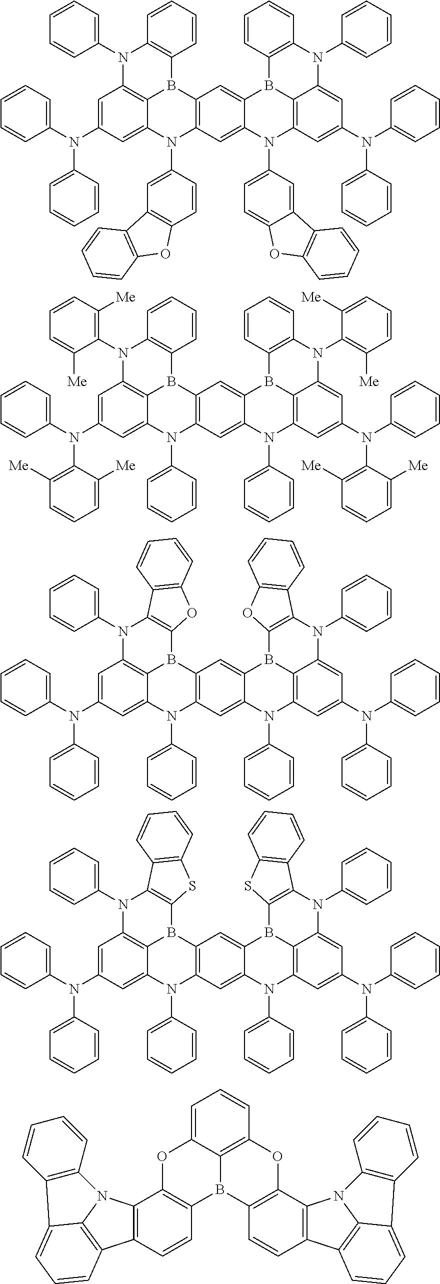

In an exemplary embodiment:
the substituent for the “substituted or unsubstituted” group in each of the above formulae is an unsubstituted alkyl group having 1 to 50 carbon atoms, an unsubstituted alkenyl group having 2 to 50 carbon atoms, an unsubstituted alkynyl group having 2 to 50 carbon atoms, an unsubstituted cycloalkyl group having 3 to 50 ring carbon atoms, —Si(R₉₀₁ₐ)(R₉₀₂ₐ)(R₉₀₃ₐ), —O—(R₉₀₄ₐ), —S—(R₉₀₅ₐ), —N(R₉₀₆ₐ)(R₉₀₇ₐ), a halogen atom, a cyano group, a nitro group, an unsubstituted aryl group having 6 to 50 ring carbon atoms, or an unsubstituted heterocyclic group having 5 to 50 ring atoms;
R₉₀₁ₐ to R₉₀₇ₐ are each independently a hydrogen atom, an unsubstituted alkyl group having 1 to 50 ring carbon atoms, an unsubstituted aryl group having 6 to 50 carbon atoms, or an unsubstituted heterocyclic group having 5 to 50 ring atoms; and
when two or more R₉₀₁ₐ are present, the two or more R₉₀₁ₐ are mutually the same or different; when two or more R₉₀₂ₐ are present, the two or more R₉₀₂ₐ are mutually the same or different; when two or more R₉₀₃ₐ are present, the two or more R₉₀₃ₐ are mutually the same or different; when two or more R₉₀₄ₐ are present, the two or more R₉₀₄ₐ are mutually the same or different; when two or more R₉₀₅ₐ are present, the two or more R₉₀₅ₐ are mutually the same or different; when two or more R₉₀₆ₐ are present, the two or more R₉₀₆ₐ are mutually the same or different; and when two or more R₉₀₇ₐ are present, the two or more R₉₀₇ₐ are mutually the same or different.

In an exemplary embodiment, the substituent for the “substituted or unsubstituted” group in each of the above formulae is an unsubstituted alkyl group having 1 to 50 carbon atoms, an unsubstituted aryl group having 6 to 50 ring carbon atoms, or an unsubstituted heterocyclic group having 5 to 50 ring atoms.

In an exemplary embodiment, the substituent for the “substituted or unsubstituted” group in each of the above formulae is an unsubstituted alkyl group having 1 to 18 carbon atoms, an unsubstituted aryl group having 6 to 18 ring carbon atoms, or an unsubstituted heterocyclic group having 5 to 18 ring atoms.

### Maximum Peak Wavelength

In the exemplary embodiment, the maximum peak wavelength of the compound M3 as the fluorescent material is preferably 480 nm or less, more preferably 475 nm or less.

In the exemplary embodiment, the maximum peak wavelength of the compound M3 as the fluorescent material is preferably 430 nm or more, more preferably 440 nm or more.

Herein, the maximum peak wavelength of fluorescence is occasionally referred to as a maximum fluorescence peak wavelength λ_{FL}.

In the organic EL device of the exemplary embodiment, the compound M3 preferably emits blue light. Herein, the blue light emission refers to a light emission in which the maximum peak wavelength of a fluorescence spectrum is in a range from 430 nm to 480 nm.

### Full Width at Half Maximum of Emission Spectrum

In the exemplary embodiment, the full width at half maximum (FWHM) of the emission spectrum of the compound M3 as the fluorescent material is preferably 40 nm or less, more preferably 30 nm or less.

In the exemplary embodiment, the full width at half maximum (FWHM) of the emission spectrum of the compound M3 as a fluorescent material is preferably 5 nm or more, more preferably 10 nm or more.

FWHM is an abbreviation of the full width at half maximum.

Herein, the maximum fluorescence peak wavelength λ_{FL} refers to the maximum peak wavelength λ_{FL} of a fluorescence spectrum exhibiting a maximum luminous intensity among fluorescence spectra measured in a toluene solution in which a measurement target compound is dissolved at a concentration ranging from 10⁻⁶ mol/1 to 10⁻⁵ mol/l. The full width at half maximum (FWHM) of the emission spectrum is a full width at half maximum at the maximum peak of the fluorescence spectrum. A fluorescence spectrum measurement apparatus is usable as an apparatus for measuring the fluorescence spectrum. For instance, a fluorescence spectrum measurement apparatus (apparatus name: FP-8300, produced by JASCO Corporation) is usable. It should be noted that the fluorescence spectrum measurement apparatus is not limited to the apparatus exemplarily given herein.

### Stokes Shift

In the exemplary embodiment, the Stokes shift of the compound M3 as the fluorescent material is preferably 25 nm or less, more preferably 20 nm or less.

In the exemplary embodiment, the Stokes shift of the compound M3 as the fluorescent material is preferably 5 nm or more, more preferably 10 nm or more.

When the Stokes shift of the compound M3 is 20 nm or less, excitation energy is reduced.

When the Stokes shift of the compound M3 is 10 nm or more, self-absorption is inhibited to reduce the loss of efficiency.

The Stokes shift can be measured by a method described below. A measurement target compound is dissolved in toluene at a concentration of 2.0×10⁻⁵ mol/L to prepare a measurement sample. The measurement sample is put into a quartz cell and is irradiated with continuous light falling within an ultraviolet-to-visible region at a room temperature (300 K) to measure an absorption spectrum (ordinate axis: absorbance, abscissa axis: wavelength). A spectrophotometer U-3900/3900H produced by Hitachi High-Tech Science Corporation is usable for the absorption spectrum measurement. Further, a measurement target compound is dissolved in toluene at a concentration of 4.9×10⁻⁶ mol/L to prepare a measurement sample. The measurement sample is put into a quartz cell and irradiated with excited light at a room temperature (300 K) to measure a fluorescence spectrum (ordinate axis: fluorescence intensity, abscissa axis: wavelength). A spectrophotofluorometer F-7000 produced by Hitachi High-Tech Science Corporation is usable for the fluorescence spectrum measurement. A difference between an absorption local-maximum wavelength and a fluorescence local-maximum wavelength is calculated from the absorption spectrum and the fluorescence spectrum to obtain a Stokes shift (SS). A unit of the Stokes shift (SS) is denoted by nm.

Relationship between Sensitizing Material and Fluorescent Material in Emitting Layer

In an exemplary embodiment, the sensitizing material (compound M2) as the first host material is a delayed fluorescent compound. In an exemplary embodiment, when the emitting layer contains a delayed fluorescent compound as the sensitizing material, the emitting layer contains no phosphorescent metal complex.

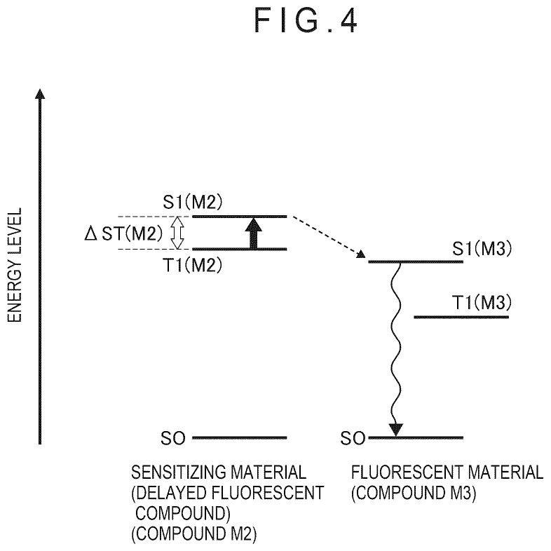

FIG. 4 illustrates an exemplary relationship in energy level between a sensitizing material and a fluorescent material, when the emitting layer contains a delayed fluorescent compound (compound M2) as the sensitizing material and the fluorescent material (compound M3). In FIG. 4, S0 represents a ground state. S1(M2) represents a lowest singlet state of the delayed fluorescent compound, and T1(M2) represents a lowest triplet state of the delayed fluorescent compound. S1(M3) represents a lowest singlet state of the fluorescent material, and T1(M3) represents a lowest triplet state of the fluorescent material. A dashed arrow directed from S1(M2) to S1(M3) in FIG. 4 represents Forster energy transfer from the lowest singlet state of the delayed fluorescent compound to the lowest singlet state of the fluorescent material.

As illustrated in FIG. 4, when a compound having a small ΔST(M2) is used as the delayed fluorescent compound, inverse intersystem crossing from the lowest triplet state T1(M2) to the lowest singlet state S1(M2) can be caused by heat energy. Subsequently, Forster energy transfer from the lowest singlet state S1(M2) of the delayed fluorescent compound to the fluorescent material occurs to generate a lowest singlet state S1(M3). Consequently, fluorescence from the lowest singlet state S1(M3) of the fluorescent material can be observed. It is inferred that the internal quantum efficiency can be theoretically raised up to 100% also by using delayed fluorescence by the TADF mechanism.

In an exemplary embodiment, when the compound M2 is a sensitizing material, a lowest singlet energy S₁(GT2) of the sensitizing material and a lowest singlet energy S₁ (D) of the fluorescent material preferably satisfy a relationship of a numerical formula (Numerical Formula 3) below.

S₁(GT2)>S₁(D) (Numerical Formula 3)

In an exemplary embodiment, when the compound M2 is a sensitizing material, an energy gap T_{77K}(GT2) at 77 K of the sensitizing material and an energy gap T_{77K}(D) at 77 K of the fluorescent material also preferably satisfy a relationship of a numerical formula (Numerical Formula 6A) below.

T_{77K}(GT2)>T_{77K}(D) (Numerical Formula 6A)

It is preferable that, when the organic EL device of the exemplary embodiment emits light, a fluorescent compound mainly emits light in the emitting layer.

The maximum peak wavelength of the light emitted from the organic EL device is measured as follows.

Voltage is applied to the organic EL device such that a current density is 10 mA/cm², where spectral radiance spectrum is measured by a spectroradiometer CS-2000 (produced by Konica Minolta, Inc.).

A peak wavelength of an emission spectrum, at which the luminous intensity of the obtained spectral radiance spectrum is at the maximum, is measured and defined as a maximum peak wavelength (unit: nm).

### Content Ratios of Compounds in Emitting Layer

For instance, content ratios of the first host material (compound M2) and the fluorescent material (compound M3) in the emitting layer preferably fall within ranges below.

The content ratio of the compound M2 falls within, preferably, in a range from 10 mass % to 80 mass %, more preferably in a range from 10 mass % to 60 mass %, and still more preferably in a range from 20 mass % to 60 mass %. The content ratio of the compound M2 may fall within in a range from 90 mass % to 99.9 mass %, in a range from 95 mass % to 99.9 mass %, or in a range from 99 mass % to 99.9 mass %.

The content ratio of the compound M3 falls within, preferably, in a range from 0.01 mass % to 10 mass %, more preferably in a range from 0.01 mass % to 5 mass %, and still more preferably in a range from 0.01 mass % to 1 mass %.

It should be noted that the emitting layer in the exemplary embodiment may contain any other material than the compound M2 and the compound M3.

The emitting layer may contain a single type of the compound M2 or may contain two or more types of the compound M2. The emitting layer may contain a single type of the compound M3 or may contain two or more types of the compound M3.

The arrangement of the organic EL device will be further described below.

### Substrate

The substrate is used as a support for the organic EL device. For instance, glass, quartz, plastics and the like are usable for the substrate. A flexible substrate is also usable. The flexible substrate, which is a bendable substrate, is exemplified by a plastic substrate. Examples of a material for the plastic substrate include polycarbonate, polyarylate, polyethersulfone, polypropylene, polyester, polyvinyl fluoride, polyvinyl chloride, polyimide, and polyethylene naphthalate. Further, an inorganic vapor deposition film is also usable.

### Anode

Metal, an alloy, an electrically conductive compound, a mixture thereof, or the like having a large work function (specifically, 4.0 eV or more) is preferably used as the anode formed on the substrate. Specific examples of the material include indium tin oxide (ITO), indium oxide-tin oxide containing silicon or silicon oxide, indium oxide-zinc oxide, indium oxide containing tungsten oxide and zinc oxide, and graphene. In addition, gold (Au), platinum (Pt), nickel (Ni), tungsten (W), chrome (Cr), molybdenum (Mo), iron (Fe), cobalt (Co), copper (Cu), palladium (Pd), titanium (Ti), and nitrides of a metal material (e.g., titanium nitride) are usable.

The material is typically formed into a film by a sputtering method. For instance, the indium oxide-zinc oxide can be formed into a film by the sputtering method using a target in which zinc oxide in a range from 1 mass % to 10 mass % is added to indium oxide. Moreover, for instance, the indium oxide containing tungsten oxide and zinc oxide can be formed by the sputtering method using a target in which tungsten oxide in a range from 0.5 mass % to 5 mass % and zinc oxide in a range from 0.1 mass % to 1 mass % are added to indium oxide. In addition, the anode may be formed by a vacuum deposition method, a coating method, an inkjet method, a spin coating method or the like.

Among the EL layers formed on the anode, since the hole injecting layer adjacent to the anode is formed of a composite material into which holes are easily injectable irrespective of the work function of the anode, a material usable as an electrode material (e.g., metal, an alloy, an electroconductive compound, a mixture thereof, and the elements belonging to the group 1 or 2 of the periodic table) is also usable for the anode.

A material having a small work function such as elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AlLi) including the alkali metal or the alkaline earth metal, a rare earth metal such as europium (Eu) and ytterbium (Yb), alloys including the rare earth metal are also usable for the anode. It should be noted that the vacuum deposition method and the sputtering method are usable for forming the anode using the alkali metal, alkaline earth metal and the alloy thereof. Further, when a silver paste is used for the anode, the coating method and the inkjet method are usable.

When the organic EL device is of a bottom emission type, the anode is preferably formed from a light-transmissive or semi-transmissive metallic material that allows light from the emitting layer to be transmitted. Herein, the light-transmissive or semi-transmissive property means the property of allowing transmissivity of 50% or more (preferably 80% or more) of the light emitted from the emitting layer. The light-transmissive or semi-transmissive metallic material can be selected in use as needed from the above materials listed in the description about the anode.

When the organic EL device is of a top emission type, the anode is a reflective electrode having a reflective layer. The reflective layer is preferably formed from a metallic material having light reflectivity. Herein, the light reflectivity means the property of reflecting 50% or more (preferably 80% or more) of the light emitted from the emitting layer. The metallic material having light reflectivity can be selected in use as needed from the above materials listed in the description about the anode.

The anode may consist of the reflective layer, or may be a multilayer structure having the reflective layer and a conductive layer (preferably a transparent conductive layer). When the anode includes the reflective layer and the conductive layer, the conductive layer is preferably provided between the reflective layer and a hole transporting zone. A material of the conductive layer can be selected in use as needed from the above materials listed in the description about the anode.

### Cathode

It is preferable to use metal, an alloy, an electroconductive compound, a mixture thereof, or the like having a small work function (specifically, 3.8 eV or less) for the cathode. Examples of the material for the cathode include elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AlLi) including the alkali metal or the alkaline earth metal, a rare earth metal such as europium (Eu) and ytterbium (Yb), and alloys including the rare earth metal.

It should be noted that the vacuum deposition method and the sputtering method are usable for forming the cathode using the alkali metal, alkaline earth metal and the alloy thereof. Further, when a silver paste is used for the cathode, the coating method and the inkjet method are usable.

By providing the electron injecting layer, various conductive materials such as Al, Ag, ITO, graphene, and indium oxide-tin oxide containing silicon or silicon oxide may be used for forming the cathode regardless of the work function. The conductive materials can be formed into a film using the sputtering method, inkjet method, spin coating method, and the like.

When the organic EL device is of a bottom emission type, the cathode is a reflective electrode. The reflective electrode is preferably formed from a metallic material having light reflectivity. The metallic material having light reflectivity can be selected in use as needed from the above materials listed in the description about the cathode.

When the organic EL device is of a top emission type, the cathode is preferably formed from a light-transmissive or semi-transmissive metallic material that allows light from the emitting layer to be transmitted. The light-transmissive or semi-transmissive metallic material can be selected in use as needed from the above materials listed in the description about the cathode.

The organic EL device according to the exemplary embodiment may be a bottom emission type organic EL device. The organic EL device according to the exemplary embodiment may be a top emission type organic EL device.

In the bottom emission type organic EL device, it is preferable that the anode is a light-transmissive electrode having light transmissivity and the cathode is a light-reflective electrode having light reflectivity.

In the top emission type organic EL device, it is preferable that the anode is a light-reflective electrode having light reflectivity and the cathode is a light-transmissive electrode having light transmissivity.

### Capping Layer

The top emission type organic EL device typically has a capping layer on the top of the cathode.

The capping layer may contain, for instance, at least one compound selected from the group consisting of a high polymer compound, metal oxide, metal fluoride, metal boride, silicon nitride, and silicon compound (e.g., silicon oxide).

In addition, the capping layer may contain, for instance, at least one compound selected from the group consisting of an aromatic amine derivative, anthracene derivative, pyrene derivative, fluorene derivative, and dibenzofuran derivative.

Moreover, a laminate obtained by layering layers that contain these substances is also usable as the capping layer.

### Hole Injecting Layer

The hole injecting layer is a layer containing a substance exhibiting a high hole injectability. Examples of the substance exhibiting a high hole injectability include molybdenum oxide, titanium oxide, vanadium oxide, rhenium oxide, ruthenium oxide, chrome oxide, zirconium oxide, hafnium oxide, tantalum oxide, silver oxide, tungsten oxide, and manganese oxide.

In addition, the examples of the substance exhibiting a high hole injectability include: aromatic amine compounds such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and 3-[N-(1-naphthyl)-N-(9-phenylcarbazole-3-yl)amino]-9-phenylcarbazole (abbreviation: PCzPCN1), those of which are low-molecule organic compounds.

In addition, a high polymer compound (e.g., oligomer, dendrimer and polymer) is usable as the substance exhibiting a high hole injectability. Examples of the high-molecule compound include poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide](abbreviation: PTPDMA), and poly[N, N′-bis(4-butylphenyl)-N, N′-bis(phenyl)benzidine](abbreviation: Poly-TPD). Moreover, an acid-added high polymer compound such as poly(3,4-ethylenedioxythiophene)/poly(styrene sulfonic acid) (PEDOT/PSS) and polyaniline/poly(styrene sulfonic acid) (PAni/PSS) are also usable.

### Hole Transporting Layer

The hole transporting layer is a layer containing a substance exhibiting a high hole transportability. An aromatic amine compound, carbazole derivative, anthracene derivative and the like are usable for the hole transporting layer. Specific examples of a material for the hole transporting layer include 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluorene-9-yl)triphenylamine (abbreviation: BAFLP), 4,4′-bis[N-(9,9-dimethylfluorene-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluorene-2-yl)-N-phenylamino]biphenyl (abbreviation: BSPB). The above-described substances mostly have a hole mobility of 10⁻⁶ cm²/Vs or more.

For the hole transporting layer, a carbazole derivative such as CBP, CzPA, and PCzPA and an anthracene derivative such as t-BuDNA, DNA, and DPAnth may be used. A high polymer compound such as poly(N-vinylcarbazole) (abbreviation: PVK) and poly(4-vinyltriphenylamine) (abbreviation: PVTPA) is also usable.

However, in addition to the above substances, any substance exhibiting a higher hole transportability than an electron transportability may be used. It should be noted that the layer containing the substance exhibiting a high hole transportability may be a single layer or a layer obtained layering two or more layers formed of the above substance(s).

### Electron Transporting Layer

The electron transporting layer is a layer containing a highly electron-transportable substance. For the electron transporting layer, 1) a metal complex such as an aluminum complex, beryllium complex, and zinc complex, 2) a hetero aromatic compound such as imidazole derivative, benzimidazole derivative, azine derivative, carbazole derivative, and phenanthroline derivative, and 3) a high polymer compound are usable. Specifically, as a low-molecule organic compound, a metal complex such as Alq, tris(4-methyl-8-quinolinato)aluminum (abbreviation: Almq₃), bis(10-hydroxybenzo[h]quinolinato)beryllium (abbreviation: BeBg₂), BAlq, Znq, ZnPBO and ZnBTZ is usable. In addition to the metal complex, a heteroaromatic compound such as 2-(4-biphenylyl)-5-(4tert-butylphenyl)-1,3,4-oxadiazole (abbreviation: PBD), 1,3-bis[5-(ptert-butylphenyl)-1,3,4-oxadiazole-2-yl]benzene (abbreviation: OX_{D}-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazole-2-yl)stilbene (abbreviation: BzOs) is usable. The above-described substances mostly have an electron mobility of 10⁻⁶ cm²/Vs or more. It should be noted that any other substance than the above substances may be used for the electron transporting layer as long as the substance exhibits a higher electron transportability than the hole transportability. The electron transporting layer may be a single layer or a layer obtained layering two or more layers formed of the above substance(s).

Further, a high polymer compound is usable for the electron transporting layer. For instance, poly[(9,9-dihexylfluorene-2,7-diyl)-co-(pyridine-3,5-diyl)](abbreviation: PF-Py), and poly[(9,9-dioctylfluorene-2,7-diyl)-co-(2,2′-bipyridine-6,6′-diyl)](abbreviation: PF-BPy) are usable.

### Electron Injecting Layer

The electron injecting layer is a layer that contains a substance exhibiting a high hole injectability. Examples of a material for the electron injecting layer include an alkali metal, alkaline earth metal and a compound thereof, examples of which include lithium (Li), cesium (Cs), calcium (Ca), lithium fluoride (LiF), cesium fluoride (CsF), calcium fluoride (CaF₂), and lithium oxide (LiOx). In addition, the alkali metal, alkaline earth metal or the compound thereof may be added to the substance exhibiting the electron transportability in use. Specifically, for instance, magnesium (Mg) added to Alq may be used. In this case, the electrons can be more efficiently injected from the cathode.

Alternatively, the electron injecting layer may be provided by a composite material in a form of a mixture of the organic compound and the electron donor. Such a composite material exhibits excellent electron injectability and electron transportability since electrons are generated in the organic compound by the electron donor. In this case, the organic compound is preferably a material excellent in transporting the generated electrons. Specifically, the above examples (e.g., the metal complex and the hetero aromatic compound) of the substance forming the electron transporting layer are usable. As the electron donor, any substance exhibiting electron donating property to the organic compound is usable. Specifically, the electron donor is preferably alkali metal, alkaline earth metal and rare earth metal such as lithium, cesium, magnesium, calcium, erbium and ytterbium. The electron donor is also preferably alkali metal oxide and alkaline earth metal oxide such as lithium oxide, calcium oxide, and barium oxide. Moreover, a Lewis base such as magnesium oxide is usable. Further, the organic compound such as tetrathiafulvalene (abbreviation: TTF) is usable.

### Layer Formation Method

A method for forming each layer of the organic EL device according to any of the above exemplary embodiments is subject to no limitation except for the above particular description. However, known methods of dry film-forming such as vacuum deposition, sputtering, plasma or ion plating and wet film-forming such as spin coating, dipping, flow coating or ink-jet are applicable.

### Film Thickness

The film thickness of each layer of the organic layer of the organic EL device according to the exemplary embodiment is not limited unless otherwise specified in the above. In general, the thickness preferably ranges from several nanometers to 1 μm because an excessively small film thickness is likely to cause defects (e.g. pin holes) and an excessively large thickness leads to the necessity of applying high voltage and consequent reduction in efficiency.

### Fourth Exemplary Embodiment

An arrangement of an organic EL device according to a fourth exemplary embodiment will be described. In the description of the fourth exemplary embodiment, the same components as those in the third exemplary embodiment are denoted by the same reference signs and names to simplify or omit description of the components. In the fourth exemplary embodiment, the same materials and compounds as described in the third exemplary embodiment are usable, unless otherwise specified.

The organic EL device according to the fourth exemplary embodiment is different from that according to the third exemplary embodiment in that the emitting layer of the fourth exemplary embodiment further contains the second host material. The rest of the arrangement of the organic EL device according to the fourth exemplary embodiment is the same as in the third exemplary embodiment.

Herein, the compound used as the second host material is occasionally referred to as a compound M1.

In an exemplary embodiment, the emitting layer contains the second host material (compound M1), the first host material (compound M2), and the fluorescent material (compound M3).

### Emitting Layer

In an exemplary embodiment, a single layer contains the second host material, the first host material, and the fluorescent material.

In an exemplary embodiment, a single layer contains the second host material, the sensitizing material as the first host material, and the fluorescent material. For instance, when the organic EL device includes a single emitting layer, the single emitting layer contains the second host material, the sensitizing material, and the fluorescent material, and when the organic EL device includes a plurality of emitting layers, one of the plurality of emitting layers contains the second host material, the sensitizing material, and the fluorescent material.

In an exemplary embodiment, when the emitting layer contains a delayed fluorescent compound as the sensitizing material, the emitting layer contains no phosphorescent metal complex.

### Second Host Material

In the fourth exemplary embodiment, the second host material is the compound M1 having, in one molecule, at least one partial structure selected from the group consisting of partial structures represented by formulae (101) to (118) below.

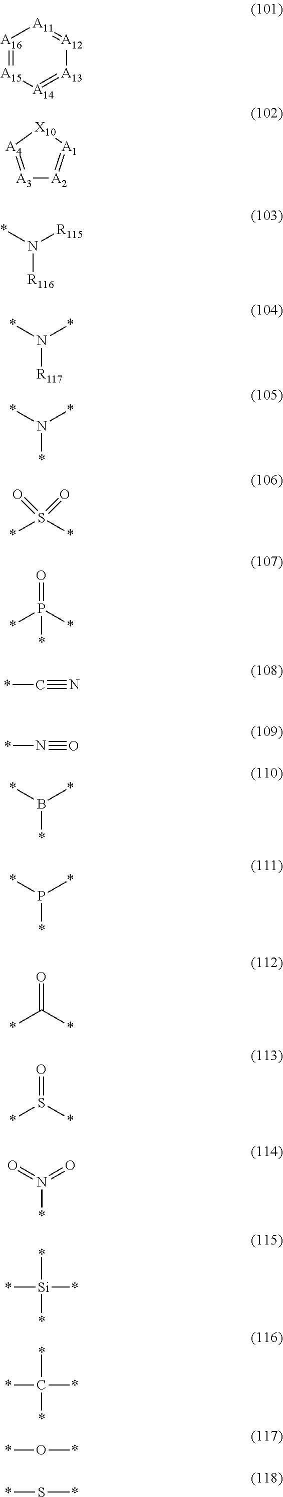

In the formula (101):
A₁₁ to A₁₆ are each independently a nitrogen atom, CR₁₁, or a carbon atom bonded to another atom or another structure in the molecule of the compound M1;
at least one of A₁₁ to A₁₆ is a carbon atom bonded to another atom or another structure in the molecule of the compound M1, and
when a plurality of R₁₁ are present, the plurality of R₁₁ are mutually the same or different, and at least one combination of adjacent two or more of R₁₁ are mutually bonded to form a substituted or unsubstituted monocyclic ring, mutually bonded to form a substituted or unsubstituted fused ring, or not mutually bonded; in the formula (102):
A₁ to A₄ are each independently a nitrogen atom, CR₁₂, or a carbon atom bonded to another atom or another structure in the molecule of the compound M1;
each R₁₂ is independently a hydrogen atom or a substituent, or at least one combination of combinations of adjacent ones of R₁₂ are mutually bonded to form a ring;
when a plurality of R₁₂ are present, the plurality of R₁₂ are mutually the same or different, and at least one combination of adjacent two or more of R₁₂ are mutually bonded to form a substituted or unsubstituted monocyclic ring, mutually bonded to form a substituted or unsubstituted fused ring, or not mutually bonded;
X₁₀ is NR₁₃, C(R₁₄)(R₁₅), Si(R₁₆)(R₁₇), an oxygen atom, a sulfur atom, a nitrogen atom bonded to another atom or another structure in the molecule of the compound M1, a carbon atom bonded to R₁₈ and to another atom or another structure in the molecule of the compound M1, or a silicon atom bonded to R₁₉ and to another atom or another structure in the molecule of the compound M1;
at least one of carbon atoms in A₁ to A₄, a nitrogen atom in X₁₀, a carbon atom in X₁₀, or a silicon atom in X₁₀ is bonded to another atom or another structure in the molecule of the compound M1;
a combination of R₁₄ and R₁₅ are mutually bonded to form a substituted or unsubstituted monocyclic ring, mutually bonded to form a substituted or unsubstituted fused ring, or not mutually bonded; and
a combination of R₁₆ and R₁₇ are mutually bonded to form a substituted or unsubstituted monocyclic ring, mutually bonded to form a substituted or unsubstituted fused ring, or not mutually bonded;
in the formula (103): a combination of R₁₁₅ and R₁₁₆ are mutually bonded to form a substituted or unsubstituted monocyclic ring, mutually bonded to form a substituted or unsubstituted fused ring, or not mutually bonded;
in the formulae (101) to (104): R₁₁, R₁₂, R₁₄, R₁₅, R₁₆, R₁₇, R₁₁₅ and R₁₁₆ forming neither the substituted or unsubstituted monocyclic ring nor the substituted or unsubstituted fused ring, and R₁₃, R₁₈, R₁₉ and R₁₁₇ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a group represented by —C(═O)R₉₀₈, a group represented by —COOR₉₀₉, a group represented by —P(═O)(R₉₁₀)(R₉₁₁), a group represented by —P(═O)(OR₉₁₂)(OR₉₁₃), a group represented by —Ge(R₉₁₄)(R₉₁₅)(R₉₁₆), a group represented by —B(R₉₁₇)(R₉₁₈), a substituted or unsubstituted aralkyl group having 7 to 50 carbon atoms, a halogen atom, a cyano group, a nitro group, a substituted or unsubstituted aryl group having 6 to 50 ring carbon atoms, or a substituted or unsubstituted heterocyclic group having 5 to 50 ring atoms; and in the formulae (103) to (118):
each * is a site bonded to another atom or another structure in the molecule of the compound M1; and
when the compound M1 includes a plurality of partial structures represented by the formula (101), a plurality of partial structures represented by the formula (102), a plurality of partial structures represented by the formula (103), and a plurality of partial structures represented by the formula (104),
the plurality of partial structures represented by the formula (101) are mutually the same or different;
the plurality of partial structures represented by the formula (102) are mutually the same or different;
the plurality of partial structures represented by the formula (103) are mutually the same or different; and
the plurality of partial structures represented by the formula (104) are mutually the same or different.

In the compound M1:
R₉₀₁ to R₉₁₈ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₆ are present, the plurality of R₉₀₆ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₉₀₈ are present, the plurality of R₉₀₈ are mutually the same or different;
when a plurality of R₉₀₆ are present, the plurality of R₉₀₆ are mutually the same or different;
when a plurality of R₉₁₀ are present, the plurality of R₉₁₀ are mutually the same or different;
when a plurality of R₉₁₁ are present, the plurality of R₉₁₁ are mutually the same or different;
when a plurality of R₉₁₂ are present, the plurality of R₉₁₂ are mutually the same or different;
when a plurality of R₉₁₃ are present, the plurality of R₉₁₃ are mutually the same or different;
when a plurality of R₉₁₄ are present, the plurality of R₉₁₄ are mutually the same or different;
when a plurality of R₉₁₅ are present, the plurality of R₉₁₅ are mutually the same or different;
when a plurality of R₉₁₆ are present, the plurality of R₉₁₆ are mutually the same or different;
when a plurality of R₉₁₇ are present, the plurality of R₉₁₇ are mutually the same or different; and
when a plurality of R₉₁₈ are present, the plurality of R₉₁₈ are mutually the same or different.

In the formula (102), when X₁₀ is a nitrogen atom bonded to another atom or another structure in the molecule of the compound M1, the formula (102) is represented by a formula (102-1) below.

In the formula (102), when X₁₀ is a carbon atom bonded to R₁₈ and to another atom or another structure in the molecule of the compound M1, the formula (102) is represented by a formula (102-2) below.

In the formula (102), when X₁₀ is a silicon atom bonded to Rig and to another atom or another structure in the molecule of the compound M1, the formula (102) is represented by a formula (102-3) below.

In the formulae (102-1) to (102-3), A₁ to A₄ each independently represent the same as A₁ to A₄ in the formula (102), and R₁₈ and R₁₉ each independently represent the same as R₁₂ in the formula (102), and each * is a site bonded to another atom or another structure in the molecule of the compound M1.

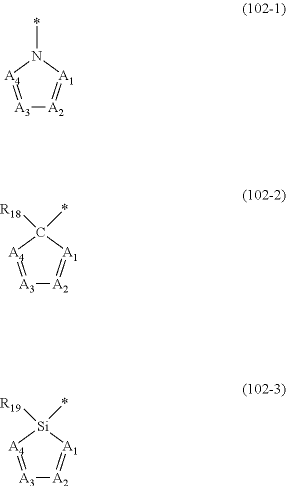

In an exemplary embodiment, the second host material has at least one partial structure represented by the formula (101).

In an exemplary embodiment, the partial structure represented by the formula (101) is at least one selected from the group consisting of partial structures represented by formulae (A11) to (A19) below.

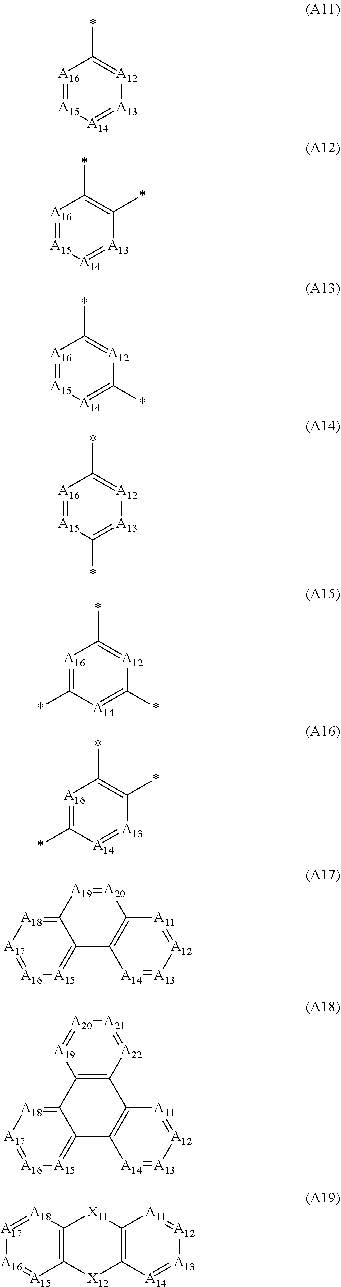

In the formulae (A11) to (A₁₆), A₁₂ to A₁₆ are each independently a nitrogen atom or CR₁₁, R₁₁ represents the same as R₁₁ in the formula (101), and each * is a site bonded to another atom or another structure in the molecule of the compound M1;
in the formulae (A17) and (A18), A₁₁ to A₂₂ are each independently a nitrogen atom, CR₁₁, or a carbon atom bonded to another atom or another structure in the molecule of the compound M1, and each Rn independently represents the same as R₁₁ in the formula (101), and at least one of A₁₁ to A₂₂ is a carbon atom bonded to another atom or another structure in the molecule of the compound M1;
in the formula (A₁₉), A₁₁ to A18 are each independently a nitrogen atom, CR₁₁, or a carbon atom bonded to another atom or another structure in the molecule of the compound M1; each R₁₁ independently represents the same as R₁₁ in the formula (101); X₁₁ and X₁₂ each independently represent the same as X₁₀ in the formula (102); and at least one of carbon atoms in A₁₁ to A18, nitrogen atoms in X₁₁ and X₁₂, carbon atoms in X₁₁ and X₁₂, or silicon atoms in X₁₁ and X₁₂ is bonded to another atom or another structure in the molecule of the compound M1; and
each * in the formulae (A11) to (A19) is a site bonded to another atom or another structure in the molecule of the compound M1.

In an exemplary embodiment, the second host material has at least one partial structure represented by the formula (102).

In an exemplary embodiment, the partial structure represented by the formula (102) is at least one selected from the group consisting of partial structures represented by formulae (B₁₁) to (B₂₄) below.

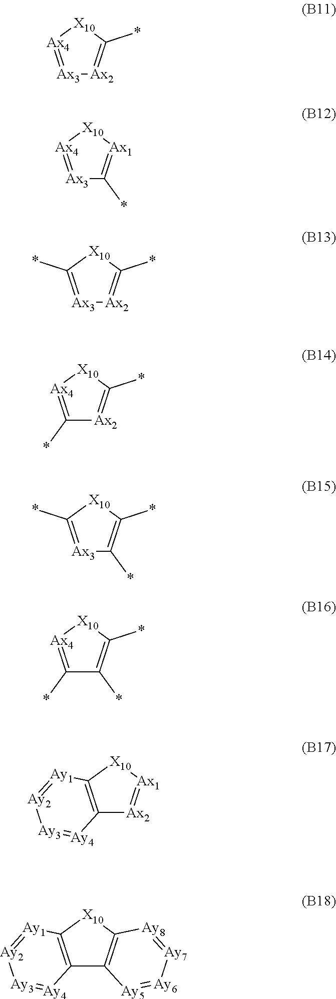

In the formulae (B₁₁) to (B₁₆): Ax₁ to Ax₄ are each independently a nitrogen atom or CR₁₂; each R₁₂ independently represents the same as R₁₂ in the formula (102); and X₁₀ represents the same as X₁₀ in the formula (102); and each * is a site bonded to another atom or another structure in the molecule of the compound M1, in the formula (B₁₇): Ax₁, Ax₂, and Ay₁ to Ay₄ are each independently a nitrogen atom, CR₁₂, or a carbon atom bonded to another atom or another structure in the molecule of the compound M1; each R₁₂ independently represents the same as R₁₂ in the formula (102); X₁₀ represents the same as X₁₀ in the formula (102); and at least one of carbon atoms in Ax₁, Ax₂, and Ay₁ to Ay₄, a nitrogen atom in X₁₀, a carbon atom in X₁₀, or a silicon atom in X₁₀ is bonded to another atom or another structure in the molecule of the compound M1,
in the formula (B₁₈): Ay₁ to Ay₈ are each independently a nitrogen atom, CR₁₂, or a carbon atom bonded to another atom or another structure in the molecule of the compound M1; each R₁₂ independently represents the same as R₁₂ in the formula (102); X₁₀ represents the same as X₁₀ in the formula (102); and at least one of carbon atoms in Ay₁ to Ay₈, a nitrogen atom in X₁₀, a carbon atom in X₁₀, or a silicon atom in X₁₀ is bonded to another atom or another structure in the molecule of the compound M1, and
each * in the formulae (B₁₁) to (B₁₈) is a site bonded to another atom or another structure in the molecule of the compound M1.

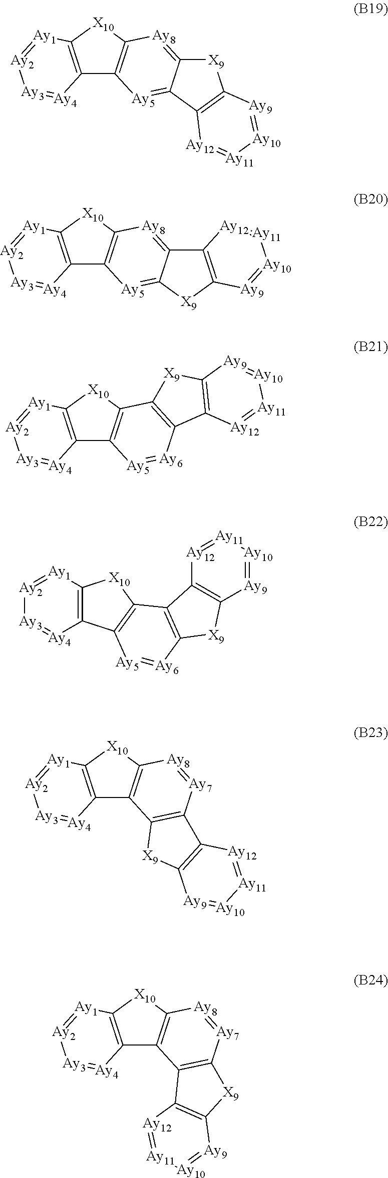

In the formulae (B₁₉) to (B₂₄), Ay₁ to Ay₈ and Ay₉ to Ay₁₂ are each independently a nitrogen atom, CR₁₂, or a carbon atom bonded to another atom or another structure in the molecule of the compound M1; each R₁₂ independently represents the same as R₁₂ in the formula (102); and X₉ and X₁₀ each independently represent the same as X₁₀ in the formula (102); and
at least one of carbon atoms in Ay₁ to Ay₈ and Ay₉ to Ay₁, nitrogen atoms in X₉ and X₁₀, carbon atoms in X₉ and X₁₀, or silicon atoms in X₉ and X₁₀ is bonded to another atom or another structure in the molecule of the compound M1.

In the compound M1 of the exemplary embodiment, R₁₁, R₁₂, and R₁₁₅ to R₁₁₇ are each independently preferably a hydrogen atom, halogen atom, cyano group, unsubstituted aryl group having 6 to 30 ring carbon atoms, unsubstituted heterocyclic group having 5 to 30 ring atoms, unsubstituted alkyl group having 1 to 30 carbon atoms, unsubstituted alkyl halide group having 1 to 30 carbon atoms, unsubstituted alkylsilyl group having 3 to 30 carbon atoms, unsubstituted arylsilyl group having 6 to 60 ring carbon atoms, unsubstituted arylphosphoryl group having 6 to 60 ring carbon atoms, unsubstituted alkoxy group having 1 to 30 carbon atoms, unsubstituted aryloxy group having 6 to 30 ring carbon atoms, amino group, unsubstituted alkylamino group having 2 to 30 carbon atoms, unsubstituted arylamino group having 6 to 60 ring carbon atoms, thiol group, unsubstituted alkylthio group having 1 to 30 carbon atoms, or unsubstituted arylthio group having 6 to 30 ring carbon atoms.

In the compound M1 of the exemplary embodiment, R₁₁, R₁₂, and R₁₁₅ to R₁₁₇ are each independently more preferably a hydrogen atom, halogen atom, cyano group, unsubstituted aryl group having 6 to 14 ring carbon atoms, unsubstituted heterocyclic group having 5 to 14 ring atoms, unsubstituted alkyl group having 1 to 6 carbon atoms, unsubstituted alkyl halide group having 1 to 6 carbon atoms, unsubstituted alkylsilyl group having 3 to 6 carbon atoms, unsubstituted arylsilyl group having 6 to 60 ring carbon atoms, unsubstituted arylphosphoryl group having 6 to 60 ring carbon atoms, unsubstituted alkoxy group having 1 to 6 carbon atoms, unsubstituted aryloxy group having 6 to 14 ring carbon atoms, amino group, unsubstituted alkylamino group having 2 to 12 carbon atoms, unsubstituted arylamino group having 6 to 60 ring carbon atoms, thiol group, unsubstituted alkylthio group having 1 to 6 carbon atoms, or unsubstituted arylthio group having 6 to 14 ring carbon atoms.

In the compound M1 of the exemplary embodiment, still more preferably, R₁₁, R₁₂, and R₁₁₅ to R₁₁₇ are each a hydrogen atom.

In the compound M1 of the exemplary embodiment, R₁₃ to R₁₉ in X₁₀ and R₁₃ to R₁₉ in X₉ (representing same as the R₁₃ to R₁₉ in X₁₀) are preferably each independently a hydrogen atom, unsubstituted aryl group having 6 to 30 ring carbon atoms, unsubstituted heterocyclic group having 5 to 30 ring atoms, unsubstituted alkyl group having 1 to 30 carbon atoms, or unsubstituted alkyl halide group having 1 to 30 carbon atoms.

In the compound M1 of the exemplary embodiment, R₁₃ to R₁₉ in X₁₀ and R₁₃ to R₁₉ in X₉ are more preferably each independently a hydrogen atom, unsubstituted aryl group having 6 to 14 ring carbon atoms, unsubstituted heterocyclic group having 5 to 14 ring atoms, unsubstituted alkyl group having 1 to 6 carbon atoms, or unsubstituted alkyl halide group having 1 to 6 carbon atoms.

In the compound M1 of the exemplary embodiment, R₁₃ to R₁₉ in X₁₀ and R₁₃ to R₁₉ in X₉ are still more preferably each independently an unsubstituted aryl group having 6 to 14 ring carbon atoms, or unsubstituted alkyl group having 1 to 6 carbon atoms.

The respective partial structures represented by the formulae (101) to (118) are exemplified by partial structures represented by formulae (A₁₀₁) to (A₁₂₁) and (B₁₀₁) to (B₁₂₅) below.

The compound M1 also preferably includes, in one molecule, at least one of the partial structures represented by the formulae (A₁₀₁) to (A121) or (B₁₀₁) to (B₁₂₅).

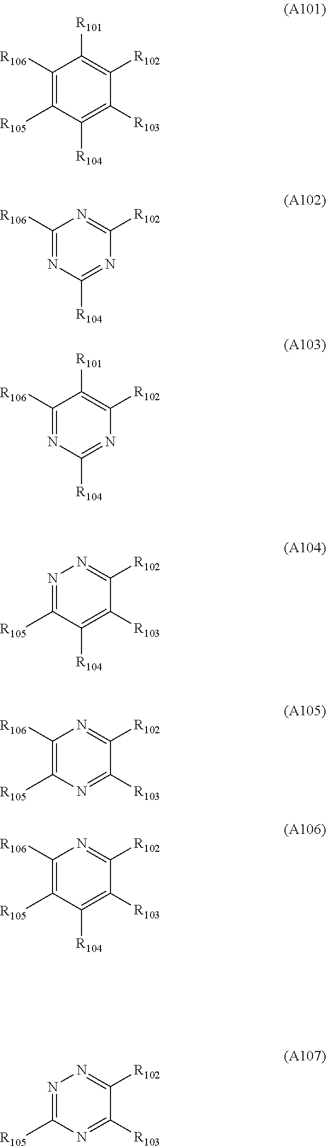

In the formulae (A₁₀₁) to (A107): Rios to Rios each independently represent the same as R₁₁ in the formula (101); and at least one of R₁₀₁ to R₁₀₆ is a single bond bonded to another atom or another structure in the molecule of the compound M1.

In the formulae (A₁₀₁) to (A107): at least one combination of a combination of adjacent R₁₀₁ and R₁₀₂, a combination of adjacent R₁₀₂ and R₁₀₃, a combination of adjacent R₁₀₃ and R₁₀₄, a combination of adjacent R₁₀₄ and R₁₀₅, a combination of adjacent R₁₀₅ and R₁₀₆, and a combination of adjacent R₁₀₆ and R₁₀₁ are mutually bonded to form a substituted or unsubstituted monocyclic ring, mutually bonded to form a substituted or unsubstituted fused ring, or not mutually bonded.

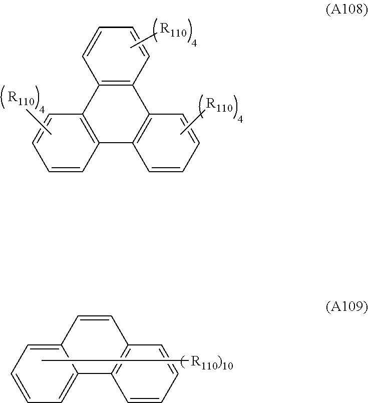

In the formulae (A108) to (A109): each R₁₁₀ independently represents the same as R₁₁ in the formula (101); at least one R₁₁₀ is a single bond bonded to another atom or another structure in the molecule of the compound M1; a plurality of R₁₁₀ are mutually the same or different; and at least one combination of adjacent two or more of a plurality of R₁₁₀ are mutually bonded to form a substituted or unsubstituted monocyclic ring, mutually bonded to form a substituted or unsubstituted fused ring, or not mutually bonded.

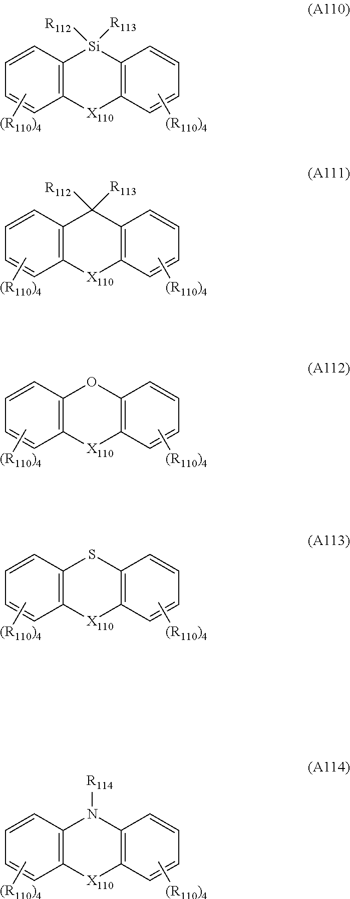

In the formulae (A110) to (A114): R₁₁₀ and R₁₁₂ to R₁₁₄ each independently represent the same as R₁₁ in the formula (101); each X₁₁₀ independently represents the same as X₁₀ in the formula (102); at least one of R₁₁₀ or R₁₁₂ to R₁₁₄ is a single bond bonded to another atom or another structure in the molecule of the compound M1; at least one of a nitrogen atom, a carbon atom, or a silicon atom in X₁₁₀ is bonded to another atom or another structure in the molecule of the compound M1; and a plurality of R₁₁₀ are mutually the same or different.

In the formulae (A110) to (A114): at least one combination of a combination of adjacent two or more of a plurality of R₁₁₀, a combination of R₁₁₂ and R₁₁₃, a combination of R₁₄ and R₁₅ in X₁₁₀ (representing the same as a combination of R₁₄ and R₁₅ in X₁₀), and a combination of R₁₆ and R₁₇ in X₁₁₀ (representing the same as a combination of R₁₆ and R₁₇ in X₁₀) are mutually bonded to form a substituted or unsubstituted monocyclic ring, mutually bonded to form a substituted or unsubstituted fused ring, or not mutually bonded.

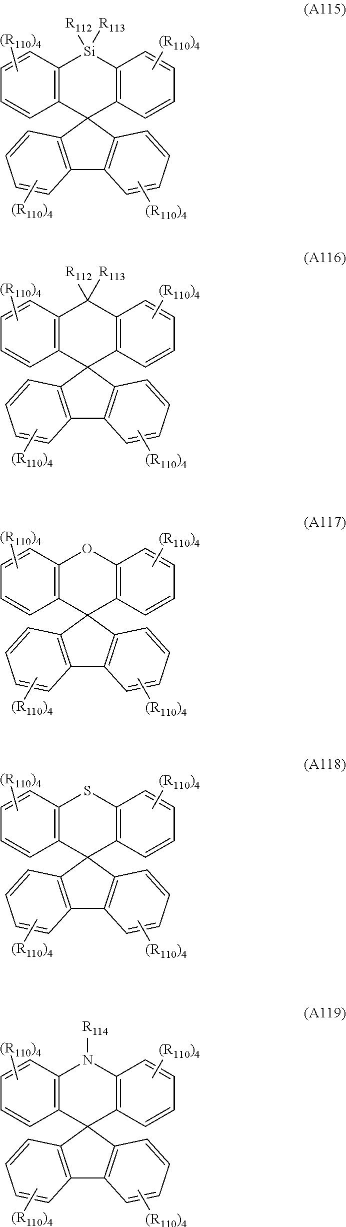

In the formulae (A115) to (A119): R₁₁₀ and R₁₁₂ to R₁₁₄ each independently represent the same as R₁₁ in the formula (101); at least one of R₁₁₀ or R₁₁₂ to R₁₁₄ is a single bond bonded to another atom or another structure in the molecule of the compound M1; and a plurality of R₁₁₀ are mutually the same or different.

In the formulae (A115) to (A119), at least one combination of a combination of adjacent two or more of a plurality of Rim, and a combination of R₁₁₂ and R₁₁₃ are mutually bonded to form a substituted or unsubstituted monocyclic ring, mutually bonded to form a substituted or unsubstituted fused ring, or not mutually bonded.

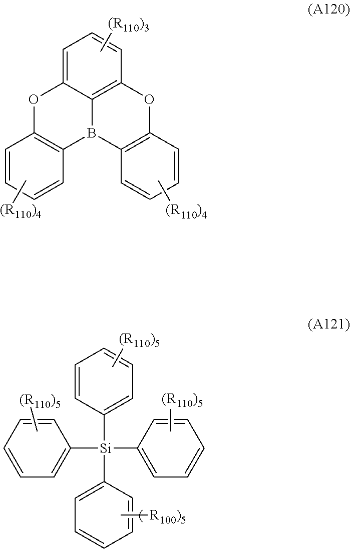

In the formulae (A120) to (A121): each R₁₁₀ independently represents the same as R₁₁ in the formula (101); at least one R₁₁₀ is a single bond bonded to another atom or another structure in the molecule of the compound M1; and a plurality of R₁₁₀ are mutually the same or different.

In the formulae (A120) to (A₁₂₁), at least one combination of adjacent two or more of a plurality of R₁₁₀ are mutually bonded to form a substituted or unsubstituted monocyclic ring, mutually bonded to form a substituted or unsubstituted fused ring, or not mutually bonded.

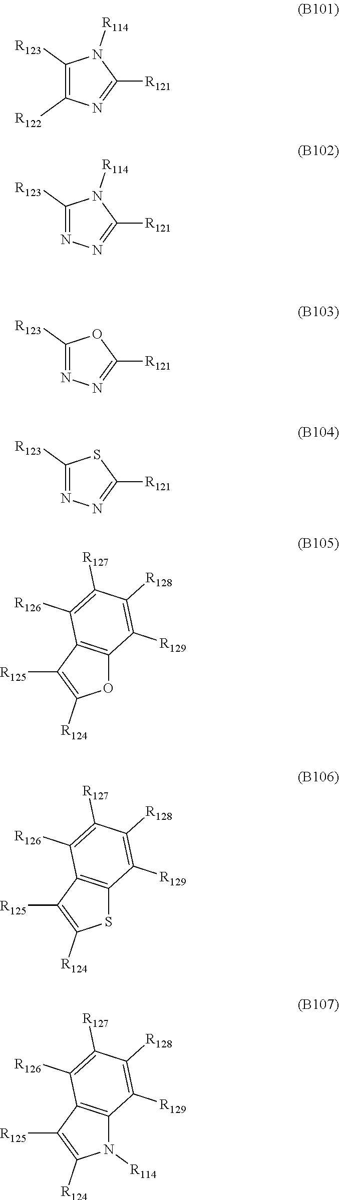

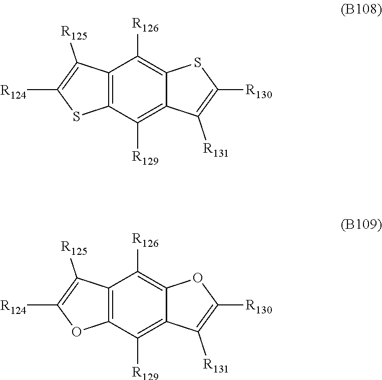

In the formulae (B101) to (B109): R₁₁₄ and R₁₂₁ to R₁₃₁ each independently represent the same as R₁₂ in the formula (102); and at least one of R₁₁₄ or R₁₂₁ to R₁₃₁ is a single bond bonded to another atom or another structure in the molecule of the compound M1.

In the formulae (B101) to (B102), at least one combination of a combination of R₁₂₂ and R₁₂₃, a combination of R₁₂₃ and R₁₁₄, and a combination of R₁₁₄ and R₁₂₁ are mutually bonded to form a substituted or unsubstituted monocyclic ring, mutually bonded to form a substituted or unsubstituted fused ring, or not mutually bonded.

In the formulae (B105) to (B106), at least one combination of a combination of R₁₂₄ and R₁₂₅, a combination of R₁₂₅ and R₁₂₆, a combination of R₁₂₆ and R₁₂₇, a combination of R₁₂₇ and R₁₂₈, and a combination of R₁₂₈ and R₁₂₉ are mutually bonded to form a substituted or unsubstituted monocyclic ring, mutually bonded to form a substituted or unsubstituted fused ring, or not mutually bonded.

In the formula (B107), at least one combination of a combination of R₁₂₄ and R₁₂₅, a combination of R₁₂₅ and R₁₂₆, a combination of R₁₂₆ and R₁₂₇, a combination of R₁₂₇ and R₁₂₈, a combination of R₁₂₈ and R₁₂₉, a combination of R₁₂₉ and R₁₁₄, and a combination of R₁₁₄ and R₁₂₄ are mutually bonded to form a substituted or unsubstituted monocyclic ring, mutually bonded to form a substituted or unsubstituted fused ring, or not mutually bonded.

In the formulae (B108) to (B109), at least one combination of a combination of R₁₂₄ and R₁₂₅, a combination of R₁₂₅ and R₁₂₆, a combination of R₁₃₀ and R₁₃₁, and a combination of R₁₃₁ and R₁₂₉ are mutually bonded to form a substituted or unsubstituted monocyclic ring, mutually bonded to form a substituted or unsubstituted fused ring, or not mutually bonded.

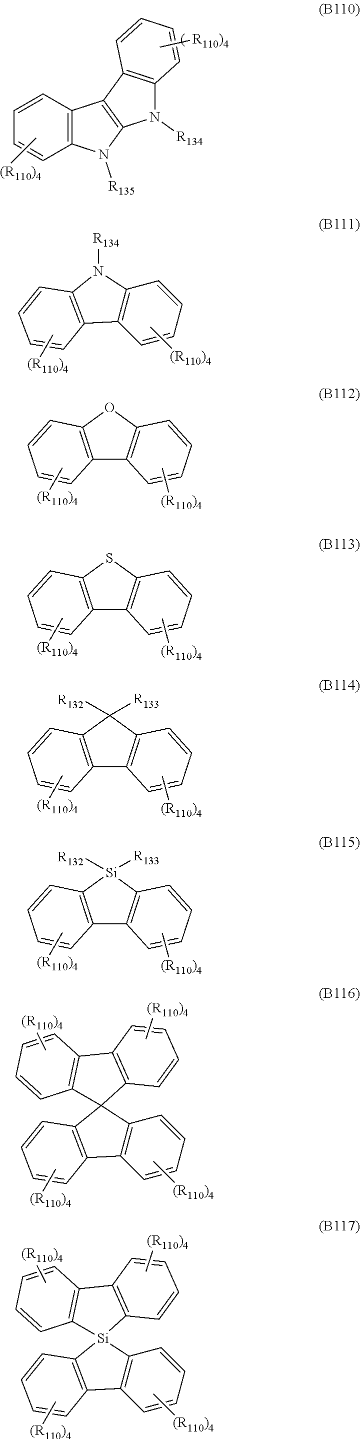

In the formulae (B110) to (B117): R₁₁₀ and R₁₃₂ to R₁₃₅ each independently represent the same as R₁₂ in the formula (102); at least one of R₁₁₀ or R₁₃₂ to R₁₃₅ is a single bond bonded to another atom or another structure in the molecule of the compound M1; and a plurality of R₁₁₀ are mutually the same or different.

In the formulae (B110) to (B117), at least one combination of a combination of adjacent two or more of a plurality of R₁₁₀, and a combination of R₁₃₂ and R₁₃₃ are mutually bonded to form a substituted or unsubstituted monocyclic ring, mutually bonded to form a substituted or unsubstituted fused ring, or not mutually bonded.

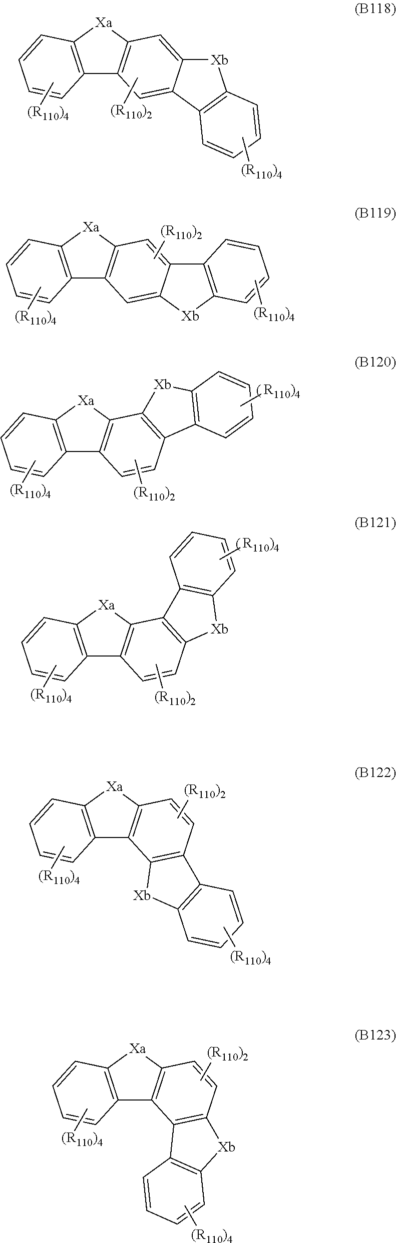

In the formulae (B118) to (B123): each R₁₁₀ independently represents the same as R₁₂ in the formula (102); Xa and Xb each independently represent the same as X₁₀ in the formula (102); at least one R₁₁₀ is a single bond bonded to another atom or another structure in the molecule of the compound M1, or at least one of nitrogen atoms, carbon atoms, or silicon atoms in Xa and Xb is bonded to another atom or another structure in the molecule of the compound M1; and a plurality of R₁₁₀ are mutually the same or different.

In the formulae (B118) to (B123): at least one combination of a combination of adjacent two or more of a plurality of R₁₁₀, a combination of R₁₄ and R₁₅ in Xa and a combination of R₁₄ and R₁₅ in Xb (representing the same as a combination of R₁₄ and R₁₅ in X₁₀), and a combination of R₁₆ and R₁₇ in Xa and a combination of R₁₆ and R₁₇ in Xb (representing the same as a combination of R₁₆ and R₁₇ in X₁₀) are mutually bonded to form a substituted or unsubstituted monocyclic ring, mutually bonded to form a substituted or unsubstituted fused ring, or not mutually bonded.

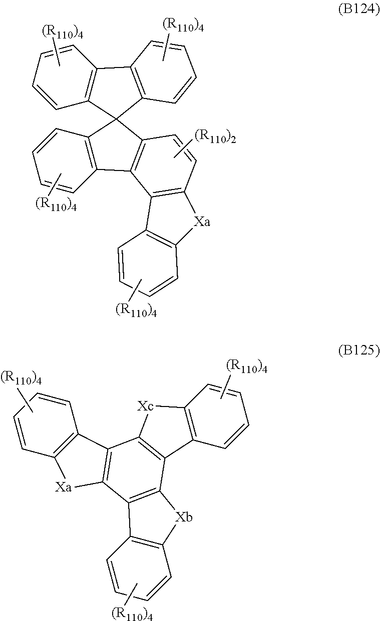

In the formulae (B124) to (B125): each R₁₁₀ independently represents the same as R₁₂ in the formula (102); Xa, Xb, and Xc each independently represent the same as X₁₀ in the formula (102); at least one R₁₁₀ is a single bond bonded to another atom or another structure in the molecule of the compound M1, or at least one of nitrogen atoms, carbon atoms, or silicon atoms in Xa, Xb, and Xc is bonded to another atom or another structure in the molecule of the compound M1; and a plurality of R₁₁₀ are mutually the same or different.

In the formulae (B124) to (B₁₂₅): at least one combination of a combination of adjacent two or more of a plurality of R₁₁₀, a combination of R₁₄ and R₁₅ in Xa, Xb, and Xc (representing the same as a combination of R₁₄ and R₁₅ in X₁₀), and a combination of R₁₆ and R₁₇ in Xa, Xb, and Xc (representing the same as R₁₆ and R₁₇ in X₁₀) are mutually bonded to form a substituted or unsubstituted monocyclic ring, mutually bonded to form a substituted or unsubstituted fused ring, or not mutually bonded.

In the formulae (A₁₀₁) to (A121) and (B₁₀₁) to (B₁₂₅), R₁₁₀, R₁₀₁ to R₁₀₆, R₁₁₂ to R₁₁₄, R₁₂₁ to R₁₃₁, and R₁₃₂ to R₁₃₅ are each independently preferably a hydrogen atom, unsubstituted aryl group having 6 to 30 ring carbon atoms, unsubstituted heterocyclic group having 5 to 30 ring atoms, unsubstituted alkyl group having 1 to 30 carbon atoms, or unsubstituted alkyl halide group having 1 to 30 carbon atoms, more preferably a hydrogen atom, unsubstituted aryl group having 6 to 14 ring carbon atoms, unsubstituted heterocyclic group having 5 to 14 ring atoms, unsubstituted alkyl group having 1 to 6 carbon atoms, or unsubstituted alkyl halide group having 1 to 6 carbon atoms, and still more preferably unsubstituted aryl group having 6 to 14 ring carbon atoms or unsubstituted alkyl group having 1 to 6 carbon atoms.

In the formulae (A₁₀₁) to (A₁₂₁) and (B₁₀₁) to (B₁₂₅), R₁₃ to R₁₉ in Xa, Xb, Xc, and X₁₁₀ (representing the same as R₁₃ to R₁₉ in X₁₀) are each independently preferably a hydrogen atom, unsubstituted aryl group having 6 to 30 ring carbon atoms, unsubstituted heterocyclic group having 5 to 30 ring atoms, unsubstituted alkyl group having 1 to 30 carbon atoms, or unsubstituted alkyl halide group having 1 to 30 carbon atoms, more preferably a hydrogen atom, unsubstituted aryl group having 6 to 14 ring carbon atoms, unsubstituted heterocyclic group having 5 to 14 ring atoms, unsubstituted alkyl group having 1 to 6 carbon atoms, or unsubstituted alkyl halide group having 1 to 6 carbon atoms, and still more preferably unsubstituted aryl group having 6 to 14 ring carbon atoms or unsubstituted alkyl group having 1 to 6 carbon atoms.

In the exemplary embodiment, the compound M1 preferably has (I) at least one of a cyano group, an amino group, a substituted or unsubstituted alkylamino group having 2 to 30 carbon atoms, or a substituted or unsubstituted arylamino group having 6 to 60 ring carbon atoms, or (II) at least one monovalent or higher-valent residue derived from any of a substituted or unsubstituted benzene, a substituted or unsubstituted naphthalene, a substituted or unsubstituted indole, a substituted or unsubstituted carbazole, a substituted or unsubstituted dibenzofuran, a substituted or unsubstituted dibenzothiophene, a substituted or unsubstituted fluorene, a substituted or unsubstituted silafluorene, a substituted or unsubstituted triazine, a substituted or unsubstituted pyrimidine, a substituted or unsubstituted pyridine, a substituted or unsubstituted pyridazine, a substituted or unsubstituted pyrazine, a substituted or unsubstituted imidazole, a substituted or unsubstituted benzimidazole, a substituted or unsubstituted phenanthrene, and a substituted or unsubstituted triphenylene.

In the exemplary embodiment, the compound M1 more preferably has (III) at least one cyano group, or (IV) at least one monovalent or higher-valent residue derived from any of a substituted or unsubstituted carbazole, a substituted or unsubstituted dibenzofuran, a substituted or unsubstituted dibenzothiophene, a substituted or unsubstituted fluorene, a substituted or unsubstituted silafluorene, a substituted or unsubstituted triazine, a substituted or unsubstituted pyrimidine, a substituted or unsubstituted pyridine, and a substituted or unsubstituted triphenylene.

In the exemplary embodiment, the compound M1 still more preferably has at least one monovalent or higher-valent residue derived from any of a substituted or unsubstituted carbazole, a substituted or unsubstituted dibenzofuran, a substituted or unsubstituted dibenzothiophene, a substituted or unsubstituted triazine, and a substituted or unsubstituted pyrimidine.

In the exemplary embodiment, the compound M1 preferably has at least one monovalent or higher-valent residue derived from a substituted or unsubstituted carbazole.

In the exemplary embodiment, the compound M1 preferably has at least one partial structure represented by a formula (15) below.

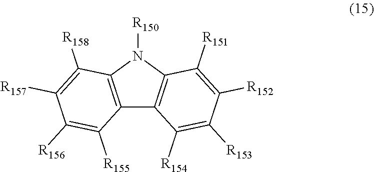

In the formula (15):
at least one of R₁₅₀ to R₁₅₈ is a single bond bonded to another atom or another structure in the molecule of the compound M1; and
R₁₅₀ to R₁₅₈ not being the single bond are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a group represented by —C(═O)R₉₀₈, a group represented by —COOR₉₀₉, a group represented by —P(═O)(R₉₁₀)(R₉₁₁), a group represented by —Ge(R₉₁₂)(R₉₁₃)(R₉₁₄), a group represented by —B(R₉₁₅)(R₉₁₆), a substituted or unsubstituted aralkyl group having 7 to 50 carbon atoms, a halogen atom, a cyano group, a nitro group, a substituted or unsubstituted aryl group having 6 to 50 ring carbon atoms, or a substituted or unsubstituted heterocyclic group having 5 to 50 ring atoms.

In the formula (15), R₁₅₀ is preferably a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, or a substituted or unsubstituted alkyl halide group having 1 to 30 carbon atoms, more preferably a substituted or unsubstituted aryl group having 6 to 30 ring carbon atoms or a substituted or unsubstituted alkyl group having 1 to 30 carbon atoms, and still more preferably a substituted or unsubstituted aryl group having 6 to 30 ring carbon atoms.

### Compound M1 Represented by Formula (161) or Formula (162)

In the exemplary embodiment, the compound M1 is also preferably a compound represented by a formula (161) or a formula (162) below.

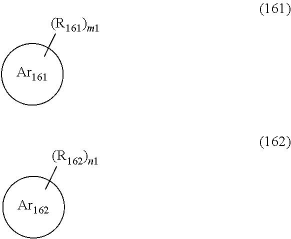

In the formula (161):
Ar₁₆₁ is a substituted or unsubstituted aromatic hydrocarbon ring having 6 to 30 ring carbon atoms, or a substituted or unsubstituted heterocycle having 5 to 30 ring atoms;
m1 is 1, 2, 3, 4, 5, or 6;
R₁₆₁ is an electron-donating group, and each R₁₆₁ is bonded to an element forming Ar_{162;}
when m1 is 2 or more, a plurality of R₁₆₁ are mutually the same or different; and
Ar₁₆₁ is not an electron-accepting aromatic hydrocarbon ring or heterocycle, and when Ar₁₆₂ has a substituent, the substituent is not an electron-accepting group, and
in the formula (162):
Ar₁₆₂ is a substituted or unsubstituted aromatic hydrocarbon ring having 6 to 30 ring carbon atoms, or a substituted or unsubstituted heterocycle having 5 to 30 ring atoms;
n1 is 1, 2, 3, 4, 5, or 6;
R₁₆₂ is an electron-accepting group, and each R₁₆₂ is bonded to an element forming Ar_{162;}
when n1 is 2 or more, a plurality of R₁₆₂ are mutually the same or different; and
Ar₁₆₂ is not an electron-donating aromatic hydrocarbon ring or heterocycle, and when Ar₁₆₂ has a substituent, the substituent is not an electron-donating group.

In the formulae (161) and (162), Ar₁₆₂ and Ar₁₆₂ are preferably each independently a monovalent or higher-valent residue derived from any of compounds represented by formulae (A61) and (A62) below.

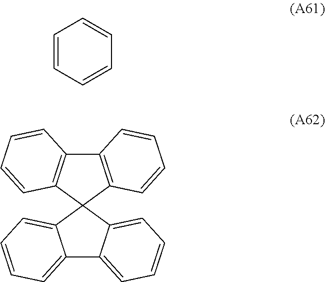

In the exemplary embodiment, it is preferable that each R₁₆₁ in the formula (161) is independently a monovalent or higher-valent residue derived from any of compounds represented by formulae (DN1) to (DN6) and (DN8) to (DN10) below, or a group represented by a formula (DN7) below.

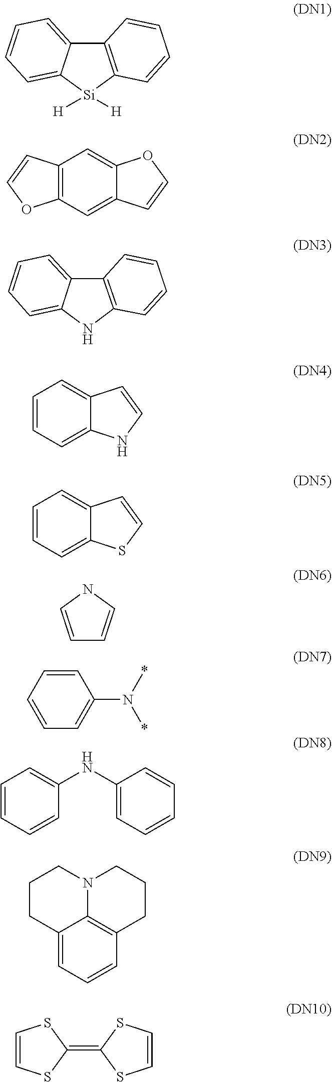

In the formula (D7), each * represents a site bonded to an element forming Ar₁₆₁.

In the exemplary embodiment, it is preferable that each R₁₆₂ in the formula (162) is independently a monovalent or higher-valent residue derived from any of compounds represented by formulae (AC4) to (AC18) and (AC22) to (AC23) below, or a group represented by one of formulae (AC1) to (AC3), (AC19) to (AC21), and (AC24) below.

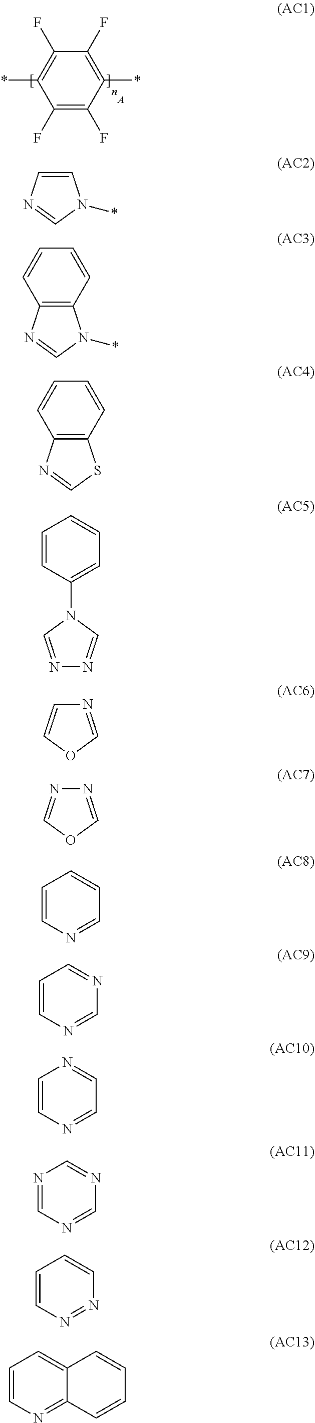

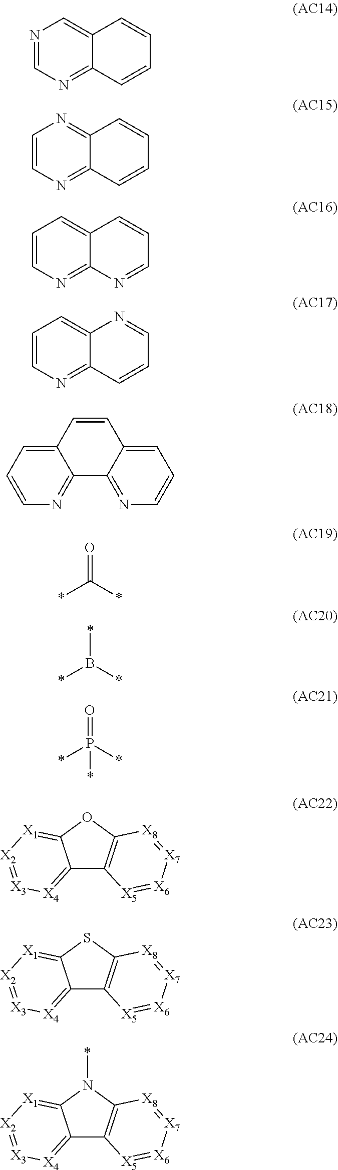

In the formula (AC1), n_{A} is 1, 2, or 3;
in the formulae (AC22) to (AC23), X₁ to X₈ are each independently CR₁₆₃ or a carbon atom bonded to another atom or another structure in the molecule of the compound M1, and at least one of carbon atoms in X₁ to X₈ is bonded to an element forming Ar_{162;}
in the formula (AC24), X₁ to X₈ are each independently a nitrogen atom, CR₁₆₃, or a carbon atom bonded to an element forming Ar_{162;}
when a plurality of R₁₆₃ are present in the formulae (AC22) to (AC24), the plurality of R₁₆₃ are mutually the same or different, and at least one combination of adjacent two or more of R₁₆₃ are mutually bonded to form a substituted or unsubstituted monocyclic ring, mutually bonded to form a substituted or unsubstituted fused ring, or not mutually bonded;
R₁₆₃ forming neither the substituted or unsubstituted monocyclic ring nor the substituted or unsubstituted fused ring each independently represent the same as R₁₂ in the formula (102); and
in the formulae (AC1) to (AC3), (AC19) to (AC21), and (AC24), each represents a site bonded to an element forming Ar₁₆₂.

In the exemplary embodiment, the compound M1 is also preferably a compound represented by a formula (13) below.

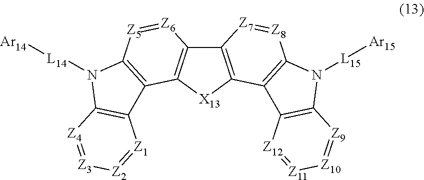

In the formula (13):
X₁₃ is an oxygen atom, a sulfur atom, or a group represented by N—Rb;
Z₁ to Z₁₂ are each independently a nitrogen atom or a group represented by C-Rc;
Ar₁₄ and Ar₁₅ are each independently a substituted or unsubstituted aryl group having 6 to 50 ring carbon atoms, or a substituted or unsubstituted heterocyclic group having 5 to 50 ring atoms;
L₁₄ and L₁₅ are each independently a single bond, a substituted or unsubstituted arylene group having 6 to 50 ring carbon atoms, or a substituted or unsubstituted divalent heterocyclic group having 5 to 50 ring atoms; and
Rb and Rc are each independently a hydrogen atom, a substituted or unsubstituted alkyl group having 1 to 50 carbon atoms, a substituted or unsubstituted haloalkyl group having 1 to 50 carbon atoms, a substituted or unsubstituted cycloalkyl group having 3 to 50 ring carbon atoms, a substituted or unsubstituted aralkyl group having 7 to 50 carbon atoms, a group represented by —Si(R₉₀₁)(R₉₀₂)(R₉₀₃), a group represented by —C(═O)R₉₀₈, a group represented by —COOR₉₀₆, a group represented by —P(═O)(R₉₁₀)(R₉₁₁), a group represented by —Ge(R₉₁₂)(R₉₁₃)(R₉₁₄), a cyano group, a nitro group, a substituted or unsubstituted aryl group having 6 to 50 ring carbon atoms, or a substituted or unsubstituted heterocyclic group having 5 to 50 ring atoms; and
when a plurality of Rc are present, the plurality of Rc are mutually the same or different.

When a group represented by —L₁₄-Ar₁₄ and a group represented by —L₁₅-Ar₁₅ are the same substituent in the compound represented by the formula (13), also preferably, Z₁ and Z₁₂ are not the same group, Z₂ and Z₁₁ are not the same group, Z₃ and Z₁₀ are not the same group, Z₄ and Z₉ are not the same group, Z₅ and Z₈ are not the same group, and Z₆ and Z₇ are not the same group. In this case, in the formula (13), a structure fused to the right side of a five-membered ring containing X₁₃ is different from a structure fused to the left side of the five-membered ring containing X₁₃, and the compound represented by the formula (13) is a compound having an asymmetric structure.

In the compound represented by the formula (13), preferably, a group represented by —L₁₄-Ar₁₄ and a group represented by —L₁₅-Ar₁₅ are mutually different groups. In this case too, the compound represented by the formula (13) is a compound having an asymmetric structure similarly above.

In the exemplary embodiment, the compound M1 is also preferably a compound represented by a formula (12) below.

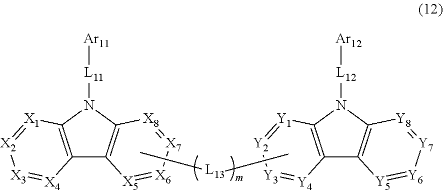

In the formula (12):
Ar₁₁ and Ar₁₂ are each independently a substituted or unsubstituted aryl group having 6 to 50 ring carbon atoms, or a substituted or unsubstituted heterocyclic group having 5 to 50 ring atoms;
L₁₁ and L₁₂ are each independently a single bond, a substituted or unsubstituted arylene group having 6 to 50 ring carbon atoms, or a substituted or unsubstituted divalent heterocyclic group having 5 to 50 ring atoms;
L₁₃ is a substituted or unsubstituted monocyclic hydrocarbon group having 6 or less ring carbon atoms, or a substituted or unsubstituted monocyclic heterocyclic group having 6 or less ring atoms;
m is 0, 1, 2, or 3, and a plurality of L₁₃ are mutually the same or different;
X₁ to X₈ and Y₁ to Y₈ are each independently N or CRa;
one of X₅ to X₈ and one of Y₁ to Y₄ are carbon atoms mutually bonded via L₁₃ or carbon atoms directly bonded;
each Ra is independently a hydrogen atom, a substituted or unsubstituted alkyl group having 1 to 50 carbon atoms, a group represented by —Si(R₉₀₁)(R₉₀₂)(R₉₀₃), a halogen atom, a substituted or unsubstituted aryl group having 6 to 50 ring carbon atoms, or a substituted or unsubstituted heterocyclic group having 5 to 50 ring atoms; and
when a plurality of Ra are present, the plurality of Ra are mutually the same or different, and
the compound represented by the formula (12) satisfies one of both of (i) and (ii) below:
(i) at least one of Ar₁₁ or Ar₁₂ is an aryl group having 6 to 50 ring carbon atoms substituted by a cyano group or a heterocyclic group having 5 to 50 ring atoms substituted by a cyano group; and
(ii) at least one of X₁ to X₄ or Y₅ to Y₈ is CRa, and at least one of Ra in X₁ to X₄ and Y₅ to Y₈ is an aryl group having 6 to 50 ring carbon atoms substituted by a cyano group or a heterocyclic group having 5 to 50 ring atoms substituted by a cyano group.

In the compound represented by the formula (12), an aromatic hydrocarbon ring group having 6 to 50 ring carbon atoms substituted by a cyano group or a heterocyclic group having 5 to 50 ring atoms substituted by a cyano group may further contain any other substituent than the cyano group.

In the compound represented by the formula (12), m is preferably 0, 1, or 2, more preferably 0 or 1. In the compound represented by the formula (12), when m is 0, one of X₅ to X₈ is directly bonded to one of Y₁ to Y₄ via a single bond.

In the compound represented by the formula (12), a combination selected from the group consisting of a combination of X₆ and Y₃, a combination of X₆ and Y₂, and a combination of X₇ and Y₃ are preferably carbon atoms mutually bonded via L₁₃ or carbon atoms directly bonded.

When a combination of X₆ and Y₃ are carbon atoms mutually bonded via L₁₃ or carbon atoms directly bonded, the compound represented by the formula (12) is represented by a formula (121) below.

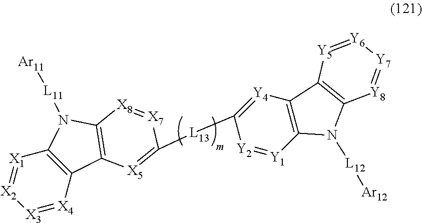

In the formula (121), Ar₁₁, Ar₁₂, L₁₁, L₁₂, L₁₃, m, X₁ to X₅, X₇ to X₈, Y₁ to Y₂, and Y₄ to Y₈ respectively represent the same as Ar₁₁, Ar₁₂, L₁₁, L₁₂, L₁₃, m, X₁ to X₅, X₇ to X₈, Y₁ to Y₂, and Y₄ to Y₈ in the formula (12), and the compound represented by the formula (121) satisfies at least one condition of (i) or (ii) above.

In the compound represented by the formula (12), preferably, a group represented by —Ar₁₁—L₁₁ and a group represented by —Ar₁₂—L₁₂ are mutually different.

A monocyclic hydrocarbon group having 6 or less ring carbon atoms for L₁₃ is preferably, for instance, at least one group selected from the group consisting of a phenylene group, cyclopentenylene group, cyclopentadienylene group, and cyclopentylene group, more preferably a phenylene group.

A monocyclic hydrocarbon group having 6 or less ring atoms for L₁₃ is preferably, for instance, at least one group selected from the group consisting of a pyrrolylene group, pyrazinylene group, pyridinylene group, furylene group, and thiophenylene group.

In an exemplary embodiment, the emitting layer may contain two or more types of the compound M1 mutually different in molecular structures. Mixing compounds different in charge-transporting properties improves a charge balance in the emitting layer, which may lead to the improvement in luminous efficiency. Further, the excitation energy is reduced by an exciplex formed by two or more types of the compound M1 (second host material), which enables the organic EL device to be driven at a lower voltage than a case where the emitting layer contains a single type of the second host material.

### Method for Producing Compound M1

The compound M1 can be produced by a known method. Further, the compound M1 can be produced based on a known method through a known alternative reaction using a known material(s) tailored for the target compound.

### Specific Examples of Compound M1

Specific examples of the compound M1 of the exemplary embodiment include compounds below. It should however be noted that the invention is not limited to these specific examples of the compound M1.

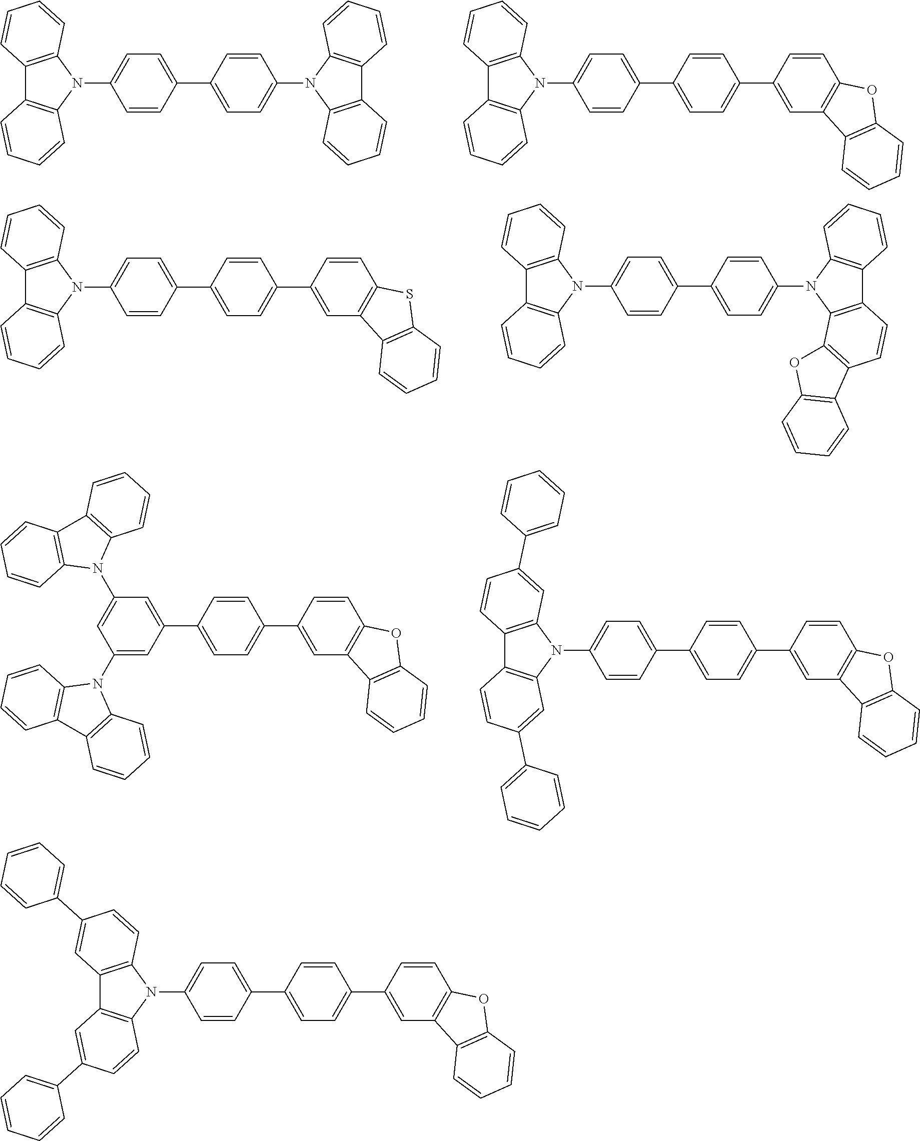

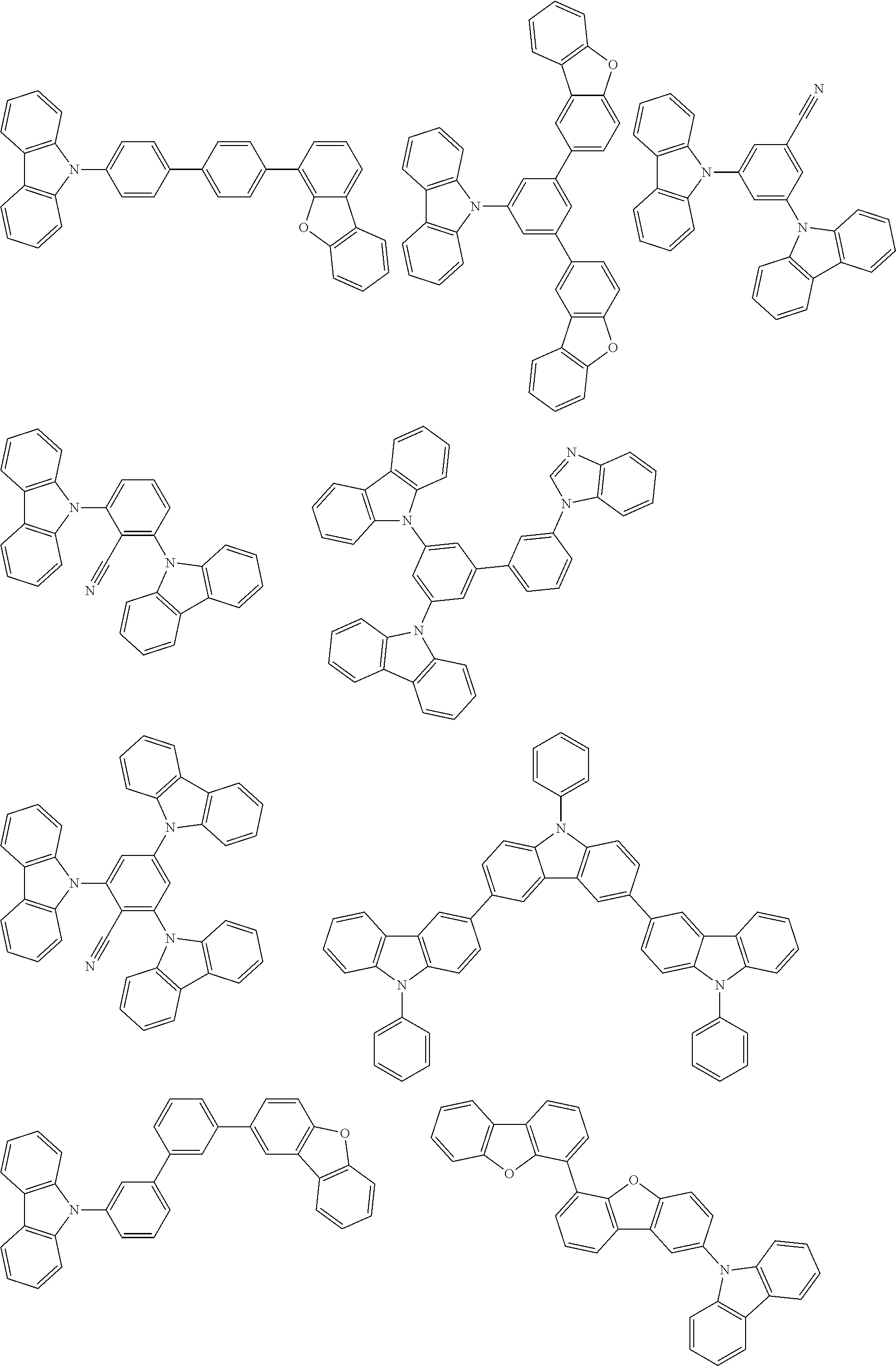

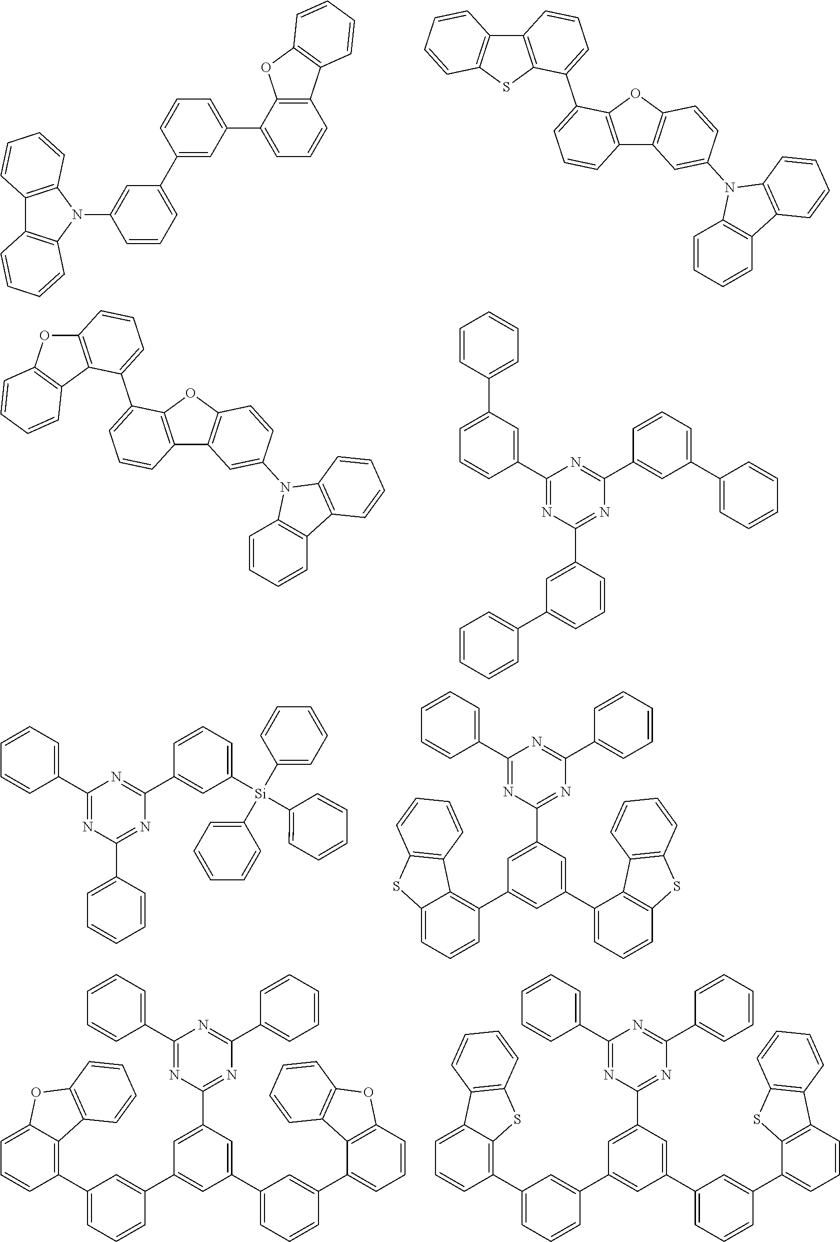

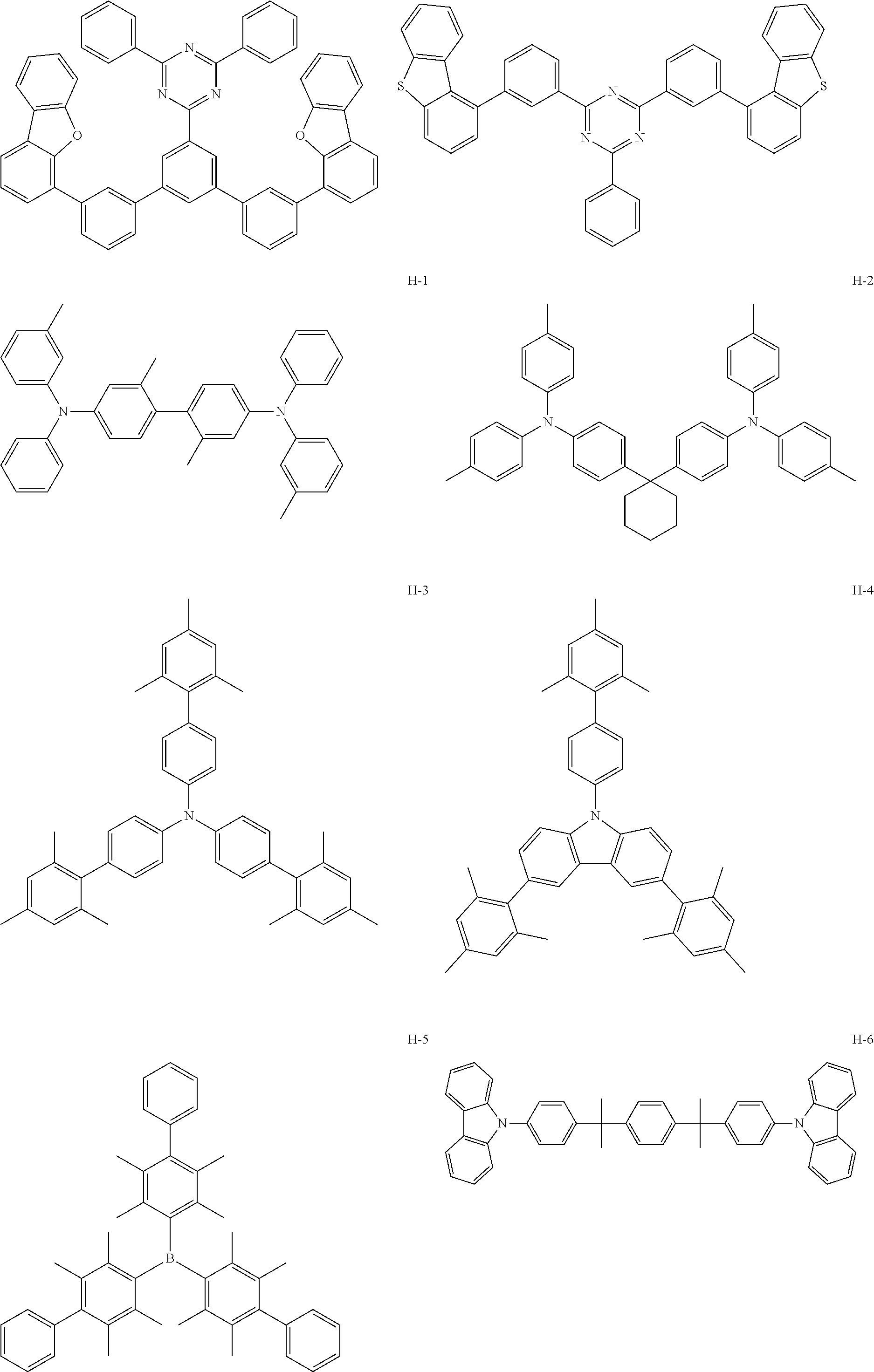

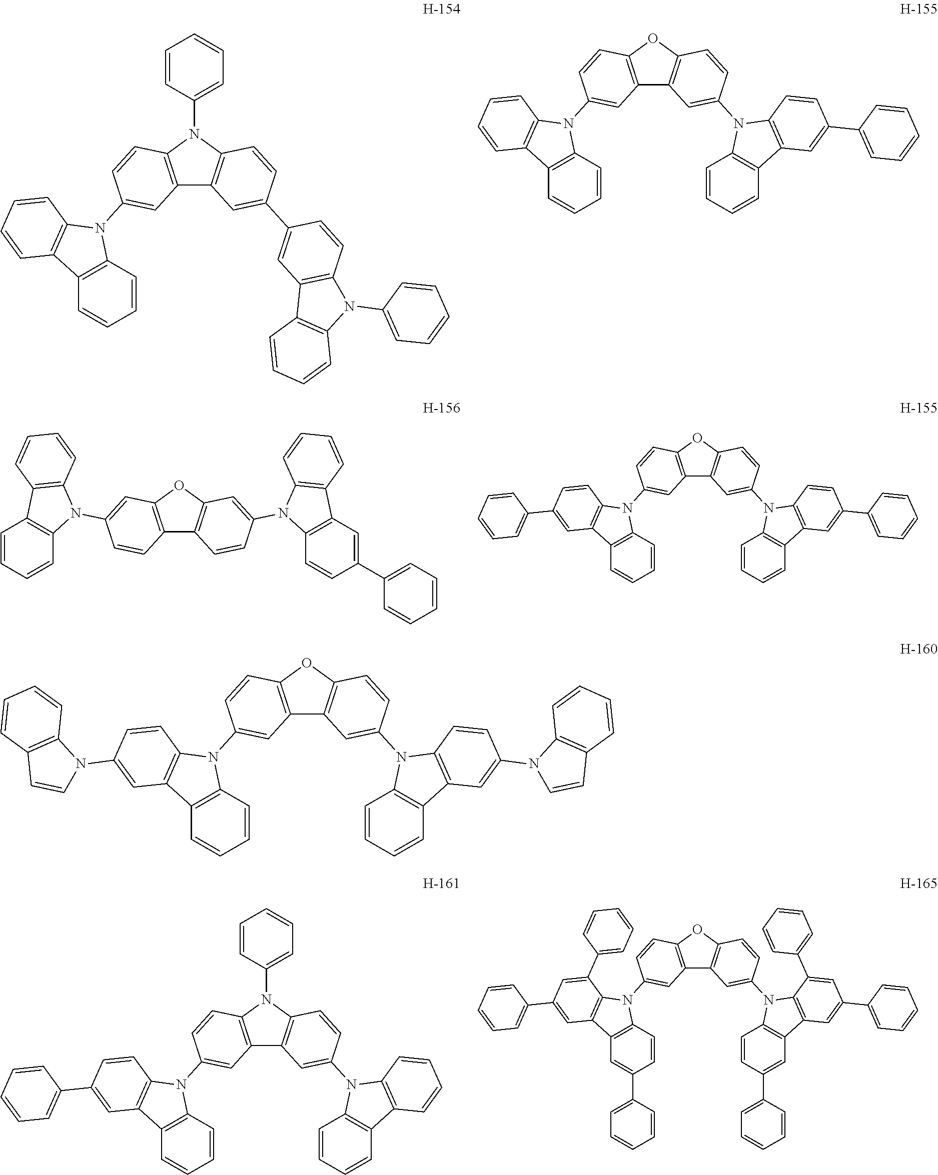

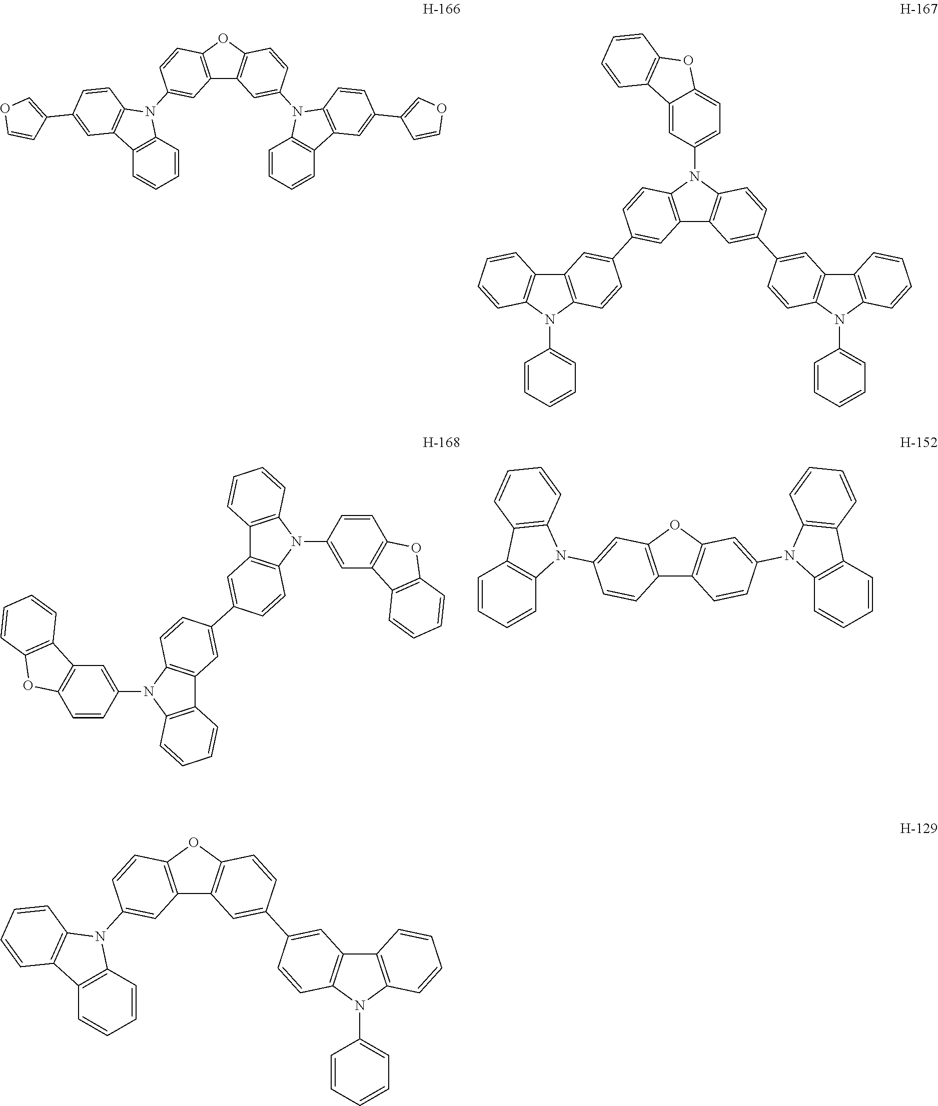

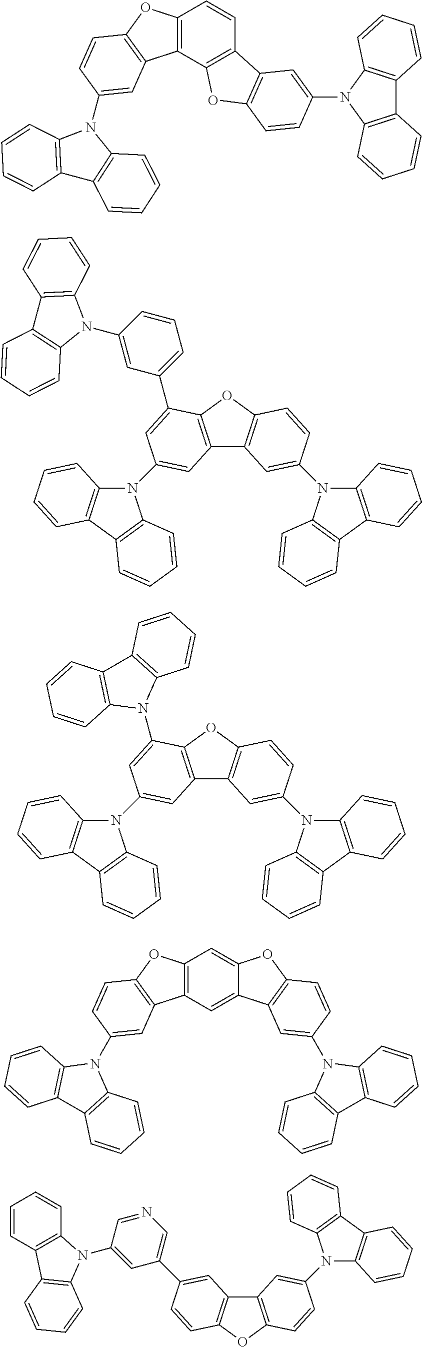

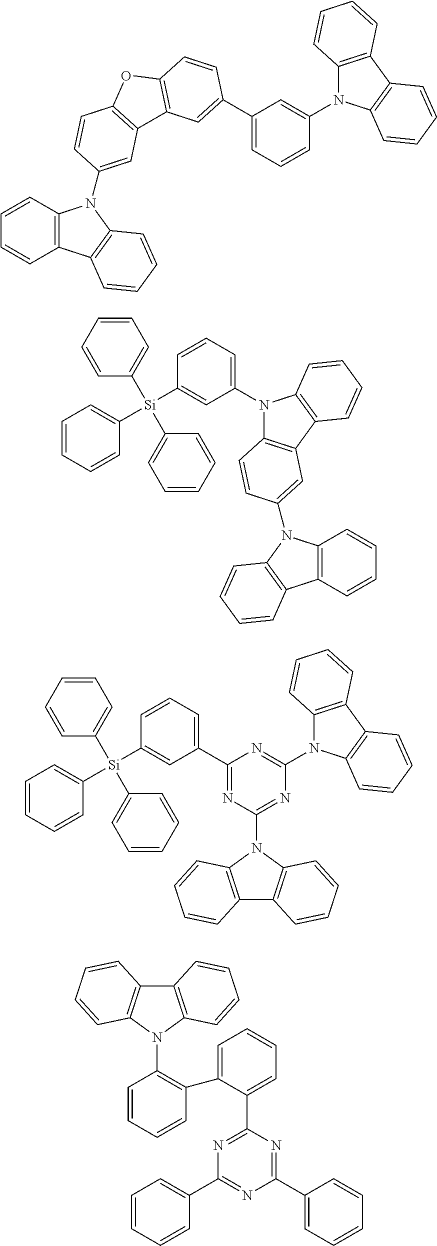

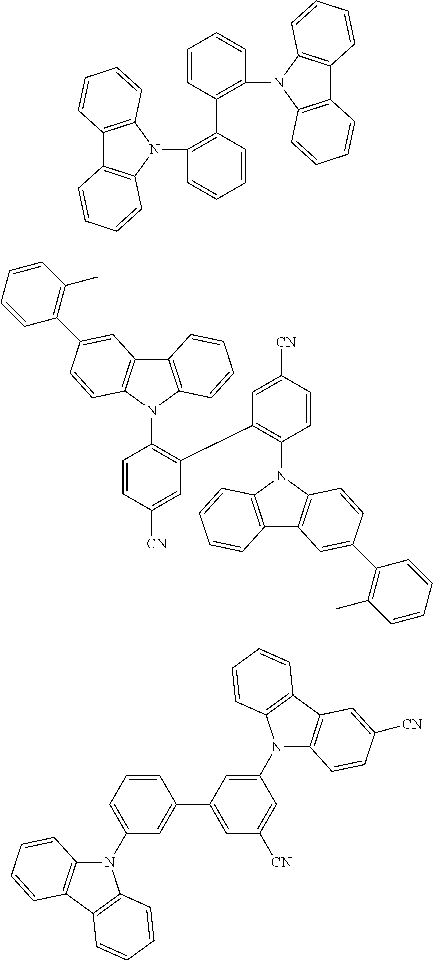 [SMILES: c1ccc(-n2c3ccccc3c3ccccc32)c(-c2ccccc2-n2c3ccccc3c3ccccc32)c1 ; Cc1ccccc1-c1ccc2c(c1)c1ccccc1n2-c1ccc(C#N)cc1-c1cc(C#N)ccc1-n1c2ccccc2c2cc(-c3ccccc3C)ccc21 ; N#Cc1cc(-c2cccc(-n3c4ccccc4c4ccccc43)c2)cc(-n2c3ccccc3c3cc(C#N)ccc32)c1]

Relationship between Second Host Material, Sensitizing Material, and Fluorescent Material in Emitting Layer

In an exemplary embodiment, the sensitizing material as the first host material is the above-described delayed fluorescent compound.

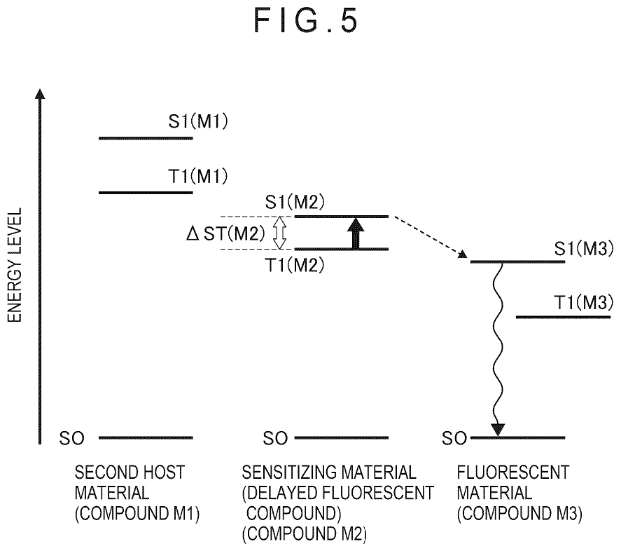

FIG. 5 illustrates an exemplary relationship in energy level between the second host material, the sensitizing material, and the fluorescent material, when the emitting layer contains the second host material (compound M1), the delayed fluorescent compound (compound M2) as the sensitizing material, and the fluorescent material (compound M3). In FIG. 5, S0 represents a ground state. S1(M1) represents a lowest singlet state of the second host material, and T1(M1) represents a lowest triplet state of the second host material. S1(M2) represents a lowest singlet state of the delayed fluorescent compound, and T1(M2) represents a lowest triplet state of the delayed fluorescent compound. S1(M3) represents a lowest singlet state of the fluorescent material, and T1(M3) represents a lowest triplet state of the fluorescent material. A dashed arrow directed from S1(M2) to S1(M3) in FIG. 5 represents Forster energy transfer from the lowest singlet state of the delayed fluorescent compound to the lowest singlet state of the fluorescent material.

As illustrated in FIG. 5, when a compound having a small ΔST(M2) is used as the delayed fluorescent compound, inverse intersystem crossing from the lowest triplet state T1(M2) to the lowest singlet state S1(M2) can be caused by heat energy. Subsequently, Forster energy transfer from the lowest singlet state S1(M2) of the delayed fluorescent compound to the fluorescent material occurs to generate a lowest singlet state S1(M3). Consequently, fluorescence from the lowest singlet state S1(M3) of the fluorescent material can be observed. It is inferred that the internal quantum efficiency can be theoretically raised up to 100% also by using delayed fluorescence by the TADF mechanism.

In an exemplary embodiment, an energy gap T_{77K}(H2) at 77 K of the second host material and an energy gap T_{77K}(G2) at 77 K of the sensitizing material preferably satisfy a relationship of a numerical formula (Numerical Formula 1) below.

T_{77K}(H2)>T_{77K}(G2) (Numerical Formula 1)

In an exemplary embodiment, a lowest singlet energy S₁(H2) of the second host material and the lowest singlet energy S₁(GT2) of the sensitizing material also preferably satisfy a relationship of a numerical formula (Numerical Formula 4A) below.

S₁(H2)>S₁(GT2) (Numerical Formula 4A)

In an exemplary embodiment, the lowest singlet energy S₁ of each of the second host material, the sensitizing material, and the fluorescent material also preferably satisfies a relationship of a numerical formula (Numerical Formula 4B) below.

S₁(H2)>S₁(GT2)>S₁(D) (Numerical Formula 4B)

In an exemplary embodiment, the energy gap T_{77K} at 77 K of each of the second host material, the sensitizing material, and the fluorescent material also preferably satisfies a relationship of a numerical formula (Numerical Formula 6B) below.

T_{77K}(H2)>T_{77K}(GT2)>T_{77K}(D) (Numerical Formula 6B)

### Content Ratios of Compounds in Emitting Layer

For instance, content ratios of the second host material (compound M1), the first host material (compound M2), and the fluorescent material (compound M3) in the emitting layer preferably fall within ranges below.

The content ratio of the compound M1 in the emitting layer is preferably 50 mass % or more, more preferably 70 mass % or more.

The content ratio of the compound M1 in the emitting layer is preferably 95 mass % or less, more preferably 90 mass % or less.

The content ratio of the compound M2 is preferably 5 mass % or more, more preferably 10 mass % or more.

The content ratio of the compound M2 is preferably 50 mass % or less, more preferably 30 mass % or less.

The content ratio of the compound M3 in the emitting layer is preferably 0.5 mass % or more, more preferably 1 mass % or more.

The content ratio of the compound M3 in the emitting layer is preferably 10 mass % or less, more preferably 5 mass % or less.

The upper limit of the total of the content ratios of the compounds M1, M2, and M3 in the emitting layer is 100 mass %. It should be noted that the emitting layer in the exemplary embodiment may contain any other material than the compounds M1, M2, and M3. In the exemplary embodiment, the emitting layer may contain a single type of the compound M1 or may contain two or more types of the compound M1. In the exemplary embodiment, the emitting layer may contain a single type of the compound M2 or may contain two or more types of the compound M2. In the exemplary embodiment, the emitting layer may contain a single type of the compound M3 or may contain two or more types of the compound M3.

According to the fourth exemplary embodiment, an organic EL device that emits light with high efficiency is achievable.

The organic EL device according to the fourth exemplary embodiment is applicable to an electronic device such as a display device and a light-emitting unit.

### Film Thickness of Emitting Layer

The film thickness of the emitting layer of the organic EL device of the exemplary embodiment is preferably in a range from 5 nm to 50 nm, more preferably in a range from 7 nm to 50 nm, and still more preferably in a range from 10 nm to 50 nm. When the film thickness of the emitting layer is 5 nm or more, the formation of the emitting layer and the adjustment of the chromaticity are likely to be easy. When the film thickness of the emitting layer is 50 nm or less, the increase in drive voltage is likely to be inhibited.

The organic EL device according to the exemplary embodiment is applicable to an electronic device such as a display device and a light-emitting unit.

### Fifth Exemplary Embodiment

### Electronic Device

An electronic device according to a fifth exemplary embodiment is installed with the organic EL device according to any of the above exemplary embodiments. The electronic device according to the fifth exemplary embodiment may be installed with any of organic EL devices according to a later-described sixth exemplary embodiment. Examples of the electronic device include a display device and a light-emitting unit. Examples of the display device include a display component (e.g., an organic EL panel module), TV, mobile phone, tablet and personal computer. Examples of the light-emitting unit include an illuminator and a vehicle light. The light-emitting unit can be also used for the display device, for instance, as a backlight of the display device.

The display device as the electronic device according to the fifth exemplary embodiment is preferably an organic EL display device installed with organic EL devices as a red pixel, a green pixel, and a blue pixel. In the organic EL display device, the blue pixel is preferably an organic EL device according to the third exemplary embodiment, the fourth exemplary embodiment, or the sixth exemplary embodiment. In the organic EL display device, the red pixel and the green pixel may be each independently an organic EL device according to the third exemplary embodiment, the fourth exemplary embodiment, or the sixth exemplary embodiment.

### Sixth Exemplary Embodiment

The organic EL device according to the sixth exemplary embodiment may include an anode, a cathode, and an emitting layer disposed between the anode and the cathode, in which the emitting layer may contain the compound M2 (a compound represented by the formula (1)) as the first host material and a phosphorescent metal complex as the dopant material.

The organic EL device according to the sixth exemplary embodiment may include an anode, a cathode, and an emitting layer disposed between the anode and the cathode, in which the emitting layer may contain the compound M2 (a compound represented by the formula (1)) as the first host material, a phosphorescent metal complex as the sensitizing material, and a fluorescent material (compound M3) as the dopant material.

### Phosphorescent Metal Complex

In an exemplary embodiment, the phosphorescent metal complex preferably contains a heavy metal atom.

In an exemplary embodiment, the phosphorescent metal complex preferably contains at least one metal atom selected from the group consisting of platinum (Pt), iridium (Ir), osmium (Os), ruthenium (Ru), rhodium (Rh), palladium (Pd), copper (Cu), silver (Ag), gold (Au), titanium (Ti), zirconium (Zr), hafnium (Hf), europium (Eu), terbium (Tb), and thulium (Tm).

In an exemplary embodiment, the phosphorescent metal complex is preferably a compound represented by a formula (21) below.

M(L₁)ₙ₁(L₂)ₙ₂ (21)

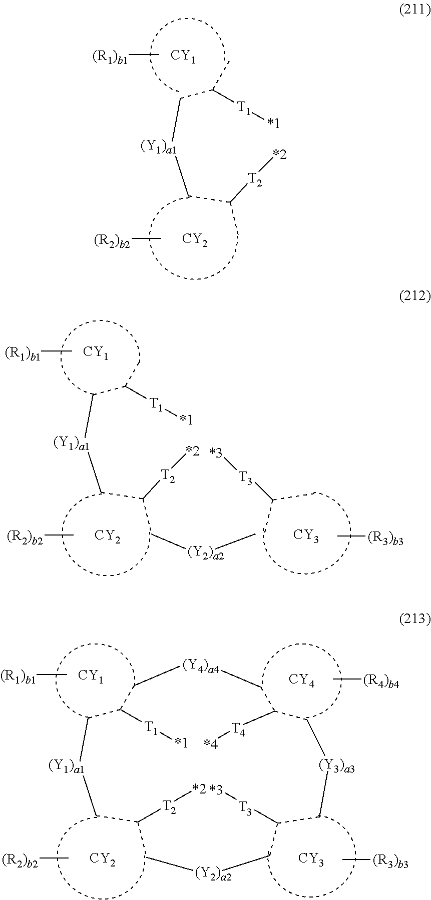

In the formulae (21), (211), (212), and (213):
M is a transition metal selected from the group consisting of a first transition metal, a second transition metal, and a third transition metal;
L₁ is at least one ligand selected from the group consisting of a ligand represented by the formula (211), a ligand represented by the formula (212), and a ligand represented by the formula (213);
n1 is 1, 2, or 3;
L₂ is at least one ligand selected from the group consisting of a monodentate ligand, a bidentate ligand, and a tridentate ligand;
n2 is 0, 1, 2, 3, or 4;
a ring CY₁, a ring CY₂, a ring CY₃, and a ring CY₄ are each independently selected from the group consisting of a carbocyclic group having 5 to 30 ring carbon atoms and a heterocyclic group having 1 to 30 ring carbon atoms;
Y₁ to Y₄ are each independently selected from the group consisting of a single bond, a double bond, a substituted or unsubstituted arylene group having 6 to 50 ring carbon atoms, a substituted or unsubstituted divalent heterocyclic group having 5 to 50 ring atoms, *a-O-*b, *a-S-*b, *a-C(═O)-*b, *a-S(═O)-*b, *a-C(R₅)(R₆)-*b, *a-C(R₅)═C(R₆)-*b, *a-C(R₅)═*b, *a-Si(R₅)(R₆)-*b, *a-B(R₅)-*b, *a-N(R₅)-*b, and *a-P(R₅)-*b;
a1, a2, and a3 are each independently 1, 2, or 3;
a4 is 0, 1, 2, or 3, and when a4 is 0, the ring CY₁ is not linked to the ring CY₄;
T1, T2, T3, and T4 are each independently selected from the group consisting of a chemical bond, *a-O-*b, *a-S-*b, *a-B(R₇)-*b, *a-N(R₇)-*b, *a-P(R₇)-*b, *a-C(R₇)(R₈)-*b, *a-Si(R₇)(R₈)-*b, *a-Ge(R₇)(R₈)-*b, *a-C(═O)-*b, and *a-C(═S)-*b
*a and *b are each independently a bonding position to an adjacent atom;
1, *2, *3, and *4 are each a bonding position to M;
R₁ to R₈ are each independently a hydrogen atom, a halogen atom, a cyano group, a nitro group, an amidino group, a hydrazino group, a hydrazone group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted heterocycloalkyl group having 3 to 50 ring atoms, a substituted or unsubstituted cycloalkenyl group having 3 to 50 ring carbon atoms, a substituted or unsubstituted heterocycloalkenyl group having 3 to 50 ring atoms, a substituted or unsubstituted aryl group having 6 to 50 ring carbon atoms, a substituted or unsubstituted heterocyclic group having 5 to 50 ring atoms, a substituted or unsubstituted monovalent non-aromatic fused polycyclic group, a substituted or unsubstituted monovalent non-aromatic hetero-fused polycyclic group, a group represented by —Si(R₂₅₁)(R₂₅₂)(R₂₅₃), a group represented by —O—(R₂₅₄), a group represented by —S—(R₂₅₅), a group represented by —N(R₂₅₆)(R₂₅₇), a group represented by —C(═O)R₂₅₈, a group represented by —C(═O)(OR₂₅₉), a group represented by —S(═O)₂(OR₂₆₀), a group represented by —O—P(═O)(OR₂₆₁)(OR₂₆₂), a group represented by —C(R₂₆₃)(R₂₆₄)(R₂₆₅), a group represented by —B(R₂₆₆)(R₂₅₇), a group represented by —P(═O)(R₂₆₈)(R₂₅₉), a group represented by —S(═O)(R₂₇₀), a group represented by —S(═O)₂(R₂₇₁), a group represented by —P(═O)(R₂₇₂)(R₂₇₃), and a group represented by —P(═S)(R₂₇₄)(R₂₇₅);
at least one combination of adjacent two or more of R₁ to R₈ are mutually bonded to form a substituted or unsubstituted monocyclic ring, mutually bonded to form a substituted or unsubstituted fused ring, or not mutually bonded;
at least one combination of adjacent two or more of R₁ to R₈ and Y₁ to Y₄ are mutually bonded to form a substituted or unsubstituted monocyclic ring, mutually bonded to form a substituted or unsubstituted fused ring, or not mutually bonded;
b1, b2, b3, and b4 are each independently 0, 1, 2, 3, 4, 5, 6, 7, 8, 9, or 10;
R₂₅₁ to R₂₇₅ are each independently selected from the group consisting of a hydrogen atom, a halogen atom, a group represented by —O—(R₂₇₆), a group represented by —N(R₂₇₇)(R₂₇₈), a cyano group, a nitro group, an amidino group, a hydrazino group, a hydrazone group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted heterocycloalkyl group having 3 to 50 ring atoms, a substituted or unsubstituted cycloalkenyl group having 3 to 50 ring carbon atoms, a substituted or unsubstituted heterocycloalkenyl group having 3 to 50 ring atoms, a substituted or unsubstituted aryl group having 6 to 50 ring carbon atoms, an aryl group having 6 to 50 ring carbon atoms substituted by a substituted or unsubstituted alkyl group having 1 to 50 carbon atoms, an aryl group having 6 to 50 ring carbon atoms substituted by a substituted or unsubstituted aryl group having 6 to 50 ring carbon atoms, a substituted or unsubstituted heterocyclic group having 5 to 50 ring atoms, a substituted or unsubstituted monovalent non-aromatic fused polycyclic group, a substituted or unsubstituted monovalent non-aromatic hetero-fused polycyclic group, a biphenylyl group, and a terphenylyl group; and
R₂₇₆ to R₂₇₈ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

The term “carbocyclic group having 5 to 30 ring carbon atoms” herein refers to a monocyclic group or a polycyclic group having 5 to 30 carbon atoms only containing carbon as the ring atoms. The carbocyclic group having 5 to 30 ring carbon atoms may be an aromatic carbocyclic group or a non-aromatic carbocyclic group. The carbocyclic group having 5 to 30 ring carbon atoms may be a ring such as benzene, a monovalent group such as a phenyl group, or a divalent group such as a phenylene group. In addition to the above, the carbocyclic group having 5 to 30 ring carbon atoms may be variedly modified, such as a trivalent group or a tetravalent group, depending on the number of substituents linked thereto.

Herein, a heterocyclic group having 1 to 30 ring carbon atoms, which has the same structure as a carbocyclic group having 5 to 30 ring carbon atoms, refers to a group having, in addition to carbon (the number of carbons may be 1 to 30), at least one heteroatom selected from N (nitrogen atom), 0 (oxygen atom), S₁(silicon atom), P (phosphorus atom), and S (sulfur atom) as a ring atom.

The term “heterocycloalkyl group having 3 to 50 ring atoms” herein refers to a monovalent monocyclic group having 3 to 50 ring atoms that contains, as a ring atom, at least one heteroatom selected from N, O, Si, P, and S, and specific examples thereof include a 1,2,3,4-oxatriazolidinyl group, a tetrahydrofuranyl group, and a tetrahydrothiophenyl group. A term “heterocycloalkylene group having 3 to 50 ring atoms” herein refers to a divalent group having the same structure as the heterocycloalkyl group having 3 to 50 ring atoms.

The term “cycloalkenyl group having 3 to 50 ring carbon atoms” herein refers to a monovalent monocyclic group having 3 to 50 ring carbon atoms that has, in the ring, at least one double bond but has no aromaticity, and specific examples thereof include a cyclopentenyl group, a cyclohexenyl group, and a cycloheptenyl group. A term “cycloalkenylene group having 3 to 50 ring carbon atoms” herein refers to a divalent group having the same structure as the cycloalkenyl group having 3 to 50 ring carbon atoms.

The term “heterocycloalkenyl group having 3 to 50 ring atoms” herein refers to a monovalent monocyclic group having 3 to 50 ring atoms that contains, as a ring atom, at least one heteroatom selected from N, O, Si, P, and S and that has, in the ring, at least one double bond. Specific examples of the heterocycloalkenyl group having 3 to 50 ring atoms include a 4,5-dihydro-1,2,3,4-oxatriazolyl group, a 2,3-dihydrofuranyl group and a 2,3-dihydrothiophenyl group. A term “heterocycloalkenylene group having 3 to 50 ring atoms” herein refers to a divalent group having the same structure as the heterocycloalkenyl group having 3 to 50 ring atoms.

In the compound represented by the formula (21) of an exemplary embodiment, a substituted or unsubstituted cycloalkyl group having 3 to 50 ring carbon atoms preferably has 3 to 10 ring carbon atoms; a substituted or unsubstituted heterocycloalkyl group having 3 to 50 ring atoms preferably has 3 to 10 ring atoms; a substituted or unsubstituted cycloalkenyl group having 3 to 50 ring carbon atoms preferably has 3 to 10 ring carbon atoms; and a substituted or unsubstituted heterocycloalkenyl group having 3 to 50 ring atoms preferably has 3 to 10 ring atoms.

The term “monovalent non-aromatic fused polycyclic group” herein refers to a monovalent group (e.g., having 8 to 60 carbon atoms) that has two or more rings fused to each other, has only carbon as ring atoms, and has non-aromaticity in the entire molecular structure. A term “divalent non-aromatic fused polycyclic group” herein refers to a divalent group having the same structure as the monovalent non-aromatic fused polycyclic group.

The term “monovalent non-aromatic hetero-fused polycyclic group” herein refers to a monovalent group (e.g., having 1 to 60 carbon atoms) that has two or more rings fused to each other, has, in addition to carbon, at least one heteroatom selected from N, O, Si, P, and S as a ring atom, and has non-aromaticity in the entire molecular structure. A term “divalent non-aromatic hetero-fused polycyclic group” herein refers to a divalent group having the same structure as the monovalent non-aromatic hetero-fused polycyclic group.

The term “biphenylyl group” herein refers to “a phenyl group substituted with a phenyl group”. The “biphenylyl group” belongs to “a substituted phenyl group” having, as a substituent, “an aryl group having 6 to 50 ring carbon atoms”.

The term “terphenylyl group” herein refers to “a phenyl group substituted with a biphenylyl group.” The “terphenylyl group” belongs to “a substituted phenyl group” having, as a substituent, “an aryl group having 6 to 50 ring carbon atoms substituted with an aryl group having 6 to 50 ring carbon atoms”.

In the compound represented by the formula (21), the chemical bond for each of T₁, T₂, T₃, and T₄ is preferably a single bond.

In the compound represented by the formula (21), M is preferably at least one metal atom selected from the group consisting of platinum (Pt), iridium (Ir), osmium (Os), ruthenium (Ru), rhodium (Rh), palladium (Pd), copper (Cu), silver (Ag), gold (Au), titanium (Ti), zirconium (Zr), hafnium (Hf), europium (Eu), terbium (Tb), and thulium (Tm), more preferably platinum (Pt) or iridium (Ir).

According to an exemplary embodiment, the ring CY₁ to the ring CY₄ in the compound represented by the formula (21) may be each independently selected from the group consisting of benzene, naphthalene, anthracene, phenanthrene, triphenylene, pyrene, chrysene, cyclopentadiene, 1,2,3,4-tetrahydronaphthalene, carbene, thiophene, furan, selenophene, indole, benzoborole, benzophosphole, indene, benzosilole, benzogermole, benzothiophene, benzoselenophene, benzofuran, carbazole, dibenzoborole, dibenzophosphole, fluorene, dibenzosilole, dibenzogermole, dibenzothiophene, dibenzoselenophene, dibenzofuran, dibenzothiophene-5-oxide, 9H-fluoren-9-one, dibenzothiophene 5,5-dioxide, azaindole, azabenzoborole, azabenzophosphole, azaindene, azabenzosilole, azabenzogermole, azabenzothiophene, azabenzoselenophene, azabenzofuran, azacarbazole, azadibenzoborole, azadibenzophosphole, azafluorene, azadibenzosilole, azadibenzogermole, azadibenzothiophene, azadibenzoselenophene, azadibenzofuran, azadibenzothiophene-5-oxide, aza-9H-fluoren-9-one, aza-dibenzothiophene 5,5-dioxide, pyridine, pyrimidine, pyrazine, pyridazine, triazine, quinoline, isoquinoline, quinoxaline, quinazoline, phenanthroline, pyrrole, pyrazole, imidazole, triazole, oxazole, isoxazole, thiazole, isothiazole, oxadiazole, thiadiazole, benzopyrazole, benzimidazole, benzoxazole, benzothiazole, benzoxadiazole, benzothiadiazole, 5,6,7,8-tetrahydroisoquinoline, and 5,6,7,8-tetrahydroquinoline.

According to an exemplary embodiment, at least one of the ring CY₁ or the ring CY₂ in the formula (211), at least one of the ring CY₁ to the ring CY₃ in the formula (212), and at least one of the ring CY₁ to the ring CY₄ in the formula (213) may each be carbene.

According to an exemplary embodiment, Y₁ to Y₄ in the formulae (211) to (213) may be each independently at least one selected from the group consisting of a single bond, a double bond, *a-O-*b, *a-S-*b, *a-C(R₅)(R₆)-*b, and *a-N(R₅)-*b.

According to an exemplary embodiment, at least one of R₁ or R₂ in the formula (211), at least one of R₁ to R₃ in the formula (212), and at least one of R₁ to R₄ in the formula (213) may each be an electron donating group.

For instance, the electron donating group may be a substituent selected from the group consisting of an isopropyl group, a tert-butyl group, and groups represented by formulae (10-1) to (10-61) below.

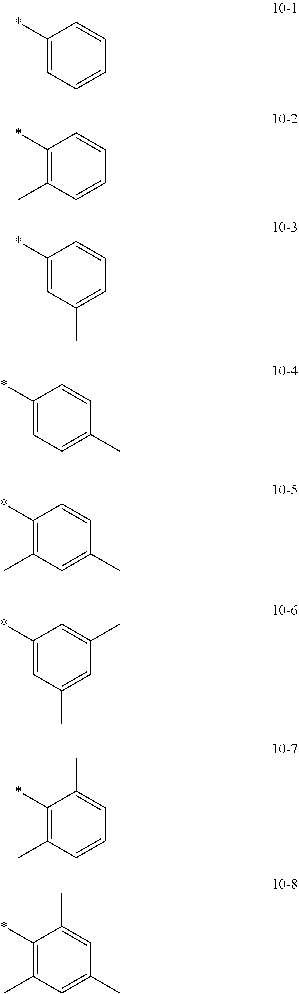

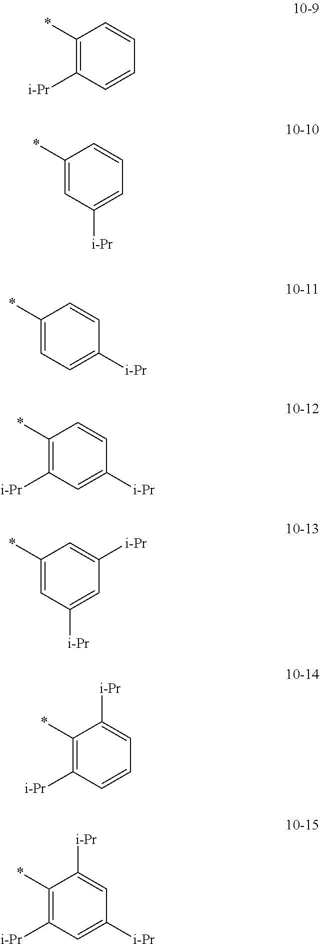

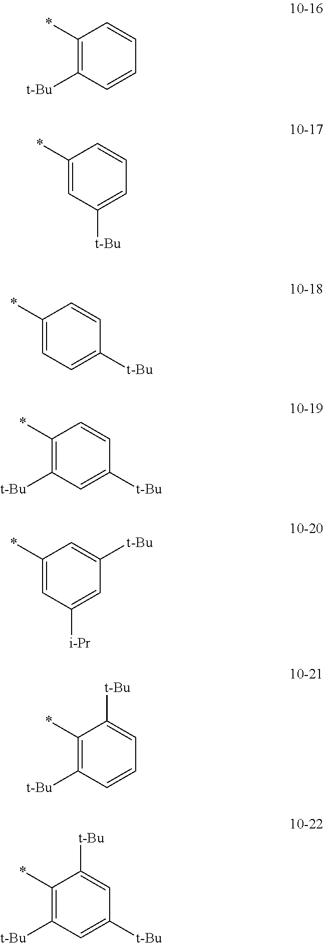

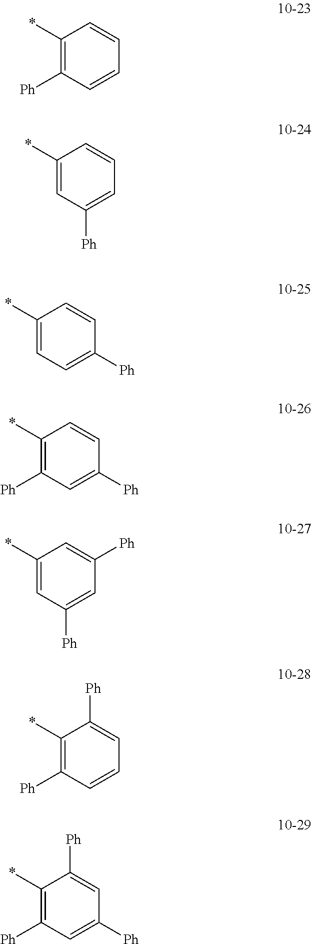

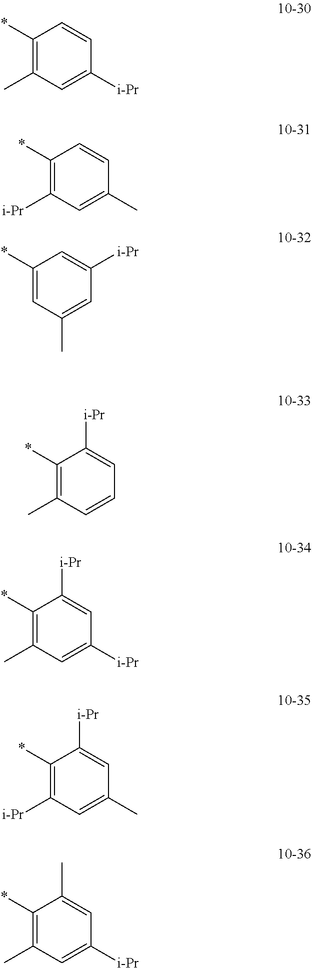

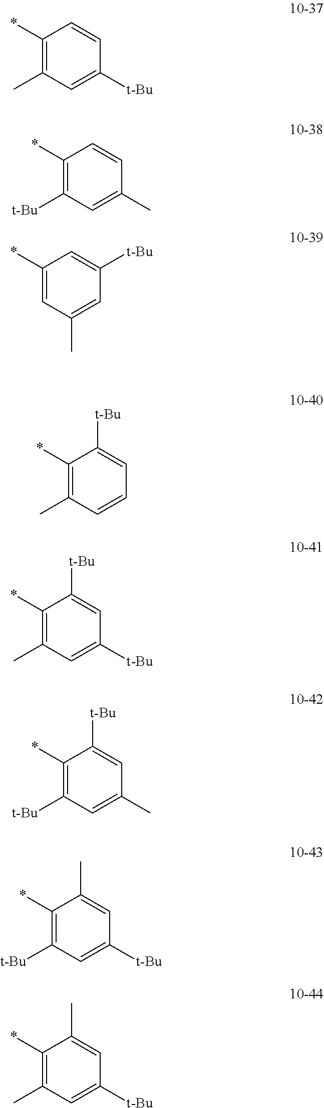

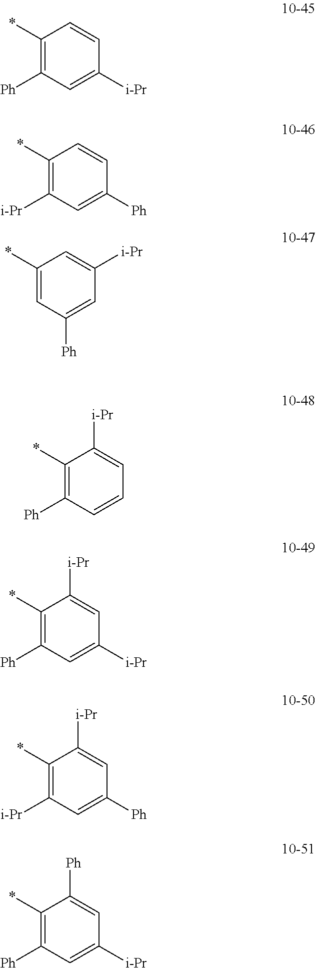

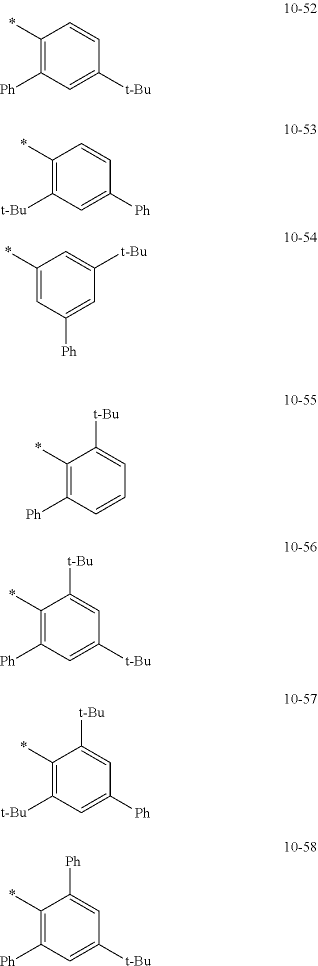

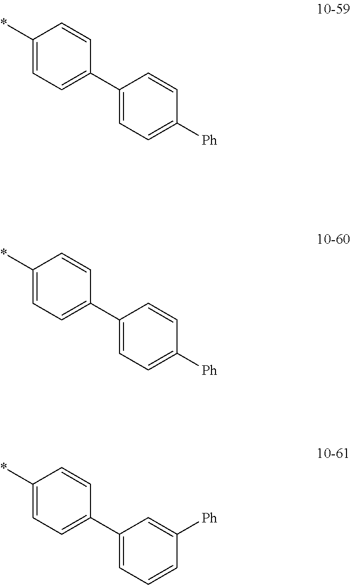 [SMILES: *c1ccc(-c2ccc(-c3ccccc3)cc2)cc1 ; *c1ccc(-c2ccc(-c3ccccc3)cc2)cc1 ; *c1ccc(-c2cccc(-c3ccccc3)c2)cc1]

In the formulae (10-1) to (10-61), * each represent a bonding position to an adjacent atom.

In the chemical formulae herein, a deuterium atom is denoted by D, and a protium atom is denoted by H or description thereof is omitted. In the chemical formulae herein, a methyl group may be denoted by Me, a phenyl group may be denoted by Ph, an isopropyl group may be denoted by i-Pr, and a tert-butyl group may be denoted by t-Bu.

In an exemplary embodiment, it is allowable that at least one of R₁ or R₂ in the formula (211) is a substituent that is not hydrogen, and/or Y₁ is *a-N(R₅)-*b, and R₅ is a substituted aryl group having 6 to 50 ring carbon atoms.

In an exemplary embodiment, it is allowable that at least one of R₁ to R₃ in the formula (212) is a substituent that is not hydrogen, and/or at least one of Y₁ or Y₂ is *a-N(R₅)-*b, and R₅ is a substituted aryl group having 6 to 50 ring carbon atoms.

In an exemplary embodiment, it is allowable that at least one of R₁ to R₄ in the formula (213) is a substituent that is not hydrogen, and/or at least one of Y₁ to Y₄ is *a-N(R₅)-*b, and R₅ is a substituted aryl group having 6 to 50 ring carbon atoms.

The organic EL device according to the sixth exemplary embodiment includes the emitting layer containing the compound M2 (a compound represented by the formula (1)), high efficiency is achievable in the organic EL device according to the sixth exemplary embodiment.

The organic EL device according to the sixth exemplary embodiment is applicable to an electronic device such as a display device and a light-emitting unit.

### Modifications of Exemplary Embodiments

The scope of the invention is not limited to the above-described exemplary embodiments but includes any modification and improvement as long as such modification and improvement are compatible with the invention.

For instance, the number of emitting layers is not limited to one, and a plurality of emitting layers may be layered. When the organic EL device includes a plurality of emitting layers, it is only necessary that at least one emitting layer should satisfy the requirements mentioned in the above exemplary embodiment(s). For instance, the rest of the emitting layers may be a fluorescent emitting layer or a phosphorescent emitting layer with the use of emission caused by electron transfer from the triplet excited state directly to the ground state.

When the organic EL device includes a plurality of emitting layers, these emitting layers may be mutually adjacently provided, or may form a so-called tandem organic EL device in which a plurality of emitting units are layered via an intermediate layer.

For instance, a blocking layer may be provided adjacent to at least one of a side of the emitting layer close to the anode or a side of the emitting layer close to the cathode. The blocking layer is preferably provided in contact with the emitting layer to block at least one of holes, electrons, or excitons.

For instance, when the blocking layer is provided in contact with the side of the emitting layer close to the cathode, the blocking layer permits transport of electrons, and blocks holes from reaching a layer provided closer to the cathode (e.g., the electron transporting layer) beyond the blocking layer. When the organic EL device includes the electron transporting layer, the blocking layer is preferably interposed between the emitting layer and the electron transporting layer.

When the blocking layer is provided in contact with the side of the emitting layer close to the anode, the blocking layer permits transport of holes and blocks electrons from reaching a layer provided closer to the anode (e.g., the hole transporting layer) beyond the blocking layer. When the organic EL device includes the hole transporting layer, the blocking layer is preferably interposed between the emitting layer and the hole transporting layer.

Alternatively, the blocking layer may be provided adjacent to the emitting layer so that the excitation energy does not leak out from the emitting layer toward neighboring layer(s). The blocking layer blocks excitons generated in the emitting layer from being transferred to a layer(s) (e.g., the electron transporting layer and the hole transporting layer) closer to the electrode(s) beyond the blocking layer.

The emitting layer is preferably bonded with the blocking layer.

The specific structure, shape, and the like of the components in the invention may be designed in any manner as long as an object of the invention can be achieved.

### EXAMPLES

The invention will be described in further detail with reference to Examples. The scope of the invention is by no means limited to Examples.

### Compounds

Structures of compounds represented by the formula (1) and used for producing organic EL devices in Examples 1 to 7 are given below.

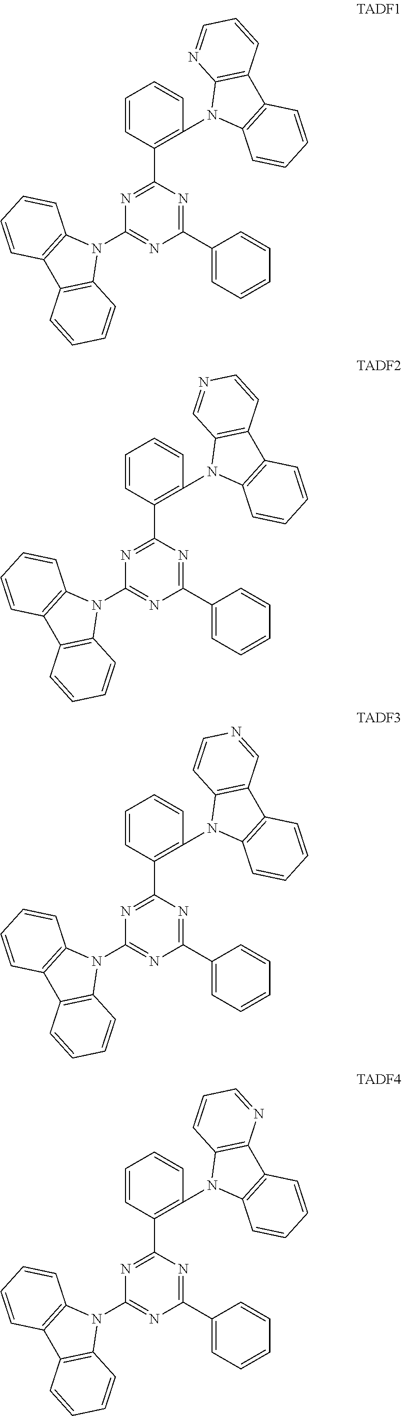 [SMILES: c1ccc(-c2nc(-c3ccccc3-n3c4ccccc4c4cccnc43)nc(-n3c4ccccc4c4ccccc43)n2)cc1 ; c1ccc(-c2nc(-c3ccccc3-n3c4ccccc4c4ccncc43)nc(-n3c4ccccc4c4ccccc43)n2)cc1 ; c1ccc(-c2nc(-c3ccccc3-n3c4ccccc4c4cnccc43)nc(-n3c4ccccc4c4ccccc43)n2)cc1 ; c1ccc(-c2nc(-c3ccccc3-n3c4ccccc4c4ncccc43)nc(-n3c4ccccc4c4ccccc43)n2)cc1]

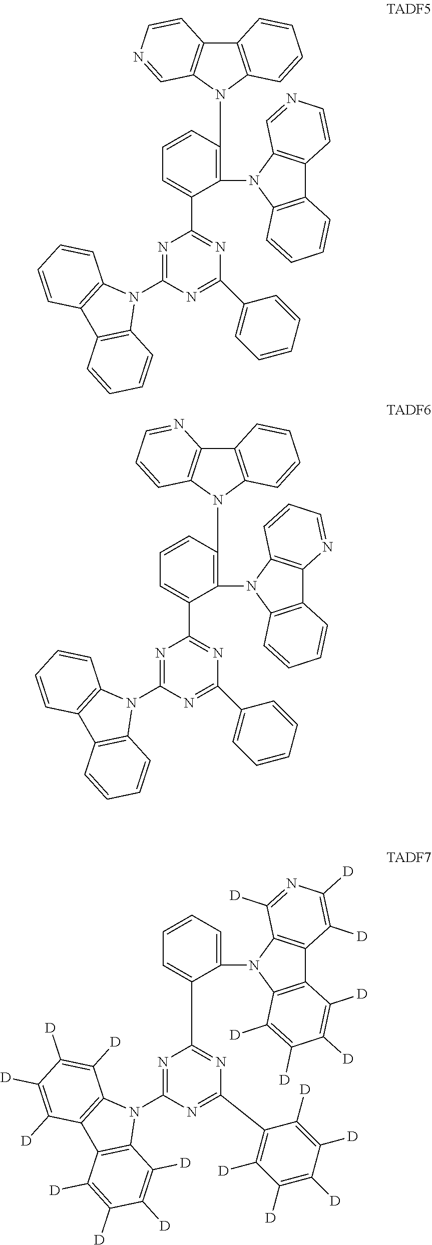

A structure of a comparative compound used for producing an organic EL device in Comparative 1 is given below.

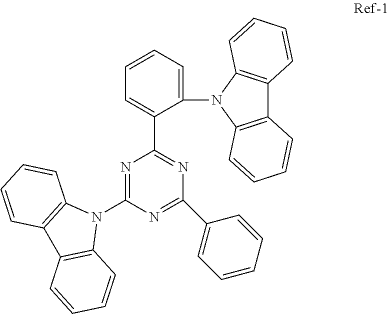 [SMILES: c1ccc(-c2nc(-c3ccccc3-n3c4ccccc4c4ccccc43)nc(-n3c4ccccc4c4ccccc43)n2)cc1]

Structures of other compounds used for producing organic EL devices in Examples 1 to 7 and Comparative 1 are given below.

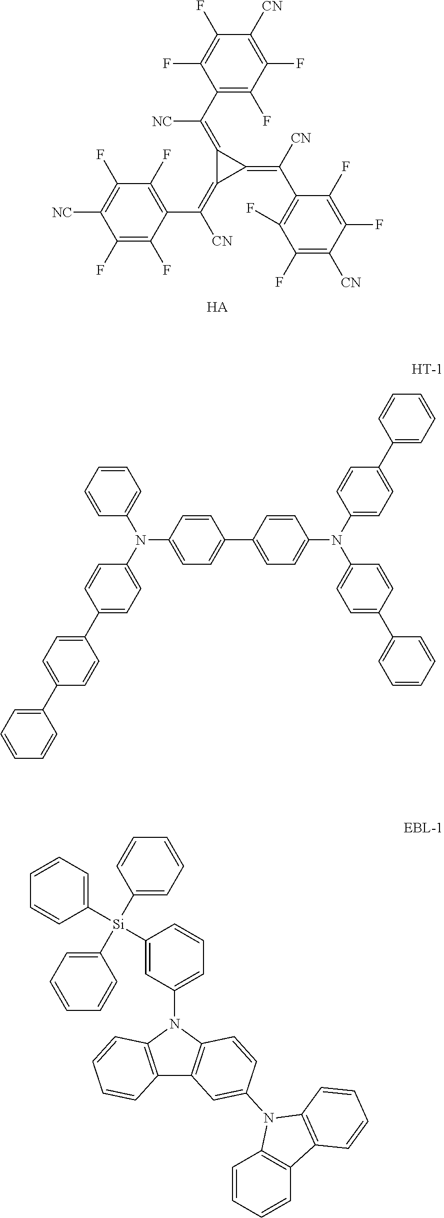 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; "HA" ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc([Si](c2ccccc2)(c2ccccc2)c2cccc(-n3c4ccccc4c4cc(-n5c6ccccc6c6ccccc65)ccc43)c2)cc1]

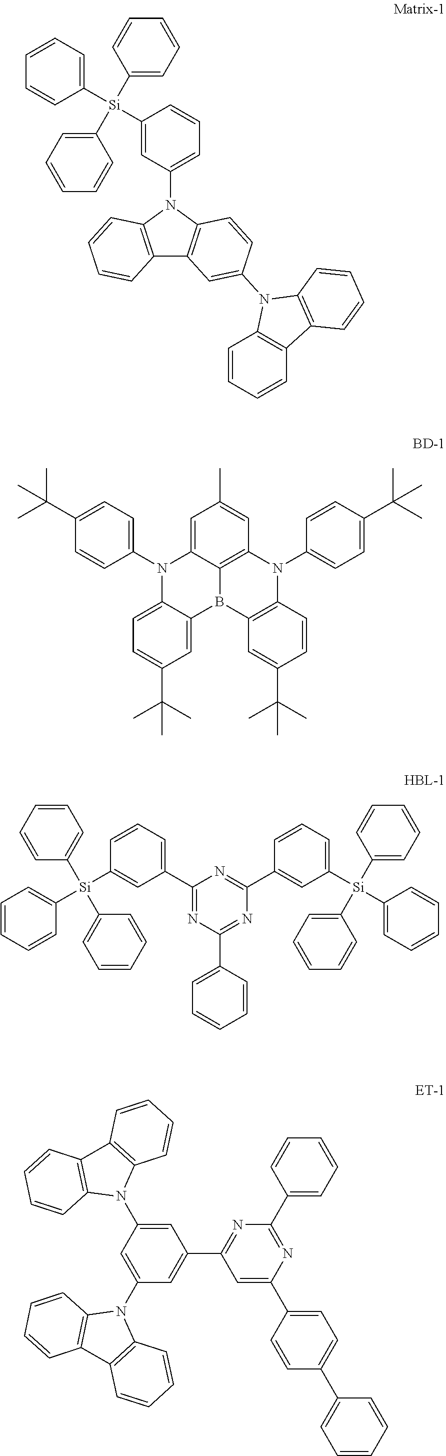 [SMILES: c1ccc([Si](c2ccccc2)(c2ccccc2)c2cccc(-n3c4ccccc4c4cc(-n5c6ccccc6c6ccccc65)ccc43)c2)cc1 ; Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1]

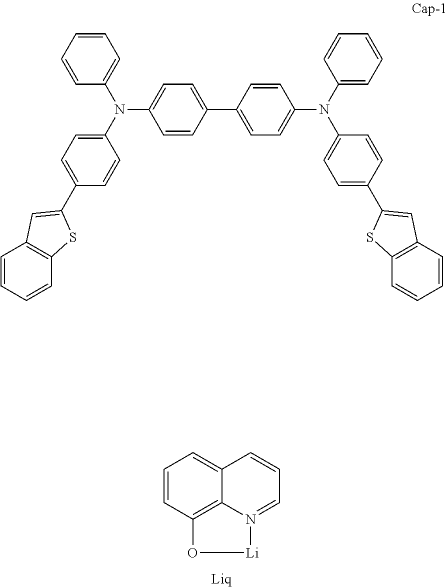

### Production (1) of Organic EL Device

The organic EL devices were produced and evaluated as follows.

### Example 1

On a glass substrate, a 100-nm-thick Ag—Pd—Cu (APC) layer, which was a silver alloy layer, and a 10-nm-thick indium oxide-zinc oxide (IZO) layer were formed in this order by sputtering. A conductive material layer made of the APC layer and the IZO layer was thus obtained. The APC layer was a light reflective layer and the IZO layer was a transparent conductive layer. IZO is a registered trademark.

Subsequently, through a normal lithography technique, the conductive material layer made of the light reflective layer and the transparent conductive layer was patterned by etching using a resist pattern as a mask to form an anode. The substrate formed with a lower electrode (anode) was ultrasonic-cleaned in isopropyl alcohol for five minutes, and then UV-ozone-cleaned for 30 minutes.

Subsequently, a compound HT-1 and a compound HA were co-deposited on the lower electrode (anode) by vacuum deposition to form a 10-nm-thick hole injecting layer. The ratios of the compound HT-1 and the compound HA in the hole injecting layer were 97 mass % and 3 mass %, respectively.

Subsequently, the compound HT-1 was vapor-deposited on the hole injecting layer to form a 115-nm-thick first hole transporting layer.

Subsequently, a compound EBL-1 was vapor-deposited on the first hole transporting layer to form a 5-nm-thick second hole transporting layer. The second hole transporting layer is occasionally referred to as an electron blocking layer.

Next, a compound Matrix-1 (compound M1) as the host material, a compound TADF1 (compound M2) as the sensitizing material, and a compound BD-1 (compound M3) as the fluorescent material were co-deposited on the second hole transporting layer to form a 31-nm-thick emitting layer. The ratios of the compound Matrix-1, the compound TADF1, and the compound BD-1 in the emitting layer were 73.8 mass %, 25 mass %, and 1.2 mass %, respectively.

Subsequently, a compound HBL-1 was vapor-deposited on the emitting layer to form a 5-nm-thick first electron transporting layer. The first electron transporting layer is occasionally referred to as a hole blocking layer.

A compound ET-1 and a compound Liq were co-deposited on the first electron transporting layer to form a 31-nm-thick second electron transporting layer. The ratios of the compound ET-1 and the compound Liq in the second electron transporting layer were 50 mass % and 50 mass %, respectively. Liq is an abbreviation of (8-quinolinolato)lithium ((8-Quinolinolato)lithium).

Ytterbium (Yb) was vapor-deposited on the second electron transporting layer to form a 1-nm-thick electron injecting layer.

Mg and Ag were vapor-deposited on the electron injecting layer into a film at a mixing ratio (mass % ratio) of 10:90, thereby forming a 13-nm-thick upper electrode (cathode) that is made of a semi-transparent MgAg alloy.

A compound Cap-1 was vacuum-deposited on the upper electrode (cathode) to form a 65-nm-thick capping layer.

A top emission type organic EL device of Example 1 was produced as described above.

A device arrangement of the organic EL device in Example 1 is roughly shown as follows.

APC(100)/IZO(10)/HT-1:HA(10,97%:3%)/HT-1(115)/EBL-1(5)/Matrix-1:TADF1:BD-1(31,73.8:25%:1.2%)/HBL-1(5)/ET-1:Liq(31,50%:50%)/Yb(1)/Mg:Ag(13,10%:90%)/Cap-1 (65)

In the above device arrangement, numerals in parentheses each represent a film thickness (nm).

In the device arrangement of Example 1, the numerals (97%:3%) represented by percentage in the same parentheses indicate a ratio (mass %) between the compound HT-1 and the compound HA in the hole injecting layer, the numerals (73.8%:25%:1.2%) represented by percentage in the same parentheses indicate a ratio (mass %) between the compound M1 (compound Matrix-1), the compound M2 (compound TADF1), and the compound M3 (compound BD-1) in the emitting layer, the numerals (50%:50%) represented by percentage in the same parentheses indicate a ratio (mass %) between the compound ET-1 and Liq in the second electron transporting layer, and the numerals (10%:90%) represented by percentage in the same parentheses indicate a mixing ratio (mass % ratio) between Mg and Ag in the upper electrode (cathode).

### Examples 2 to 7

The organic EL devices in Examples 2 to 7 were produced as in Example 1 except that the compound TADF1 as the sensitizing material used for forming the emitting layer in Example 1 was replaced with compounds shown in Table 1.

### Comparative 1

The organic EL device in Comparative 1 was produced as in Example 1 except that the compound TADF1 as the sensitizing material used for forming the emitting layer in Example 1 was replaced with a compound shown in Table 1.

### Evaluation on Organic EL Devices

The produced organic EL devices were evaluated as follows. Table 1 shows the evaluation results.

### L/J/CIEy

Voltage was applied to the organic EL device produced such that a current density was 10 mA/cm², where spectral radiance spectrum was measured by a spectroradiometer CS-1000 (produced by Konica Minolta, Inc.). Chromaticity CIEx, CIEy, and a current efficiency L/J (unit: cd/A) were calculated from the obtained spectral radiance spectrum.

A value of “L/J/CIEy” was calculated by dividing a value of the current efficiency L/J by a value of CIEy. The value of “L/J/CIEy” is occasionally referred to as Blue Index (BI). In this evaluation, a value of Blue Index (BI) was used as an index of luminous efficiency.

“BI (relative value)” (unit: %) shown in Table 1 was calculated based on the value of BI in each Example (Examples 1 to 7 and Comparative 1) according to a numerical formula (Numerical Formula X₁) below.

BI(relative value)═(BI of each Example/BI of Comparative 1)×100 (Numerical Formula X₁)

TABLE 1

| | Emitting layer | | | |
|---|---|---|---|---|
| | Host material | Sensitizing material | Fluorescent material | Device evaluation |
| | (Compound M1) | (Compound M2) | (Compound M3) | BI |
| | Name | Name | Name | (Relative value) |
| Ex. 1 | Matrix-1 | TADF1 | BD-1 | 113% |
| Ex. 2 | Matrix-1 | TADF2 | BD-1 | 118% |
| Ex. 3 | Matrix-1 | TADF3 | BD-1 | 115% |
| Ex. 4 | Matrix-1 | TADF4 | BD-1 | 120% |
| Ex. 5 | Matrix-1 | TADF5 | BD-1 | 121% |
| Ex. 6 | Matrix-1 | TADF6 | BD-1 | 124% |
| Ex. 7 | Matrix-1 | TADF7 | BD-1 | 119% |
| Comp. 1 | Matrix-1 | Ref-1 | BD-1 | 100% |

In Examples 1 to 7, compounds TADF1 to TADF7 used as the sensitizing material each have a structure in which an azine ring having a donor group (a carbazolyl group) and a donor group having a nitrogen-containing six-membered cyclic structure (an azacarbazolyl group) are bonded to a benzene ring. The compound Ref-1 used as the sensitizing material in Comparative 1 is a compound in which the azacarbazolyl group in each of the compounds TADF1 to TADF4 is substituted with the carbazolyl group. In addition, the compound Ref-1 used as the sensitizing material in Comparative 1 is a compound in which the azacarbazolyl group in the compound TADF7 is substituted with the carbazolyl group and a deuterium atom is replaced with a protium atom.

The organic EL devices in Examples 1 to 7 emitted light with higher efficiency than the organic EL device in Comparative 1.

### Evaluation on Compounds

The following evaluation was conducted on the compounds.

### Lowest Singlet Energy S₁

A toluene solution of a measurement target compound at a concentration of 10 μmol/L was prepared and put in a quartz cell. An absorption spectrum (ordinate axis: absorption intensity, abscissa axis: wavelength) of the thus-obtained sample was measured at a normal temperature (300 K). A tangent was drawn to the fall of the absorption spectrum close to the long-wavelength region, and a wavelength value λ_{edge}[nm] at an intersection of the tangent and the abscissa axis was assigned to a conversion equation (F2) below to calculate a lowest singlet energy.

$${S}_{1} [ eV ] = 1239.85 / λ edge \qquad Conversion Equation ( F2 )$$

A spectrophotometer (U3310 produced by Hitachi, Ltd.) was used for measuring absorption spectrum.

The tangent to the fall of the absorption spectrum close to the long-wavelength region is drawn as follows. While moving on a curve of the absorption spectrum from the local maximum value closest to the long-wavelength region, among the local maximum values of the absorption spectrum, in a long-wavelength direction, a tangent at each point on the curve is checked. An inclination of the tangent is decreased and increased in a repeated manner as the curve falls (i.e., a value of the ordinate axis is decreased). A tangent drawn at a point where the inclination of the curve is the local minimum closest to the long-wavelength region (except when absorbance is 0.1 or less) is defined as the tangent to the fall of the absorption spectrum close to the long-wavelength region.

The local maximum absorbance of 0.2 or less is not counted as the above-mentioned local maximum absorbance closest to the long-wavelength region.

### Energy Gap T_{77K} at 77 K

A measurement target compound was dissolved in EPA (diethylether:isopentane:ethanol=5:5:2 in volume ratio) at a concentration of 10 μmol/L, and the obtained solution was put in a quartz cell to provide a measurement sample. A phosphorescent spectrum (ordinate axis:phosphorescent luminous intensity, abscissa axis:wavelength) of the measurement sample was measured at a low temperature (77 K). A tangent was drawn to the rise of the phosphorescent spectrum close to the short-wavelength region. An energy amount was calculated by a conversion equation (F1) below based on a wavelength value λ_{edge}[nm] at an intersection of the tangent and the abscissa axis and was defined as an energy gap T_{77K} at 77 K.

$${T}_{77 K} [ eV ] = 1239.85 / {λ}_{edge} \qquad Conversion Equation ( F1 )$$

The tangent to the rise of the phosphorescence spectrum close to the short-wavelength region is drawn as follows. While moving on a curve of the phosphorescence spectrum from the short-wavelength region to the local maximum value closest to the short-wavelength region among the local maximum values of the phosphorescence spectrum, a tangent is checked at each point on the curve toward the long-wavelength of the phosphorescence spectrum. An inclination of the tangent is increased along the rise of the curve (i.e., a value of the ordinate axis is increased). A tangent drawn at a point of the local maximum inclination (i.e., a tangent at an inflection point) is defined as the tangent to the rise of the phosphorescence spectrum close to the short-wavelength region.

A local maximum point where a peak intensity is 15% or less of the maximum peak intensity of the spectrum is not counted as the above-mentioned local maximum peak intensity closest to the short-wavelength region. The tangent drawn at a point that is closest to the local maximum peak intensity closest to the short-wavelength region and where the inclination of the curve is the local maximum is defined as a tangent to the rise of the phosphorescence spectrum close to the short-wavelength region.

For phosphorescence measurement, a spectrophotofluorometer body F-4500 produced by Hitachi High-Technologies Corporation was used.

### ΔST

ΔST=S1−T_{77K} was calculated based on the measured values of the lowest singlet energy S₁ and the energy gap T_{77K}.

It was confirmed that ΔST in each of the compounds TADF1 to TADF7 and the comparative compound Ref-1 was less than 0.3 eV.

### Delayed Fluorescence of Compound

Delayed fluorescence was confirmed by measuring transient PL using an apparatus illustrated in FIG. 2. The compound TADF1 was dissolved in toluene to prepare a dilute solution with an absorbance of 0.05 or less at the excitation wavelength to eliminate the contribution of self-absorption. In order to prevent quenching due to oxygen, the sample solution was frozen and degassed and then sealed in a cell with a lid under an argon atmosphere to obtain an oxygen-free sample solution saturated with argon.

The fluorescence spectrum of the sample solution was measured with a spectrofluorometer FP-8600 (produced by JASCO Corporation), and the fluorescence spectrum of a 9,10-diphenylanthracene ethanol solution was measured under the same conditions. Using the fluorescence area intensities of both spectra, the total fluorescence quantum yield was calculated by Equation (1) in Morris et al. J. Phys. Chem. 80 (1976) 969.

Prompt emission was observed immediately when the excited state was achieved by exciting the compound TADF1 with a pulse beam (i.e., a beam emitted from a pulse laser) having a wavelength to be absorbed by the compound TADF1, and Delay emission was observed not immediately when the excited state was achieved but after the excited state was achieved. The delayed fluorescence in Examples herein means that an amount of Delay emission is 5% or more with respect to an amount of Prompt emission. Specifically, provided that the amount of Prompt emission is denoted by X_{P} and the amount of Delay emission is denoted by X_{D}, the delayed fluorescence means that a value of X_{D}/X_{P} is 0.05 or more.

The amount of Prompt emission, the amount of Delay emission and the ratio between their amounts can be obtained according to the same method as described in “Nature 492, 234 to 238, 2012” (Reference Document 1). The amount of Prompt emission and the amount of Delay emission may be calculated using any other apparatus than one described in Reference Document 1 or one illustrated in FIG. 2.

Measurement on the compounds TADF2 to TADF7 and the comparative compound Ref-1 was performed similarly as the compound TADF1.

It was confirmed in the compounds TADF1 to TADF7 and the comparative compound Ref-1 that the amount of Delay emission was 5% or more with respect to the amount of Prompt emission. Specifically, the value of X_{D}/X_{P} was 0.05 or more in the compounds TADF1 to TADF7 and the comparative compound Ref-1.

### Maximum Fluorescence Peak Wavelength λ_{FL} and Full Width at Half Maximum (FWHM) of Emission Spectrum

A measurement target compound was dissolved in toluene to prepare a solution of 5.0×10⁻⁶ mol/L. The obtained solution was put into a quartz cell (optical path length: 1.0 cm). The maximum fluorescence peak wavelength λ_{FL} (unit: nm) and the full width at half maximum (FWHM) of emission spectrum (unit: nm) when the solution was excited at 400 nm were measured using a fluorescence spectrum measurement apparatus “fluorospectrophotometer FP-8300” (manufactured by JASCO Corporation).

The maximum peak wavelength λ_{FL} of the compound BD-1 was 455 nm, and the full width at half maximum (FWHM) of emission spectrum was 23 nm.

### Synthesis Examples

### 1. Synthesis of Compound TADF1

### (1) Synthesis of Compound (1-2)

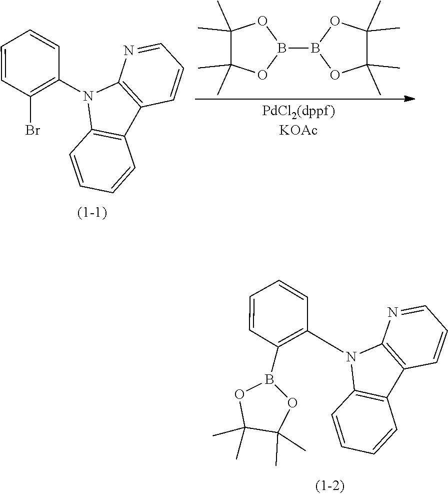

Under an argon atmosphere, 5.00 g (15.5 mmol) of a compound (1-1), 4.32 g (17.0 mmol) of bis(pinacolato)diboron, 0.227 g (0.310 mmol) of [1,1′-bis(diphenylphosphino)ferrocene]dichloropalladium (II), 2.28 g (23.3 mmol) of potassium acetate, and 50 mL of 1,4-dioxane were put into a flask, and the obtained solution was heated with stirring at 90 degrees C. for 18 hours. After the completion of reaction, water and ethyl acetate were added to the reactant to extract an organic layer. A crude product from which a solvent was distilled off was purified by column chromatography. Accordingly, 3.44 g of a white solid (a yield of 60%) was obtained. The obtained solid was a target compound (1-2). As a result of mass spectroscopy analysis, the obtained solid had m/e=370 while a molecular weight was 370.

### (2) Synthesis of Compound TADF1

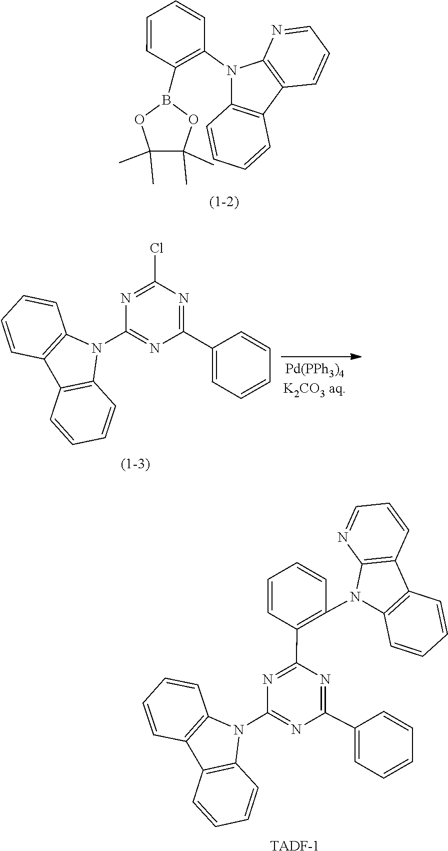

Under an argon atmosphere, 2.41 g (6.51 mmol) of the compound (1-2), 2.11 g (5.92 mmol) of a compound (1-3), 0.205 g (0.178 mmol) of tetrakistriphenylphosphine palladium, 2M sodium carbonate aqueous solution (10 mL), and 60 mL of 1,4-dioxane were put into a flask, and the obtained solution was heated with stirring at 90 degrees C. for 15 hours. After the completion of reaction, the reactant was filtrated and washed with water and methanol to obtain a crude product. The obtained crude product was purified by column chromatography to obtain 2.71 g of a white solid (a yield of 81%). The obtained solid was the target compound TADF1. As a result of mass spectroscopy analysis, the obtained solid had m/e=564 while a molecular weight was 564.

### 2. Synthesis of Compound TADF2

### (1) Synthesis of Compound (2-2)

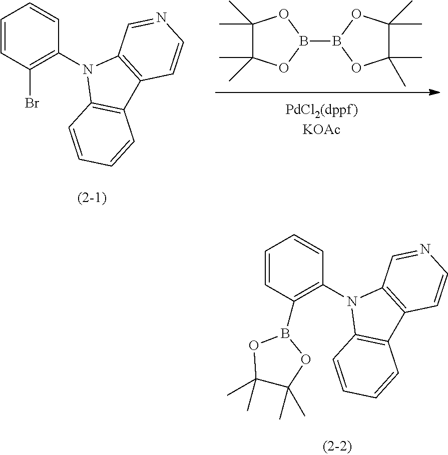

Under an argon atmosphere, 4.50 g (13.9 mmol) of a compound (2-1), 3.89 g (15.3 mmol) of bis(pinacolato)diboron, 0.204 g (0.278 mmol) of [1,1′-bis(diphenylphosphino)ferrocene]dichloropalladium (II), 2.05 g (20.9 mmol) of potassium acetate, and 50 mL of 1,4-dioxane were put into a flask, and the obtained solution was heated with stirring at 90 degrees C. for 20 hours. After the completion of reaction, water and ethyl acetate were added to the reactant to extract an organic layer. A crude product from which a solvent was distilled off was purified by column chromatography. Accordingly, 2.99 g of a white solid (a yield of 58%) was obtained. The obtained solid was a target compound (2-2). As a result of mass spectroscopy analysis, the obtained solid had m/e=370 while a molecular weight was 370.

### (2) Synthesis of Compound TADF2

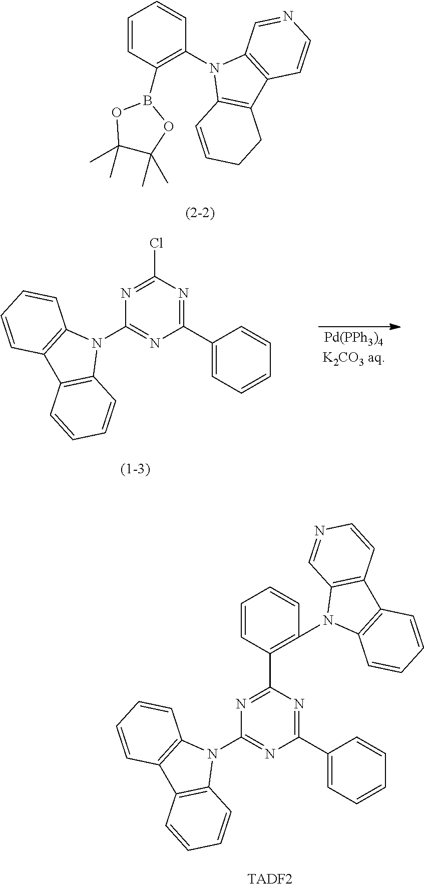

Under an argon atmosphere, 2.69 g (7.27 mmol) of the compound (2-2), 2.36 g (6.60 mmol) of the compound (1-3), 0.229 g (0.198 mmol) of tetrakistriphenylphosphine palladium, 2M sodium carbonate aqueous solution (11 mL), and 66 mL of 1,4-dioxane were put into a flask, and the obtained solution was heated with stirring at 90 degrees C. for 17 hours. After the completion of reaction, the reactant was filtrated and washed with water and methanol to obtain a crude product. The obtained crude product was purified by column chromatography to obtain 2.65 g of a white solid (a yield of 71%). The obtained solid was the target compound TADF2. As a result of mass spectroscopy analysis, the obtained solid had m/e=564 while a molecular weight was 564.

### 3. Synthesis of Compound TADF3

### (1) Synthesis of Compound (3-2)

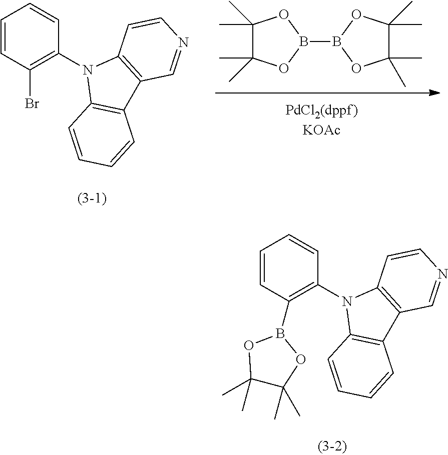

Under an argon atmosphere, 4.65 g (14.4 mmol) of a compound (3-1), 4.02 g (15.8 mmol) of bis(pinacolato)diboron, 0.210 g (0.288 mmol) of [1,1′-bis(diphenylphosphino)ferrocene]dichloropalladium (II), 2.12 g (21.6 mmol) of potassium acetate, and 50 mL of 1,4-dioxane were put into a flask, and the obtained solution was heated with stirring at 90 degrees C. for 18 hours. After the completion of reaction, water and ethyl acetate were added to the reactant to extract an organic layer. A crude product from which a solvent was distilled off was purified by column chromatography. Accordingly, 3.46 g of a white solid (a yield of 65%) was obtained. The obtained solid was a target compound (3-2). As a result of mass spectroscopy analysis, the obtained solid had m/e=370 while a molecular weight was 370.

### (2) Synthesis of Compound TADF3

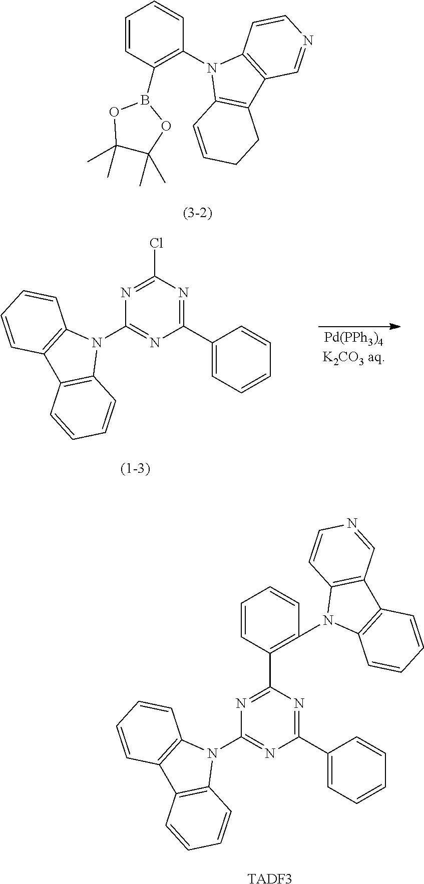

Under an argon atmosphere, 3.33 g (8.99 mmol) of the compound (3-2), 2.92 g (8.18 mmol) of the compound (1-3), 0.283 g (0.245 mmol) of tetrakistriphenylphosphine palladium, 2M sodium carbonate aqueous solution (13 mL), and 80 mL of 1,4-dioxane were put into a flask, and the obtained solution was heated with stirring at 90 degrees C. for 18 hours. After the completion of reaction, the reactant was filtrated and washed with water and methanol to obtain a crude product. The obtained crude product was purified by column chromatography to obtain 3.32 g of a white solid (a yield of 72%). The obtained solid was the target compound TADF3. As a result of mass spectroscopy analysis, the obtained solid had m/e=564 while a molecular weight was 564.

### 4. Synthesis of Compound TADF4

### (1) Synthesis of Compound (4-2)

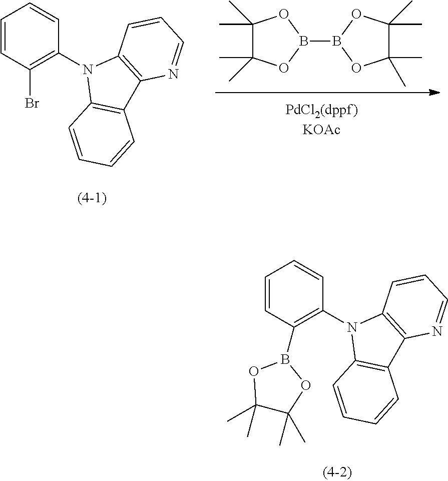

Under an argon atmosphere, 5.20 g (16.1 mmol) of a compound (4-1), 4.49 g (17.7 mmol) of bis(pinacolato)diboron, 0.235 g (0.322 mmol) of [1,1′-bis(diphenylphosphino)ferrocene]dichloropalladium (II), 2.37 g (24.1 mmol) of potassium acetate, and 55 mL of 1,4-dioxane were put into a flask, and the obtained solution was heated with stirring at 90 degrees C. for 20 hours. After the completion of reaction, water and ethyl acetate were added to the reactant to extract an organic layer. A crude product from which a solvent was distilled off was purified by column chromatography. Accordingly, 3.93 g of a white solid (a yield of 66%) was obtained. The obtained solid was a target compound (4-2). As a result of mass spectroscopy analysis, the obtained solid had m/e=370 while a molecular weight was 370.

### (2) Synthesis of Compound TADF4

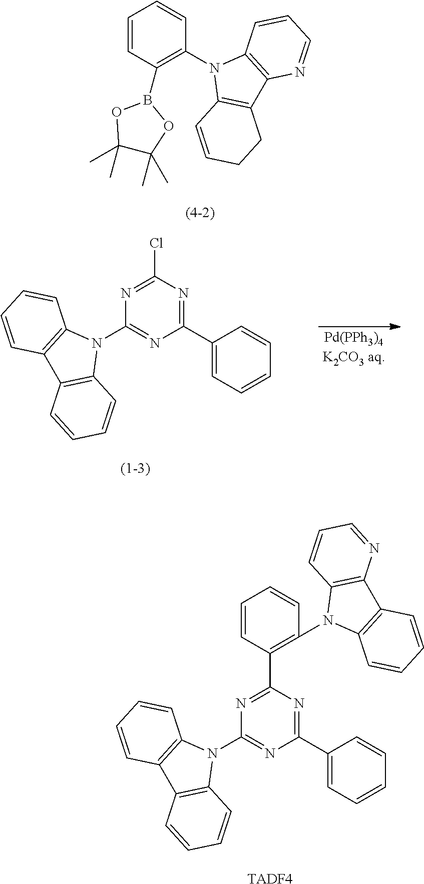

Under an argon atmosphere, 2.97 g (8.02 mmol) of the compound (4-2), 2.60 g (7.29 mmol) of the compound (1-3), 0.253 g (0.219 mmol) of tetrakistriphenylphosphine palladium, 2M sodium carbonate aqueous solution (12 mL), and 73 mL of 1,4-dioxane were put into a flask, and the obtained solution was heated with stirring at 90 degrees C. for 18 hours. After the completion of reaction, the reactant was filtrated and washed with water and methanol to obtain a crude product. The obtained crude product was purified by column chromatography to obtain 3.25 g of a white solid (a yield of 79%). The obtained solid was the target compound TADF4. As a result of mass spectroscopy analysis, the obtained solid had m/e=564 while a molecular weight was 564.

### 5. Synthesis of Compound TADF5

### (1) Synthesis of Compound (5-2)

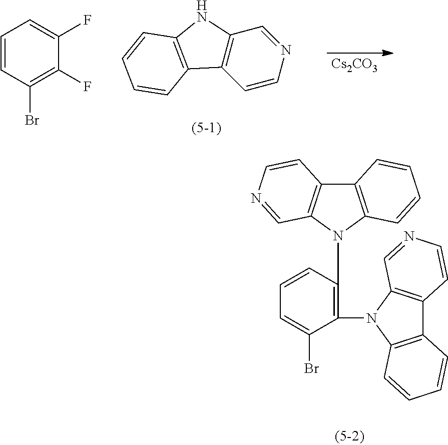

Under an argon atmosphere, 5.35 g (27.7 mmol) of 1-bromo-2,3-difluorobenzene, 10.3 g (B1.0 mmol) of a compound (5-1), 22.6 g (B9.3 mmol) of cesium carbonate, and 200 mL of N,N-dimethylformamide were put into a flask, and the obtained solution was heated with stirring at 150 degrees C. for 10 hours. After the completion of reaction, water and ethyl acetate were added to the reactant to extract an organic layer. A crude product from which a solvent was distilled off was purified by column chromatography. Accordingly, 8.55 g of a white solid (a yield of 63%) was obtained. The obtained solid was a target compound (5-2). As a result of mass spectroscopy analysis, the obtained solid had m/e=489 while a molecular weight was 489.

### (2) Synthesis of Compound (5-3)

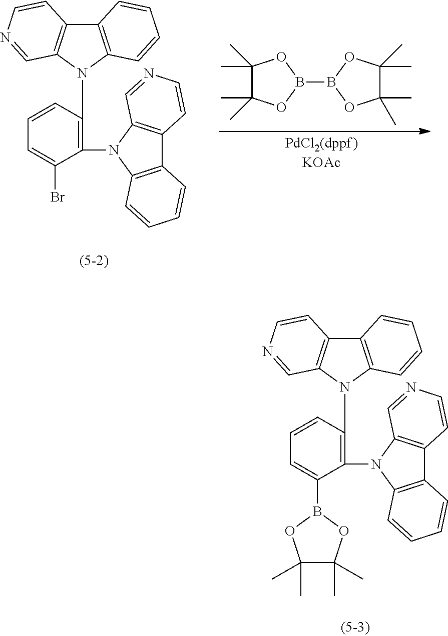

Under an argon atmosphere, 4.60 g (9.40 mmol) of the compound (5-2), 2.63 g (10.3 mmol) of bis(pinacolato)diboron, 0.137 g (0.188 mmol) of [1,1′-bis(diphenylphosphino)ferrocene]dichloropalladium (II), 1.38 g (14.1 mmol) of potassium acetate, and 30 mL of 1,4-dioxane were put into a flask, and the obtained solution was heated with stirring at 90 degrees C. for 24 hours. After the completion of reaction, water and ethyl acetate were added to the reactant to extract an organic layer. A crude product from which a solvent was distilled off was purified by column chromatography. Accordingly, 3.08 g of a white solid (a yield of 61%) was obtained. The obtained solid was a target compound (5-3). As a result of mass spectroscopy analysis, the obtained solid had m/e=536 while a molecular weight was 536.

### (3) Synthesis of Compound TADF5

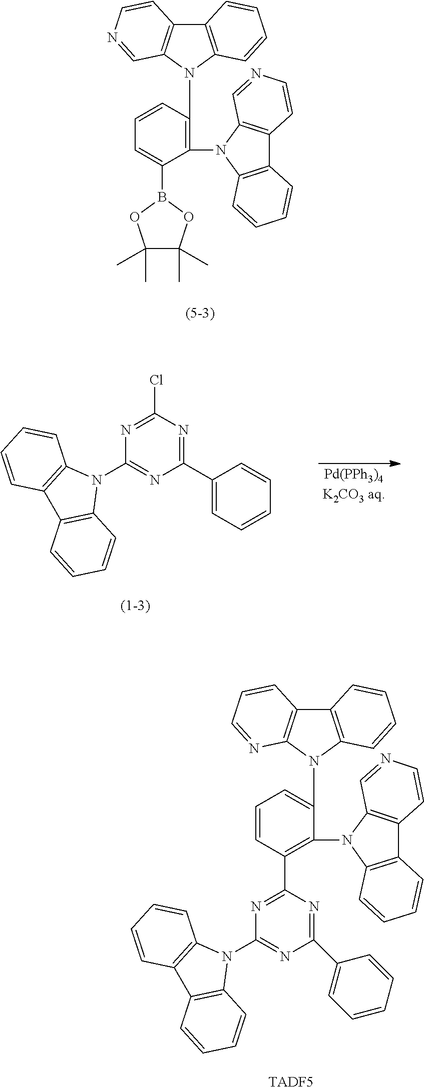

Under an argon atmosphere, 2.61 g (4.87 mmol) of the compound (5-3), 1.58 g (4.42 mmol) of the compound (1-3), 0.153 g (0.133 mmol) of tetrakistriphenyiphosphine palladium, 2M sodium carbonate aqueous solution (7 mL), and 44 mL of 1,4-dioxane were put into a flask, and the obtained solution was heated with stirring at 90 degrees C. for 18 hours. After the completion of reaction, the reactant was filtrated and washed with water and methanol to obtain a crude product. The obtained crude product was purified by column chromatography to obtain 1.91 g of a white solid (a yield of 59%). The obtained solid was the target compound TADF5. As a result of mass spectroscopy analysis, the obtained solid had m/e=730 while a molecular weight was 730.

### 6. Synthesis of Compound TADF6

### (1) Synthesis of Compound (B1-2)

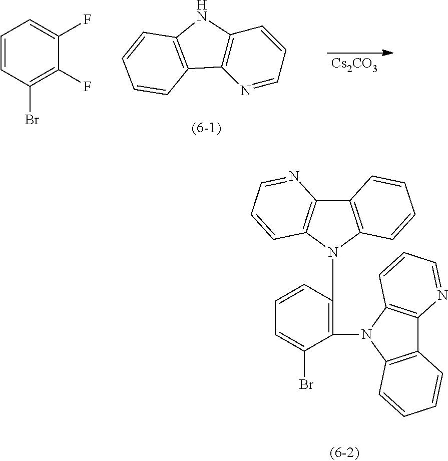

Under an argon atmosphere, 4.97 g (25.8 mmol) of 1-bromo-2,3-difluorobenzene, 9.53 g (56.7 mmol) of a compound (B1-1), 21.0 g (B4.4 mmol) of cesium carbonate, and 200 mL of N,N-dimethylformamide were put into a flask, and the obtained solution was heated with stirring at 150 degrees C. for 12 hours. After the completion of reaction, water and ethyl acetate were added to the reactant to extract an organic layer. A crude product from which a solvent was distilled off was purified by column chromatography. Accordingly, 7.31 g of a white solid (a yield of 58%) was obtained. The obtained solid was a target compound (6-2). As a result of mass spectroscopy analysis, the obtained solid had m/e=489 while a molecular weight was 489.

### (2) Synthesis of Compound (B1-3)

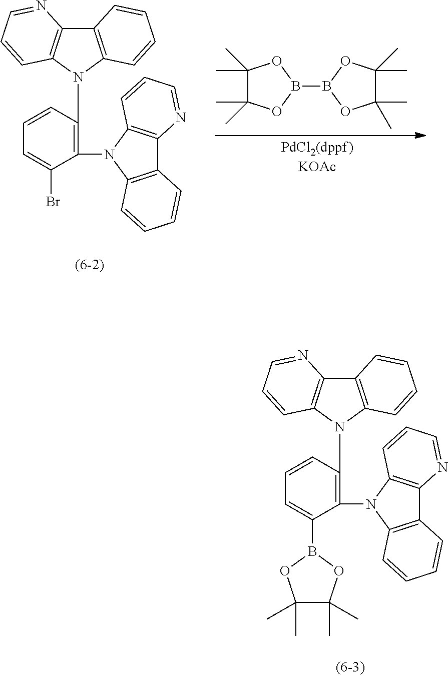

Under an argon atmosphere, 3.93 g (8.03 mmol) of the compound (B1-2), 2.24 g (8.83 mmol) of bis(pinacolato)diboron, 0.117 g (0.161 mmol) of [1,1′-bis(diphenylphosphino)ferrocene]dichloropalladium (II), 1.18 g (12.1 mmol) of potassium acetate, and 25 mL of 1,4-dioxane were put into a flask, and the obtained solution was heated with stirring at 90 degrees C. for 21 hours. After the completion of reaction, water and ethyl acetate were added to the reactant to extract an organic layer. A crude product from which a solvent was distilled off was purified by column chromatography. Accordingly, 2.46 g of a white solid (a yield of 57%) was obtained. The obtained solid was a target compound (6-3). As a result of mass spectroscopy analysis, the obtained solid had m/e=819 while a molecular weight was 819.

### (3) Synthesis of Compound TADF6

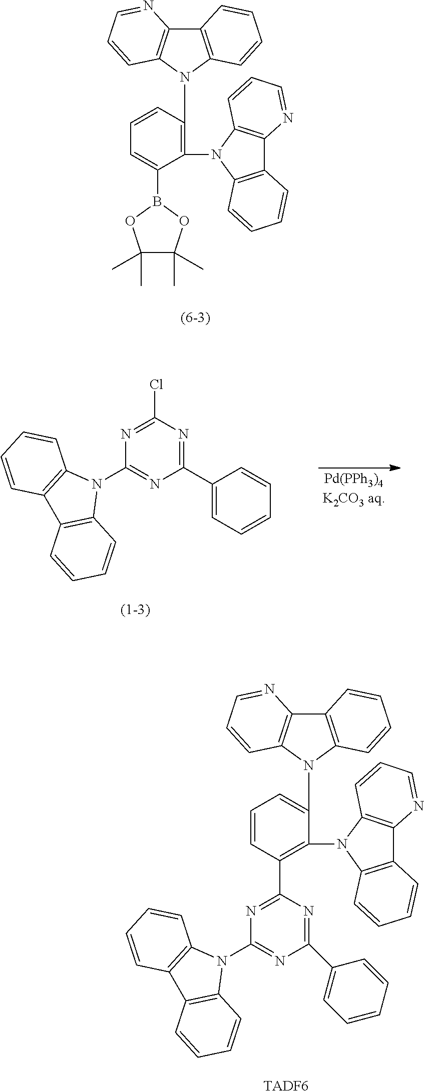

Under an argon atmosphere, 2.12 g (3.95 mmol) of the compound (B1-3), 1.28 g (3.59 mmol) of the compound (1-3), 0.125 g (0.108 mmol) of tetrakistriphenylphosphine palladium, 2M sodium carbonate aqueous solution (6 mL), and 36 mL of 1,4-dioxane were put into a flask, and the obtained solution was heated with stirring at 90 degrees C. for 19 hours. After the completion of reaction, the reactant was filtrated and washed with water and methanol to obtain a crude product. The obtained crude product was purified by column chromatography to obtain 1.73 g of a white solid (a yield of 66%). The obtained solid was the target compound TADF6. As a result of mass spectroscopy analysis, the obtained solid had m/e=730 while a molecular weight was 730.

### 7. Synthesis of Compound TADF7

### (1) Synthesis of Compound (7-2)

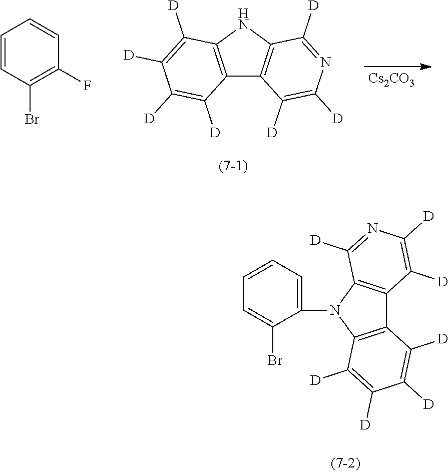

Under an argon atmosphere, 6.02 g (34.6 mmol) of 1-bromo-2-fluorobenzene, 13.33 g (76.1 mmol) of a compound (7-1), 28.2 g (86.5 mmol) of cesium carbonate, and 240 mL of N,N-dimethylformamide were put into a flask, and the obtained solution was heated with stirring at 150 degrees C. for 12 hours. After the completion of reaction, water and ethyl acetate were added to the reactant to extract an organic layer. A crude product from which a solvent was distilled off was purified by column chromatography. Accordingly, 7.97 g of a white solid (a yield of 70%) was obtained. The obtained solid was a target compound (7-2). As a result of mass spectroscopy analysis, the obtained solid had m/e=329 while a molecular weight was 329.

### (2) Synthesis of Compound (7-3)

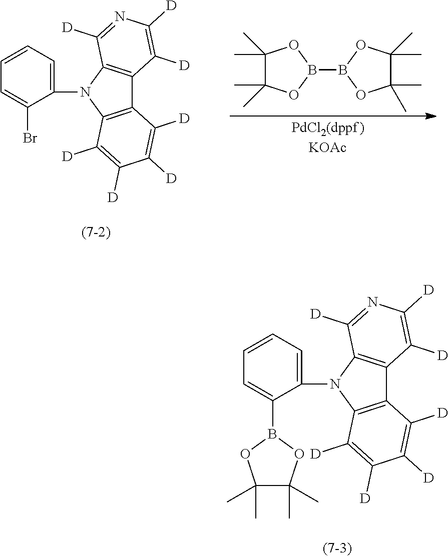

Under an argon atmosphere, 4.25 g (12.9 mmol) of the compound (7-2), 3.61 g (14.2 mmol) of bis(pinacolato)diboron, 0.189 g (0.258 mmol) of [1,1′-bis(diphenylphosphino)ferrocene]dichloropalladium (II), 1.90 g (19.4 mmol) of potassium acetate, and 40 mL of 1,4-dioxane were put into a flask, and the obtained solution was heated with stirring at 90 degrees C. for 21 hours. After the completion of reaction, water and ethyl acetate were added to the reactant to extract an organic layer. A crude product from which a solvent was distilled off was purified by column chromatography. Accordingly, 2.83 g of a white solid (a yield of 58%) was obtained. The obtained solid was a target compound (7-3). As a result of mass spectroscopy analysis, the obtained solid had m/e=377 while a molecular weight was 377.

### (3) Synthesis of Compound TADF7

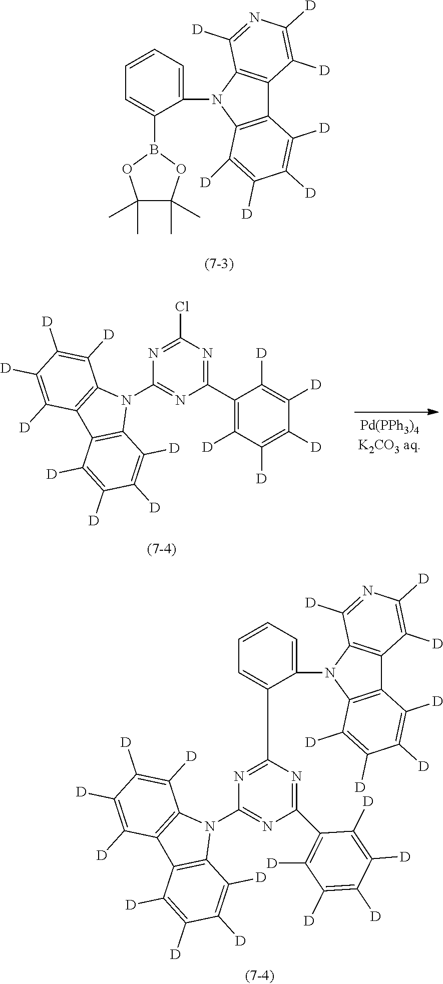 [SMILES: [2H]c1nc([2H])c2c(c1[2H])c1c([2H])c([2H])c([2H])c([2H])c1n2-c1ccccc1B1OC(C)(C)C(C)(C)O1 ; [2H]c1c([2H])c([2H])c(-c2nc(Cl)nc(-n3c4c([2H])c([2H])c([2H])c([2H])c4c4c([2H])c([2H])c([2H])c([2H])c43)n2)c([2H])c1[2H] ; [2H]c1nc([2H])c2c(c1[2H])c1c([2H])c([2H])c([2H])c([2H])c1n2-c1ccccc1-c1nc(-c2c([2H])c([2H])c([2H])c([2H])c2[2H])nc(-n2c3c([2H])c([2H])c([2H])c([2H])c3c3c([2H])c([2H])c([2H])c([2H])c32)n1]

Under an argon atmosphere, 2.03 g (5.38 mmol) of the compound (7-3), 1.81 g (4.89 mmol) of a compound (7-4), 0.170 g (0.147 mmol) of tetrakistriphenylphosphine palladium, 2M sodium carbonate aqueous solution (8 mL), and 50 mL of 1,4-dioxane were put into a flask, and the obtained solution was heated with stirring at 90 degrees C. for 17 hours. After the completion of reaction, the reactant was filtrated and washed with water and methanol to obtain a crude product. The obtained crude product was purified by column chromatography to obtain 2.06 g of a white solid (a yield of 72%). The obtained solid was the target compound TADF7. As a result of mass spectroscopy analysis, the obtained solid had m/e=584 while a molecular weight was 584.

What is claimed is:

1. A compound represented by a formula (1) below, 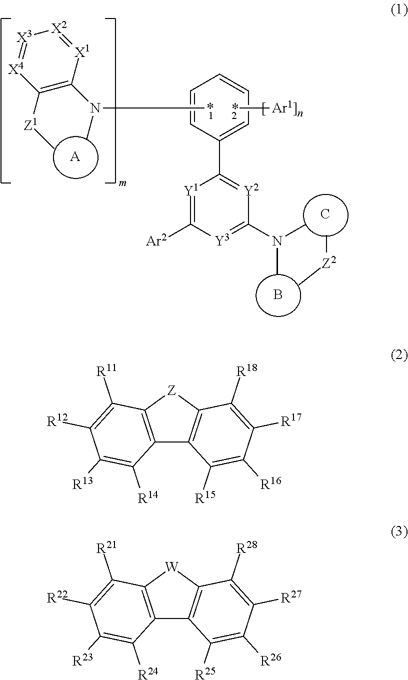
where, in the formula (1):
X¹ to X⁴ are each independently a nitrogen atom or CRx;
at least one of X¹ to X⁴ is a nitrogen atom;
when a plurality of Rx are present, the plurality of Rx are mutually the same or different;
Y¹ to Y³ are each independently a nitrogen atom or CH;
two or more of Y¹ to Y³ are each a nitrogen atom;
a ring A, a ring B, and a ring C are each independently a substituted or unsubstituted aromatic hydrocarbon ring having 6 to 60 ring carbon atoms, or a substituted or unsubstituted heterocycle having 5 to 60 ring atoms;
Z¹ and Z² are each independently a single bond, C(R₁)(R²), NR³, an oxygen atom, or a sulfur atom;
Ar¹ is a hydrogen atom, a substituted or unsubstituted aryl group having 6 to 50 ring carbon atoms, or a group represented by a formula (2) above;
Ar² is a substituted or unsubstituted aryl group having 6 to 50 ring carbon atoms, or a group represented by a formula (3) above;
m is 1, 2, 3, 4, or 5;
n is 0, 1, 2, 3, or 4;
m+n=5 is satisfied;
when m is 2, 3, 4, or 5,
a plurality of X¹ are mutually the same or different;
a plurality of X² are mutually the same or different;
a plurality of X³ are mutually the same or different;
a plurality of X⁴ are mutually the same or different;
a plurality of rings A are mutually the same or different; and
a plurality of Z¹ are mutually the same or different; and
when n is 2, 3, or 4,
a plurality of Ar¹ are mutually the same or different; and
*1 and *2 each represent a bonding position to a benzene ring,
where, in the formula (2):
Z is C(R_{1A})(R^{2A}), NR^{3A}, an oxygen atom, or a sulfur atom;
at least one combination of adjacent two or more of R₁₁ to R₁₈ are mutually bonded to form a substituted or unsubstituted monocyclic ring, mutually bonded to form a substituted or unsubstituted fused ring, or not mutually bonded; and
when Ar¹ is a group represented by the formula (2), one of R₁₁ to R₁₈ and R^{3A} is a single bond with a benzene ring in the formula (1),
where, in the formula (3):
W is C(R^{21A})(R^{22A}), an oxygen atom, or a sulfur atom;
at least one combination of adjacent two or more of R²¹ to R²⁸ are mutually bonded to form a substituted or unsubstituted monocyclic ring, mutually bonded to form a substituted or unsubstituted fused ring, or not mutually bonded;
when Ar² is a group represented by the formula (3), one of R²¹ to R²⁸ is a single bond with a carbon atom between Y¹ and Y³ in the formula (1); and
Rx, R₁, R², R³, R⁴, R^{2A}, R^{21A}, R^{22A}, and R^{3A}, R₁₁ to R₁₈ and R²¹ to R²⁸ not being the single bond with the benzene ring, not being the single bond with the carbon atom between Y¹ and Y³, and forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms, and
in the compound represented by the formula (1), R₉₀₆ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

2. The compound according to claim 1, wherein Z¹ and Z² are each a single bond.

3. The compound according to claim 1, wherein the ring A, the ring B, and the ring C are each independently a substituted or unsubstituted benzene ring, or a substituted or unsubstituted dibenzothiophene ring.

4. The compound according to claim 1, wherein one of X¹ to X³ is a nitrogen atom and X¹ to X⁴ not being the nitrogen atom are each CRx.

5. The compound according to claim 1, wherein X² or X⁴ is a nitrogen atom and X¹ to X⁴ not being the nitrogen atom are each CRx.

6. The compound according to claim 1, wherein Y² and Y³ are each a nitrogen atom and Y¹ is CH; or Y¹ to Y³ are each a nitrogen atom.

7. The compound according to claim 1, wherein Y¹ to Y³ are each a nitrogen atom.

8. The compound according to claim 1, wherein Ar¹ is a hydrogen atom, or a substituted or unsubstituted phenyl group, a substituted or unsubstituted biphenyl group, a substituted or unsubstituted fluorenyl group, a substituted or unsubstituted carbazolyl group, a substituted or unsubstituted dibenzofuranyl group, or a substituted or unsubstituted dibenzothiophenyl group.

9. The compound according to claim 1, wherein Ar² is a substituted or unsubstituted phenyl group, a substituted or unsubstituted biphenyl group, a substituted or unsubstituted fluorenyl group, a substituted or unsubstituted dibenzofuranyl group, or a substituted or unsubstituted dibenzothiophenyl group.

10. The compound according to claim 1, wherein, in the compound represented by the formula (1), a partial structure represented by a formula (1A) below is a group represented by one of formulae (11A) to (14A) below, 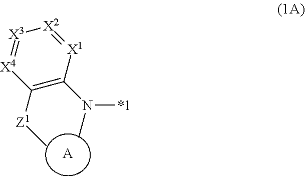
where, in the formula (1A):
a ring A, X¹ to X⁴, and Z¹ respectively represent the same as the ring A, X¹ to X⁴, and Z¹ in the formula (1);
when a plurality of partial structures represented by the formula (1A) are present, the partial structures represented by the formula (1A) are mutually the same or different; and
*1 represents the same as *1 in the formula (1), 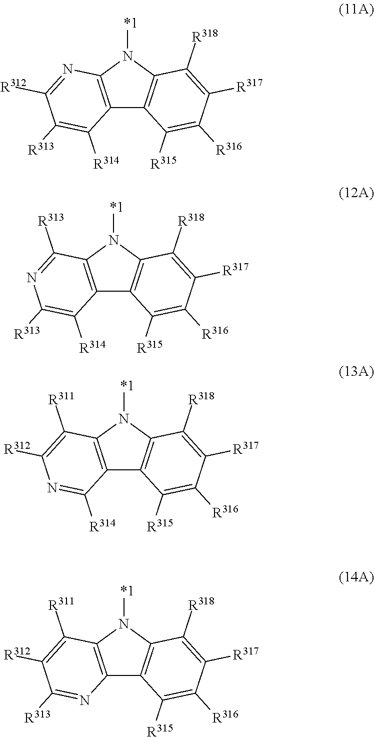
where, in the formulae (11A) to (14A):
at least one combination of adjacent two or more of R₃₁₁ to R₃₁₈ are mutually bonded to form a substituted or unsubstituted monocyclic ring, mutually bonded to form a substituted or unsubstituted fused ring, or not mutually bonded;
R₃₁₁ to R₃₁₈ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
R₉₀₁ to R₉₀₇ in the formulae (11A) to (14A) respectively represent the same as R₉₀₁ to R₉₀₇ in the formula (1); and
*1 represents the same as *1 in the formula (1A).

11. The compound according to claim 1, wherein, in the compound represented by the formula (1), a partial structure represented by a formula (1B) below is a group represented by one of formulae (11B) to (13B) below, 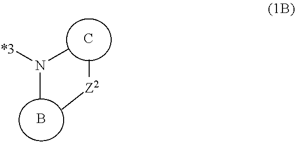
where, in the formula (1B):
a ring B, a ring C, and Z² respectively represent the same as the ring B, the ring C, and Z² in the formula (1); and
*3 represents a bonding position to a carbon atom between Y² and Y³ in the formula (1), 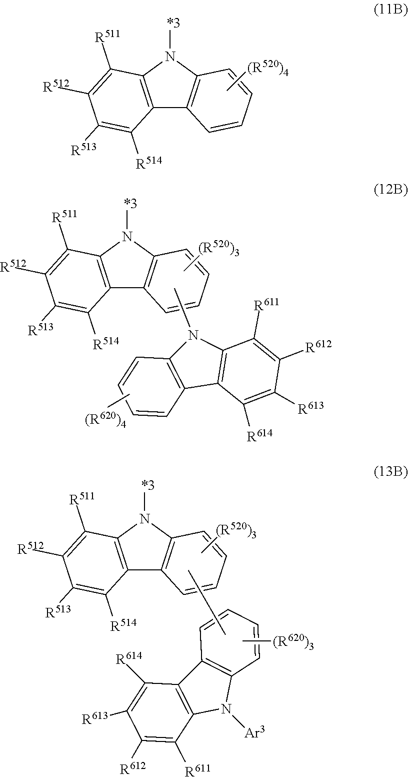
where, in the formulae (11B) to (13B):
at least one combination of adjacent two or more of R⁵¹¹ to R⁵¹⁴ and R⁵²⁰ are mutually bonded to form a substituted or unsubstituted monocyclic ring, mutually bonded to form a substituted or unsubstituted fused ring, or not mutually bonded;
at least one combination of adjacent two or more of R⁶¹¹ to R⁶¹⁴ and R⁶²⁰ are mutually bonded to form a substituted or unsubstituted monocyclic ring, mutually bonded to form a substituted or unsubstituted fused ring, or not mutually bonded;
a plurality of R⁵²⁰ are mutually the same or different;
a plurality of R⁶²⁰ are mutually the same or different;
Ar³ is a substituted or unsubstituted aryl group having 6 to 50 ring carbon atoms, or a substituted or unsubstituted heterocyclic group having 5 to 50 ring atoms;
R⁵¹¹ to R⁵¹⁴, R⁵²⁰, R⁶¹¹ to R⁶¹⁴, and R⁶²⁰ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄₎, a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
R₉₀₁ to R₉₀₇ in the formulae (11B) to (13B) respectively represent the same as R₉₀₆ to R₉₀₇ in the formula (1); and
*3 represents the same as *3 in the formula (1B).

12. The compound according to claim 1, wherein m is 1.

13. The compound according to claim 1, wherein Rx, R₁, R², R³, R^{1A}, R^{2A}, R^{3A}, R^{21A}, R^{22A}, R₁₁ to R₁₈, and R²¹ to R²⁸ are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

14. An organic-electroluminescence-device material comprising the compound according to claim 1.

15. An organic electroluminescence device, comprising:
a cathode;
an anode; and
an organic layer disposed between the cathode and the anode, wherein
the organic layer comprises, as a compound M2, the compound according to claim 1.

16. The organic electroluminescence device according to claim 15, wherein
the organic layer comprises at least one emitting layer, and
the emitting layer comprises the compound M2.

17. The organic electroluminescence device according to claim 15, wherein a difference ΔST(M2) between a lowest singlet energy S₁(M2) of the compound M2 and an energy gap T_{77K}(M2) at 77 K of the compound M2 satisfy a relationship of a numerical formula (Numerical Formula 1) below,
ΔST(M2)=S₁(M2)−T_{77K}(M2)<0.3 eV (Numerical Formula 1).

18. The organic electroluminescence device according to claim 15, wherein the emitting layer comprises the compound M2 as a sensitizing material.

19. The organic electroluminescence device according to claim 18, wherein the sensitizing material is a delayed fluorescent compound.

20. The organic electroluminescence device according to claim 16, wherein the emitting layer further comprises a fluorescent material, and the fluorescent material is a compound represented by a formula (41) below, 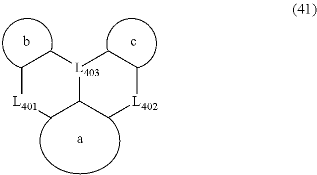
where, in the formula (41):
a ring a, a ring b and a ring c are each independently a substituted or unsubstituted aromatic hydrocarbon ring having 6 to 50 ring carbon atoms, or a substituted or unsubstituted heterocycle having 5 to 50 ring atoms;
L₄₀₁ and L₄₀₂ are each independently O, S, Se, NR₄₀, C(R₄₁)(R₄₂), or Si(R₄₃)(R₄₄);
L₄₀₃ is B, P, or P═O;
R₄₀ to R₄₄ are each independently bonded with the ring a, ring b, or ring c to form a substituted or unsubstituted monocyclic ring, bonded with the ring a, ring b, or ring c to form a substituted or unsubstituted fused ring, or bonded neither with the ring a, ring b, nor ring c;
R₄₁ and R₄₂ are mutually bonded to form a substituted or unsubstituted monocyclic ring, mutually bonded to form a substituted or unsubstituted fused ring, or not mutually bonded;
R₄₃ and R₄₄ are mutually bonded to form a substituted or unsubstituted monocyclic ring, mutually bonded to form a substituted or unsubstituted fused ring, or not mutually bonded;
R₄₀ to R₄₄ forming neither the substituted or unsubstituted monocyclic ring nor the substituted or unsubstituted fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, an iminyl group represented by —CR₄₅═N, a substituted or unsubstituted aryl group having 6 to 50 ring carbon atoms, or a substituted or unsubstituted heterocyclic group having 5 to 50 ring atoms;
R₄₅ is a substituted or unsubstituted aryl group having 6 to 60 ring carbon atoms, a substituted or unsubstituted heterocyclic group having 5 to 60 ring atoms, a substituted or unsubstituted alkyl group having 1 to 20 carbon atoms, or a substituted or unsubstituted cycloalkyl group having 3 to 20 ring carbon atoms;
when a plurality of R₄₀ are present, the plurality of R₄₀ are mutually the same or different;
when a plurality of R₄₁ are present, the plurality of R₄₁ are mutually the same or different;
when a plurality of R₄₂ are present, the plurality of R₄₂ are mutually the same or different;
when a plurality of R₄₃ are present, the plurality of R₄₃ are mutually the same or different;
when a plurality of R₄₄ are present, the plurality of R₄₄ are mutually the same or different; and
when a plurality of R₄₅ are present, the plurality of R₄₅ are mutually the same or different.

21. The organic electroluminescence device according to claim 16, wherein the emitting layer comprises the compound M2 as a host material.

22. The organic electroluminescence device according to claim 21, wherein the host material is a delayed fluorescent compound.

23. The organic electroluminescence device according to claim 16, wherein the emitting layer comprises no metal complex.

24. The organic electroluminescence device according to claim 16, wherein the emitting layer comprises no phosphorescent material.

25. The organic electroluminescence device according to claim 16, further comprising a hole transporting layer between the anode and the emitting layer.

26. The organic electroluminescence device according to claim 16, further comprising an electron transporting layer between the cathode and the emitting layer.

27. An electronic device comprising the organic electroluminescence device according to claim 15.
